(19) United States
(12) Patent Application Publication
(10) Pub. No.: US 2022/0372017 A1
(43) Pub. Date: Nov. 24, 2022

(54) HCK DEGRADERS AND USES THEREOF

(71) Applicant: DANA-FARBER CANCER INSTITUTE, INC., Boston, MA (US)

(72) Inventors: Nathanael S. Gray, Stanford, CA (US); Steven P. Treon, Jamaica Plain, MA (US); Jinhua Wang, Winchester, MA (US); Guang Yang, Natick, MA (US); Sara Jean Buhrlage, Somerville, MA (US); Li Tan, Shanghai (CN)

(73) Assignee: DANA-FARBER CANCER INSTITUTE, INC., Boston, MA (US)

(21) Appl. No.: 17/621,057

(22) PCT Filed: Jun. 24, 2020
(86) PCT No.: PCT/US2020/039304

§ 371 Date: Dec. 20, 2021

Related U.S. Application Data
(60) Provisional application No. 62/865,780, filed on Jun. 24, 2019.

(51) Int. Cl. C07D 401/14; C07D 487/04

(52) U.S. Cl. CPC C07D 401/14; C07D 487/04

(57) ABSTRACT

Provided herein are bifunctional compounds with a moiety (e.g., lenalidomide, thalidomide) that is a binder of an E3 ubiquitin ligase (e.g., Cereblon) and another moiety that is a binder of a kinase (e.g., HCK, BTK) to induce degradation of the kinase (e.g., HCK, BTK). Also provided are pharmaceutical compositions comprising the bifunctional compounds, and methods of treating and/or preventing diseases (e.g., proliferative diseases (e.g., non-Hodgkin's lymphoma, Burkitt's lymphoma, Waldenstrom macroglobulinemia, MYD88-mutated Waldenstrom macroglobulinemia, activated B-cell diffuse large B-cell lymphoma, leukemia)), inflammatory disease, or other diseases associated with MYD88 mutations). Provided also are methods of inducing the degradation of a kinase (e.g., HCK, BTK) in a cell in a biological sample or subject by administering the bifunctional compound or composition described herein.

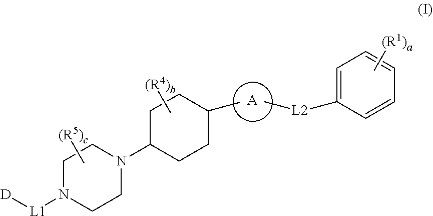

### RELATED APPLICATIONS

This application claims priority under 35 U.S.C. § 119(e) to U.S. Provisional Application, U.S. Ser. No. 62/865,780, filed Jun. 24, 2019, which is incorporated herein by reference.

### BACKGROUND OF THE INVENTION

The hematopoietic cell kinase (HCK) is a member of the SRC family of cytoplasmic tyrosine kinases (SFK's), and is expressed in cells of the myeloid and B-lymphocyte cell lineages. It is reported that HCK is a downstream target of mutated MYD88, is activated by IL-6, and triggers pro-survival signaling including PI3K/AKT, MAPK/ERK, and BTK in MYD88-mutated cells. HCK is also a target of ibrutinib and represents a novel target for therapeutic development in MYD88-mutated Waldenstrom macroglobulinemia (WM) and activated B-cell (ABC) lymphoma, diffuse large B-cell lymphoma (DLBCL), and potentially other diseases associated with MYD88 mutations. Compounds that degrade HCK protein instead of inhibiting HCK have the potential to be more potent than known inhibitors of HCK. There is a need for compounds that induce degradation of HCK and thereby treat diseases associated with HCK (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations.

### SUMMARY OF THE INVENTION

The present disclosure stems from the discovery of a strategy for inducing the degradation of hematopoietic cell kinase (HCK) (e.g., the selective degradation of HCK). The bifunctional compounds described herein include three components: a moiety for binding the kinase (e.g., HCK, BTK, FYN, SRC, BLK, LCK, CSK, ABL1, ABL2, LIMK1, LATS1), a linker, and an E3 ubiquitin ligase-binding moiety that recruits the ubiquitination machinery which ultimately induces proteasomal degradation of the target kinase (e.g., HCK, BTK, FYN, SRC, BLK, LCK, CSK, ABL1, ABL2, LIMK1, LATS1). The bifunctional compounds described herein include three components: a HCK binding moiety, a linker, and an E3 ubiquitin ligase-binding moiety that recruits the ubiquitination machinery which ultimately induces proteasomal degradation of the target kinase (e.g., HCK, thereby degrading BTK). The bifunctional compounds comprise two functional components: a HCK binding moiety, and an E3 ubiquitin ligase binding moiety is based on an imide drug (e.g., lenalidomide, thalidomide, ligand that binds to von Hippel-Lindau protein (VHL ligand), or a derivative thereof). The disclosure therefore provides new compounds, compositions, and methods for the treatment of various diseases (e.g., proliferative diseases, such as non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) associated with the target HCK based on this discovery. The invention therefore provides a new therapeutic strategy for targeting and degrading HCK and/or BTK, and treating various diseases and conditions, particularly those associated with HCK.

Described herein are bifunctional compounds of Formula (I). The compounds described herein include a component that binds to the target kinase (e.g., HCK, BTK, FYN, SRC, BLK, LCK, CSK, ABL1, ABL2, LIMK1, LATS1) and a component that binds an E3 ubiquitin ligase (e.g., lenalidomide, thalidomide) and therefore may be useful in promoting and/or inducing the degradation of a kinase (e.g., HCK, BTK, FYN, SRC, BLK, LCK, CSK, ABL1, ABL2, LIMK1, LATS1). In certain embodiments, degradation of HCK activity blocks downstream BTK activity (e.g., BTK phosphorylation). The compounds described herein include a component that binds to the target HCK and a component that binds an E3 ubiquitin ligase (e.g., lenalidomide, thalidomide) and therefore may be useful in promoting and/or inducing the degradation of HCK. The compounds may be useful in treating and/or preventing diseases and conditions, such as a proliferative disease associated with a target kinase (e.g., HCK, BTK, FYN, SRC, BLK, LCK, CSK, ABL1, ABL2, LIMK1, LATS1) (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. The compounds may be useful in treating and/or preventing diseases and conditions, such as a proliferative disease associated with HCK (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. Also provided are pharmaceutical compositions and kits including a compound described herein.

In one aspect, the present disclosure provides compounds of Formula (I):

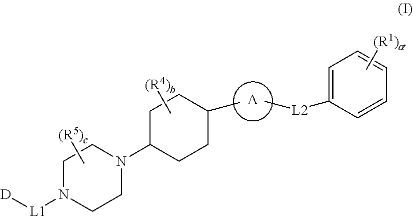

and pharmaceutically acceptable salts, solvates, hydrates, polymorphs, co-crystals, tautomers, stereoisomers, isotopically labeled derivatives, and prodrugs thereof, wherein R¹, R⁴, R⁵, a, b, c, L1, L2, D, and Ring A are as defined herein.

In Formula (I), D is a E3 ubiquitin ligase binding moiety. In certain embodiments, D is derived from an immunomodulatory imide drug. In certain embodiments, D is derived from lenalidomide. In certain embodiments, D is derived from thalidomide. In certain embodiments, D is an E3 ubiquitin ligase binding moiety, wherein D is of Formula (IA) or (IB), or a compound that binds to von Hippel-Lindau protein (a “VHL ligand”). In certain embodiments, D is derived from a VHL ligand. In certain embodiments, D binds to a E3 ubiquitin ligase or von Hippel-Lindau protein.

In certain embodiments, D is of Formula (IA):

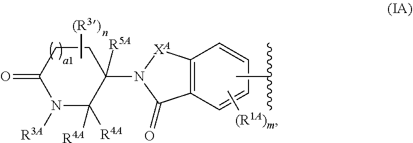

wherein R^{1A}, R^{3A}, R^{4A}, R^{5A}, R^{3′}, X^{A}, a1, m, and n are as defined herein.

In certain embodiments, D is of Formula (IB):

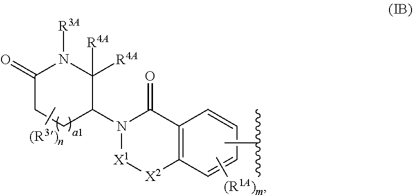

wherein R^{1A}, R^{3A}, R^{4A}, R^{3′}, X¹, X², a1, m, and n are as defined herein.

In certain embodiments, D is of formula:

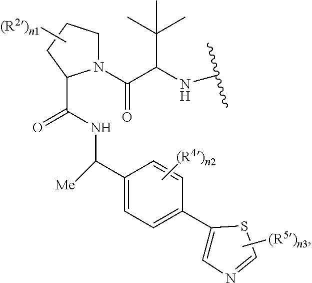

wherein R^{2′}, R^{4′}, R^{5′}, n1, n2, and n3 are as defined herein.

In certain embodiments, D is of formula:

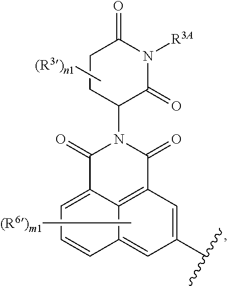

wherein R^{3A}, R^{3′}, R^{6′}, n1, and m1 are as defined herein.

Exemplary compounds of Formula (I) include, but are not limited to:

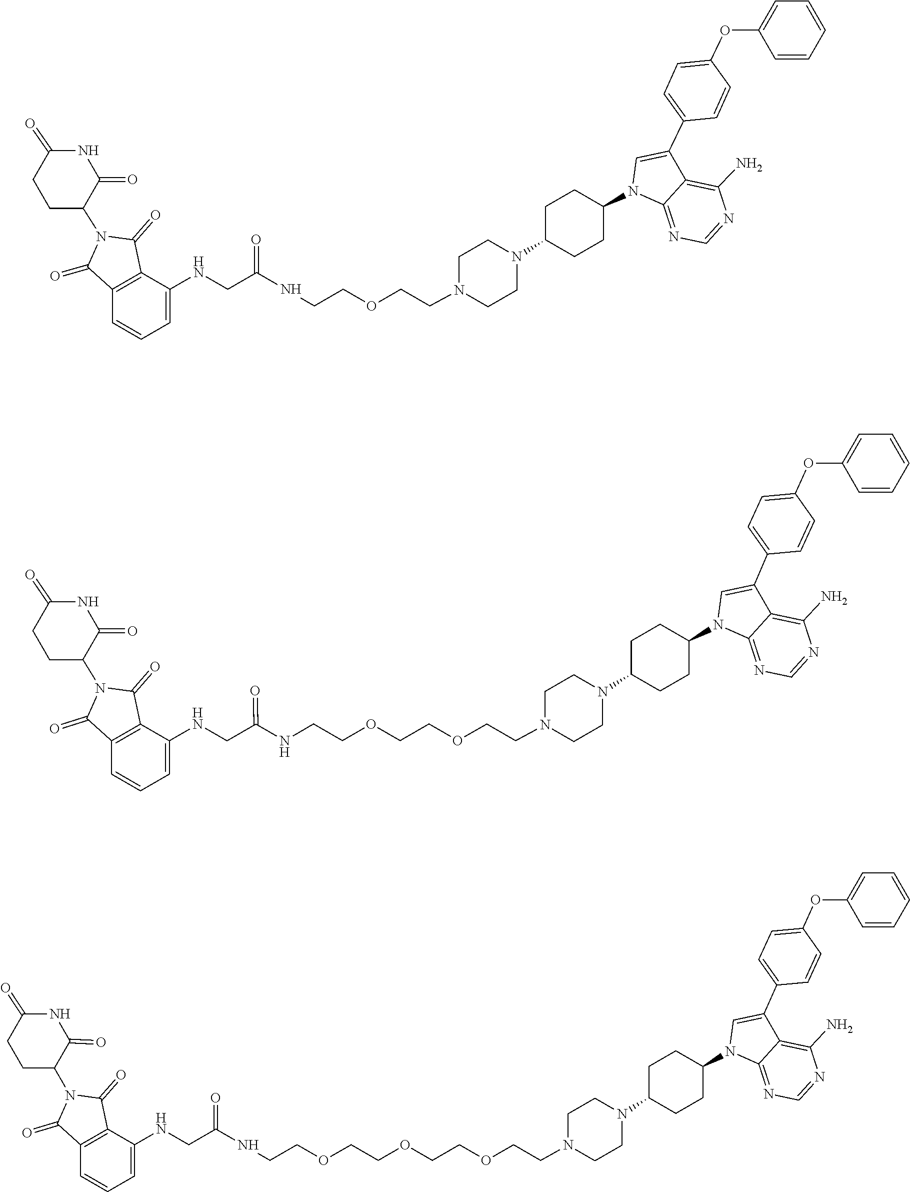 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCC(=O)NC3=O)C4=O)CC2)CC1]

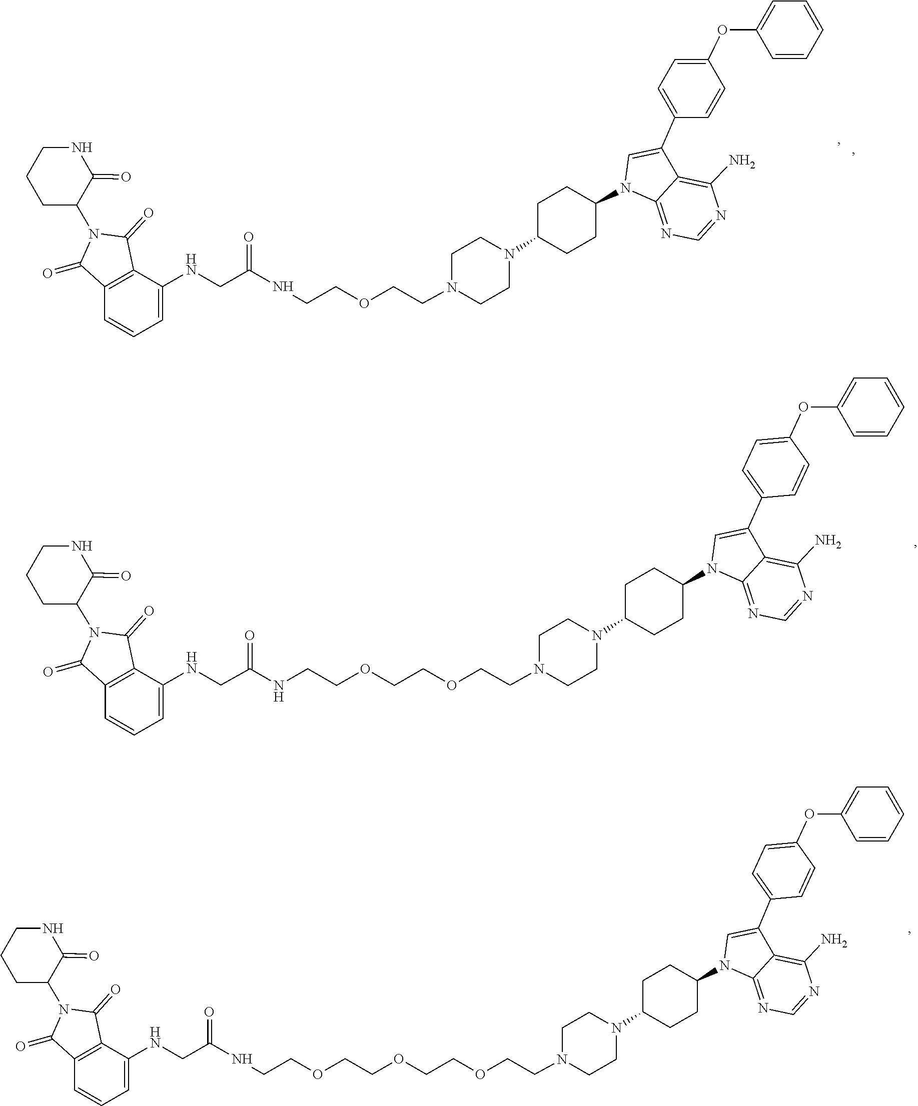 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCCNC3=O)C4=O)CC2)CC1]

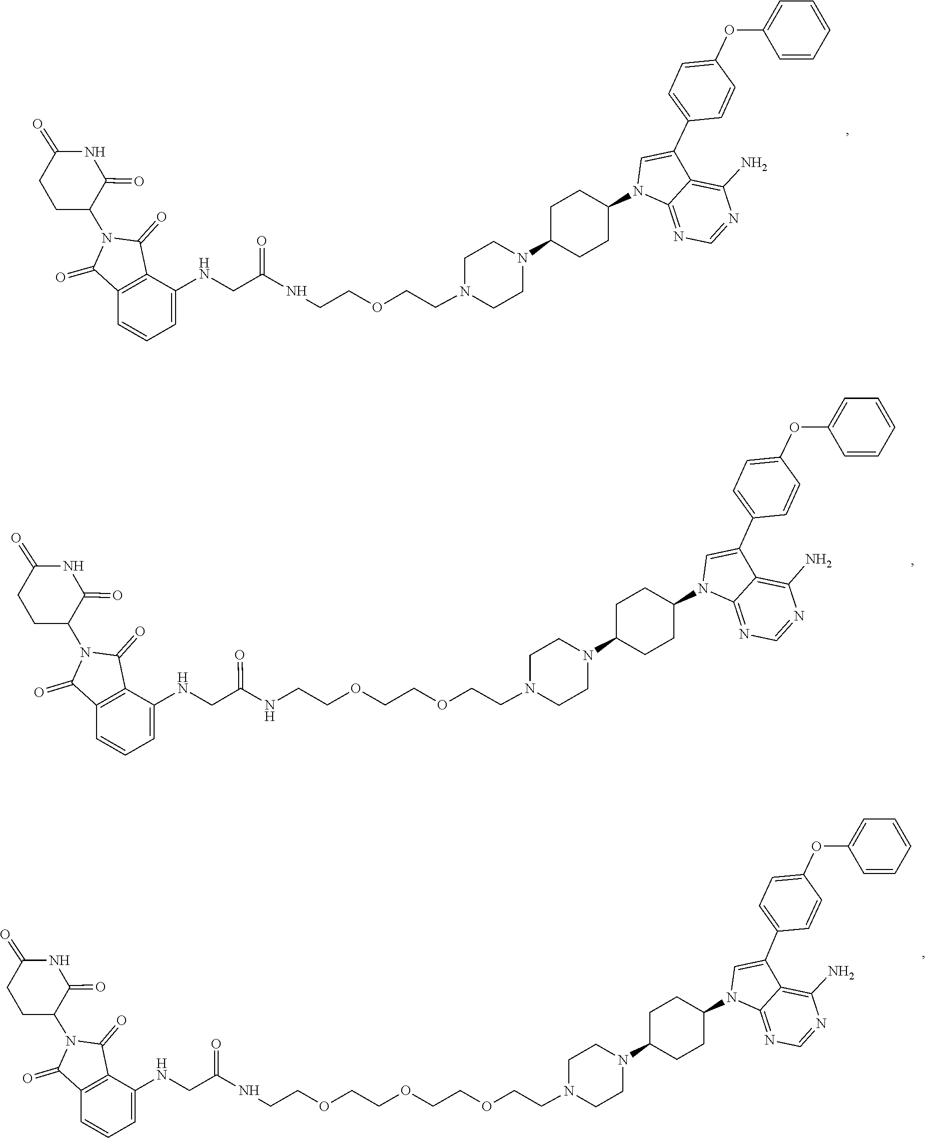 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCC(=O)NC3=O)C4=O)CC2)CC1]

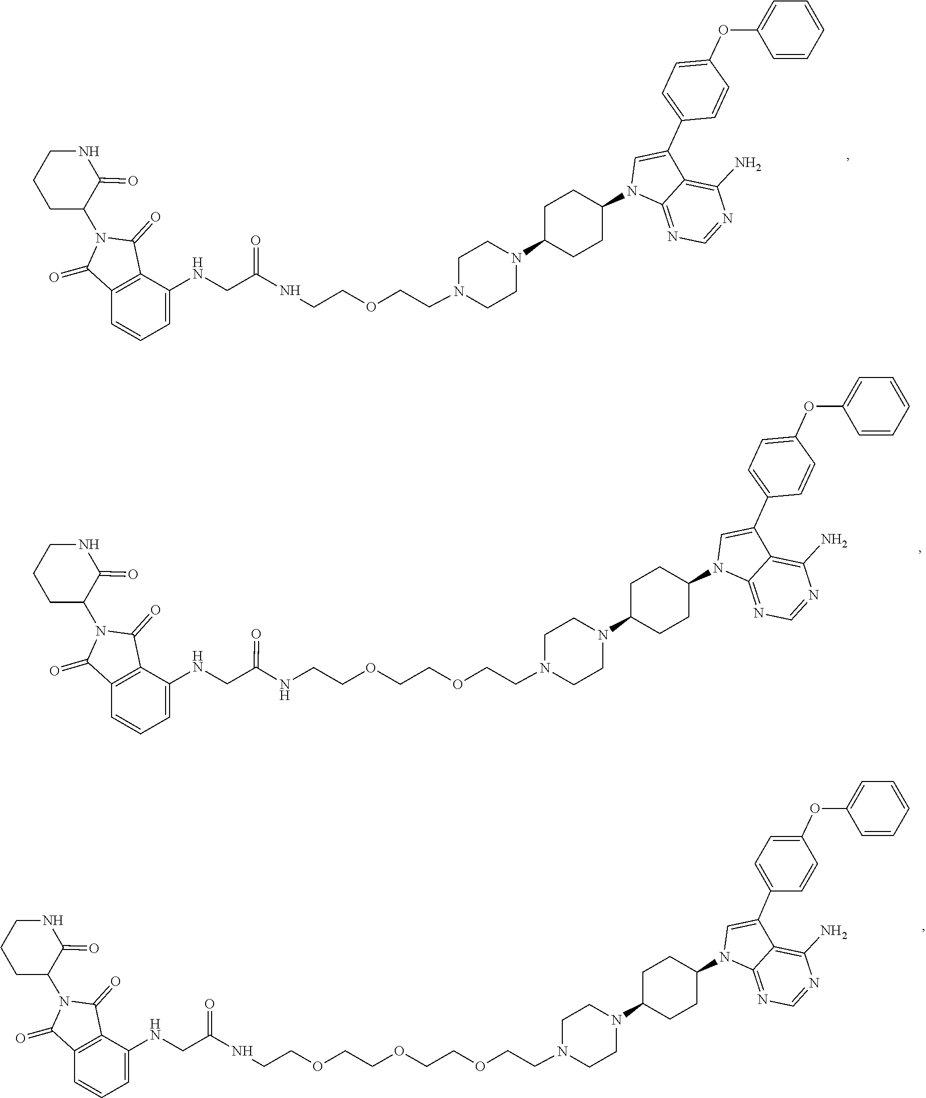 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCCNC3=O)C4=O)CC2)CC1]

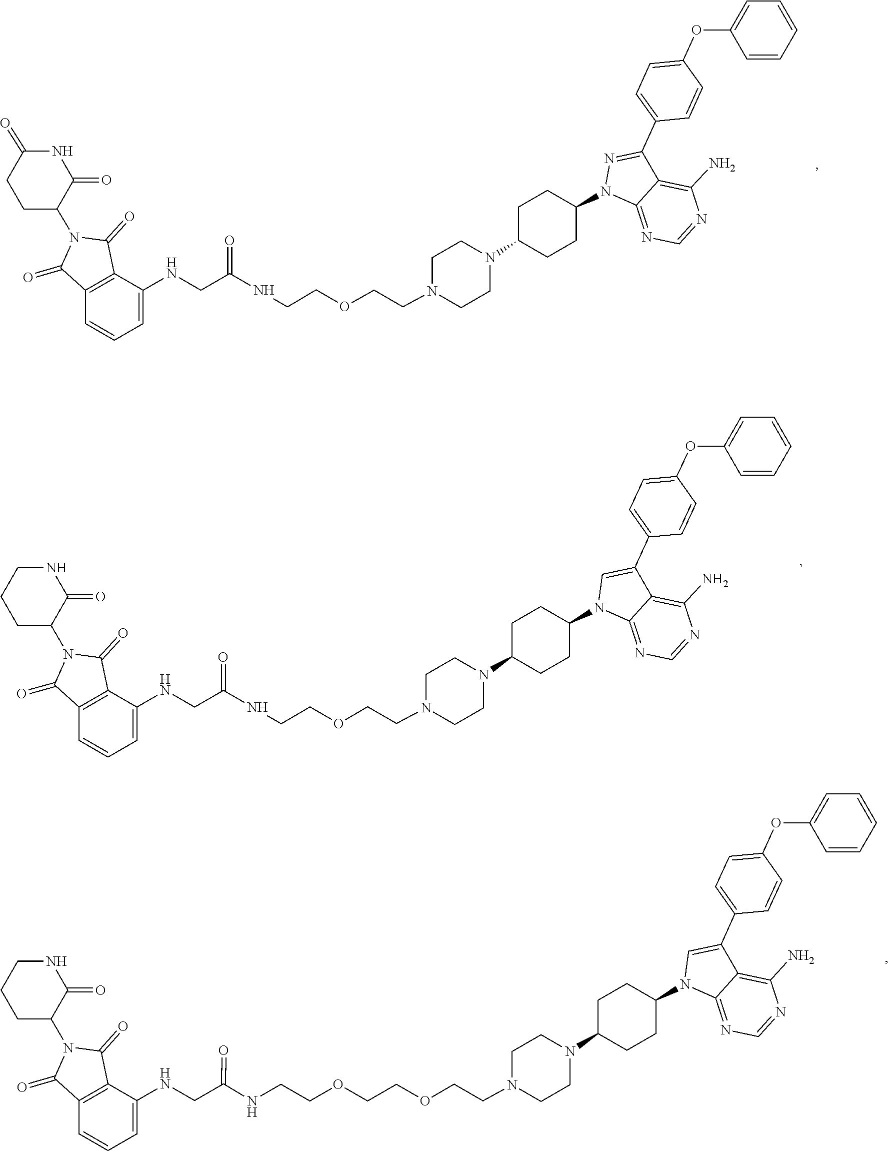 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCCNC3=O)C4=O)CC2)CC1]

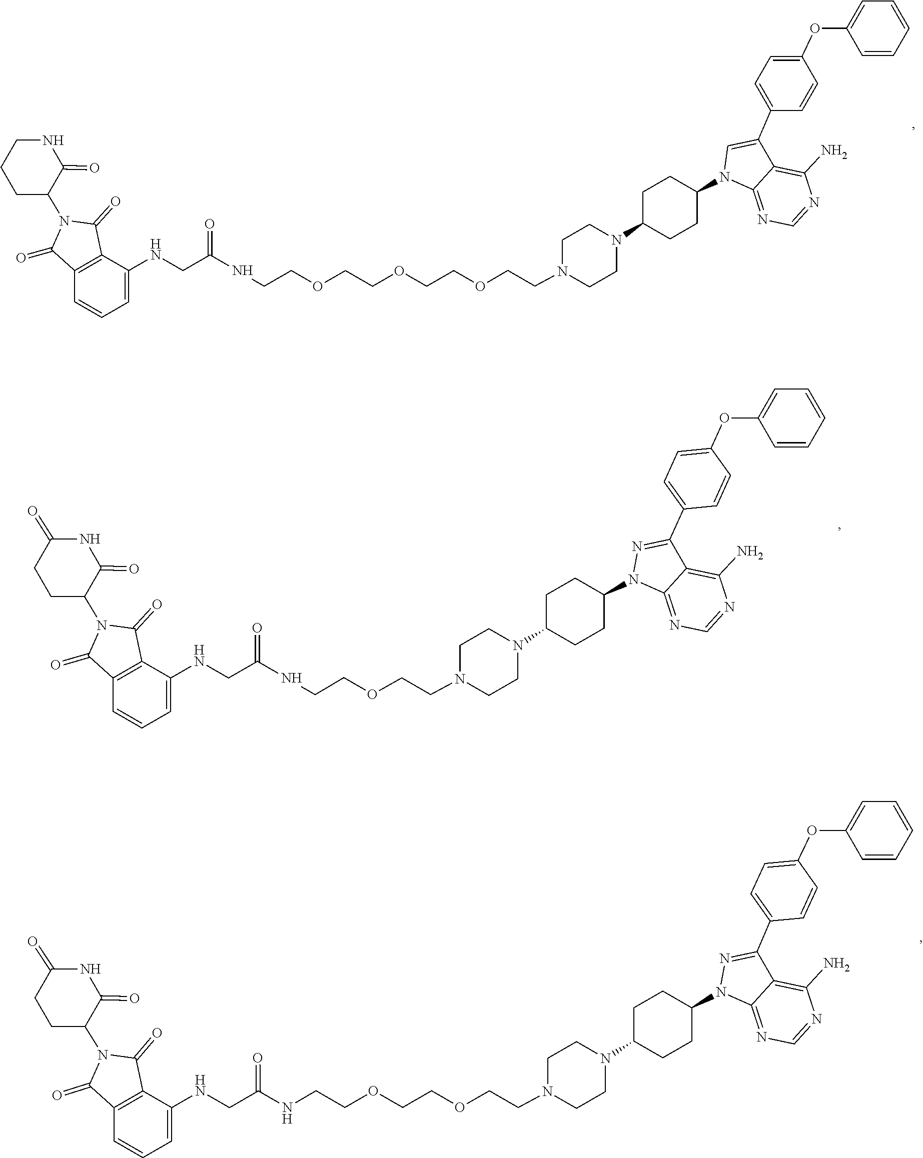 [SMILES: Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCC(=O)NC3=O)C4=O)CC2)CC1]

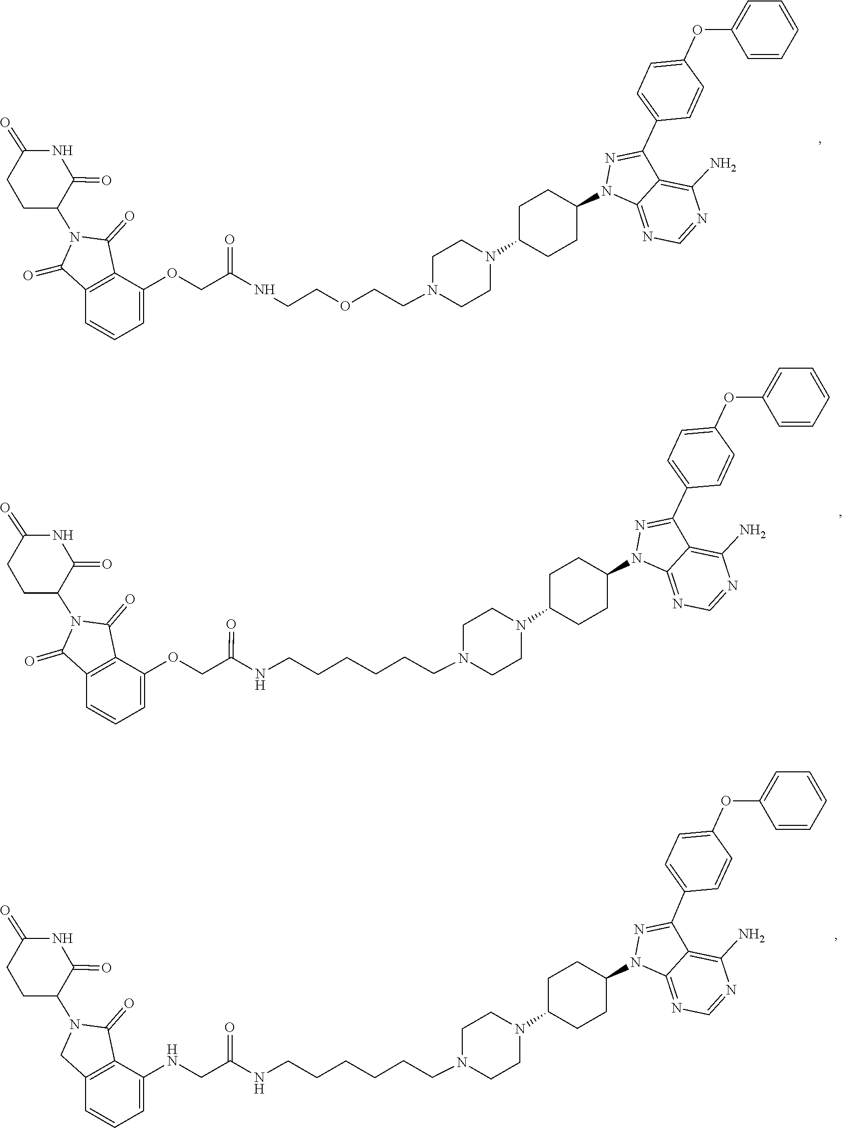 [SMILES: Nc1ncnc2c1c(-c1ccc(Oc3ccccc3)cc1)nn2[C@H]1CC[C@H](N2CCN(CCOCCNC(=O)COc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4)CC2)CC1]

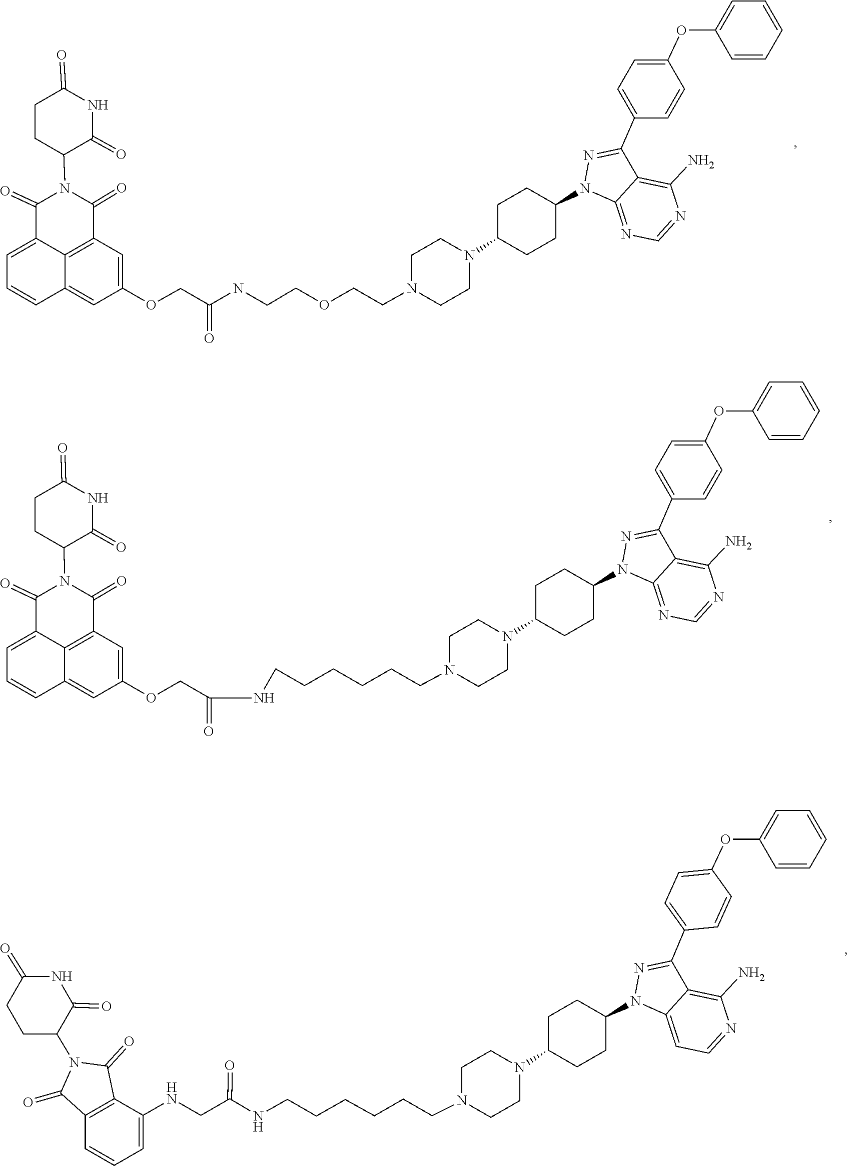

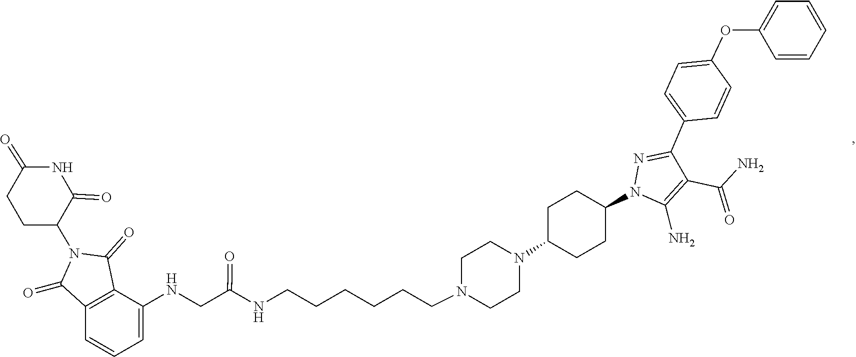 [SMILES: NC(=O)c1c(-c2ccc(Oc3ccccc3)cc2)nn([C@H]2CC[C@H](N3CCN(CCCCCCNC(=O)CNc4cccc5c4C(=O)N(C4CCC(=O)NC4=O)C5=O)CC3)CC2)c1N]

and pharmaceutically acceptable salts, solvates, hydrates, polymorphs, co-crystals, tautomers, stereoisomers, isotopically labeled derivatives, and prodrugs thereof.

Exemplary compounds of Formula (I) include, but are not limited to, compounds disclosed in Examples 1 and 2.

In still another aspect, described herein are methods of making the compounds described herein. In certain embodiments, the compounds described herein are synthesized according to the conditions described in Example 1.

In another aspect, described herein are pharmaceutical compositions including a compound described herein, and optionally a pharmaceutically acceptable excipient. In certain embodiments, a pharmaceutical composition described herein includes a therapeutically or prophylactically effective amount of a compound described herein. The pharmaceutical compositions may be useful in inducing the degradation of the kinase (e.g., HCK, BTK, FYN, SRC, BLK, LCK, CSK, ABL1, ABL2, LIMK1, LATS1) in a subject or cell, in treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof, or in preventing a disease in a subject in need thereof. In certain embodiments, the compound being administered or used induces the degradation of a kinase (e.g., HCK, BTK, FYN, SRC, BLK, LCK, CSK, ABL1, ABL2, LIMK1, LATS1) in a subject or cell, in treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma)), inflammatory disease, autoimmune disease, or other diseases associated with MYD88 mutations), disease associated with a MYD88 mutation) in a subject in need thereof, or in preventing a disease in a subject in need thereof. In certain embodiments, the compound being administered or used induces the degradation of a kinase (e.g., HCK, BTK) in a subject or cell, in treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma)), inflammatory disease, autoimmune disease, or other diseases associated with MYD88 mutations) in a subject in need thereof, or in preventing a disease in a subject in need thereof.

In still another aspect, described herein are kits including a container with a compound or pharmaceutical composition described herein. A kit described herein may include a single dose or multiple doses of the compound or pharmaceutical composition. The described kits may be useful in inducing the degradation of the kinase (e.g., HCK, BTK, FYN, SRC, BLK, LCK, CSK, ABL1, ABL2, LIMK1, LATS1). The described kits may be useful in inducing the degradation of the kinase (e.g., HCK, BTK). In certain embodiments, a kit described herein further includes instructions for using the compound or pharmaceutical composition included in the kit. A kit described herein may also include information (e.g. prescribing information) as required by a regulatory agency, such as the U.S. Food and Drug Administration (FDA).

In certain embodiments, the compound being administered or used induces the degradation of the kinase (e.g., HCK, BTK, FYN, SRC, BLK, LCK, CSK, ABL1, ABL2, LIMK1, LATS1). In certain embodiments, the compound being administered or used induces the degradation of the kinase (e.g., HCK, BTK). In certain embodiments, the compound being administered or used induces the degradation of HCK. In certain embodiments, the compound being administered or used induces the degradation of the kinase (e.g., BTK).

Another aspect of the present disclosure relates to methods of treating a disease in a subject in need thereof comprising administering to the subject a therapeutically effective amount of a compound or pharmaceutical composition described herein. In another aspect, the present disclosure provides methods of preventing a disease in a subject in need thereof comprising administering to the subject a prophylactically effective amount of a compound or pharmaceutical composition described herein.

In yet another aspect, the present disclosure provides compounds and pharmaceutical compositions described herein for use in a method of the disclosure (e.g., a method of inducing the degradation of the kinase (e.g., HCK, BTK, FYN, SRC, BLK, LCK, CSK, ABL1, ABL2, LIMK1, LATS1), a method of treating and/or preventing a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations). In another aspect, the present disclosure provides compounds and pharmaceutical compositions described herein for use in a method of the disclosure (e.g., a method of inducing the degradation of the kinase (e.g., HCK, BTK), a method of treating and/or preventing a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations).

The details of one or more embodiments of the invention are set forth herein. Other features, objects, and advantages of the invention will be apparent from the Detailed Description, Examples, Figures, and Claims.

### Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987. The disclosure is not intended to be limited in any manner by the exemplary listing of substituents described herein.

Compounds described herein can comprise one or more asymmetric centers, and thus can exist in various isomeric forms, e.g., enantiomers and/or diastereomers. For example, the compounds described herein can be in the form of an individual enantiomer, diastereomer, or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, N Y, 1962); and Wilen, Tables of Resolving Agents and Optical Resolutions p. 268 (E. L. Eliel, Ed., Univ. of Notre Dame Press, Notre Dame, Ind. 1972). The invention additionally encompasses compounds described herein as individual isomers substantially free of other isomers, and alternatively, as mixtures of various isomers.

When a range of values is listed, it is intended to encompass each value and sub-range within the range. For example, “C₁₋₆” is intended to encompass C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆.

“Hydrocarbon chain” refers to a substituted or unsubstituted divalent alkyl, alkenyl, or alkynyl group. A hydrocarbon chain includes at least one chain, each node (“carbon unit”) of which including at least one carbon atom, between the two radicals of the hydrocarbon chain. For example, hydrocarbon chain —C^{A}H(C^{B}H₂C^{C}H₃)— includes only one carbon unit C^{A}. The term “Cₓ hydrocarbon chain,” wherein x is a positive integer, refers to a hydrocarbon chain that includes x number of carbon unit(s) between the two radicals of the hydrocarbon chain. If there is more than one possible value of x, the smallest possible value of x is used for the definition of the hydrocarbon chain. For example, —CH(C₂H₅)— is a C₁ hydrocarbon chain, and

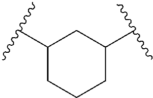 [SMILES: *C1CCCC(*)C1]

is a C₃ hydrocarbon chain. When a range of values is used, e.g., a C₁₋₆ hydrocarbon chain, the meaning of the range is as described herein. A hydrocarbon chain may be saturated (e.g., —(CH₂)₄—). A hydrocarbon chain may also be unsaturated and include one or more C═C and/or C≡C bonds anywhere in the hydrocarbon chain. For instance, —CH═CH—(CH₂)₂—, —CH₂—C≡C—CH₂—, and —C≡C—CH═CH— are all examples of a unsubstituted and unsaturated hydrocarbon chain. In certain embodiments, the hydrocarbon chain is unsubstituted (e.g., —(CH₂)₄—). In certain embodiments, the hydrocarbon chain is substituted (e.g., —CH(C₂H₅)— and —CF₂—). Any two substituents on the hydrocarbon chain may be joined to form an optionally substituted carbocyclyl, optionally substituted heterocyclyl, optionally substituted aryl, or optionally substituted heteroaryl ring. For instance,

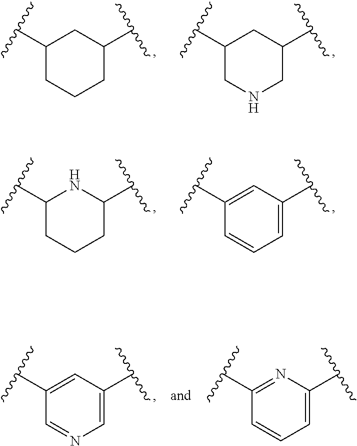 [SMILES: *C1CCCC(*)C1 ; *C1CNCC(*)C1 ; *C1CCCC(*)N1 ; *c1cccc(*)c1 ; *c1cncc(*)c1 ; *c1cccc(*)n1]

are all examples of a hydrocarbon chain. In contrast, in certain embodiments

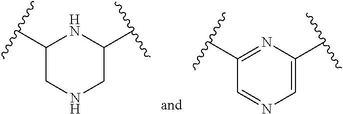 [SMILES: *C1CNCC(*)N1 ; *c1cncc(*)n1]

are not within the scope of the hydrocarbon chains described herein.

“Alkyl” refers to a radical of a straight-chain or branched saturated hydrocarbon group having from 1 to 20 carbon atoms (“C₁₋₂₀ alkyl”). In some embodiments, an alkyl group has 1 to 10 carbon atoms (“C₁₋₁₀ alkyl”). In some embodiments, an alkyl group has 2 to 20 carbon atoms (“C₂₋₂₀ alkyl”) or 2 to 10 carbon atoms (“C₂₋₁₀ alkyl”). In some embodiments, an alkyl group has 2 to 20 carbon atoms (“C₂₋₂₀ alkyl”). In some embodiments, an alkyl group has 2 to 10 carbon atoms (“C₂₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently optionally substituted, i.e., unsubstituted (an “unsubstituted alkyl”) or substituted (a “substituted alkyl”) with one or more substituents. In certain embodiments, the alkyl group is unsubstituted C₁₋₁₀ alkyl (e.g., —CH₃). In certain embodiments, the alkyl group is substituted C₁₋₁₀ alkyl.

“Alkenyl” refers to a radical of a straight-chain or branched hydrocarbon group having from 2 to 20 carbon atoms, one or more carbon-carbon double bonds, and no triple bonds (“C₂₋₂₀ alkenyl”). In some embodiments, an alkenyl group has 2 to 10 carbon atoms (“C₂-10 alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently optionally substituted, i.e., unsubstituted (an “unsubstituted alkenyl”) or substituted (a “substituted alkenyl”) with one or more substituents. In certain embodiments, the alkenyl group is unsubstituted C₂₋₁₀ alkenyl. In certain embodiments, the alkenyl group is substituted C₂₋₁₀ alkenyl.

“Alkynyl” refers to a radical of a straight-chain or branched hydrocarbon group having from 2 to 20 carbon atoms, one or more carbon-carbon triple bonds, and optionally one or more double bonds (“C₂₋₂₀ alkynyl”). In some embodiments, an alkynyl group has 2 to 10 carbon atoms (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently optionally substituted, i.e., unsubstituted (an “unsubstituted alkynyl”) or substituted (a “substituted alkynyl”) with one or more substituents. In certain embodiments, the alkynyl group is unsubstituted C₂₋₁₀ alkynyl. In certain embodiments, the alkynyl group is substituted C₂₋₁₀ alkynyl.

“Carbocyclyl” or “carbocyclic” refers to a radical of a non-aromatic cyclic hydrocarbon group having from 3 to 10 ring carbon atoms (“C₃₋₁₀ carbocyclyl”) and _{ww}ero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or contain a fused, bridged or spiro ring system such as a bicyclic system (“bicyclic carbocyclyl”) and can be saturated or can be partially unsaturated. “Carbocyclyl” also includes ring systems wherein the carbocyclic ring, as defined above, is fused with one or more aryl or heteroaryl groups wherein the point of attachment is on the carbocyclic ring, and in such instances, the number of carbons continue to designate the number of carbons in the carbocyclic ring system. Unless otherwise specified, each instance of a carbocyclyl group is independently optionally substituted, i.e., unsubstituted (an “unsubstituted carbocyclyl”) or substituted (a “substituted carbocyclyl”) with one or more substituents. In certain embodiments, the carbocyclyl group is unsubstituted C₃₋₁₀ carbocyclyl. In certain embodiments, the carbocyclyl group is a substituted C₃₋₁₀ carbocyclyl.

In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅_cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is unsubstituted C₃₋₁₀ cycloalkyl. In certain embodiments, the cycloalkyl group is substituted C₃₋₁₀ cycloalkyl.

“Heterocyclyl” or “heterocyclic” refers to a radical of a 3- to 10-membered non-aromatic ring system having ring carbon atoms and 1 to 4 ring heteroatoms, wherein each heteroatom is independently selected from the group consisting of nitrogen, oxygen, sulfur, boron, phosphorus, and silicon (“3-10 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclic ring, as defined above, is fused with one or more carbocyclyl groups wherein the point of attachment is either on the carbocyclyl or heterocyclic ring, or ring systems wherein the heterocyclic ring, as defined above, is fused with one or more aryl or heteroaryl groups, wherein the point of attachment is on the heterocyclic ring, and in such instances, the number of ring members continue to designate the number of ring members in the heterocyclic ring system. Unless otherwise specified, each instance of heterocyclyl is independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-10 membered heterocyclyl. In certain embodiments, the heterocyclyl group is substituted 3-10 membered heterocyclyl.

In some embodiments, a heterocyclyl group is a 5-10 membered non-aromatic ring system having ring carbon atoms and 1-4 ring heteroatoms, wherein each heteroatom is independently selected from the group consisting of nitrogen, oxygen, sulfur, boron, phosphorus, and silicon (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from the group consisting of nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from the group consisting of nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has one ring heteroatom selected from nitrogen, oxygen, and sulfur.

Exemplary 3-membered heterocyclyl groups containing one heteroatom include, without limitation, azirdinyl, oxiranyl, and thiiranyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl, tetrahydropyranyl, dihydropyridinyl, and thianyl. Exemplary 6-membered heterocyclyl groups containing two heteroatoms include, without limitation, piperazinyl, morpholinyl, dithianyl, and dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclic ring) include, without limitation, indolinyl, isoindolinyl, dihydrobenzofuranyl, dihydrobenzothienyl, benzoxazolinonyl, and the like. Exemplary 6-membered heterocyclyl groups fused to an aryl ring (also referred to herein as a 6,6-bicyclic heterocyclic ring) include, without limitation, tetrahydroquinolinyl, tetrahydroisoquinolinyl, and the like.

“Aryl” refers to a radical of a monocyclic or polycyclic (e.g., bicyclic or tricyclic) 4n+2 aromatic ring system (e.g., having 6, 10, or 14 pi electrons shared in a cyclic array) having 6-14 ring carbon atoms and zero heteroatoms provided in the aromatic ring system (“C₆₋₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently optionally substituted, i.e., unsubstituted (an “unsubstituted aryl”) or substituted (a “substituted aryl”) with one or more substituents. In certain embodiments, the aryl group is unsubstituted C₆₋₁₄ aryl. In certain embodiments, the aryl group is substituted C₆₋₁₄ aryl.

“Aralkyl” is a subset of alkyl and aryl and refers to an optionally substituted alkyl group substituted by an optionally substituted aryl group. In certain embodiments, the aralkyl is optionally substituted benzyl. In certain embodiments, the aralkyl is benzyl. In certain embodiments, the aralkyl is optionally substituted phenethyl. In certain embodiments, the aralkyl is phenethyl.

“Heteroaryl” refers to a radical of a 5-10 membered monocyclic or bicyclic 4n+2 aromatic ring system (e.g., having 6 or 10 pi electrons shared in a cyclic array) having ring carbon atoms and 1-4 ring heteroatoms provided in the aromatic ring system, wherein each heteroatom is independently selected from the group consisting of nitrogen, oxygen, and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl).

In some embodiments, a heteroaryl group is a 5-10 membered aromatic ring system having ring carbon atoms and 1-4 ring heteroatoms provided in the aromatic ring system, wherein each heteroatom is independently selected from the group consisting of nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from the group consisting of nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from the group consisting of nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently optionally substituted, i.e., unsubstituted (an “unsubstituted heteroaryl”) or substituted (a “substituted heteroaryl”) with one or more substituents. In certain embodiments, the heteroaryl group is unsubstituted 5-14 membered heteroaryl. In certain embodiments, the heteroaryl group is substituted 5-14 membered heteroaryl.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl.

“Heteroaralkyl” is a subset of alkyl and heteroaryl and refers to an optionally substituted alkyl group substituted by an optionally substituted heteroaryl group.

“Partially unsaturated” refers to a group that includes at least one double or triple bond. A “partially unsaturated” ring system is further intended to encompass rings having multiple sites of unsaturation but is not intended to include aromatic groups (e.g., aryl or heteroaryl groups) as defined herein. Likewise, “saturated” refers to a group that does not contain a double or triple bond, i.e., contains all single bonds.

Alkyl, alkenyl, alkynyl, carbocyclyl, heterocyclyl, aryl, and heteroaryl groups, which are divalent bridging groups are further referred to using the suffix -ene, e.g., alkylene, alkenylene, alkynylene, carbocyclylene, heterocyclylene, arylene, and heteroarylene.

The term “optionally substituted” refers to substituted or unsubstituted.

Alkyl, alkenyl, alkynyl, carbocyclyl, heterocyclyl, aryl, and heteroaryl groups are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{aa})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

each instance of R is, independently, selected from hydrogen, C₁₋₁₀ alkyl, C₁₋₁₀ perhaloalkyl, C₂₋₁₀ alkenyl, C₂₋₁₀ alkynyl, heteroC₁₋₁₀ alkyl, heteroC₂₋₁₀ alkenyl, heteroC₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₆ alkyl, C₁₋₆ perhaloalkyl, C₂₋₆ alkenyl, C₂₋₆ alkynyl, heteroC₁₋₆ alkyl, heteroC₂₋₆alkenyl, heteroC₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{f} is, independently, selected from hydrogen, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, 3-10 membered heterocyclyl, C₆₋₁₀ aryl, and 5-10 membered heteroaryl, or two R^{f} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups; and

each instance of R^{gg} is, independently, selected from halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃ -C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkynyl, heteroC₁₋₆alkyl, heteroC₂₋₆alkenyl, heteroC₂₋₆alkynyl, C₃₋₁₀ carbocyclyl, C₆₋₁₀ aryl, 3-10 membered heterocyclyl, and 5-10 membered heteroaryl; or two geminal R^{gg} substituents can be joined to form ═O or ═S; wherein X⁻ is a counterion.

A “counterion” or “anionic counterion” is a negatively charged group associated with a positively charged group in order to maintain electronic neutrality. An anionic counterion may be monovalent (i.e., including one formal negative charge). An anionic counterion may also be multivalent (i.e., including more than one formal negative charge), such as divalent or trivalent. Exemplary counterions include halide ions (e.g., F⁻, Cl⁻, Br⁻, I⁻), NO₃⁻, ClO₄⁻, OH⁻, H₂PO₄⁻, HCO₃⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), carboxylate ions (e.g., acetate, propanoate, benzoate, glycerate, lactate, tartrate, glycolate, gluconate, and the like), BF₄⁻, PF₄⁻, PF₆⁻, AsF₆⁻, SbF₆⁻, B[3,5-(CF₃)₂C₆H₃]₄]⁻, B(C₆F5)₄⁻, BPh₄⁻, Al(OC(CF₃)₃)₄⁻, and carborane anions (e.g., CB₁₁H₁₂⁻ or (HCB₁₁Me₅Br₆)⁻). Exemplary counterions which may be multivalent include CO₃²⁻, HPO₄²⁻, PO₄³⁻, B₄O₇²⁻, SO₄²⁻, S₂O₃²⁻, carboxylate anions (e.g., tartrate, citrate, fumarate, maleate, malate, malonate, gluconate, succinate, glutarate, adipate, pimelate, suberate, azelate, sebacate, salicylate, phthalates, aspartate, glutamate, and the like), and carboranes.

“Halo” or “halogen” refers to fluorine (fluoro, —F), chlorine (chloro, —Cl), bromine (bromo, —Br), or iodine (iodo, —I).

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NR^{X1})OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

“Alkoxy” or “alkoxyl” refers to a radical of the formula: —O-alkyl.

Nitrogen atoms can be substituted or unsubstituted as valency permits, and include primary, secondary, tertiary, and quaternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(OR^{cc})₂, —P(═O)(R^{aa})₂, —P(═O)(N(R^{cc})₂)₂, C₁₋₁₀ alkyl, C₁₋₁₀ perhaloalkyl, C₂₋₁₀ alkenyl, C₂₋₁₀ alkynyl, heteroC₁₋₁₀alkyl, heteroC₂₋₁₀alkenyl, heteroC₂₋₁₀alkynyl, C₃₋₁₀ carbocyclyl, 3-14 membered heterocyclyl, C₆₋₁₄ aryl, and 5-14 membered heteroaryl, or two R^{cc} groups attached to an N atom are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on a nitrogen atom is a nitrogen protecting group (also referred to as an amino protecting group). Nitrogen protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc})R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, Rb, R^{cc} and R^{dd} are as defined herein. Nitrogen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

For example, nitrogen protecting groups such as amide groups (e.g., —C(═O)R^{aa}) include, but are not limited to, formamide, acetamide, chloroacetamide, trichloroacetamide, trifluoroacetamide, phenylacetamide, 3-phenylpropanamide, picolinamide, 3-pyridylcarboxamide, N-benzoylphenylalanyl derivative, benzamide, p-phenylbenzamide, o-nitophenylacetamide, o-nitrophenoxyacetamide, acetoacetamide, (N′-dithiobenzyloxyacylamino)acetamide, 3-(p-hydroxyphenyl)propanamide, 3-(o-nitrophenyl)propanamide, 2-methyl-2-(o-nitrophenoxy)propanamide, 2-methyl-2-(o-phenylazophenoxy)propanamide, 4-chlorobutanamide, 3-methyl-3-nitrobutanamide, o-nitrocinnamide, N-acetylmethionine derivative, o-nitrobenzamide, and o-(benzoyloxymethyl)benzamide.

Nitrogen protecting groups such as carbamate groups (e.g., —C(═O)OR^{aa}) include, but are not limited to, methyl carbamate, ethyl carbamante, 9-fluorenylmethyl carbamate (Fmoc), 9-(2-sulfo)fluorenylmethyl carbamate, 9-(2,7-dibromo)fluoroenylmethyl carbamate, 2,7-di-t-butyl-[9-(10,10-dioxo-10,10,10,10-tetrahydrothioxanthyl)]methyl carbamate (DBD-Tmoc), 4-methoxyphenacyl carbamate (Phenoc), 2,2,2-trichloroethyl carbamate (Troc), 2-trimethylsilylethyl carbamate (Teoc), 2-phenylethyl carbamate (hZ), 1-(1-adamantyl)-1-methylethyl carbamate (Adpoc), 1,1-dimethyl-2-haloethyl carbamate, 1,1-dimethyl-2,2-dibromoethyl carbamate (DB-t-BOC), 1,1-dimethyl-2,2,2-trichloroethyl carbamate (TCBOC), 1-methyl-1-(4-biphenylyl)ethyl carbamate (Bpoc), 1-(3,5-di-t-butylphenyl)-1-methylethyl carbamate (t-Bumeoc), 2-(2′- and 4′-pyridyl)ethyl carbamate (Pyoc), 2-(N,N-dicyclohexylcarboxamido)ethyl carbamate, t-butyl carbamate (BOC), 1-adamantyl carbamate (Adoc), vinyl carbamate (Voc), allyl carbamate (Alloc), 1-isopropylallyl carbamate (Ipaoc), cinnamyl carbamate (Coc), 4-nitrocinnamyl carbamate (Noc), 8-quinolyl carbamate, N-hydroxypiperidinyl carbamate, alkyldithio carbamate, benzyl carbamate (Cbz), p-methoxybenzyl carbamate (Moz), p-nitobenzyl carbamate, p-bromobenzyl carbamate, p-chlorobenzyl carbamate, 2,4-dichlorobenzyl carbamate, 4-methylsulfinylbenzyl carbamate (Msz), 9-anthrylmethyl carbamate, diphenylmethyl carbamate, 2-methylthioethyl carbamate, 2-methylsulfonylethyl carbamate, 2-(p-toluenesulfonyl)ethyl carbamate, [2-(1,3-dithianyl)]methyl carbamate (Dmoc), 4-methylthiophenyl carbamate (Mtpc), 2,4-dimethylthiophenyl carbamate (Bmpc), 2-phosphonioethyl carbamate (Peoc), 2-triphenylphosphonioisopropyl carbamate (Ppoc), 1,1-dimethyl-2-cyanoethyl carbamate, m-chloro-p-acyloxybenzyl carbamate, p-(dihydroxyboryl)benzyl carbamate, 5-benzisoxazolylmethyl carbamate, 2-(trifluoromethyl)-6-chromonylmethyl carbamate (Tcroc), m-nitrophenyl carbamate, 3,5-dimethoxybenzyl carbamate, o-nitrobenzyl carbamate, 3,4-dimethoxy-6-nitrobenzyl carbamate, phenyl(o-nitrophenyl)methyl carbamate, t-amyl carbamate, S-benzyl thiocarbamate, p-cyanobenzyl carbamate, cyclobutyl carbamate, cyclohexyl carbamate, cyclopentyl carbamate, cyclopropylmethyl carbamate, p-decyloxybenzyl carbamate, 2,2-dimethoxyacylvinyl carbamate, o-(N,N-dimethylcarboxamido)benzyl carbamate, 1,1-dimethyl-3-(N,N-dimethylcarboxamido)propyl carbamate, 1,1-dimethylpropynyl carbamate, di(2-pyridyl)methyl carbamate, 2-furanylmethyl carbamate, 2-iodoethyl carbamate, isoborynl carbamate, isobutyl carbamate, isonicotinyl carbamate, p-(p′-methoxyphenylazo)benzyl carbamate, 1-methylcyclobutyl carbamate, 1-methylcyclohexyl carbamate, 1-methyl-1-cyclopropylmethyl carbamate, 1-methyl-1-(3,5-dimethoxyphenyl)ethyl carbamate, 1-methyl-1-(p-phenylazophenyl)ethyl carbamate, 1-methyl-1-phenylethyl carbamate, 1-methyl-1-(4-pyridyl)ethyl carbamate, phenyl carbamate, p-(phenylazo)benzyl carbamate, 2,4,6-tri-t-butylphenyl carbamate, 4-(trimethylammonium)benzyl carbamate, and 2,4,6-trimethylbenzyl carbamate.

Nitrogen protecting groups such as sulfonamide groups (e.g., —S(═O)₂R^{aa}) include, but are not limited to, p-toluenesulfonamide (Ts), benzenesulfonamide, 2,3,6,-trimethyl-4-methoxybenzenesulfonamide (Mtr), 2,4,6-trimethoxybenzenesulfonamide (Mtb), 2,6-dimethyl-4-methoxybenzenesulfonamide (Pme), 2,3,5,6-tetramethyl-4-methoxybenzenesulfonamide (Mte), 4-methoxybenzenesulfonamide (Mbs), 2,4,6-trimethylbenzenesulfonamide (Mts), 2,6-dimethoxy-4-methylbenzenesulfonamide (iMds), 2,2,5,7,8-pentamethylchroman-6-sulfonamide (Pmc), methanesulfonamide (Ms), (3-trimethylsilylethanesulfonamide (SES), 9-anthracenesulfonamide, 4-(4′,8′-dimethoxynaphthylmethyl)benzenesulfonamide (DNMBS), benzylsulfonamide, trifluoromethylsulfonamide, and phenacylsulfonamide.

Other nitrogen protecting groups include, but are not limited to, phenothiazinyl-(10)-acyl derivative, N′-p-toluenesulfonylaminoacyl derivative, N′-phenylaminothioacyl derivative, N-benzoylphenylalanyl derivative, N-acetylmethionine derivative, 4,5-diphenyl-3-oxazolin-2-one, N-phthalimide, N-dithiasuccinimide (Dts), N-2,3-diphenylmaleimide, N-2,5-dimethylpyrrole, N-1,1,4,4-tetramethyldisilylazacyclopentane adduct (STABASE), 5-substituted 1,3-dimethyl-1,3,5-triazacyclohexan-2-one, 5-substituted 1,3-dibenzyl-1,3,5-triazacyclohexan-2-one, 1-substituted 3,5-dinitro-4-pyridone, N-methylamine, N-allylamine, N-[2-(trimethylsilyl)ethoxy]methylamine (SEM), N-3-acetoxypropylamine, N-(1-isopropyl-4-nitro-2-oxo-3-pyroolin-3-yl)amine, quaternary ammonium salts, N-benzylamine, N-di(4-methoxyphenyl)methylamine, N-5-dibenzosuberylamine, N-triphenylmethylamine (Tr), N-[(4-methoxyphenyl)diphenylmethyl]amine (MMTr), N-9-phenylfluorenylamine (PhF), N-2,7-dichloro-9-fluorenylmethyleneamine, N-ferrocenylmethylamino (Fcm), N-2-picolylamino N′-oxide, N-1,1-dimethylthiomethyleneamine, N-benzylideneamine, N-p-methoxybenzylideneamine, N-diphenylmethyleneamine, N-[(2-pyridyl)mesityl]methyleneamine, N—(N′,N′-dimethylaminomethylene)amine, N,N′-isopropylidenediamine, N-p-nitrobenzylideneamine, N-salicylideneamine, N-5-chlorosalicylideneamine, N-(5-chloro-2-hydroxyphenyl)phenylmethyleneamine, N-cyclohexylideneamine, N-(5,5-dimethyl-3-oxo-1-cyclohexenyl)amine, N-borane derivative, N-diphenylborinic acid derivative, N-[phenyl(pentaacylchromium- or tungsten)acyl]amine, N-copper chelate, N-zinc chelate, N-nitroamine, N-nitrosoamine, amine N-oxide, diphenylphosphinamide (Dpp), dimethylthiophosphinamide (Mpt), diphenylthiophosphinamide (Ppt), dialkyl phosphoramidates, dibenzyl phosphoramidate, diphenyl phosphoramidate, benzenesulfenamide, o-nitrobenzenesulfenamide (Nps), 2,4-dinitrobenzenesulfenamide, pentachlorobenzenesulfenamide, 2-nitro-4-methoxybenzenesulfenamide, triphenylmethylsulfenamide, and 3-nitropyridinesulfenamide (Npys).

In certain embodiments, the substituent present on an oxygen atom is an oxygen protecting group (also referred to herein as an “hydroxyl protecting group”). Oxygen protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR^{cc})₃⁺X⁻, —P(═O)(R^{aa})₂, —P(═O)(OR^{cc})₂, and —P(═O)(N(R^{bb})₂)₂, wherein X⁻, R^{aa}, R^{bb}, and R^{cc} are as defined herein. Oxygen protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

Exemplary oxygen protecting groups include, but are not limited to, methyl, methoxylmethyl (MOM), methylthiomethyl (MTM), t-butylthiomethyl, (phenyldimethylsilyl)methoxymethyl (SMOM), benzyloxymethyl (BOM), p-methoxybenzyloxymethyl (PMBM), (4-methoxyphenoxy)methyl (p-AOM), guaiacolmethyl (GUM), t-butoxymethyl, 4-pentenyloxymethyl (POM), siloxymethyl, 2-methoxyethoxymethyl (MEM), 2,2,2-trichloroethoxymethyl, bis(2-chloroethoxy)methyl, 2-(trimethylsilyl)ethoxymethyl (SEMOR), tetrahydropyranyl (THP), 3-bromotetrahydropyranyl, tetrahydrothiopyranyl, 1-methoxycyclohexyl, 4-methoxytetrahydropyranyl (MTHP), 4-methoxytetrahydrothiopyranyl, 4-methoxytetrahydrothiopyranyl S,S-dioxide, 1-[(2-chloro-4-methyl)phenyl]-4-methoxypiperidin-4-yl (CTMP), 1,4-dioxan-2-yl, tetrahydrofuranyl, tetrahydrothiofuranyl, 2,3,3a,4,5,6,7,7a-octahydro-7,8,8-trimethyl-4,7-methanobenzofuran-2-yl, 1-ethoxyethyl, 1-(2-chloroethoxy)ethyl, 1-methyl-1-methoxyethyl, 1-methyl-1-benzyloxyethyl, 1-methyl-1-benzyloxy-2-fluoroethyl, 2,2,2-trichloroethyl, 2-trimethylsilylethyl, 2-(phenylselenyl)ethyl, t-butyl, allyl, p-chlorophenyl, p-methoxyphenyl, 2,4-dinitrophenyl, benzyl (Bn), p-methoxybenzyl, 3,4-dimethoxybenzyl, o-nitrobenzyl, p-nitrobenzyl, p-halobenzyl, 2,6-dichlorobenzyl, p-cyanobenzyl, p-phenylbenzyl, 2-picolyl, 4-picolyl, 3-methyl-2-picolyl N-oxido, diphenylmethyl, p,p′-dinitrobenzhydryl, 5-dibenzosuberyl, triphenylmethyl, α-naphthyldiphenylmethyl, p-methoxyphenyldiphenylmethyl, di(p-methoxyphenyl)phenylmethyl, tri(p-methoxyphenyl)methyl, 4-(4′-bromophenacyloxyphenyl)diphenylmethyl, 4,4′,4″-tris(4,5-dichlorophthalimidophenyl)methyl, 4,4′,4″-tris(levulinoyloxyphenyl)methyl, 4,4′,4″-tris(benzoyloxyphenyl)methyl, 3-(imidazol-1-yl)bis(4′,4″-dimethoxyphenyl)methyl, 1,1-bis(4-methoxyphenyl)-1′-pyrenylmethyl, 9-anthryl, 9-(9-phenyl)xanthenyl, 9-(9-phenyl-10-oxo)anthryl, 1,3-benzodisulfuran-2-yl, benzisothiazolyl S,S-dioxido, trimethylsilyl (TMS), triethylsilyl (TES), triisopropylsilyl (TIPS), dimethylisopropylsilyl (IPDMS), diethylisopropylsilyl (DEIPS), dimethylthexylsilyl, t-butyldimethylsilyl (TBDMS), t-butyldiphenylsilyl (TBDPS), tribenzylsilyl, tri-p-xylylsilyl, triphenylsilyl, diphenylmethylsilyl (DPMS), t-butylmethoxyphenylsilyl (TBMPS), formate, benzoylformate, acetate, chloroacetate, dichloroacetate, trichloroacetate, trifluoroacetate, methoxyacetate, triphenylmethoxyacetate, phenoxyacetate, p-chlorophenoxyacetate, 3-phenylpropionate, 4-oxopentanoate (levulinate), 4,4-(ethylenedithio)pentanoate (levulinoyldithioacetal), pivaloate, adamantoate, crotonate, 4-methoxycrotonate, benzoate, p-phenylbenzoate, 2,4,6-trimethylbenzoate (mesitoate), alkyl methyl carbonate, 9-fluorenylmethyl carbonate (Fmoc), alkyl ethyl carbonate, alkyl 2,2,2-trichloroethyl carbonate (Troc), 2-(trimethylsilyl)ethyl carbonate (TMSEC), 2-(phenylsulfonyl) ethyl carbonate (Psec), 2-(triphenylphosphonio) ethyl carbonate (Peoc), alkyl isobutyl carbonate, alkyl vinyl carbonate alkyl allyl carbonate, alkyl p-nitrophenyl carbonate, alkyl benzyl carbonate, alkyl p-methoxybenzyl carbonate, alkyl 3,4-dimethoxybenzyl carbonate, alkyl o-nitrobenzyl carbonate, alkyl p-nitrobenzyl carbonate, alkyl S-benzyl thiocarbonate, 4-ethoxy-1-napththyl carbonate, methyl dithiocarbonate, 2-iodobenzoate, 4-azidobutyrate, 4-nitro-4-methylpentanoate, o-(dibromomethyl)benzoate, 2-formylbenzenesulfonate, 2-(methylthiomethoxy)ethyl, 4-(methylthiomethoxy)butyrate, 2-(methylthiomethoxymethyl)benzoate, 2,6-dichloro-4-methylphenoxyacetate, 2,6-dichloro-4-(1,1,3,3-tetramethylbutyl)phenoxyacetate, 2,4-bis(1,1-dimethylpropyl)phenoxyacetate, chlorodiphenylacetate, isobutyrate, monosuccinoate, (E)-2-methyl-2-butenoate, o-(methoxyacyl)benzoate, α-naphthoate, nitrate, alkyl N,N,N′,N′-tetramethylphosphorodiamidate, alkyl N-phenylcarbamate, borate, dimethylphosphinothioyl, alkyl 2,4-dinitrophenylsulfenate, sulfate, methanesulfonate (mesylate), benzylsulfonate, and tosylate (Ts).

In certain embodiments, the substituent present on a sulfur atom is a sulfur protecting group (also referred to as a “thiol protecting group”). Sulfur protecting groups include, but are not limited to, —R^{aa}, —N(R^{bb})₂, —C(═O)SR^{aa}, —C(═O)R^{aa}, —CO₂R^{aa}, —C(═O)N(R^{bb})₂, —C(═NR^{bb})R^{aa}, —C(═NR^{bb})OR^{aa}, —C(═NR^{bb})N(R^{bb})₂, —S(═O)R^{aa}, —SO₂R^{aa}, —Si(R^{aa})₃, —P(R^{cc})₂, —P(R^{cc})₃⁺X⁻, —P(OR^{cc})₂, —P(OR)₃⁺X⁻, —P(═O)(R^{aa})₂, —P(═O)(OR^{cc})₂, and —P(═O)(N(R^{bb})₂)₂, wherein R^{aa}, R^{bb}, and R^{cc} are as defined herein. Sulfur protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

As used herein, a “leaving group” (LG) is an art-understood term referring to a molecular fragment that departs with a pair of electrons in a heterolytic bond cleavage, wherein the molecular fragment is an anion or neutral molecule. As used herein, a leaving group can be an atom or a group capable of being displaced by a nucleophile. See, for example, Smith, March Advanced Organic Chemistry 6th ed. (501-502). Exemplary leaving groups include, but are not limited to, halo (e.g., chloro, bromo, iodo) and activated substituted hydroxyl groups (e.g., —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OP(R^{aa})₂, —OP(R^{aa})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein). Examples of suitable leaving groups include, but are not limited to, halogen (such as F, Cl, Br, or I (iodine)), alkoxycarbonyloxy, aryloxycarbonyloxy, alkanesulfonyloxy, arenesulfonyloxy, alkyl-carbonyloxy (e.g., acetoxy), arylcarbonyloxy, aryloxy, methoxy, N,O-dimethylhydroxylamino, pixyl, and haloformates. In some cases, the leaving group is a sulfonic acid ester, such as toluenesulfonate (tosylate, —OTs), methanesulfonate (mesylate, —OMs), p-bromobenzenesulfonyloxy (brosylate, —OBs), or trifluoromethanesulfonate (triflate, —OTf). In some cases, the leaving group is a brosylate, such as p-bromobenzenesulfonyloxy. In some cases, the leaving group is a nosylate, such as 2-nitrobenzenesulfonyloxy. In some embodiments, the leaving group is a sulfonate-containing group. In some embodiments, the leaving group is a tosylate group. The leaving group may also be a phosphineoxide (e.g., formed during a Mitsunobu reaction) or an internal leaving group such as an epoxide or cyclic sulfate. Other non-limiting examples of leaving groups are water, amines, ammonia, alcohols, ether moieties, sulfur-containing moieties, thioether moieties, zinc halides, magnesium moieties, diazonium salts, and copper moieties.

The term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al. describe pharmaceutically acceptable salts in detail in J. Pharmaceutical Sciences, 1977, 66, 1-19, incorporated herein by reference. Pharmaceutically acceptable salts of the compounds of this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods known in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺(C₁₋₄ alkyl)₄ salts. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formula (I) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound that is associated with water. Typically, the number of the water molecules contained in a hydrate of a compound is in a definite ratio to the number of the compound molecules in the hydrate. Therefore, a hydrate of a compound may be represented, for example, by the general formula R-x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R-0.5 H₂O)), and polyhydrates (x is a number greater than 1, e.g., dihydrates (R-2 H₂O) and hexahydrates (R-6 H₂O)).

The term “tautomers” refer to compounds that are interchangeable forms of a particular compound structure, and that vary in the displacement of hydrogen atoms and electrons. Thus, two structures may be in equilibrium through the movement of J electrons and an atom (usually H). For example, enols and ketones are tautomers because they are rapidly interconverted by treatment with either acid or base. Another example of tautomerism is the aci- and nitro- forms of phenylnitromethane, that are likewise formed by treatment with acid or base.

Tautomeric forms may be relevant to the attainment of the optimal chemical reactivity and biological activity of a compound of interest.

It is also to be understood that compounds that have the same molecular formula but differ in the nature or sequence of bonding of their atoms or the arrangement of their atoms in space are termed “isomers.” Isomers that differ in the arrangement of their atoms in space are termed “stereoisomers.”

Stereoisomers that are not mirror images of one another are termed “diastereomers” and those that are non-superimposable mirror images of each other are termed “enantiomers.” When a compound has an asymmetric center, for example, it is bonded to four different groups, a pair of enantiomers is possible. An enantiomer can be characterized by the absolute configuration of its asymmetric center and is described by the R- and S-sequencing rules of Cahn and Prelog, or by the manner in which the molecule rotates the plane of polarized light and designated as dextrorotatory or levorotatory (i.e., as (+) or (−)-isomers respectively). A chiral compound can exist as either individual enantiomer or as a mixture thereof. A mixture containing equal proportions of the enantiomers is called a “racemic mixture.”

The term “polymorphs” refers to a crystalline form of a compound (or a salt, hydrate, or solvate thereof) in a particular crystal packing arrangement. All polymorphs have the same elemental composition. Different crystalline forms usually have different X-ray diffraction patterns, infrared spectra, melting points, density, hardness, crystal shape, optical and electrical properties, stability, and solubility. Recrystallization solvent, rate of crystallization, storage temperature, and other factors may cause one crystal form to dominate. Various polymorphs of a compound can be prepared by crystallization under different conditions.

The term “prodrugs” refer to compounds, including derivatives of the compounds of Formula (I), which have cleavable groups and become by solvolysis or under physiological conditions the compounds of Formula (I) which are pharmaceutically active in vivo. Such examples include, but are not limited to, ester derivatives and the like. Other derivatives of the compounds of this invention have activity in both their acid and acid derivative forms, but in the acid sensitive form often offers advantages of solubility, tissue compatibility, or delayed release in the mammalian organism (see, Bundgard, H., Design of Prodrugs, pp. 7-9, 21-24, Elsevier, Amsterdam 1985). Prodrugs include acid derivatives well known to practitioners of the art, such as, for example, esters prepared by reaction of the parent acid with a suitable alcohol, or amides prepared by reaction of the parent acid compound with a substituted or unsubstituted amine, or acid anhydrides, or mixed anhydrides. Simple aliphatic or aromatic esters, amides, and anhydrides derived from acidic groups pendant on the compounds of this invention are particular prodrugs.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

The terms “administer,” “administering,” or “administration” refer to implanting, absorbing, ingesting, injecting, inhaling, or otherwise introducing an inventive compound, or a pharmaceutical composition thereof.

The terms “treatment,” “treat,” and “treating” refer to reversing, alleviating, delaying the onset of, or inhibiting the progress of a “pathological condition” (e.g., a disease, disorder, or condition, or one or more signs or symptoms thereof) described herein. In some embodiments, treatment may be administered after one or more signs or symptoms have developed or have been observed. In other embodiments, treatment may be administered in the absence of signs or symptoms of the disease or condition. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence.

The terms “condition,” “disease,” and “disorder” are used interchangeably.

An “effective amount” of a compound of Formula (I) refers to an amount sufficient to elicit the desired biological response, i.e., treating the condition. As will be appreciated by those of ordinary skill in this art, the effective amount of a compound of Formula (I) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formula (I) is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms or causes of the condition, or enhances the therapeutic efficacy of another therapeutic agent.

The term “angiogenesis” refers to the formation and the growth of new blood vessels. Normal angiogenesis occurs in the healthy body of a subject for healing wounds and for restoring blood flow to tissues after injury. The healthy body controls angiogenesis through a number of means, e.g., angiogenesis-stimulating growth factors and angiogenesis inhibitors. Many disease states, such as cancer, diabetic blindness, age-related macular degeneration, rheumatoid arthritis, and psoriasis, are characterized by abnormal (i.e., increased or excessive) angiogenesis. Abnormal or pathological angiogenesis refers to angiogenesis greater than that in a normal body, especially angiogenesis in an adult not related to normal angiogenesis (e.g., menstruation or wound healing). Abnormal angiogenesis can provide new blood vessels that feed diseased tissues and/or destroy normal tissues, and in the case of cancer, the new vessels can allow tumor cells to escape into the circulation and lodge in other organs (tumor metastases). In certain embodiments, the angiogenesis is pathological angiogenesis.

The term “biological sample” refers to any sample including tissue samples (such as tissue sections and needle biopsies of a tissue); cell samples (e.g., cytological smears (such as Pap or blood smears) or samples of cells obtained by microdissection); samples of whole organisms (such as samples of yeasts or bacteria); or cell fractions, fragments, or organelles (such as obtained by lysing cells and separating the components thereof by centrifugation or otherwise). Other examples of biological samples include blood, serum, urine, semen, fecal matter, cerebrospinal fluid, interstitial fluid, mucus, tears, sweat, pus, biopsied tissue (e.g., obtained by a surgical biopsy or needle biopsy), nipple aspirates, milk, vaginal fluid, saliva, swabs (such as buccal swabs), or any material containing biomolecules that is derived from a first biological sample. Biological samples also include those biological samples that are transgenic, such as a transgenic oocyte, sperm cell, blastocyst, embryo, fetus, donor cell, or cell nucleus, or cells or cell lines derived from biological samples.

The term “tissue” refers to any biological tissue of a subject (including a group of cells, a body part, or an organ) or a part thereof, including blood and/or lymph vessels, which is the object to which a compound, particle, and/or composition of the invention is delivered. A tissue may be an abnormal or unhealthy tissue, which may need to be treated. A tissue may also be a normal or healthy tissue that is under a higher than normal risk of becoming abnormal or unhealthy, which may need to be prevented. In certain embodiments, the tissue is the central nervous system. In certain embodiments, the tissue is the brain.

The term “administer,” “administering,” or “administration” refers to implanting, absorbing, ingesting, injecting, inhaling, or otherwise introducing a compound described herein, or a composition thereof, in or on a subject.

The terms “treatment,” “treat,” and “treating” refer to reversing, alleviating, delaying the onset of, or inhibiting the progress of a disease described herein. In some embodiments, treatment may be administered after one or more signs or symptoms of the disease have developed or have been observed. In other embodiments, treatment may be administered in the absence of signs or symptoms of the disease. For example, treatment may be administered to a susceptible subject prior to the onset of symptoms (e.g., in light of a history of symptoms). Treatment may also be continued after symptoms have resolved, for example, to delay or prevent recurrence.

The terms “condition,” “disease,” and “disorder” are used interchangeably.

An “effective amount” of a compound described herein refers to an amount sufficient to elicit the desired biological response. An effective amount of a compound described herein may vary depending on such factors as the desired biological endpoint, the pharmacokinetics of the compound, the condition being treated, the mode of administration, and the age and health of the subject. In certain embodiments, an effective amount is a therapeutically effective amount. In certain embodiments, an effective amount is a prophylactic treatment. In certain embodiments, an effective amount is the amount of a compound described herein in a single dose. In certain embodiments, an effective amount is the combined amounts of a compound described herein in multiple doses.

A “therapeutically effective amount” of a compound described herein is an amount sufficient to provide a therapeutic benefit in the treatment of a condition or to delay or minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces, or avoids symptoms, signs, or causes of the condition, and/or enhances the therapeutic efficacy of another therapeutic agent. In certain embodiments, a therapeutically effective amount is an amount sufficient for binding a kinase (e.g., HCK, BTK, FYN, SRC, BLK, LCK, CSK, ABL1, ABL2, LIMK1, LATS1). In certain embodiments, a therapeutically effective amount is an amount sufficient for treating a proliferative disease (e.g., cancer). In certain embodiments, a therapeutically effective amount is an amount sufficient for binding and/or inducing the ubiquitination of a kinase (e.g., HCK, BTK, FYN, SRC, BLK, LCK, CSK, ABL1, ABL2, LIMK1, LATS1) and/or inducing the degradation of the kinase (e.g., HCK, BTK, FYN, SRC, BLK, LCK, CSK, ABL1, ABL2, LIMK1, LATS1). In certain embodiments, a therapeutically effective amount is an amount sufficient for binding and/or inducing the ubiquitination of a kinase (e.g., HCK, BTK).

A “prophylactically effective amount” of a compound described herein is an amount sufficient to prevent a condition, or one or more signs or symptoms associated with the condition, or prevent its recurrence. A prophylactically effective amount of a compound means an amount of a therapeutic agent, alone or in combination with other agents, which provides a prophylactic benefit in the prevention of the condition. The term “prophylactically effective amount” can encompass an amount that improves overall prophylaxis or enhances the prophylactic efficacy of another prophylactic agent. In certain embodiments, a prophylactically effective amount is an amount sufficient for binding a kinase (e.g., HCK, BTK, FYN, SRC, BLK, LCK, CSK, ABL1, ABL2, LIMK1, LATS1) and/or inducing the degradation of the kinase (e.g., HCK, BTK, FYN, SRC, BLK, LCK, CSK, ABL1, ABL2, LIMK1, LATS1). In certain embodiments, a prophylactically effective amount is an amount sufficient for binding a kinase (e.g., HCK) and/or inducing the degradation of the kinase (e.g., HCK, BTK). In certain embodiments, a prophylactically effective amount is an amount sufficient for treating a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations).

In certain embodiments, a prophylactically effective amount is an amount sufficient for binding a kinase (e.g., HCK, BTK, FYN, SRC, BLK, LCK, CSK, ABL1, ABL2, LIMK1, LATS1) and/or inducing the degradation of kinase (e.g., HCK, BTK, FYN, SRC, BLK, LCK, CSK, ABL1, ABL2, LIMK1, LATS1), and treating and/or preventing a disease (e.g., proliferative disease (e.g., non-non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations). In certain embodiments, a prophylactically effective amount is an amount sufficient for binding a kinase (e.g., HCK) and/or inducing the degradation of kinase (e.g., HCK, BTK), and treating and/or preventing a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations).

A “proliferative disease” refers to a disease that occurs due to abnormal growth or extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); or 4) the pathological angiogenesis as in proliferative retinopathy and tumor metastasis. Exemplary proliferative diseases include cancers (i.e., “malignant neoplasms”), benign neoplasms, lymphoma, non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, and autoimmune disease. Exemplary proliferative diseases include cancers (i.e., “malignant neoplasms”), benign neoplasms, angiogenesis, inflammatory diseases, autoinflammatory diseases, and autoimmune diseases.

The terms “neoplasm” and “tumor” are used herein interchangeably and refer to an abnormal mass of tissue wherein the growth of the mass surpasses and is not coordinated with the growth of a normal tissue. A neoplasm or tumor may be “benign” or “malignant,” depending on the following characteristics: degree of cellular differentiation (including morphology and functionality), rate of growth, local invasion, and metastasis. A “benign neoplasm” is generally well differentiated, has characteristically slower growth than a malignant neoplasm, and remains localized to the site of origin. In addition, a benign neoplasm does not have the capacity to infiltrate, invade, or metastasize to distant sites. Exemplary benign neoplasms include, but are not limited to, lipoma, chondroma, adenomas, acrochordon, senile angiomas, seborrheic keratoses, lentigos, and sebaceous hyperplasias. In some cases, certain “benign” tumors may later give rise to malignant neoplasms, which may result from additional genetic changes in a subpopulation of the tumor's neoplastic cells, and these tumors are referred to as “pre-malignant neoplasms.” An exemplary pre-malignant neoplasm is a teratoma. In contrast, a “malignant neoplasm” is generally poorly differentiated (anaplasia) and has characteristically rapid growth accompanied by progressive infiltration, invasion, and destruction of the surrounding tissue. Furthermore, a malignant neoplasm generally has the capacity to metastasize to distant sites. The term “metastasis,” “metastatic,” or “metastasize” refers to the spread or migration of cancerous cells from a primary or original tumor to another organ or tissue and is typically identifiable by the presence of a “secondary tumor” or “secondary cell mass” of the tissue type of the primary or original tumor and not of that of the organ or tissue in which the secondary (metastatic) tumor is located. For example, a prostate cancer that has migrated to bone is said to be metastasized prostate cancer and includes cancerous prostate cancer cells growing in bone tissue.

The term “cancer” refers to a malignant neoplasm (Stedman's Medical Dictionary, 25th ed.; Hensyl ed.; Williams & Wilkins: Philadelphia, 1990). Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), MYD88-mutated Waldenstrom macroglobulinemia, activated B-cell (ABC) diffuse large B-cell lymphoma, mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenström's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrinetumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic andenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

The term “inflammatory disease” refers to a disease caused by, resulting from, or resulting in inflammation. The term “inflammatory disease” may also refer to a dysregulated inflammatory reaction that causes an exaggerated response by macrophages, granulocytes, and/or T-lymphocytes leading to abnormal tissue damage and/or cell death. An inflammatory disease can be either an acute or chronic inflammatory condition and can result from infections or non-infectious causes. Inflammatory diseases include, without limitation, atherosclerosis, arteriosclerosis, autoimmune disorders, multiple sclerosis, systemic lupus erythematosus, polymyalgia rheumatica (PMR), gouty arthritis, degenerative arthritis, tendonitis, bursitis, psoriasis, cystic fibrosis, arthrosteitis, rheumatoid arthritis, inflammatory arthritis, Sjogren's syndrome, giant cell arteritis, progressive systemic sclerosis (scleroderma), ankylosing spondylitis, polymyositis, dermatomyositis, pemphigus, pemphigoid, diabetes (e.g., Type I), myasthenia gravis, Hashimoto's thyroiditis, Graves' disease, Goodpasture's disease, mixed connective tissue disease, sclerosing cholangitis, inflammatory bowel disease, Crohn's disease, ulcerative colitis, pernicious anemia, inflammatory dermatoses, usual interstitial pneumonitis (UIP), asbestosis, silicosis, bronchiectasis, berylliosis, talcosis, pneumoconiosis, sarcoidosis, desquamative interstitial pneumonia, lymphoid interstitial pneumonia, giant cell interstitial pneumonia, cellular interstitial pneumonia, extrinsic allergic alveolitis, Wegener's granulomatosis and related forms of angiitis (temporal arteritis and polyarteritis nodosa), inflammatory dermatoses, hepatitis, delayed-type hypersensitivity reactions (e.g., poison ivy dermatitis), pneumonia, respiratory tract inflammation, Adult Respiratory Distress Syndrome (ARDS), encephalitis, immediate hypersensitivity reactions, asthma, hayfever, allergies, acute anaphylaxis, rheumatic fever, glomerulonephritis, pyelonephritis, cellulitis, cystitis, chronic cholecystitis, ischemia (ischemic injury), reperfusion injury, allograft rejection, host-versus-graft rejection, appendicitis, arteritis, blepharitis, bronchiolitis, bronchitis, cervicitis, cholangitis, chorioamnionitis, conjunctivitis, dacryoadenitis, dermatomyositis, endocarditis, endometritis, enteritis, enterocolitis, epicondylitis, epididymitis, fasciitis, fibrositis, gastritis, gastroenteritis, gingivitis, ileitis, iritis, laryngitis, myelitis, myocarditis, nephritis, omphalitis, oophoritis, orchitis, osteitis, otitis, pancreatitis, parotitis, pericarditis, pharyngitis, pleuritis, phlebitis, pneumonitis, proctitis, prostatitis, rhinitis, salpingitis, sinusitis, stomatitis, synovitis, testitis, tonsillitis, urethritis, urocystitis, uveitis, vaginitis, vasculitis, vulvitis, vulvovaginitis, angitis, chronic bronchitis, osteomyelitis, optic neuritis, temporal arteritis, transverse myelitis, necrotizing fasciitis, and necrotizing enterocolitis. An ocular inflammatory disease includes, but is not limited to, post-surgical inflammation.

A “protein,” “peptide,” or “polypeptide” comprises a polymer of amino acid residues linked together by peptide bonds. The term refers to proteins, polypeptides, and peptides of any size, structure, or function. Typically, a protein will be at least three amino acids long. A protein may refer to an individual protein or a collection of proteins. Inventive proteins preferably contain only natural amino acids, although non-natural amino acids (i.e., compounds that do not occur in nature but that can be incorporated into a polypeptide chain) and/or amino acid analogs as are known in the art may alternatively be employed. Also, one or more of the amino acids in a protein may be modified, for example, by the addition of a chemical entity such as a carbohydrate group, a hydroxyl group, a phosphate group, a farnesyl group, an isofarnesyl group, a fatty acid group, a linker for conjugation or functionalization, or other modification. A protein may also be a single molecule or may be a multi-molecular complex. A protein may be a fragment of a naturally occurring protein or peptide. A protein may be naturally occurring, recombinant, synthetic, or any combination of these.

The term “therapeutic agent” refers to any substance having therapeutic properties that produce a desired, usually beneficial, effect. For example, therapeutic agents may treat, ameliorate, and/or prevent disease. Therapeutic agents, as disclosed herein, may be biologics or small molecule therapeutics.

The term “E3 ubiquitin ligase” or “E3 ligase” refers to any protein that recruits an E2 ubiquitin-conjugating enzyme that has been loaded with ubiquitin, recognizes a protein substrate, and assists or directly catalyzes the transfer of ubiquitin from the E2 protein to the protein substrate. For E3 ubiquitin ligase, an exemplary sequence from GenBank: ACH72645.1 (Homo sapiens) is: MESGGRPSLC QFILLGTTSV VTAALYSVYR QKARVSQELK GAKKVHLGED LKSILSEAPG KCVPYAVIEG AVRSVKETLN SQFVENCKGV IQRLTLQEHK MVWNRTTHLW NDCSKIIHQR TNTVPFDLVP HEDGVDVAVR VLKPLDSVDL GLETVYEKFH PSIQSFTDVI GHYISGERPK GIQETEEMLK VGATLTGVGE LVLDNNSVRL QPPKQGMQYY LSSQDFDSLL QRQESSVRLW KVLALVFGFA TCATLFFILR KQYLQRQERL RLKQMQEEFQ EHEAQLLSRA KPEDRESLKS ACVVCLSSFK SCVFLECGHV CSCTECYRAL PEPKKCPICR QAITRVIPPY NS (SEQ ID NO: 1). For E3 ubiquitin ligase, another exemplary sequence from GenBank is: AAP47175.1 (Homo sapiens), which is: MEEGNNNEEV IHLNNFHCHR GQEWINLRDG PITISDSSDE ERIPMLVTPA PQQHEEEDLD DDVILTETNK PQRSRPNLIK PAAQWQDLKR LGEERPKKSR AAFESDKSSY FSVCNNPLFD SGAQDDSEDD YGEFLDLGPP GISEFTKPSG QTEREPKPGP SHNQAANDIV NPRSEQKVII LEEGSLLYTE SDPLETQNQS SEDSETELLS NLGESAALAD DQAIEEDCWL DHPYFQSLNQ QPREITNQVV PQERQPEAEL GRLLFQHEFP GPAFPRPEPQ QGGISGPSSP QPAHPLGEFE DQQLASDDEE PGPAFPMQES QEPNLENIWG QEAAEVDQEL VELLVKETEA RFPDVANGFI EEIIHFKNYY DLNVLCNFLL ENPDYPKRED RIIINPSSSL LASQDETKLP KIDFFDYSKL TPLDQRCFIQ AADLLMADFK VLSSQDIKWA LHELKGHYAI TRKALSDAIK KWQELSPETS GKRKKRKQMN QYSYIDFKFE QGDIKIEKRM FFLENKRRHC RSYDRRALLP AVQQEQEFYE QKIKEMAEHE DFLLALQMNE EQYQKDGQLI ECRCCYGEFP FEELTQCADA HLFCKECLIR YAQEAVFGSG KLELSCMEGS CTCSFPTSEL EKVLPQTILY KYYERKAEEE VAAAYADELV RCPSCSFPAL LDSDVKRFSC PNPHCRKETC RKCQGLWKEH NGLTCEELAE KDDIKYRTSI EEKMTAARIR KCHKCGTGLI KSEGCNRMSC RCGAQMCYLC RVSINGYDHF CQHPRSPGAP CQECSRCSLW TDPTEDDEKL IEEIQKEAEE EQKRKNGENT FKRIGPPLEK PVEKVQRVEA LPRPVPQNLP QPQMPPYAFA HPPFPLPPVR PVFNNFPLNM GPIPAPYVPP LPNVRVNYDF GPIHMPLEHN LPMHFGPQPR HRF (SEQ ID NO: 2). For E3 ubiquitin ligase, another exemplary sequence from GenBank is: AAP47174.1 (Homo sapiens) which is:

(SEQ ID NO: 3)
MEEGNNNEEV IHLNNFHCHR GQEWINLRDG PITISDSSDE
ERIPMLVTPA PQQHEEEDLD DDVILTEDDS EDDYGEFLDL
GPPGISEFTK PSGQTEREPK PGPSHNQAAN DIVNPRSEQK
VIILEEGSLL YTESDPLETQ NQSSEDSETE LLSNLGESAA
LADDQAIEED CWLDHPYFQS LNQQPREITN QVVPQERQPE
AELGRLLFQH EFPGPAFPRP EPQQGGISGP SSPQPAHPLG
EFEDQQLASD DEEPGPAFPM QESQEPNLEN IWGQEAAEVD
QELVELLVKE TEARFPDVAN GFIEEIIHFK NYYDLNVLCN
FLLENPDYPK REDRIIINPS SSLLASQDET KLPKIDFFDY
SKLTPLDQRC FIQAADLLMA DFKVLSSQDI KWALHELKGH
YAITRKALSD AIKKWQELSP ETSGKRKKRK QMNQYSYIDF
KFEQGDIKIE KRMFFLENKR RHCRSYDRRA LLPAVQQEQE
FYEQKIKEMA EHEDFLLALQ MNEEQYQKDG QLIECRCCYG
EFPFEELTQC ADAHLFCKEC LIRYAQEAVF GSGKLELSCM
EGSCTCSFPT SELEKVLPQT ILYKYYERKA EEEVAAAYAD
ELVRCPSCSF PALLDSDVKR FSCPNPHCRK ETCRKCQGLW
KEHNGLTCEE LAEKDDIKYR TSIEEKMTAA RIRKCHKCGT
GLIKSEGCNR MSCRCGAQMC YLCRVSINGY DHFCQHPRSP
GAPCQECSRC SLWTDPTEDD EKLIEEIQKE AEEEQKRKNG
ENTFKRIGPP LEKPVEKVQR VEALPRPVPQ NLPQPQMPPY
AFAHPPFPLP PVRPVFNNFP LNMGPIPAPY VPPLPNVRVN
YDFGPIHMPL EHNLPMHFGP QPRHRF.

The term “binder” refers to a compound that binds to a protein. The binder binds to a protein with a K_{d} of less than 50,000 nM, less than 20,000 nM, less than 10,000 nM, less than 5,000 nM, less than 2,500 nM, less than 1,000 nM, less than 900 nM, less than 800 nM, less than 700 nM, less than 600 nM, less than 500 nM, less than 400 nM, less than 300 nM, less than 200 nM, less than 100 nM, less than 90 nM, less than 80 nM, less than 70 nM, less than 60 nM, less than 50 nM, less than 40 nM, less than 30 nM, less than 20 nM, less than 10 nM, less than 5 nM, less than 4 nM, less than 3 nM, less than 2 nM, or less than 1 nM.

The term “proteasome” refers to a protease complex for carrying out degradation of proteins. Specifically, the proteasome is a multisubunit enzyme complex, which can also play a key role regulating proteins that control cell-cycle progression and apoptosis. The proteasome conducts proteolysis of selected proteins.

The term “HCK” refers to a hematopoietic cell kinase. Hematopoietic cell kinase (HCK) is a member of the SRC family of cytoplasmic tyrosine kinases (SFK's), and is expressed in cells of the myeloid and B-lymphocyte cell lineages. HCK is a downstream target of mutated MYD88, is activated by IL-6, and triggers pro-survival signaling including PI3K/AKT, MAPK/ERK, and BTK in MYD88-mutated cells. HCK is thus a target for therapeutic development in diseases associated with MYD88-mutatations including cancers, such as, but not limited to, non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), cancers associated with HCK, and other diseases associated with mutated MYD88. In humans, HCK comprises the p61HCK and p59HCK isoforms consisting of 525 amino acids and 504 amino acids, respectively. In mice, HCK comprises p59HCK consisting of 503 amino acids and p56HCK consisting of 482 amino acids.

The term “VHL” refers to von Hippel-Lindau protein. Von Hippel-Lindau protein (VHL) is a substrate recognition subunit of an E3 ligase and plays a role in regulating cell growth. VHL is a component of the protein complex which includes elongin B, elongin C, and cullin-2, and possesses ubiquitin ligase E3 activity. VHL is involved in the ubiquitination and degradation of hypoxia-inducible-factor (HIF), where HIF is a transcription factor that plays a significant role in regulating gene expression relating to oxygen levels. Thus, VHL is a target for therapeutic development in proliferative diseases including cancer, and inflammatory diseases (e.g., anemia, and ischemia). For VHL, an exemplary sequence from GenBank: is: AAB64200.1 (Homo sapiens).

The term “BTK” refers to Bruton's tyrosine kinase. Bruton's tyrosine kinase (BTK) is a cytoplasmic tyrosine kinase with important functions in B-lymphocyte (B-cell) development, B-cell differentiation, and B-cell signaling. Accordingly, BTK is an important target in oncology therapy, for example, but not limited to, treating B-cell malignancies (e.g., leukemia, Waldenström's Macroglobulinemia (e.g., Myd88-associated diseases), activated B-cell (ABC) lymphoma, diffuse large B-cell lymphoma (DLBCL)) and/or solid tumors. For BTK, an exemplary sequence from GenBank is: 565324225 (Homo sapiens). For BTK, another exemplary sequence from GenBank is: 4557377 (Homo sapiens). For BTK, another exemplary sequence from GenBank is: 565324227 (Homo sapiens).

### BRIEF DESCRIPTION OF THE DRAWINGS

FIGS. 1A-1B show EC₅₀ values (molecular concentration) in MYD88-mutated Waldenstrom macroglobulinemia (WM) cell lines (BCWM.1, MWCL-1), ABC DLBCL cell lines (TMD-8, HBL-1), and MYD88 wild-type GCB DLBCL cell lines (OCI-Ly7, OCI-Ly19), Burkitt's lymphoma cell line (Ramos), as well as multiple myeloma cell line (RPMI-8226). FIG. 1A shows the EC₅₀ values for compound A419259-based degraders. FIG. 1B shows the EC₅₀ values for compound SB1-G-112-based degraders. Compound A419259 is of the formula:

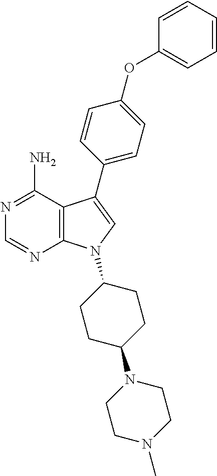 [SMILES: CN1CCN([C@H]2CC[C@H](n3cc(-c4ccc(Oc5ccccc5)cc4)c4c(N)ncnc43)CC2)CC1]

and compound SB1-G-112 is of the formula:

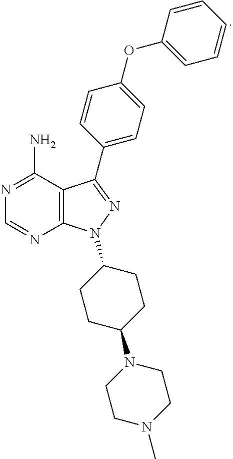 [SMILES: CN1CCN([C@H]2CC[C@H](n3nc(-c4ccc(Oc5ccccc5)cc4)c4c(N)ncnc43)CC2)CC1]

FIGS. 2A-2D show the dose response curves for A419259 based degraders in MYD88 mutated WM cell lines (BCWM.1, MWCL-1), ABC DLBCL cell lines (TMD-8, HBL-1), and MYD88 wild-type GCB DLBCL cell lines (OCI-Ly7, OCI-Ly19), Burkitt's lymphoma cell line (Ramos), as well as multiple myeloma cell line (RPMI-8226). FIG. 2A shows the dose response curves for A419259 based degraders. FIG. 2B shows the dose response curves for SB1-G-175-P1 (SB1-G-175)-based degraders. FIG. 2C shows the dose response curves for SB1-G-176-P1 (SB1-G-176)-based degraders. FIG. 2D shows the dose response curves for SB1-G-177-P1 (SB1-G-177)-based degraders.

FIGS. 3A-3G show the dose response curves for SB1-G-112 based degraders in MYD88 mutated WM cell lines (BCWM.1, MWCL-1), ABC DLBCL cell lines (TMD-8, HBL-1), and MYD88 wild-type GCB DLBCL (a type of activated B-cell (ABC) lymphoma, diffuse large B-cell lymphoma (DLBCL)) cell lines (OCI-Ly7, OCI-Ly19), Burkitt's lymphoma cell line (Ramos), as well as multiple myeloma cell line (RPMI-8226). GCB DLBCL is a type of activated B-cell (ABC) lymphoma, diffuse large B-cell lymphoma (DLBCL). FIG. 3A shows the dose response curves for SB1-G-112-P1 based degraders. FIG. 3B shows the dose response curves for SB1-G-181-P1 based degraders. FIG. 3C shows the dose response curves for SB1-G-182-P1 based degraders. FIG. 3D shows the dose response curves for SB1-G-185-P1 based degraders. FIG. 3E shows the dose response curves for SB1-G-200-P1 based degraders. FIG. 3F shows the dose response curves for SB1-G-212-P1 based degraders. FIG. 3G shows the dose response curves for SB1-G-214-P1 based degraders. Compounds SB1-G-181-P1 (SB1-G-181), SB1-G-182-P1 (SB1-G-182), SB1-G-183-P1 (SB1-G-183), SB1-G-184-P1 (SB1-G-184), SB1-G-185-P1 (SB1-G-185), SB1-G-186-P1 (SB1-G-186), SB1-G-199-P1 (SB1-G-199), SB1-G-200-P1 (SB1-G-200), SB1-G-212-P1 (SB1-G-212), SB1-G-213-P1 (SB1-G-213), and SB1-G-214-P1 (SB1-G-214) were assayed in FIGS. 3B-3G.

FIG. 4 shows the protein degradation assessments for HCK and BTK by Western blot following treatment of MYD88 mutated BCWM.1 cells with SB1-G-112 compound-based HCK degraders (SB1-G-181-P1 (SB1-G-181), SB1-G-182-P1 (SB1-G-182), SB1-G-185-P1 (SB1-G-185)) for 6 hours. SK-6-1 was used as a degrader control compound. Exemplary compounds SB1-G-181-P1, SB1-G-182-P1, and SB1-G-185-P1 showed effective degradation of HCK and BTK in BCWM.1 cells.

FIG. 5 shows the protein degradation assessments for HCK and BTK by Western blot following treatment using vector only, BTK wild type (BTK^{WT}), or BTK^{C481S} mutant (BTK^{C481S}) transduced BCWM.1 WM cells with SB1-G-112 based HCK degraders (SB1-G-185-P1) for 6 hours. SK-6-1 was used as a degrader control compound. SB1-G-185-P1 showed effective degradation of HCK in all cell lines, and blocked BTK phosphorylation at Tyr-223, even the degradation of BTK was diminished by BTK^{C481S} mutation.

FIG. 6 shows protein degradation assessments for HCK and BTK by Western blot following treatment using vector only, BTK wild type (BTK^{WT}), or BTK^{C481S} mutant (BTK^{C481S}) transduced TMD8 ABC-DLBCL cells with SB1-G-112 based HCK degraders (SB1-G-185-P1) for 6 hours. SK-6-1 was used as a degrader control compound. Exemplary compound SB1-G-185-P1 showed effective degradation of HCK and BTK in all cell lines, and blocked BTK phosphorylation at Tyr-223. “ABC-DLBCL” is activated B-cell (ABC) lymphoma, diffuse large B-cell lymphoma (DLBCL).

FIGS. 7A-7B show proteomics data on the degradation of target kinases in cell line MOLT4 (human T lymphoblast; acute lymphoblastic leukemia) using treatment with exemplary compounds SB1-G-181 and SB1-G-200. FIG. 7A indicates in the bottom left corner the exemplary kinase targets (FYN, SRC, BLK, LCK, CSK, ABL1, ABL2, LIMK1, LATS1) in cell line MOLT4 which were degraded by exemplary compound SB1-G-181. FIG. 7B indicates in the bottom left corner the exemplary kinase targets (FYN, SRC, BLK, LCK, CSK, ABL1, ABL2, LIMK1, LATS1) in cell line MOLT4 which were degraded by exemplary compound SB1-G-200.

### DETAILED DESCRIPTION OF CERTAIN EMBODIMENTS OF THE INVENTION

The bifunctional compounds described herein interact with a kinase, e.g., HCK, BTK, FYN, SRC, BLK, LCK, CSK, ABL1, ABL2, LIMK1, LATS1) and an E3 ubiquitin ligase (e.g., Cereblon). As described herein, without wishing to be bound by any particular theory, the therapeutic effect may be the result of degradation, modulation, inhibition, or binding of a kinase (e.g., HCK, BTK, FYN, SRC, BLK, LCK, CSK, ABL1, ABL2, LIMK1, LATS1) by a compound described herein. The therapeutic effect may be a result of the bifunctional compound, which includes a binder of an E3 ubiquitin ligase (e.g., Cereblon) and a binder of a target (e.g., kinase (e.g., HCK, BTK, FYN, SRC, BLK, LCK, CSK, ABL1, ABL2, LIMK1, LATS1)), thereby inducing the degradation of the target kinase. The compounds may be used to induce degradation of the kinase (e.g., HCK, BTK, FYN, SRC, BLK, LCK, CSK, ABL1, ABL2, LIMK1, LATS1), for treating and/or preventing diseases (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations), for treating and/or preventing diseases associated with the kinase (e.g., HCK, BTK, FYN, SRC, BLK, LCK, CSK, ABL1, ABL2, LIMK1, LATS1). The compounds may be used to induce degradation of the kinase (e.g., HCK, BTK), for treating and/or preventing diseases (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations), for treating and/or preventing diseases associated with the kinase (e.g., HCK, BTK).

In one aspect, disclosed are compounds of Formula (I):

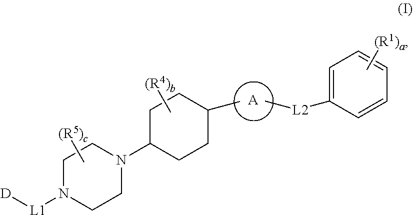

or a pharmaceutically acceptable salt, co-crystal, tautomer, stereoisomer, solvate, hydrate, polymorph, isotopically enriched derivative, or prodrug thereof, wherein:

each instance of R¹ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;

R^{D1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;

each occurrence of R^{D1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;

each instance of R² is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;

each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;

each instance of R⁴ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;

each instance of R⁵ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;

L1 is a linker;

L2 is a bond or

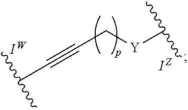

Ring A is of formula:

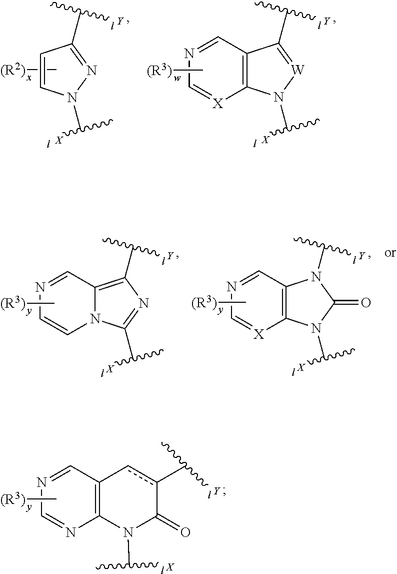

and

[symbol] is a single bond or a double bond, as valency permits;

W is ═C(R^{A})— or ═N—;

X is ═C(R^{A})— or ═N—;

Y is O, —N(R^{Y})—, or S;

each instance of R^{A} is independently hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A1}, —N(R^{A1a})₂ or —SR^{A1}; R^{A1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;

each occurrence of R^{A1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{A1a} are taken together with their intervening atoms to form a substituted or unsubstituted heterocyclic or substituted or unsubstituted heteroaryl ring;

R^{Y} is hydrogen, optionally substituted acyl, optionally substituted alkyl, optionally substituted alkenyl, optionally substituted alkynyl, or a nitrogen protecting group;

a is 0, 1, 2, 3, 4, or 5;

b is 0, 1, 2, 3, 4, 5, 6, 7, 8, 9, or 10;

c is 0, 1, 2, 3, 4, 5, 6, 7, or 8;

p is 0, 1, 2, or 3;

w is 0, 1, 2, 3, or 4;

x is 0, 1, or 2;

y is 0, 1, 2, or 3;

D is an E3 ubiquitin ligase binding moiety;

l^{X} indicates the point of attachment to the moiety of formula

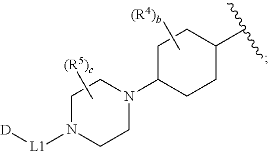

l^{Y} indicates the point of attachment to L2

l^{W} indicates the point of attachment to Ring A; and l^{Z} indicates the point of attachment to the moiety of formula

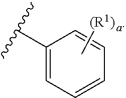

### Moiety D

Formula (I) includes moiety D. In certain embodiments, D is an E3 ubiquitin ligase binding moiety. D includes all moieties that bind, or can bind, any E3 ubiquitin ligase. For example, in certain embodiments, D is capable of binding an E3 ubiquitin ligase, such as Cereblon. In certain embodiments, D is capable of binding to multiple different E3 ubiquitin ligases. In certain embodiments, D binds to Cereblon. In certain embodiments, D is based on an immunomodulatory imide drug. In certain embodiments, D comprises or is derived from lenalidomide. In certain embodiments, D comprises or is derived from thalidomide.

Human Cereblon (CRBN) is a protein of 442 amino acids with an apparent molecular weight of ˜51 kDa (GenBank: AAH17419). (For the CRBN protein sequence see: Higgins et al., Neurology. 2004, 63, 1927-31. For additional information related to the CRBN structure, see Hartmann et al., PLoS One. 2015, 10, e0128342.) Human CRBN contains the N-terminal part (237-amino acids from 81 to 317) of ATP-dependent Lon protease domain without the conserved Walker A and Walker B motifs, 11 casein kinase II phosphorylation sites, 4 protein kinase C phosphorylation sites, 1 N-linked glycosylation site, and 2 myristoylation sites. CRBN is widely expressed in testis, spleen, prostate, liver, pancreas, placenta, kidney, lung, skeletal muscle, ovary, small intestine, peripheral blood leukocyte, colon, brain, and retina. CRBN is located in the cytoplasm, nucleus, and peripheral membrane. (Chang et al., Int. J. Biochem. Mol. Biol. 2011, 2, 287-94.)

Cereblon is an E3 ubiquitin ligase, and it forms an E3 ubiquitin ligase complex with damaged DNA binding protein 1 (DDB1), Cullin-4A (CUL4A), and regulator of cullins 1 (ROC1). This complex ubiquitinates a number of other proteins. Through a mechanism which has not been completely elucidated, Cereblon ubiquitination of target proteins results in increased levels of fibroblast growth factor 8 (FGF8) and fibroblast growth factor 10 (FGF10). FGF8, in turn, regulates a number of developmental processes, such as limb and auditory vesicle formation.

In certain embodiments, D is a modulator, binder, inhibitor, or ligand of Cereblon. In certain embodiments, D is a modulator of Cereblon. In certain embodiments, D is a binder of Cereblon. In certain embodiments, D is an inhibitor of Cereblon. In certain embodiments, D is a ligand of Cereblon. In certain embodiments, D is any modulator, binder, inhibitor, or ligand of Cereblon disclosed in U.S. patent application U.S. Ser. No. 14/792,414, filed Jul. 6, 2015, published as U.S. Patent Application Publication No. 2016-0058872, on Mar. 3, 2016; U.S. patent application U.S. Ser. No. 14/707,930, filed May 8, 2015, issued as U.S. Pat. No. 9,694,084 on Jul. 4, 2017; and International Patent Application, PCT/US2013/054663, filed Aug. 13, 2013, published as International Patent Application Publication No. WO 2014/02844, on Feb. 20, 2014; each of which is incorporated herein by reference. In certain embodiments, D has a binding affinity (K_{d}) to Cereblon of below 20 μM. In certain embodiments, D has a binding affinity (K_{d}) to Cereblon of below 15 μM. In certain embodiments, D has a K_{d} to Cereblon of below 10 μM. In certain embodiments, D has a K_{d} to Cereblon of below 5 μM. In certain embodiments, D has a binding affinity (K_{d}) to Cereblon of about 1-10 μM. In certain embodiments, D has a K_{d} to Cereblon of about 3 μM. In certain embodiments, D has a binding affinity (K_{d}) to Cereblon as disclosed in U.S. patent application U.S. Ser. No. 14/792,414, filed Jul. 6, 2015, published as U.S. Patent Application Publication No. 2016-0058872, on Mar. 3, 2016, which is incorporated herein by reference.

In certain embodiments, D is a modulator, binder, inhibitor, or ligand of a Cereblon variant. In certain embodiments, D is a modulator, binder, inhibitor, or ligand of a Cereblon isoform.

In certain embodiments, D comprises an optionally substituted heteroaryl ring. In certain embodiments, D comprises an optionally substituted fused bicyclic heteroaryl ring. In certain embodiments, D comprises an optionally substituted fused bicyclic heteroaryl ring and a heterocyclic ring. In certain embodiments, D comprises an optionally substituted fused bicyclic heteroaryl ring and a heterocyclic ring, where the heterocyclic ring contains at least one nitrogen. In certain embodiments, D comprises an optionally substituted fused bicyclic heteroaryl ring and a heterocyclic ring, where the fused bicyclic heteroaryl ring and heterocyclic ring each contain at least one nitrogen. In certain embodiments, D comprises an optionally substituted fused bicyclic heteroaryl ring and a heterocyclic ring, where the fused bicyclic heteroaryl ring and heterocyclic ring each contain one nitrogen. In certain embodiments, D comprises an optionally substituted phthalimido group, or an analogue or derivative thereof. In certain embodiments, D comprises an optionally substituted phthalimido-glutarimide group, or an analogue or derivative thereof.

In certain embodiments, D is of Formula (E-I):

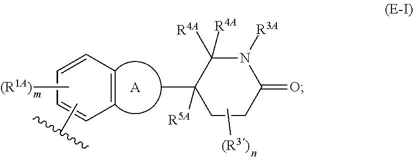

wherein:

Ring A is a substituted or unsubstituted heterocyclyl, or substituted or unsubstituted heteroaryl ring;

each R^{1A} is, independently, halogen, —OH, C₁-C₆ alkyl, or C₁-C₆ alkoxy;

each R^{3A} is, independently, H or C₁-C₃ alkyl;

each R^{Y} is, independently, C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, F, or Cl;

m is 0, 1, 2, or 3; and

n is 1 or 2.

In certain embodiments, Formula (E-I) is an immunomodulatory imide drug (e.g., lenalidomide or thalidomide). In certain embodiments, Formula (E-I) comprises an immunomodulatory imide drug (e.g., lenalidomide or thalidomide). In certain embodiments, Formula (E-I) is derived from an immunomodulatory imide drug (e.g., lenalidomide or thalidomide). In certain embodiments, Formula (E-I) is of Formula (IA) or Formula (IB), below. In certain embodiments, the compounds of Formula (IA) or Formula (IB) are optionally further substituted.

In certain embodiments, D is of Formula (IA):

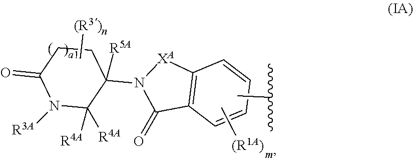

wherein:

X^{A} is C(O) or C(R^{3A})₂;

each R^{1A} is independently halogen, —OH, C₁-C₆ alkyl, or C₁-C₆ alkoxy;

R^{3A} is H or C₁-C₃ alkyl;

each R^{3′} is independently C₁-C₃ alkyl;

each R^{1A} is independently H or C₁-C₃ alkyl; or two R⁴, together with the carbon atom to which they are attached, form a C(O), C₃-C₆carbocycle, or a 4-, 5-, or 6-membered heterocycle comprising 1 or 2 heteroatoms selected from N and O;

R^{1A} is H, C₁-C₃ alkyl, or halogen;

m is 0, 1, 2, or 3;

n is 0, 1 or 2; and

a1 is 0 or 1.

In certain embodiments, D is of Formula (IA-a):

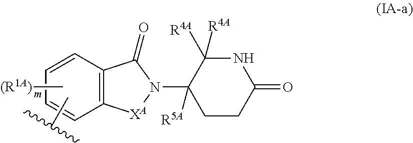

wherein:

X^{A} is C(O) or C(R^{3A})₂;

each R^{1A} is, independently, halogen, —OH, C₁-C₆ alkyl, or C₁-C₆ alkoxy;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, F, or Cl; and

m is 0, 1, 2, or 3.

In certain embodiments, D is of Formula (IA-b):

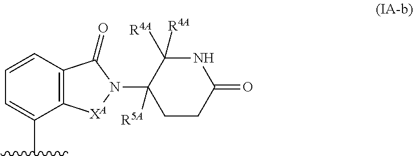

wherein:

X^{A} is C(O) or C(R^{3A})₂;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is H, C₁-C₃ alkyl, F, or Cl.

In certain embodiments, D is of Formula (IA-c):

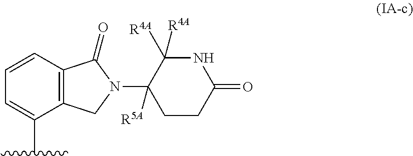

wherein:

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is H, C₁-C₃ alkyl, F, or Cl.

In certain embodiments, D is of Formula (IA-d):

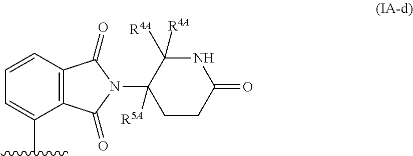

wherein:

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O; and

R^{5A} is H, C₁-C₃ alkyl, F, or Cl.

In certain embodiments, D is of Formula (IB):

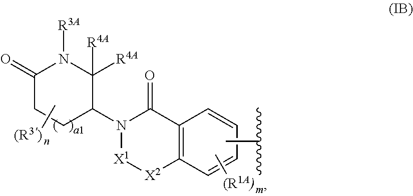

wherein:

—X¹—X²— is C(R^{3A})═N or C(R^{3A})₂—C(R^{3A})₂;

each R^{1A} is independently halogen, —OH, C₁-C₆ alkyl, or C₁-C₆ alkoxy;

R^{3A} is H or C₁-C₃ alkyl;

each R^{3′} is independently C₁-C₃ alkyl;

each R^{4A} is independently H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, or halogen;

m is 0, 2, or 3;

n is 0, 1, or 2; and

a1 is 0 or 1.

In certain embodiments, D is of Formula (IB-a):

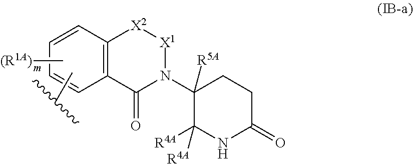

wherein:

X¹—X² is C(R^{3A})═N or C(R^{3A})₂—C(R^{3A})₂;

each R^{1A} is, independently, halogen, —OH, C₁-C₆ alkyl, or C₁-C₆ alkoxy;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, F, or Cl; and

m is 0, 1, 2, or 3.

In certain embodiments, D is of Formula (IB-b):

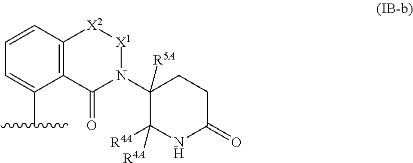

wherein:

X¹—X² is C(R^{3A})═N or C(R^{3A})₂—C(R^{3A})₂;

each R^{3A} is, independently, H or C₁-C₃ alkyl;

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, F, or Cl.

In certain embodiments, D is of Formula (IB-c):

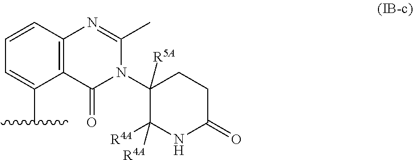

wherein:

each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;

R^{5A} is H, C₁-C₃ alkyl, F, or Cl.

Formula (IA), (IA-a), and (IA-b) include substituent X^{A}. In certain embodiments, X^{A} is C(O). In certain embodiments, X^{A} is C(R^{3A})₂. In certain embodiments, R^{3A} is hydrogen. In certain embodiments, X^{A} is —CH₂—.

Formula (IB), (IB-a), and (IB-b) include substituents —X¹—X²—. In certain embodiments, —X¹—X²— is —C(R^{3A})═N—. In certain embodiments, —X¹—X²— is —C(H)═N—. In certain embodiments, —X¹—X²— is —C(C₁-C₃ alkyl)═N—. In certain embodiments, —X¹—X²— is —C(R^{3A})₂—C(R^{3A})₂—. In certain embodiments, —X¹—X²— is —C(H)₂—C(H)₂—. In certain embodiments, —X¹—X²— is —C(H)₂—C(C₁-C₃ alkyl)₂-. In certain embodiments, —X¹—X²— is —C(H)₂—C(C₁-C₃ alkyl)₂-. In certain embodiments, —X¹—X²— is —C(H)₂—C(C₁-C₃ alkyl)₂-. In certain embodiments, —X¹—X²— is —C(C₁-C₃ alkyl)₂-C(C₁-C₃ alkyl)₂-. In certain embodiments, a1 is 0. In certain embodiments, a1 is 1.

In certain embodiments, each R^{4A} is, independently, H or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O. In certain embodiments, at least one instance of R^{4A} is hydrogen. In certain embodiments, both instances of R^{4A} are hydrogen. In certain embodiments, at least one instance of R^{4A} is C₁-C₃ alkyl. In certain embodiments, two R^{4A}, together with the carbon atom to which they are attached, form a C(O). In certain embodiments, there are zero instances of R^{1A} on the compound of Formula (IA) or (IB). In certain embodiments, there are one or more instances of R^{1A} on the compound of Formula (IA) or (IB). In certain embodiments, m is 0. In certain embodiments, m is 1. In certain embodiments, m is 2. In certain embodiments, m is 3.

In certain embodiments, there are zero instances of R^{3′} on the compound of Formula (IA) or (IB). In certain embodiments, there are one or more instances of R^{3′} on the compound of Formula (IA) or (IB). In certain embodiments, n is 0. In certain embodiments, n is 1. In certain embodiments, n is 2. In certain embodiments, D is of the formula:

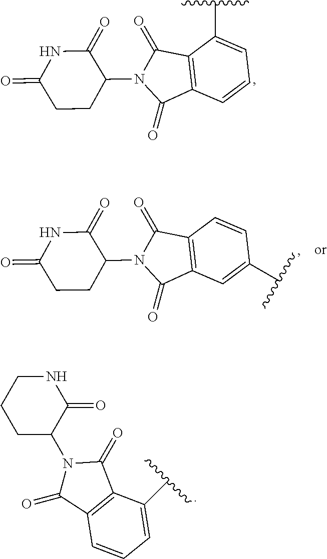 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1C(=O)N(C1CCCNC1=O)C2=O]

In certain embodiments, D is of the formula:

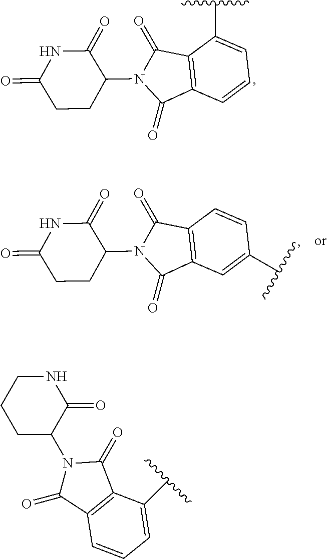 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1C(=O)N(C1CCCNC1=O)C2=O]

and binds an E3 ligase (e.g., Cereblon).

In certain embodiments, D is of formula:

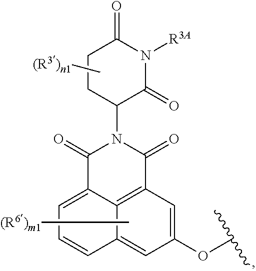

wherein:

R^{3A} is hydrogen or C₁-C₃ alkyl;

each R^{3′} is independently C₁-C₃ alkyl;

each R^{6′} is independently halogen, —OH, C₁-C₆ alkyl, or C₁-C₆ alkoxy;

n1 is 0, 1, 2, 3, 4, or 5; and

m1 is 0, 1, 2, 3, 4, or 5.

In certain embodiments, R^{3A} is hydrogen. In certain embodiments, R^{3A} is C₁-C₃ alkyl (e.g., methyl, ethyl, propyl). In certain embodiments, at least one instance of R^{3′} is C₁-C₃ alkyl (e.g., methyl, ethyl, propyl). In certain embodiments, at least one instance of R^{6′} is halogen (e.g., F, Cl, Br, or I). In certain embodiments, at least one instance of R^{6′} is —OH. In certain embodiments, at least one instance of R^{6′} is C₁-C₆ alkyl (e.g., methyl, ethyl, propyl). In certain embodiments, at least one instance of R^{6′} is C₁-C₆ alkoxy (e.g., —O(methyl), —O(ethyl), —O(propyl)).

In certain embodiments, there are zero instances of R^{3′} on the compound of formula:

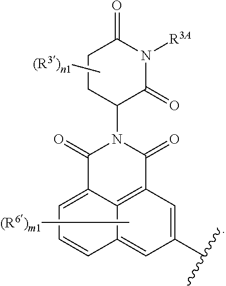

In certain embodiments, there are one or more instances of R^{3′} on the compound of formula:

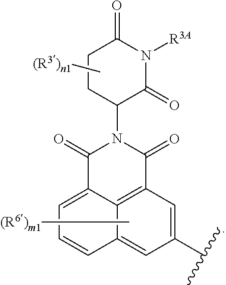

In certain embodiments, there are zero instances of R^{6′} on the compound of formula:

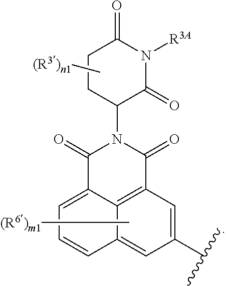

In certain embodiments, there are one or more instances of R^{6′} on the compound of formula:

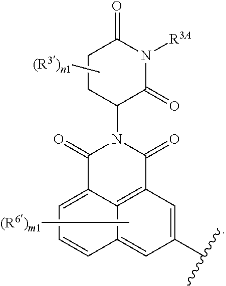

In certain embodiments, n1 is 0. In certain embodiments, n1 is 1. In certain embodiments, n1 is 2. In certain embodiments, n1 is 3. In certain embodiments, n1 is 4. In certain embodiments, n1 is 5. In certain embodiments, m1 is 0. In certain embodiments, m1 is 1. In certain embodiments, m1 is 2. In certain embodiments, m1 is 3. In certain embodiments, m1 is 4. In certain embodiments, m1 is 5. In certain embodiments, m1 is 0 and n1 is 0. In certain embodiments, D is of formula:

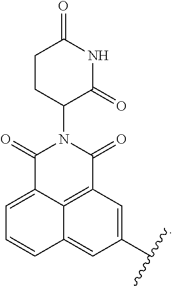 [SMILES: *c1cc2c3c(cccc3c1)C(=O)N(C1CCC(=O)NC1=O)C2=O]

In certain embodiments, D is a compound based on a ligand that binds to von Hippel-Lindau protein (a “VHL ligand”). In certain embodiments, D is derived from a VHL ligand. In certain embodiments, D is a VHL ligand disclosed in or is derived from a VHL ligand disclosed in U.S. patent application U.S. Ser. No. 16/224,088, filed Dec. 18, 2018, published as U.S. Patent Application Publication No. 2019-0127359, on May 2, 2019, which is a continuation of U.S. patent application U.S. Ser. No. 14/371,956, published as U.S. Patent Application Publication No. 2014-0356322, on Dec. 4, 2014, which claims priority to International Patent Application, PCT/US2013/021136, filed Jan. 11, 2013, published as International Patent Application Publication No. WO 2013/106643, on Jul. 18, 2013, which are each incorporated herein by reference. In certain embodiments, D is of the formula:

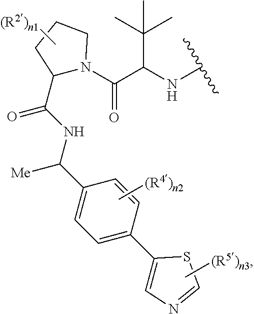

wherein:

each R^{2′} is independently halogen, —OH, C₁-C₆ alkyl, or C₁-C₆ alkoxy;

each R^{4′} is independently halogen, —OH, C₁-C₆ alkyl, or C₁-C₆ alkoxy;

each R^{5′} is independently halogen, —OH, C₁-C₆ alkyl, or C₁-C₆ alkoxy;

n1′ is 0, 1, 2, 3, 4, 5, or 6;

n2′ is 0, 1, 2, 3, or 4; and

n3′ is 0, 1, or 2.

In certain embodiments, D has zero instances of R^{2′}. In certain embodiments, D has one or more instances of R^{2′}. In certain embodiments, n1′ is 0. In certain embodiments, n1′ is 1. In certain embodiments, n1′ is 2. In certain embodiments, n1′ is 3. In certain embodiments, n1′ is 4. In certain embodiments, n1′ is 5. In certain embodiments, n1′ is 6. In certain embodiments, each instance of R^{2′} is independently halogen, —OH, C₁-C₆ alkyl, or C₁-C₆ alkoxy. In certain embodiments, at least one instance of R^{2′} is halogen (e.g., F, Cl, Br, or I). In certain embodiments, at least one instance of R^{2′} is —OH. In certain embodiments, at least one instance of R^{2′} is unsubstituted C₁-C₆ alkyl (e.g., unsubstituted methyl, unsubstituted ethyl, or unsubstituted n-propyl). In certain embodiments, at least one instance of R^{2′} is C₁-C₆ alkoxy (e.g., —O(unsubstituted C₁-C₆ alkyl)). In certain embodiments, at least one instance of R^{2′} is —O(Me). In certain embodiments, at least one instance of R^{2′} is —O(Et). In certain embodiments, at least one instance of R^{2′} is —O(n-propyl). In certain embodiments, at least one instance of R^{2′} is —O(isopropyl). In certain embodiments, at least one instance of R^{2′} is —O(n-butyl).

In certain embodiments, D has zero instances of R^{4′}. In certain embodiments, D has one or more instances of R^{4′}. In certain embodiments, n2′ is 0. In certain embodiments, n2′ is 1. In certain embodiments, n2′ is 2. In certain embodiments, n2′ is 3. In certain embodiments, n2′ is 4. In certain embodiments, each instance of R^{4′} is independently halogen, —OH, C₁-C₆alkyl, or C₁-C₆ alkoxy. In certain embodiments, at least one instance of R^{4′} is halogen (e.g., F, Cl, Br, or I). In certain embodiments, at least one instance of R^{2′} is —OH. In certain embodiments, at least one instance of R^{4′} is unsubstituted C₁-C₆ alkyl (e.g., unsubstituted methyl, unsubstituted ethyl, or unsubstituted n-propyl). In certain embodiments, at least one instance of R^{4′} is C₁-C₆ alkoxy (e.g., —O(unsubstituted C₁-C₆ alkyl)). In certain embodiments, at least one instance of R^{4′} is —O(Me). In certain embodiments, at least one instance of R^{4′} is —O(Et). In certain embodiments, at least one instance of R^{4′} is —O(n-propyl). In certain embodiments, at least one instance of R^{4′} is —O(isopropyl). In certain embodiments, at least one instance of R^{4′} is —O(n-butyl).

In certain embodiments, D has zero instances of R^{5′}. In certain embodiments, D has one or more instances of R^{5′}. In certain embodiments, n3′ is 0. In certain embodiments, n3′ is 1. In certain embodiments, n3′ is 2. In certain embodiments, n3′ is 3. In certain embodiments, each instance of R^{5′} is independently halogen, —OH, C₁-C₆ alkyl, or C₁-C₆ alkoxy. In certain embodiments, at least one instance of R^{5′} is halogen (e.g., F, Cl, Br, or I). In certain embodiments, at least one instance of R^{5′} is —OH. In certain embodiments, at least one instance of R^{5′} is C₁-C₆ alkyl. In certain embodiments, at least one instance of R^{5′} is unsubstituted C₁-C₆ alkyl (e.g., unsubstituted methyl, unsubstituted ethyl, or unsubstituted n-propyl). In certain embodiments, at least one instance of R^{5′} is unsubstituted methyl. In certain embodiments, at least one instance of R^{5′} is unsubstituted ethyl. In certain embodiments, at least one instance of R^{5′} is unsubstituted n-propyl. In certain embodiments, at least one instance of R^{5′} is C₁-C₆ alkoxy (e.g., —O(unsubstituted C₁-C₆ alkyl)). In certain embodiments, at least one instance of R^{5′} is —O(Me). In certain embodiments, at least one instance of R^{5′} is —O(Et). In certain embodiments, at least one instance of R^{5′} is —O(n-propyl). In certain embodiments, at least one instance of R^{5′} is —O(isopropyl). In certain embodiments, at least one instance of R^{5′} is —O(n-butyl).

In certain embodiments, D is of the formula:

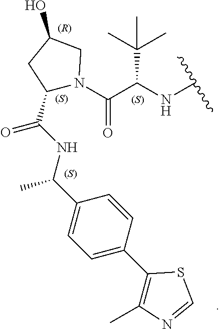

Substituents R¹, R⁴, and R⁵

Formula (I) includes zero or more instances of substituent R¹ on the phenyl ring. In certain embodiments, Formula (I) includes one instance of substituent R¹ on the phenyl ring. In certain embodiments, a is 0. In certain embodiments, a is 1. In certain embodiments, a is 2. In certain embodiments, a is 3. In certain embodiments, a is 4. In certain embodiments, a is 5. In certain embodiments, at least one instance of R¹ is halogen (e.g., F, Cl, Br, or I). In certain embodiments, at least one instance of R¹ is Cl. In certain embodiments, at least one instance of R¹ is optionally substituted acyl (e.g., —C(═O)Me). In certain embodiments, at least one instance of R¹ is optionally substituted alkyl (e.g., substituted or unsubstituted C₁₋₆ alkyl). In certain embodiments, at least one instance of R¹ is optionally substituted C₁₋₆ alkyl. In certain embodiments, at least one instance of R¹ is substituted or unsubstituted methyl. In certain embodiments, at least one instance of R¹ is substituted methyl. In certain embodiments, at least one instance of R¹ is unsubstituted methyl. In certain embodiments, at least one instance of R¹ is substituted or unsubstituted ethyl. In certain embodiments, at least one instance of R¹ is substituted or unsubstituted propyl. In certain embodiments, at least one instance of R¹ is optionally substituted alkenyl (e.g., substituted or unsubstituted C₂₋₆ alkenyl). In certain embodiments, at least one instance of R¹ is optionally substituted alkynyl (e.g., substituted or unsubstituted C₂₋₆ alkynyl). In certain embodiments, at least one instance of R¹ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R¹ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R¹ is optionally substituted aryl (e.g., substituted or unsubstituted, 6- to 10-membered aryl). In certain embodiments, at least one instance of R¹ is benzyl. In certain embodiments, at least one instance of R¹ is substituted or unsubstituted phenyl. In certain embodiments, at least one instance of R¹ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R¹ is —CN. In certain embodiments, at least one instance of R¹ is —OR^{D1} (e.g., —OH or —OMe). In certain embodiments, at least one instance of R¹ is —O(optionally substituted phenyl). In certain embodiments, at least one instance of R¹ is —O(unsubstituted phenyl). In certain embodiments, at least one instance of R¹ is —N(R^{D1a})₂ (e.g., —NMe₂). In certain embodiments, at least one instance of R¹ is —SR^{D1} (e.g., —SMe). In certain embodiments, at least one instance of R¹ is —NO₂. In certain embodiments, at least one instance of R¹ is —SCN.

In certain embodiments, at least one instance of R¹ is —OR^{D}, —N(R^{D1a})₂ or —SR^{D1} and R^{D1} is as defined herein. In certain embodiments, R^{D1} is hydrogen. In certain embodiments, R^{D1} is optionally substituted acyl (e.g., —C(═O)Me). In certain embodiments, R^{D1} is optionally substituted alkyl (e.g., substituted or unsubstituted C₁₋₆ alkyl). In certain embodiments, R^{D1} is substituted or unsubstituted methyl. In certain embodiments, R^{D1} is substituted or unsubstituted ethyl. In certain embodiments, R^{D1} is substituted or unsubstituted propyl. In certain embodiments, R^{D1} is optionally substituted alkenyl (e.g., substituted or unsubstituted C₂₋₆ alkenyl). In certain embodiments, R^{D1} is optionally substituted alkynyl (e.g., substituted or unsubstituted C₂₋₆ alkynyl). In certain embodiments, R^{D1} is optionally substituted carbocyclyl (e.g., substituted or unsubstituted, 3- to 7-membered, monocyclic carbocyclyl comprising zero, one, or two double bonds in the carbocyclic ring system). In certain embodiments, R^{D1} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, R^{D1} is optionally substituted aryl (e.g., substituted or unsubstituted, 6- to 10-membered aryl). In certain embodiments, R^{D1} is benzyl. In certain embodiments, R^{D1} is optionally substituted phenyl. In certain embodiments, R^{D1} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, R^{D1} is an oxygen protecting group when attached to an oxygen atom. In certain embodiments, R^{D1} is a sulfur protecting group when attached to a sulfur atom.

In certain embodiments, at least one instance of R^{D1a} is hydrogen. In certain embodiments, at least one instance of R^{D1a} is optionally substituted acyl (e.g., —C(═O)Me). In certain embodiments, at least one R^{D1a} is optionally substituted alkyl (e.g., substituted or unsubstituted C₁₋₆ alkyl). In certain embodiments, at least one instance of R^{D1a} is substituted or unsubstituted methyl. In certain embodiments, at least one instance of R^{D1a} is substituted or unsubstituted ethyl. In certain embodiments, at least one instance of R^{D1a} is substituted or unsubstituted propyl. In certain embodiments, at least one instance of R^{D1a} is optionally substituted alkenyl (e.g., substituted or unsubstituted C₂₋₆ alkenyl). In certain embodiments, at least one instance of R^{D1a} is optionally substituted alkynyl (e.g., substituted or unsubstituted C₂₋₆ alkynyl). In certain embodiments, at least one instance of R^{D1a} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{D1a} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{D1a} is optionally substituted aryl (e.g., substituted or unsubstituted, 6- to 10-membered aryl). In certain embodiments, at least one instance of R^{D1a} is benzyl. In certain embodiments, at least one instance of R^{D1a} is optionally substituted phenyl. In certain embodiments, at least one instance of R^{D1a} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{D1a} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)). In certain embodiments, two instances of R^{D1a} are taken together with their intervening atoms to form a substituted or unsubstituted heterocyclic ring (e.g., substituted or unsubstituted, 5- to 10-membered monocyclic or bicyclic heterocyclic ring, wherein one or two atoms in the heterocyclic ring are independently nitrogen, oxygen, or sulfur) or substituted or unsubstituted heteroaryl ring (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur).

Formula (I) includes zero or more instances of substituent R⁴ on the cyclohexyl ring. In certain embodiments, Formula (I) includes one instance of substituent R⁴ on the cyclohexyl ring. In certain embodiments, b is 0. In certain embodiments, b is 1. In certain embodiments, b is 2. In certain embodiments, b is 3. In certain embodiments, b is 4. In certain embodiments, b is 5. In certain embodiments, b is 6. In certain embodiments, b is 7. In certain embodiments, b is 8. In certain embodiments, b is 9. In certain embodiments, b is 10.

In certain embodiments, at least one instance of R⁴ is halogen (e.g., F, Cl, Br, or I). In certain embodiments, at least one instance of R¹ is Cl. In certain embodiments, at least one instance of R⁴ is optionally substituted acyl (e.g., —C(═O)Me). In certain embodiments, at least one instance of R⁴ is optionally substituted alkyl (e.g., substituted or unsubstituted C₁₋₆ alkyl). In certain embodiments, at least one instance of R⁴ is optionally substituted C₁₋₆ alkyl. In certain embodiments, at least one instance of R⁴ is substituted or unsubstituted methyl. In certain embodiments, at least one instance of R⁴ is substituted methyl. In certain embodiments, at least one instance of R⁴ is unsubstituted methyl. In certain embodiments, at least one instance of R⁴ is substituted or unsubstituted ethyl. In certain embodiments, at least one instance of R⁴ is substituted or unsubstituted propyl. In certain embodiments, at least one instance of R¹ is optionally substituted alkenyl (e.g., substituted or unsubstituted C₂₋₆ alkenyl). In certain embodiments, at least one instance of R⁴ is optionally substituted alkynyl (e.g., substituted or unsubstituted C₂₋₆ alkynyl). In certain embodiments, at least one instance of R⁴ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R⁴ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R⁴ is optionally substituted aryl (e.g., substituted or unsubstituted, 6- to 10-membered aryl). In certain embodiments, at least one instance of R⁴ is benzyl. In certain embodiments, at least one instance of R⁴ is substituted or unsubstituted phenyl. In certain embodiments, at least one instance of R⁴ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R⁴ is —CN. In certain embodiments, at least one instance of R⁴ is —OR^{D1} (e.g., —OH or —OMe). In certain embodiments, at least one instance of R⁴ is —N(R^{D1a})₂ (e.g., —NMe₂). In certain embodiments, at least one instance of R⁴ is —SR^{D1} (e.g., —SMe). In certain embodiments, at least one instance of R⁴ is —NO₂. In certain embodiments, at least one instance of R⁴ is —SCN.

Formula (I) includes zero or more instances of substituent R⁵ on the piperazine ring. In certain embodiments, Formula (I) includes one instance of substituent R⁵ on the piperazine ring. In certain embodiments, c is 0. In certain embodiments, c is 1. In certain embodiments, c is 2. In certain embodiments, c is 3. In certain embodiments, c is 4. In certain embodiments, c is 5. In certain embodiments, c is 6. In certain embodiments, c is 7. In certain embodiments, c is 8. In certain embodiments, at least one instance of R⁵ is halogen (e.g., F, Cl, Br, or I). In certain embodiments, at least one instance of R⁵ is Cl. In certain embodiments, at least one instance of R⁵ is optionally substituted acyl (e.g., —C(═O)Me). In certain embodiments, at least one instance of R⁵ is optionally substituted alkyl (e.g., substituted or unsubstituted C₁₋₆ alkyl). In certain embodiments, at least one instance of R⁵ is optionally substituted C₁₋₆ alkyl. In certain embodiments, at least one instance of R⁵ is substituted or unsubstituted methyl. In certain embodiments, at least one instance of R⁵ is substituted methyl. In certain embodiments, at least one instance of R⁵ is unsubstituted methyl. In certain embodiments, at least one instance of R⁵ is substituted or unsubstituted ethyl. In certain embodiments, at least one instance of R⁵ is substituted or unsubstituted propyl. In certain embodiments, at least one instance of R⁵ is optionally substituted alkenyl (e.g., substituted or unsubstituted C₂₋₆ alkenyl). In certain embodiments, at least one instance of R⁵ is optionally substituted alkynyl (e.g., substituted or unsubstituted C₂₋₆ alkynyl). In certain embodiments, at least one instance of R⁵ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R⁵ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R⁵ is optionally substituted aryl (e.g., substituted or unsubstituted, 6- to 10-membered aryl). In certain embodiments, at least one instance of R⁵ is benzyl. In certain embodiments, at least one instance of R⁵ is substituted or unsubstituted phenyl. In certain embodiments, at least one instance of R⁵ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R⁵ is —CN. In certain embodiments, at least one instance of R⁵ is —OR^{D1} (e.g., —OH or —OMe). In certain embodiments, at least one instance of R⁵ is —N(R^{D1a})₂ (e.g., —NMe₂). In certain embodiments, at least one instance of R⁵ is —SR^{D1} (e.g., —SMe). In certain embodiments, at least one instance of R⁵ is —NO₂. In certain embodiments, at least one instance of R⁵ is —SCN.

Ring A; Substituents R² and R³

Formula (I) includes Ring A. In certain embodiments, Ring A is of formula:

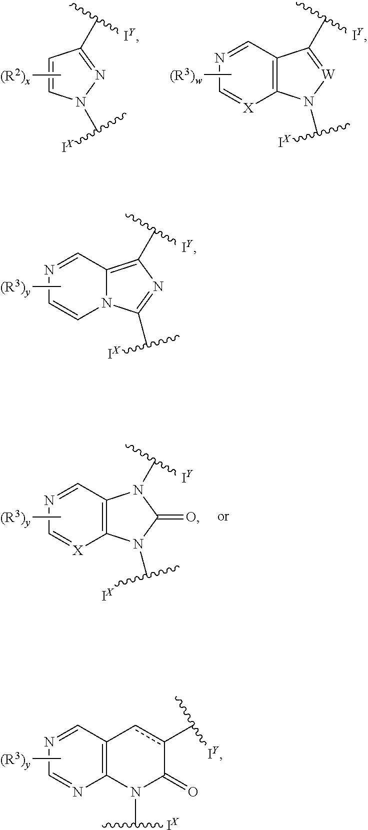

In certain embodiments, Ring A is of formula:

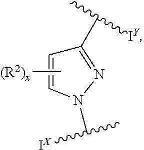

wherein each instance of R² is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}; and x is 0, 1, or 2. In certain embodiments, x is 0. In certain embodiments, x is 1. In certain embodiments, x is 2. In certain embodiments, Ring A is of formula:

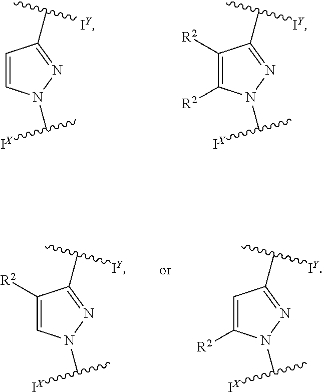

In certain embodiments, Ring A is of formula:

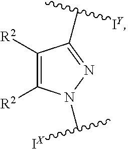

and each instance of R² is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D}, —N(R^{D1a})₂ or —SR^{D1}. In certain embodiments, Ring A is of formula:

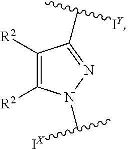

and each instance of R² is independently optionally substituted acyl, optionally substituted alkyl, and —N(R^{D1a})₂.

In certain embodiments, Ring A is of formula:

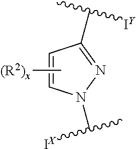

and includes zero or more instances of substituent R². In certain embodiments, Ring A is of formula:

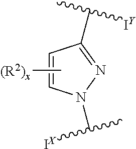

and includes one or more instances of substituent R². In certain embodiments, x is 0. In certain embodiments, x is 1. In certain embodiments, x is 2. In certain embodiments, at least one instance of R² is halogen (e.g., F, Cl, Br, or I). In certain embodiments, at least one instance of R² is Cl. In certain embodiments, at least one instance of R² is optionally substituted acyl (e.g., —C(═O)Me). In certain embodiments, at least one instance of R² is optionally substituted acyl. In certain embodiments, at least one instance of R² is —C(═O)N(R^{D1a})₂, and each occurrence of R^{D1a} is hydrogen, optionally substituted acyl, or optionally substituted alkyl. In certain embodiments, at least one instance of R² is —C(═O)NH₂. In certain embodiments, at least one instance of R² is —C(═O)NMe₂. In certain embodiments, at least one instance of R² is optionally substituted alkyl (e.g., substituted or unsubstituted C₁₋₆ alkyl). In certain embodiments, at least one instance of R² is optionally substituted C₁₋₆ alkyl. In certain embodiments, at least one instance of R² is substituted or unsubstituted methyl. In certain embodiments, at least one instance of R² is substituted methyl. In certain embodiments, at least one instance of R² is unsubstituted methyl. In certain embodiments, at least one instance of R² is substituted or unsubstituted ethyl. In certain embodiments, at least one instance of R² is substituted or unsubstituted propyl. In certain embodiments, at least one instance of R² is optionally substituted alkenyl (e.g., substituted or unsubstituted C₂₋₆ alkenyl). In certain embodiments, at least one instance of R² is optionally substituted alkynyl (e.g., substituted or unsubstituted C₂₋₆ alkynyl). In certain embodiments, at least one instance of R² is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R² is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R² is optionally substituted aryl (e.g., substituted or unsubstituted, 6- to 10-membered aryl). In certain embodiments, at least one instance of R² is benzyl. In certain embodiments, at least one instance of R² is substituted or unsubstituted phenyl. In certain embodiments, at least one instance of R² is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R² is —CN. In certain embodiments, at least one instance of R² is —OR^{D1} (e.g., —OH or —OMe). In certain embodiments, at least one instance of R² is —N(R^{D1a})₂ (e.g., —NMe₂). In certain embodiments, at least one instance of R² is —N(R^{D1a})₂, and each occurrence of R^{D1a} is hydrogen, optionally substituted acyl, or optionally substituted alkyl. In certain embodiments, at least one instance of R² is —NH₂. In certain embodiments, at least one instance of R² is —NMe₂. In certain embodiments, at least one instance of R² is —SR^{D1} (e.g., —SMe). In certain embodiments, at least one instance of R² is —NO₂. In certain embodiments, at least one instance of R² is —SCN.

In certain embodiments, Ring A is of formula:

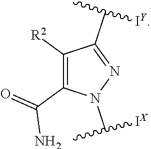

In certain embodiments, Ring A is of formula:

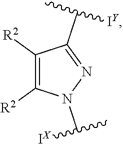

one instance of R² is —C(═O)NH₂, and the other instance of R² is —NH₂. In certain embodiments, Ring A is of formula:

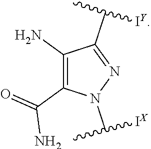

In certain embodiments, Ring A is of formula:

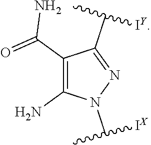

In certain embodiments, Ring A is of formula:

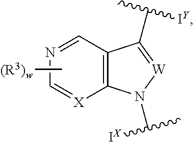

and includes zero or more instances of substituent R³. In certain embodiments, Ring A is of formula:

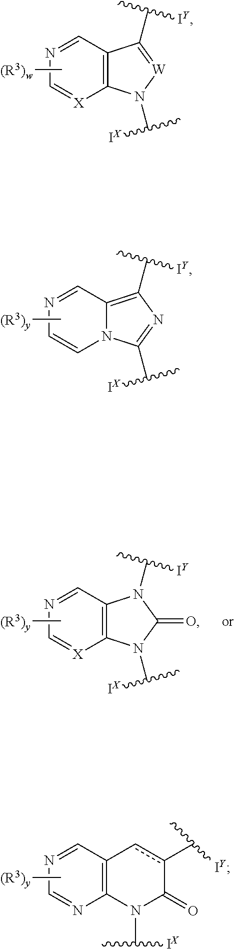

and includes one or more instances of substituent R³. In certain embodiments, w is 0. In certain embodiments, w is 1. In certain embodiments, w is 2. In certain embodiments, w is 3. In certain embodiments, w is 4. In certain embodiments, y is 0. In certain embodiments, y is 1. In certain embodiments, y is 2. In certain embodiments, y is 3. In certain embodiments, in Ring A, W is ═C(R^{A})— or ═N—; and X is ═C(R^{A})— or ═N—; wherein each instance of R^{A} is independently hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A1}, —N(R^{A1a})₂ or —SR^{A1}; R^{A1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom; and each occurrence of R^{A1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{A1a} are taken together with their intervening atoms to form a substituted or unsubstituted heterocyclic or substituted or unsubstituted heteroaryl ring. In certain embodiments, at least one instance of R^{A} is hydrogen. In certain embodiments, at least one instance of R^{A} is halogen (e.g., F, Cl, Br, or I). In certain embodiments, at least one instance of R^{A} is optionally substituted acyl (e.g., —C(═O)Me). In certain embodiments, at least one instance of R^{A} is optionally substituted alkyl (e.g., substituted or unsubstituted C₁₋₆ alkyl). In certain embodiments, at least one instance of R^{A} is optionally substituted alkenyl (e.g., substituted or unsubstituted C₂₋₆ alkenyl). In certain embodiments, at least one instance of R^{A} is optionally substituted alkynyl (e.g., substituted or unsubstituted C₂₋₆ alkynyl). In certain embodiments, at least one instance of R^{A} is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R^{A} is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R^{A} is optionally substituted aryl (e.g., substituted or unsubstituted, 6- to 10-membered aryl). In certain embodiments, at least one instance of R^{A} is substituted or unsubstituted phenyl. In certain embodiments, at least one instance of R^{A} is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R^{A} is —CN. In certain embodiments, at least one instance of R^{A} is —OR^{A1} (e.g., —OH or —OMe). In certain embodiments, at least one instance of R^{A} is —N(R^{A1a})₂ (e.g., —NH₂, —NMe₂). In certain embodiments, at least one instance of R^{A} is —SR^{A1} (e.g., —SMe). In certain embodiments, at least one instance of R^{A1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom. In certain embodiments, at least one instance of R^{A1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{A1a} are taken together with their intervening atoms to form a substituted or unsubstituted heterocyclic or substituted or unsubstituted heteroaryl ring.

In certain embodiments, Ring A includes one or more instances of substituent R³. In certain embodiments, at least one instance of R³ is halogen (e.g., F, Cl, Br, or I). In certain embodiments, at least one instance of R³ is Cl. In certain embodiments, at least one instance of R³ is optionally substituted acyl (e.g., —C(═O)Me). In certain embodiments, at least one instance of R³ is optionally substituted alkyl (e.g., substituted or unsubstituted C₁₋₆ alkyl). In certain embodiments, at least one instance of R³ is optionally substituted C₁₋₆ alkyl. In certain embodiments, at least one instance of R³ is substituted or unsubstituted methyl. In certain embodiments, at least one instance of R³ is substituted methyl. In certain embodiments, at least one instance of R³ is unsubstituted methyl. In certain embodiments, at least one instance of R³ is substituted or unsubstituted ethyl. In certain embodiments, at least one instance of R³ is substituted or unsubstituted propyl. In certain embodiments, at least one instance of R³ is optionally substituted alkenyl (e.g., substituted or unsubstituted C₂₋₆ alkenyl). In certain embodiments, at least one instance of R³ is optionally substituted alkynyl (e.g., substituted or unsubstituted C₂₋₆ alkynyl). In certain embodiments, at least one instance of R³ is optionally substituted carbocyclyl (e.g., substituted or unsubstituted, 3- to 7-membered, monocyclic carbocyclyl comprising zero, one, or two double bonds in the carbocyclic ring system). In certain embodiments, at least one instance of R³ is optionally substituted heterocyclyl (e.g., substituted or unsubstituted, 5- to 10-membered monocyclic or bicyclic heterocyclic ring, wherein one or two atoms in the heterocyclic ring are independently nitrogen, oxygen, or sulfur). In certain embodiments, at least one instance of R³ is optionally substituted aryl (e.g., substituted or unsubstituted, 6- to 10-membered aryl). In certain embodiments, at least one instance of R³ is benzyl. In certain embodiments, at least one instance of R³ is substituted or unsubstituted phenyl. In certain embodiments, at least one instance of R³ is optionally substituted heteroaryl (e.g., substituted or unsubstituted, 5- to 6-membered, monocyclic heteroaryl, wherein one, two, three, or four atoms in the heteroaryl ring system are independently nitrogen, oxygen, or sulfur; or substituted or unsubstituted, 9- to 10-membered, bicyclic heteroaryl, wherein one, two, three, or four atoms in the heteroaryl ring system are independently nitrogen, oxygen, or sulfur). In certain embodiments, at least one instance of R³ is —CN. In certain embodiments, at least one instance of R³ is —OR^{D1} (e.g., —OH or —OMe). In certain embodiments, at least one instance of R³ is —N(R^{D1a})₂ (e.g., —NMe₂). In certain embodiments, at least one instance of R³ is —N(R^{D1a})₂, and each occurrence of R^{D1a} is hydrogen, optionally substituted acyl, or optionally substituted alkyl. In certain embodiments, at least one instance of R³ is —NH₂. In certain embodiments, at least one instance of R³ is —NMe₂. In certain embodiments, at least one instance of R³ is —SR^{D1} (e.g., —SMe). In certain embodiments, at least one instance of R³ is —NO₂. In certain embodiments, at least one instance of R³ is —SCN.

In certain embodiments, Ring A is of formula:

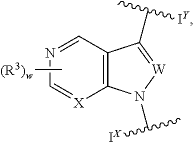

wherein W is ═C(R^{A})— or ═N—; and X is ═C(R^{A})— or ═N—; each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂ or —SR^{D1}; and w is 0, 1, 2, 3, or 4. In certain embodiments, w is 0. In certain embodiments, w is 1. In certain embodiments, w is 2. In certain embodiments, w is 3. In certain embodiments, w is 4. In certain embodiments, Ring A is of formula:

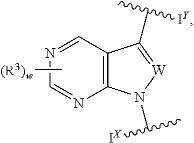

wherein W is ═C(R^{A})— or ═N—.

In certain embodiments, Ring A is of formula:

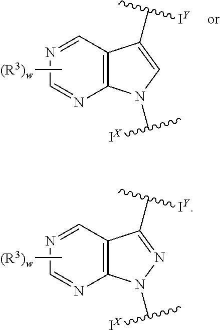

In certain embodiments, Ring A is of formula:

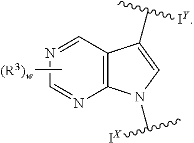

In certain embodiments, Ring A is of formula:

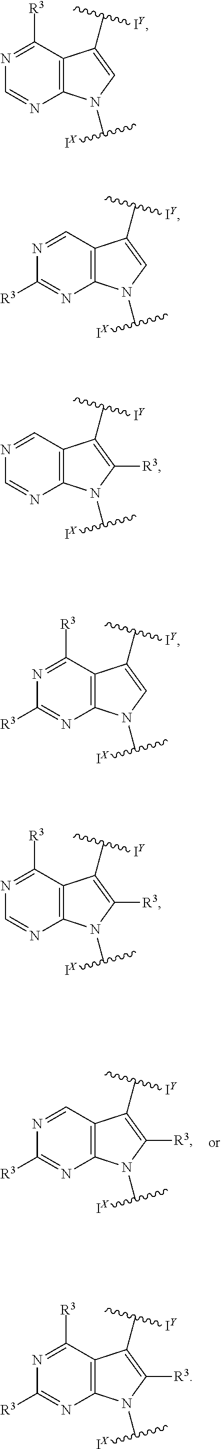

In certain embodiments, Ring A is of formula:

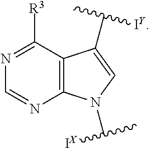

In certain embodiments, Ring A is of formula:

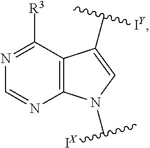

wherein R³ is —N(R^{3a})₂, and each instance of R^{3a} is independently hydrogen, optionally substituted alkyl, optionally substituted alkenyl, optionally substituted alkynyl, or a nitrogen protecting group. In certain embodiments, Ring A is of formula:

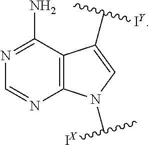

In certain embodiments, Ring A is of formula:

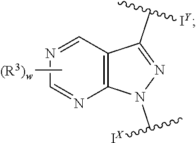

each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂ or —SR^{D1}; and w is 0, 1, 2, 3, or 4. In certain embodiments, w is 0. In certain embodiments, w is 1. In certain embodiments, w is 2. In certain embodiments, w is 3. In certain embodiments, w is 4. In certain embodiments, Ring A is of formula:

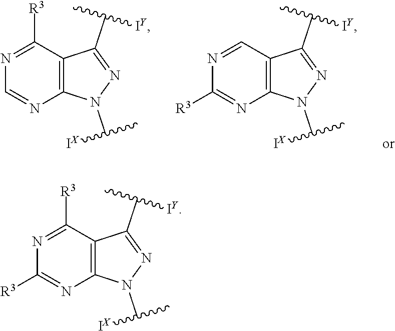

In certain embodiments, Ring A is of formula:

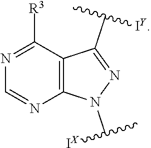

In certain embodiments, Ring A is of formula:

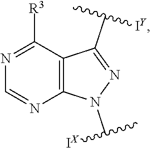

wherein R³ is —N(R^{3a})₂, and each instance of R^{3a} is independently hydrogen, optionally substituted alkyl, optionally substituted alkenyl, optionally substituted alkynyl, or a nitrogen protecting group. In certain embodiments, Ring A is of formula:

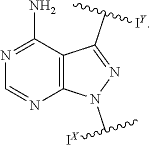

In certain embodiments, Ring A is of formula:

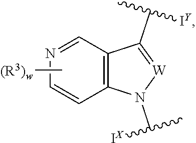

wherein X is ═C(R^{A})— or ═N—; each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, or —SR^{D1}; and w is 0, 1, 2, 3, or 4. In certain embodiments, w is 0. In certain embodiments, w is 1. In certain embodiments, w is 2. In certain embodiments, w is 3. In certain embodiments, w is 4. In certain embodiments, Ring A is of formula:

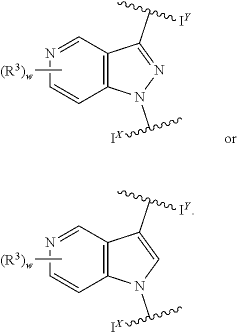

In certain embodiments, Ring A is of formula:

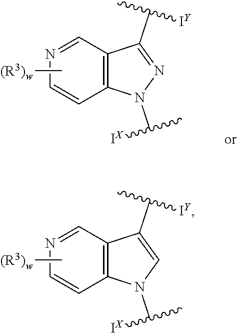

wherein each instance of R³ is independently acyl or —N(R^{3a})₂, and each instance of R^{3a} is independently hydrogen, optionally substituted alkyl, optionally substituted alkenyl, optionally substituted alkynyl, or a nitrogen protecting group. In certain embodiments, Ring A is of formula:

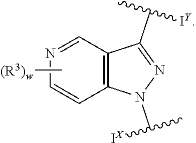

In certain embodiments, Ring A is of formula:

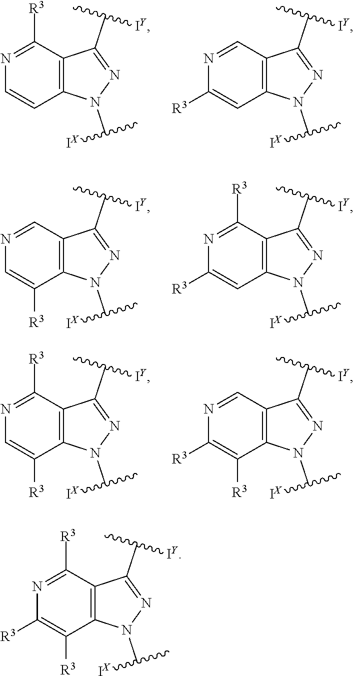

In certain embodiments, Ring A is of formula:

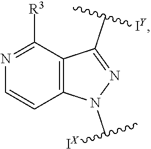

wherein R³ is acyl or —N(R^{3a})₂, and each instance of R^{3a} is independently hydrogen, optionally substituted alkyl, optionally substituted alkenyl, optionally substituted alkynyl, or a nitrogen protecting group. In certain embodiments, Ring A is of formula:

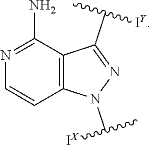

In certain embodiments, Ring A is of formula:

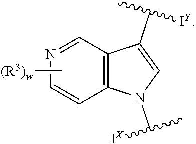

In certain embodiments, Ring A is of formula:

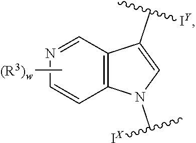

wherein w is 0, 1, or 2. In certain embodiments, Ring A is of formula:

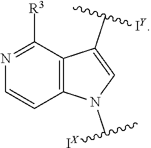

In certain embodiments, Ring A is of formula:

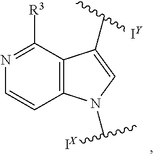

wherein R³ is acyl or —N(R^{3a})₂, and each instance of R^{3a} is independently hydrogen, optionally substituted alkyl, optionally substituted alkenyl, optionally substituted alkynyl, or a nitrogen protecting group. In certain embodiments, Ring A is of formula:

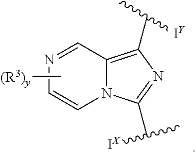

In certain embodiments, Ring A is of formula:

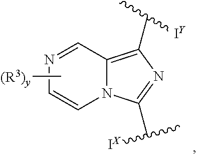

wherein y is 0, 1, or 2.

In certain embodiments, Ring A is of formula:

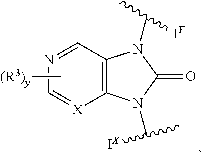

wherein X is ═C(R^{A})— or ═N—; each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, or —SR^{D1}; and y is 0, 1, 2, or 3. In certain embodiments, y is 0. In certain embodiments, y is 1. In certain embodiments, y is 2. In certain embodiments, y is 3. In certain embodiments, Ring A is of formula:

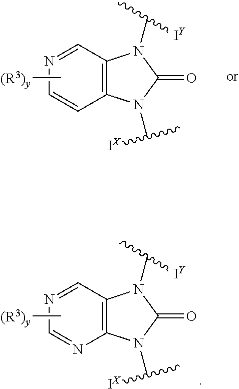

In certain embodiments, Ring A is of formula:

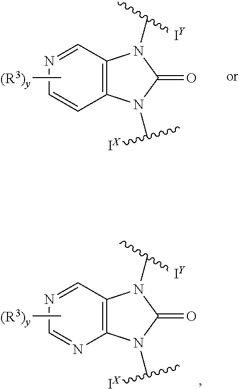

wherein y is 0. In certain embodiments, Ring A is of formula:

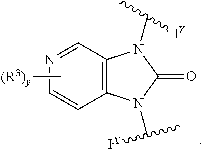

In certain embodiments, Ring A is of formula:

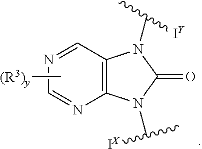

In certain embodiments, Ring A is of formula:

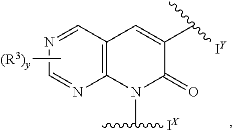

wherein each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, or —SR^{D1}; and y is 0, 1, or 2. In certain embodiments, Ring A is of formula:

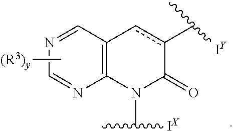

In certain embodiments, Ring A is of formula:

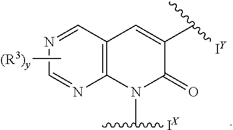

In certain embodiments, Ring A is of formula:

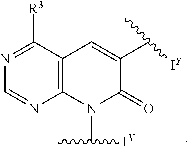

In certain embodiments, Ring A is of formula:

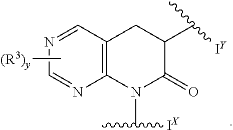

### Linkers L1 and L2

In Formula (I), L1 is a divalent moiety linking the group D to the piperazine moiety of Formula (I). In Formula (I), L1 is a divalent moiety. In certain embodiments, L1 is a substituted or unsubstituted C₁₋₅₀ hydrocarbon chain as the shortest path between D and the piperazine moiety of Formula (I), optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is a substituted or unsubstituted C₁₋₃₀ hydrocarbon chain, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₃₀ hydrocarbon chain, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is a substituted or unsubstituted C₁₋₂₄ hydrocarbon chain, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₂₄ hydrocarbon chain, optionally wherein one or more chain atoms of the hydrocarbon chain are independently replaced with —C(═O)—, —O—, —NR^{b}—, —S—, or a cyclic moiety, wherein R^{b} is independently hydrogen, substituted or unsubstituted C₁₋₆ alkyl, or a nitrogen protecting group. In certain embodiments, L1 is a substituted or unsubstituted C₁₋₂₀ hydrocarbon chain, optionally wherein one or more chain atoms of the hydrocarbon chain are independently replaced with —C(═O)—, —O—, —NR^{b}—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₂₀ hydrocarbon chain, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₁₆ hydrocarbon chain, optionally wherein one or more chain atoms of the hydrocarbon chain are independently replaced with —C(═O)—, —O—, —NR—, or a cyclic moiety, wherein R^{b} is independently hydrogen, substituted or unsubstituted C₁₋₆ alkyl, or a nitrogen protecting group. In certain embodiments, at least one chain atom of the hydrocarbon chain of L1 is independently replaced with —O—. In certain embodiments, L1 is any “L0” group or “Linker” group recited in U.S. patent application U.S. Ser. No. 14/707,930, filed May 8, 2015, issued as U.S. Pat. No. 9,694,084 on Jul. 4, 2017, which is incorporated herein by reference. In certain embodiments, L1 is any “L” group recited in U.S. patent application U.S. Ser. No. 14/792,414, filed Jul. 6, 2015, published as U.S. Patent Application Publication No. 2016-0058872, on Mar. 3, 2016, which is incorporated herein by reference.

In certain embodiments, the chain of linker L1 comprises up to 50 consecutive covalently bonded atoms in length as the shortest path between D and the piperazine moiety of Formula (I), excluding hydrogen atoms and substituents. In certain embodiments, the chain of linker L1 comprises up to 50 consecutive covalently bonded atoms in length, excluding hydrogen atoms and substituents. In certain embodiments, L1 comprises up to 46 consecutive covalently bonded atoms in length, excluding hydrogen atoms and substituents. In certain embodiments, L1 comprises up to 45 consecutive covalently bonded atoms in length, excluding hydrogen atoms and substituents. In certain embodiments, L1 comprises up to 40 consecutive covalently bonded atoms in length, excluding hydrogen atoms and substituents. In certain embodiments, L1 comprises up to 35 consecutive covalently bonded atoms in length, excluding hydrogen atoms and substituents. In certain embodiments, L1 comprises up to 32 consecutive covalently bonded atoms in length, excluding hydrogen atoms and substituents. In certain embodiments, L1 comprises up to 30 consecutive covalently bonded atoms in length, excluding hydrogen atoms and substituents. In certain embodiments, L1 comprises up to 25 consecutive covalently bonded atoms in length, excluding hydrogen atoms and substituents. In certain embodiments, L1 comprises up to 25 consecutive covalently bonded atoms in length, excluding hydrogen atoms and substituents. In certain embodiments, L1 comprises up to 23 consecutive covalently bonded atoms in length, excluding hydrogen atoms and substituents. In certain embodiments, L1 comprises up to 20 consecutive covalently bonded atoms in length, excluding hydrogen atoms and substituents. In certain embodiments, L1 comprises up to 14 consecutive covalently bonded atoms in length, excluding hydrogen atoms and substituents. In certain embodiments, L1 comprises up to 15 consecutive covalently bonded atoms in length, excluding hydrogen atoms and substituents. In certain embodiments, L1 comprises up to 12 consecutive covalently bonded atoms in length, excluding hydrogen atoms and substituents. In certain embodiments, L1 comprises up to 11 consecutive covalently bonded atoms in length, excluding hydrogen atoms and substituents. In certain embodiments, L1 comprises up to 10 consecutive covalently bonded atoms in length, excluding hydrogen atoms and substituents. In certain embodiments, L1 comprises up to 9 consecutive covalently bonded atoms in length, excluding hydrogen atoms and substituents. In certain embodiments, L1 comprises up to 8 consecutive covalently bonded atoms in length, excluding hydrogen atoms and substituents. In certain embodiments, L1 comprises up to 7 consecutive covalently bonded atoms in length, excluding hydrogen atoms and substituents. In certain embodiments, L1 comprises up to 6 consecutive covalently bonded atoms in length, excluding hydrogen atoms and substituents. In certain embodiments, L1 comprises up to 5 consecutive covalently bonded atoms in length, excluding hydrogen atoms and substituents. In certain embodiments, L1 comprises up to 3 consecutive covalently bonded atoms in length, excluding hydrogen atoms and substituents.

In certain embodiments, any of the atoms in L1 can be substituted. In certain embodiments, none of the atoms in the linker L1 are substituted. In certain embodiments, none of the carbon atoms in the linker are substituted.

In certain embodiments, L1 is a linker that contains an asymmetric carbon/stereocenter, i.e., an sp³ hybridized carbon atom bearing 4 different groups attached thereto. In certain embodiments, the compound comprising such an L1 group is enantiomerically enriched or substantially enantiomerically enriched. In certain embodiments, the compound comprising such an L1 group is enantiomerically pure. In certain embodiments, the compound comprising such an L1 group is racemic.

In certain embodiments, L1 comprises substituted or unsubstituted carbocyclylene, substituted or unsubstituted heterocyclylene, substituted or unsubstituted arylene, substituted or unsubstituted heteroarylene, or substituted or unsubstituted heteroalkylene, or combinations thereof. In certain embodiments, L1 is substituted or unsubstituted carbocyclylene, substituted or unsubstituted heterocyclylene, substituted or unsubstituted arylene, substituted or unsubstituted heteroarylene, or substituted or unsubstituted heteroalkylene. In certain embodiments, L1 is a linker selected from the group consisting of the following divalent moieties: substituted and unsubstituted alkylene, substituted and unsubstituted alkenylene, substituted and unsubstituted alkynylene, substituted and unsubstituted heteroalkylene, substituted and unsubstituted heteroalkenylene, substituted and unsubstituted heteroalkynylene, substituted and unsubstituted heterocyclylene, substituted and unsubstituted carbocyclylene, substituted and unsubstituted arylene, substituted and unsubstituted heteroarylene, and combinations thereof.

Reference to L1 being a combination of at least two instances of the divalent moieties described herein refers to a linker consisting of at least one instance of a first divalent moiety and at least one instance of a second divalent moiety, wherein the first and second divalent moieties are the same or different and are within the scope of the divalent moieties described herein, and the instances of the first and second divalent moieties are consecutive covalently attached to each other. For example, when L1 is a combination of alkylene and heteroalkylene linkers -alkylene-heteroalkylene-, -alkylene-(heteroalkylene)₂-, and -heteroalkylene-alkylene-heteroalkylene- are all within the scope of L, wherein each instance of alkylene in any one of the linkers may be the same or different, and each instance of heteroalkylene in any one of the linkers may be the same or different.

In certain embodiments, L1 comprises at least one instance of substituted or unsubstituted alkylene, e.g., substituted or unsubstituted C₁₋₆alkylene, substituted or unsubstituted C₁₋₂alkylene, substituted or unsubstituted C₂₋₃alkylene, substituted or unsubstituted C₃₋₄alkylene, substituted or unsubstituted C₄₋₅alkylene, substituted or unsubstituted C₅₋₆alkylene, substituted or unsubstituted C₃₋₆alkylene, or substituted or unsubstituted C₄-6alkylene. Exemplary alkylene groups include unsubstituted alkylene groups, such as methylene (—CH₂—), ethylene (—(CH₂)₂—), n-propylene (—(CH₂)₃—), n-butylene (—(CH₂)₄—), n-pentylene (—(CH₂)₅—), and n-hexylene (—(CH₂)₆—).

In certain embodiments, L1 comprises at least one instance of substituted or unsubstituted alkenylene, e.g., substituted or unsubstituted C₂₋₄alkenylene, substituted or unsubstituted C₂₋₃alkenylene, substituted or unsubstituted C₃₋₄alkenylene, substituted or unsubstituted C₄₋₅alkenylene, or substituted or unsubstituted C₅₋₄alkenylene.

In certain embodiments, L1 comprises at least one instance of substituted or unsubstituted alkynylene, e.g., substituted or unsubstituted C₂₋₆alkynylene, substituted or unsubstituted C₂₋₃alkynylene, substituted or unsubstituted C₃₋₄alkynylene, substituted or unsubstituted C₄₋₅alkynylene, or substituted or unsubstituted C₅₋₆alkynylene.

In certain embodiments, L1 comprises at least one instance of substituted or unsubstituted heteroalkylene, e.g., substituted or unsubstituted heteroC₁₋₆alkylene, substituted or unsubstituted heteroC₁₋₂alkylene, substituted or unsubstituted heteroC₂₋₃alkylene, substituted or unsubstituted heteroC₃₋₄alkylene, substituted or unsubstituted heteroC₄₋₅ alkylene, or substituted or unsubstituted heteroC₅₋₆alkylene. Exemplary heteroalkylene groups include unsubstituted heteroalkylene groups, such as —(CH₂)₂—O(CH₂)₂—, —OCH₂—, —CH₂O—, —O(CH₂)₂—, —(CH₂)₂O—, —O(CH₂)₃—, —(CH₂)₃O—, —O(CH₂)₄—, —(CH₂)₄O—, —O(CH₂)₅—, —(CH₂)₅O—, —O(CH₂)₆—, and —O(CH₂)₆O—, and amide groups (e.g., —NH—C(═O)— and —C(═O)NH—).

In certain embodiments, L1 comprises at least one instance of substituted or unsubstituted heteroalkenylene, e.g., substituted or unsubstituted heteroC₂₋₆alkenylene, substituted or unsubstituted heteroC₂₋₃alkenylene, substituted or unsubstituted heteroC₃₋₄alkenylene, substituted or unsubstituted heteroC₄₋₅alkenylene, or substituted or unsubstituted heteroC₅₋₆alkenylene.

In certain embodiments, L1 comprises at least one instance of substituted or unsubstituted heteroalkynylene, e.g., substituted or unsubstituted heteroC₂₋₆alkynylene, substituted or unsubstituted heteroC₂₋₃alkynylene, substituted or unsubstituted heteroC₃₋₄alkynylene, substituted or unsubstituted heteroC₄₋₅alkynylene, or substituted or unsubstituted heteroC₅₋₆alkynylene.

In certain embodiments, L1 comprises at least one instance of substituted or unsubstituted carbocyclylene, e.g., substituted or unsubstituted C₃₋₆carbocyclylene, substituted or unsubstituted C₃₋₄carbocyclylene, substituted or unsubstituted C₄₋₅ carbocyclylene, or substituted or unsubstituted C₅₋₆ carbocyclylene.

In certain embodiments, L1 comprises at least one instance of substituted or unsubstituted heterocyclylene, e.g., substituted or unsubstituted 3-6 membered heterocyclylene, substituted or unsubstituted 3-4 membered heterocyclylene, substituted or unsubstituted 4-5 membered heterocyclylene, or substituted or unsubstituted 5-6 membered heterocyclylene. In certain embodiments, at least one chain atom of the hydrocarbon chain of L1 is independently replaced with a 5-8 membered heterocyclyl group with 1-4 ring heteroatoms selected from the group consisting of nitrogen, oxygen, and sulfur. In certain embodiments, at least one chain atom of the hydrocarbon chain of L1 is independently replaced with a six-membered heterocyclyl group with 1-3 ring heteroatoms selected from the group consisting of nitrogen and oxygen. In certain embodiments, at least one chain atom of the hydrocarbon chain of L1 is independently replaced with piperidine or piperazine. In certain embodiments, at least one chain atom of the hydrocarbon chain of L1 is independently replaced with piperidine. In certain embodiments, at least one chain atom of the hydrocarbon chain of L1 is independently replaced with piperazine. In certain embodiments, at least one chain atom of the hydrocarbon chain of L1 is independently replaced with morpholine.

In certain embodiments, L1 comprises at least one instance of substituted or unsubstituted arylene, e.g., substituted or unsubstituted phenylene. In certain embodiments, at least one chain atom of the hydrocarbon chain of L1 is independently replaced with an optionally substituted phenyl group. In certain embodiments, L1 comprises at least one instance of substituted or unsubstituted heteroarylene, e.g., substituted or unsubstituted 5- to 6-membered heteroarylene.

In certain embodiments, L1 is an unsubstituted hydrocarbon chain, optionally wherein one or more chain atoms of the hydrocarbon chain are independently replaced with —NR^{b}—, and each instance of R^{b} is independently hydrogen, substituted or unsubstituted C₁₋₆ alkyl, or a nitrogen protecting group, or optionally two instances of R^{b} are taken together with their intervening atoms to form a substituted or unsubstituted heterocyclic or substituted or unsubstituted heteroaryl ring. In certain embodiments, at least one instance of R^{b} is hydrogen. In certain embodiments, at least one instance of R^{b} is substituted or unsubstituted C₁₋₆ alkyl (e.g., substituted or unsubstituted methyl or ethyl). In certain embodiments, at least one instance of R^{b} is a nitrogen protecting group (e.g., benzyl (Bn), t-butyl carbonate (BOC or Boc), benzyl carbamate (Cbz), 9-fluorenylmethyl carbonate (Fmoc), trifluoroacetyl, triphenylmethyl, acetyl, or p-toluenesulfonamide (Ts)).

In certain embodiments, L1 is an optionally substituted C₁₋₄₅ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR^{b}—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₄₅ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR^{b}—, —S—, or a cyclic moiety, wherein R^{b} is independently hydrogen, substituted or unsubstituted C₁₋₆ alkyl, or a nitrogen protecting group. In certain embodiments, L1 is an optionally substituted C₁₋₂₄ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR^{b}—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₂₄hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an optionally substituted C₁₋₂₀ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₂₀ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an optionally substituted C₁₋₁₆ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an unsubstituted C₁₋₁₆ hydrocarbon chain as the shortest path between D and the piperazine moiety of Formula (I), excluding hydrogen atoms and substituents, optionally wherein one or more chain atoms of the hydrocarbon chain are independently replaced with —C(═O)—, —O—, —NR—, —S—, or a cyclic moiety, wherein R^{b} is independently hydrogen, substituted or unsubstituted C₁₋₆ alkyl, or a nitrogen protecting group. In certain embodiments, L1 is an optionally substituted C₁₋₃₀ hydrocarbon chain, wherein one or more chain atoms of the hydrocarbon chain are independently replaced with —O— or —NR^{b}—. In certain embodiments, L1 is an unsubstituted C₁₋₃₀ hydrocarbon chain, wherein one or more chain atoms of the hydrocarbon chain are independently replaced with —O— or —NR^{b}—. In certain embodiments, L1 is an unsubstituted C₁₋₃₀ hydrocarbon chain, wherein at least one chain atom of the hydrocarbon chain is independently replaced with —O—. In certain embodiments, L1 is an unsubstituted C₁₋₁₆ hydrocarbon chain, wherein at least one chain atom of the hydrocarbon chain is independently replaced with —O—. In certain embodiments, L1 is an unsubstituted C₁₋₂₆ hydrocarbon chain, wherein one or more chain atoms of the hydrocarbon chain are independently replaced with —C(═O)—, —O—, or —NR^{b}—. In certain embodiments, L1 is an unsubstituted C₁₋₂₀ hydrocarbon chain, wherein one or more chain atoms of the hydrocarbon chain are independently replaced with —O—. In certain embodiments, L1 is an unsubstituted C₅₋₂₆ hydrocarbon chain, wherein one or more chain atoms of the hydrocarbon chain are independently replaced with —C(═O)—, —O—, or —NR^{b}—. In certain embodiments, L1 is an unsubstituted C₅₋₂₆ hydrocarbon chain, wherein one or more chain atoms of the hydrocarbon chain are independently replaced with —O—. In certain embodiments, L1 is an unsubstituted C₅₋₂₀ hydrocarbon chain, wherein one or more chain atoms of the hydrocarbon chain are independently replaced with —C(═O)—, —O—, or —NR^{b}—. In certain embodiments, L1 is an unsubstituted C₅₋₂₀ hydrocarbon chain, wherein one or more chain atoms of the hydrocarbon chain are independently replaced with —O—, or —NR^{b}—. In certain embodiments, L1 is an unsubstituted C₅₋₁₅ hydrocarbon chain, wherein one or more chain atoms of the hydrocarbon chain are independently replaced with —C(═O)—, —O—, or —NR^{b}—. In certain embodiments, L1 is an unsubstituted C₁₅₋₂₀ hydrocarbon chain, wherein one or more chain atoms of the hydrocarbon chain are independently replaced with —C(═O)—, —O—, or —NR^{b}—. In certain embodiments, L1 is an unsubstituted C₂₀₋₂₅ hydrocarbon chain, wherein one or more chain atoms of the hydrocarbon chain are independently replaced with —C(═O)—, —O—, or —NR^{b}—. In certain embodiments, L1 is a substituted or unsubstituted C₁₋₄₅ hydrocarbon chain. In certain embodiments, L1 is a substituted or unsubstituted C₅₋₄₀ hydrocarbon chain. In certain embodiments, one or more chain atoms of the hydrocarbon chain of L1 are independently replaced with —C(═O)—, —O—, —S—, —NR^{b}—, —N═, or ═N—. In certain embodiments, one or more chain atoms of the hydrocarbon chain of L1 are independently replaced with —C(═O)—, —O—, or —NR^{b}—, wherein R^{b} is independently hydrogen, substituted or unsubstituted C₁₋₆ alkyl, or a nitrogen protecting group. In certain embodiments, L1 is an unsubstituted C₁₋₂₆ hydrocarbon chain, wherein at least one chain atom of the hydrocarbon chain is independently replaced with —O—.

In certain embodiments, L1 is an all-carbon, substituted or unsubstituted C₁₋₄₅ hydrocarbon chain as the shortest path between D and the piperazine moiety of Formula (I), excluding hydrogen atoms and substituents. In certain embodiments, L1 is an all-carbon, substituted or unsubstituted C₁₋₃₀ hydrocarbon chain as the shortest path between D and the piperazine moiety of Formula (I), excluding hydrogen atoms and substituents. In certain embodiments, L1 is an all-carbon, substituted or unsubstituted C₁₋₂₆ hydrocarbon chain as the shortest path between D and the piperazine moiety of Formula (I), excluding hydrogen atoms and substituents. In certain embodiments, L1 is an all-carbon, substituted or unsubstituted C₁-24 hydrocarbon chain as the shortest path between D and the piperazine moiety of Formula (I), excluding hydrogen atoms and substituents. In certain embodiments, L1 is an all-carbon, substituted or unsubstituted C₁₋₂₀ hydrocarbon chain as the shortest path between D and the piperazine moiety of Formula (I), excluding hydrogen atoms and substituents. In certain embodiments, L1 is an all-carbon, substituted or unsubstituted C₁₋₂₀ hydrocarbon chain as the shortest path between D and the piperazine moiety of Formula (I), excluding hydrogen atoms and substituents. In certain embodiments, L1 is an all-carbon, substituted or unsubstituted C₁-16 hydrocarbon chain as the shortest path between D and the piperazine moiety of Formula (I), excluding hydrogen atoms and substituents.

In certain embodiments, L1 is a bond.

In certain embodiments, L1 includes the moiety

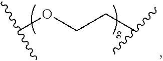

wherein g is 1, 2, 3, 4, 5, or 6. In certain embodiments, g is 1. In certain embodiments, g is 2. In certain embodiments, g is 3. In certain embodiments, g is 4. In certain embodiments, g is 5. In certain embodiments, g is 6.

In certain embodiments, L1 includes the moiety —NHC(═O)—. In certain embodiments, at least one chain atom of the hydrocarbon chain of L1 is independently replaced with —NHC(═O)—.

In certain embodiments, L1 includes the moiety —NH—. In certain embodiments, at least one chain atom of the hydrocarbon chain of L1 is independently replaced with —NH—.

In certain embodiments, L1 includes the moiety —C(═O)—. In certain embodiments, at least one chain atom of the hydrocarbon chain of L1 is independently replaced with —C(═O)—.

In certain embodiments, L1 is of formula,

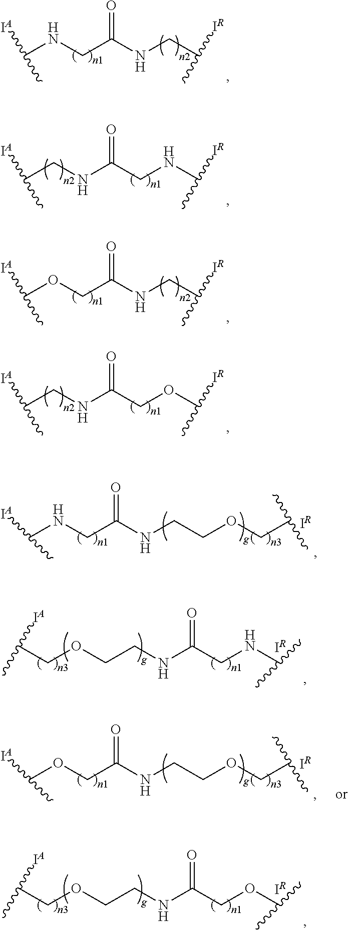

indicates the point of attachment to the moiety of formula:

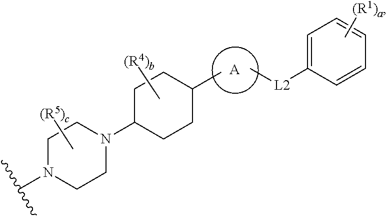

and l^{A} indicates the point of attachment to D; n1 is 1, 2, 3, 4, 5, or 6; n2 is 1, 2, 3, 4, 5, 6, 7, 8, 9, or 10; n3 is 1, 2, 3, 4, 5, or 6; and g is 1, 2, 3, 4, 5, or 6. In certain embodiments, n1 is 1. In certain embodiments, n1 is 2. In certain embodiments, n1 is 3. In certain embodiments, n1 is 4. In certain embodiments, n1 is 5. In certain embodiments, n1 is 6. In certain embodiments, n2 is 1. In certain embodiments, n2 is 2. In certain embodiments, n2 is 3. In certain embodiments, n2 is 4. In certain embodiments, n2 is 5. In certain embodiments, n2 is 6. In certain embodiments, n2 is 7. In certain embodiments, n2 is 8. In certain embodiments, n2 is 9. In certain embodiments, n2 is 10. In certain embodiments, n3 is 1. In certain embodiments, n3 is 2. In certain embodiments, n3 is 3. In certain embodiments, n3 is 4. In certain embodiments, n3 is 5. In certain embodiments, n3 is 6. In certain embodiments, g is 1. In certain embodiments, g is 2. In certain embodiments, g is 3. In certain embodiments, g is 4. In certain embodiments, g is 5. In certain embodiments, g is 6.

In certain embodiments, L1 is of formula:

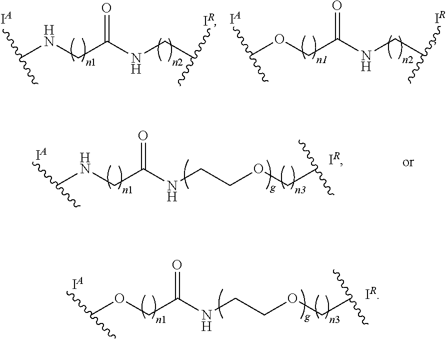

In certain embodiments, L1 is of formula:

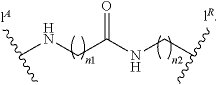

In certain embodiments, L1 is of formula:

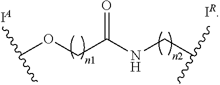

In certain embodiments, L1 is of formula:

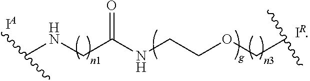

In certain embodiments, L1 is of formula:

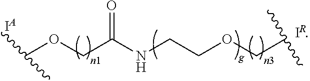

In certain embodiments, L1 is of formula:

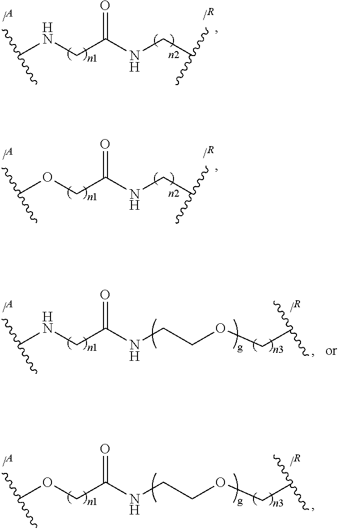

wherein: n1 is 1, 2, or 3; n2 is 4, 5, 6, 7, 8, or 9; n3 is 1, 2, 3, or 4; and g is 1, 2, 3, 4, or 5. In certain embodiments, L1 is of formula:

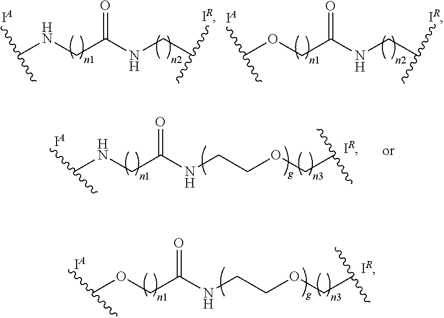

wherein: n1 is 1, 2, or 3; n2 is 4, 5, 6, 7, 8; n3 is 2, 3, or 4; and g is 1, 2, or 3. In certain embodiments, n1 is 1. In certain embodiments, n1 is 2. In certain embodiments, n1 is 3. In certain embodiments, n2 is 4. In certain embodiments, n2 is 5. In certain embodiments, n2 is 6. In certain embodiments, n2 is 7. In certain embodiments, n2 is 8. In certain embodiments, n2 is 9. In certain embodiments, n3 is 1. In certain embodiments, n3 is 2. In certain embodiments, n3 is 3. In certain embodiments, n3 is 4. In certain embodiments, g is 1. In certain embodiments, g is 2. In certain embodiments, g is 3. In certain embodiments, g is 4. In certain embodiments, g is 5.

In certain embodiments, L1 is of formula:

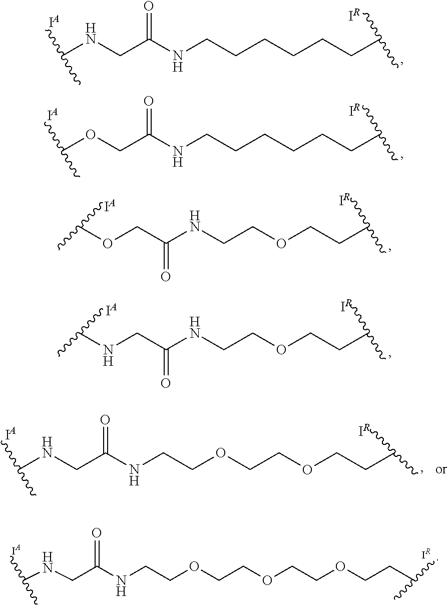

In certain embodiments, L1 is of formula:

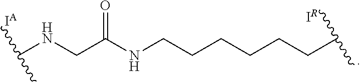

In certain embodiments, L1 is of formula:

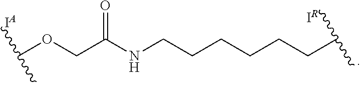

In certain embodiments, L1 is of formula:

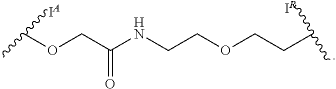

In certain embodiments, L1 is of formula:

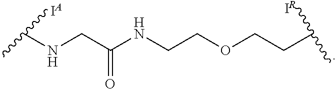

In certain embodiments, L1 is of formula:

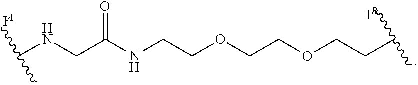

In certain embodiments, L1 is of formula:

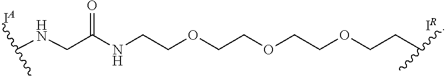

In certain embodiments, L2 is a bond; Ring A is of formula:

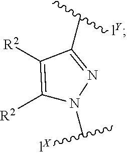

and each instance of R² is independently optionally substituted acyl, optionally substituted alkyl, and —N(R^{D1a})₂; L1 is an unsubstituted C₁₋₂₄ hydrocarbon chain, optionally wherein one or more chain atoms of the hydrocarbon chain are independently replaced with —C(═O)—, —O—, —NR^{b}—, or a cyclic moiety, wherein R^{b} is independently hydrogen, substituted or unsubstituted C₁₋₆ alkyl, or a nitrogen protecting group; a is 1; b is 0; c is 0; and D is of the formula:

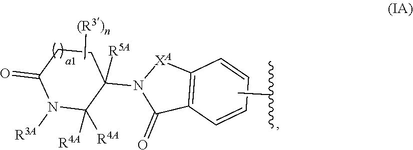

wherein X^{A}, R^{1A}, R^{3A}, R^{3′}, R^{4A}, R^{5A}, m, n, and a1 are as defined herein. In certain embodiments, L2 is a bond; Ring A is of formula:

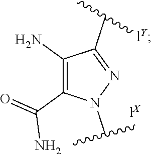 [SMILES: *c1nn(*)c(C(N)=O)c1N]

L1 is of formula:

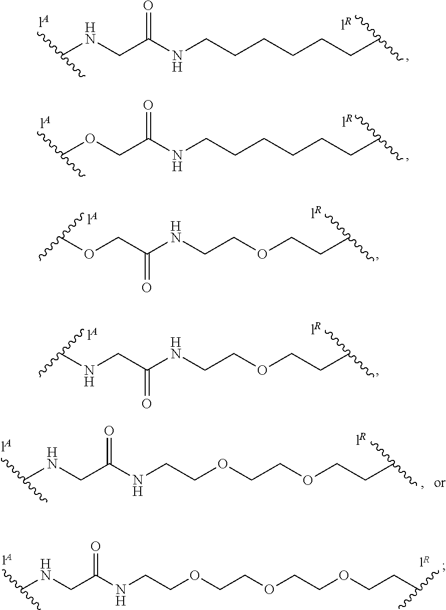

a is 1; b is 0; c is 0; and D is of the formula:

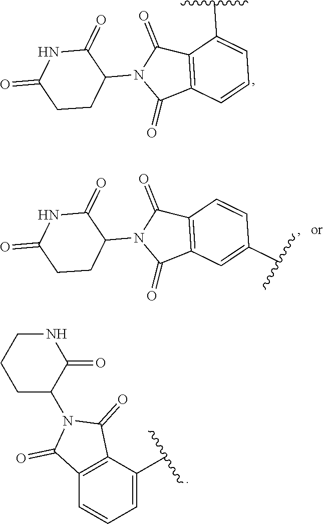

Linker L2 connects the phenyl moiety of formula

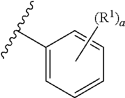

in Formula (I) and Ring A. In certain embodiments, L2 is a bond. In certain embodiments, L2 is of formula

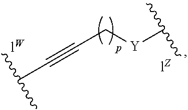

wherein Y is O, —N(R^{Y})—, or S; R^{Y} is hydrogen, optionally substituted acyl, optionally substituted alkyl, optionally substituted alkenyl, optionally substituted alkynyl, or a nitrogen protecting group; p is 0, 1, 2, or 3; l^{Y} indicates the point of attachment to Ring A, and l^{Z} indicates the point of attachment to the phenyl moiety of formula

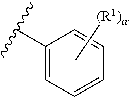

In certain embodiments, L2 is of formula

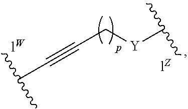

wherein Y is O; and p is 1 or 2. In certain embodiments, L2 is of formula

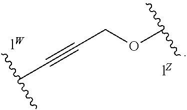 [SMILES: *C#CCO*]

In certain embodiments, the compound of Formula (I) is of the formula:

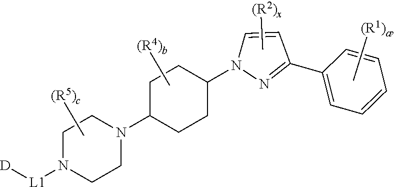

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

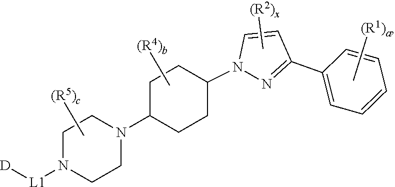

or a pharmaceutically acceptable salt, co-crystal, tautomer, stereoisomer, solvate, hydrate, polymorph, isotopically enriched derivative, or prodrug thereof; L1 is of the formula:

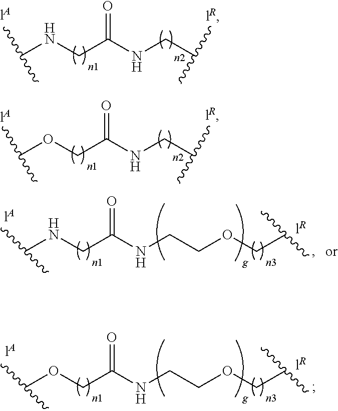

a is 1; b is 0; c is 0; and D is of the formula:

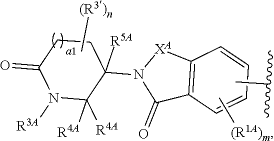

wherein X^{A}, R^{1A}, R^{3A}, R^{3′}, R^{4A}, R^{5A}, m, n, and a1 are as defined herein.

In certain embodiments, the compound of Formula (I) is of the formula:

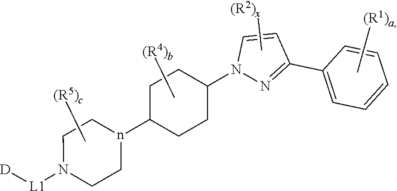

or a pharmaceutically acceptable salt, co-crystal, tautomer, stereoisomer, solvate, hydrate, polymorph, isotopically enriched derivative, or prodrug thereof; L1 is of the formula:

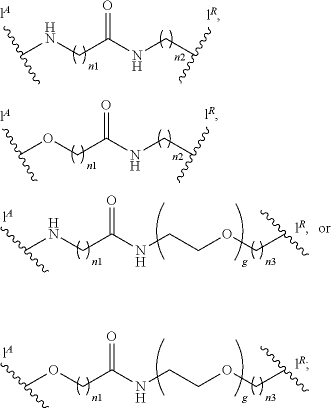

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

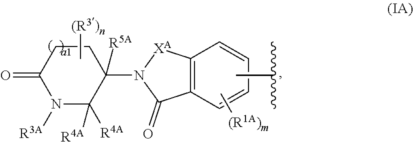

wherein X^{A}, R^{1A}, R^{A}, R^{3′}, R^{4A}, R^{5A}, m, n, and a1 are as defined herein.

In certain embodiments, the compound of Formula (I) is of the formula:

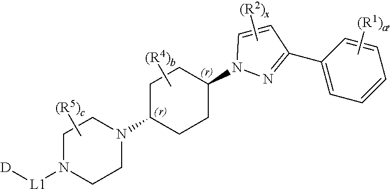

or a pharmaceutically acceptable salt, co-crystal, tautomer, stereoisomer, solvate, hydrate, polymorph, isotopically enriched derivative, or prodrug thereof; L1 is of the formula:

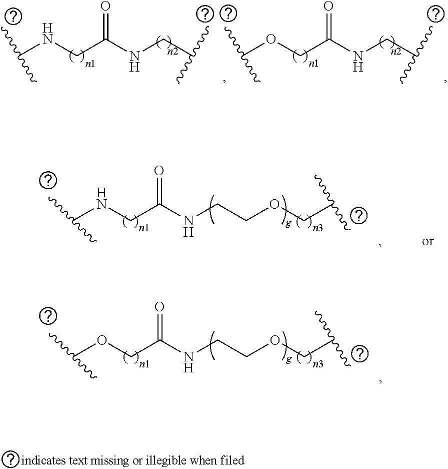

wherein: n is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

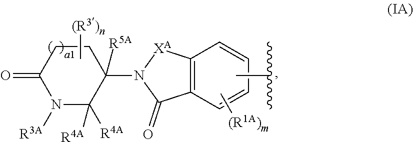

wherein X^{A}, R^{1A}, R^{3A}, R^{3′}, R^{4A}, R^{5A}, m, n, and a1 are as defined herein.

In certain embodiments, the compound of Formula (I) is of the formula:

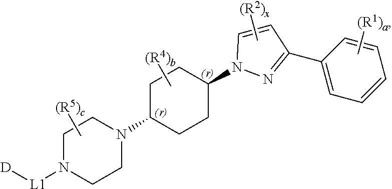

or a pharmaceutically acceptable salt, co-crystal, tautomer, stereoisomer, solvate, hydrate, polymorph, isotopically enriched derivative, or prodrug thereof; L1 is of the formula

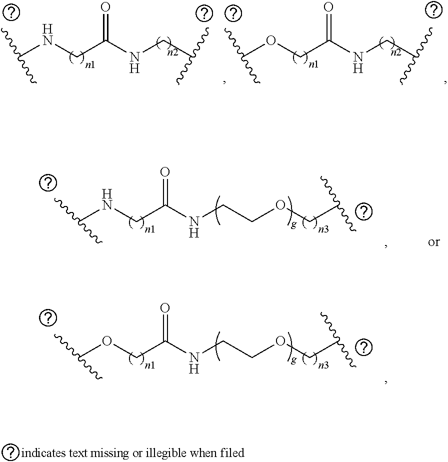

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

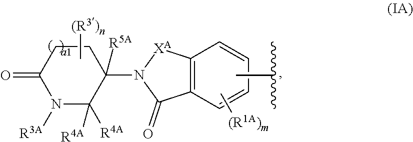

wherein X^{A}, R^{1A}, R^{3A}, R^{3′}, R^{4A}, R^{5A}, m, n, and a1 are as defined herein.

In certain embodiments, the compound of Formula (I) is of the formula:

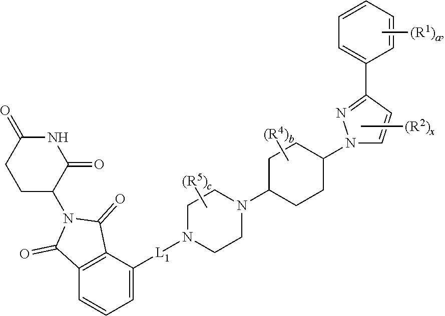

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

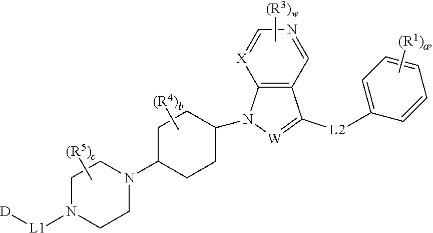

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

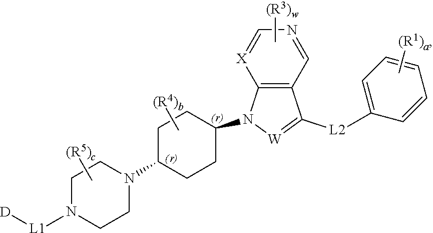

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

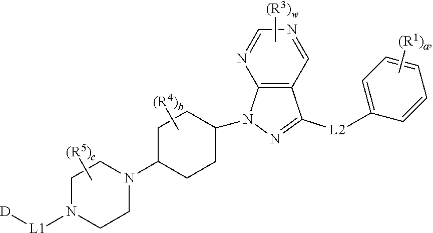

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

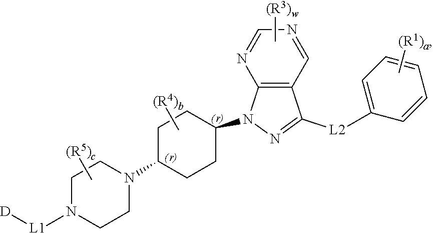

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

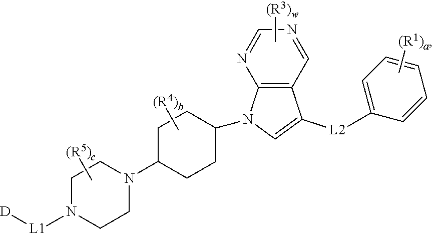

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

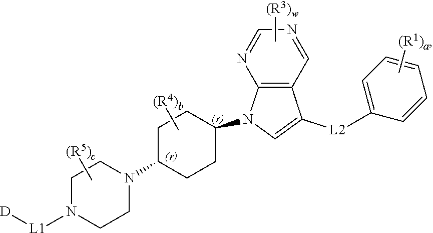

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

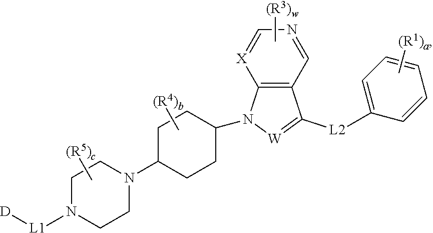

or a pharmaceutically acceptable salt, co-crystal, tautomer, stereoisomer, solvate, hydrate, polymorph, isotopically enriched derivative, or prodrug thereof; L1 is of the formula:

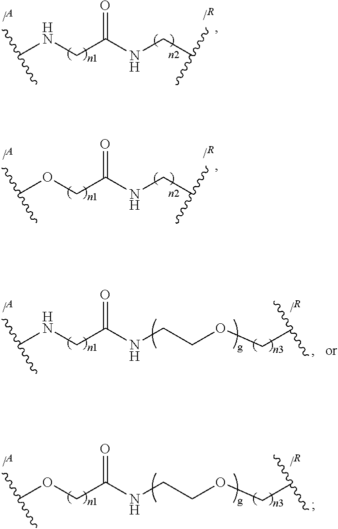

a is 1; b is 0; c is 0; and D is of the formula:

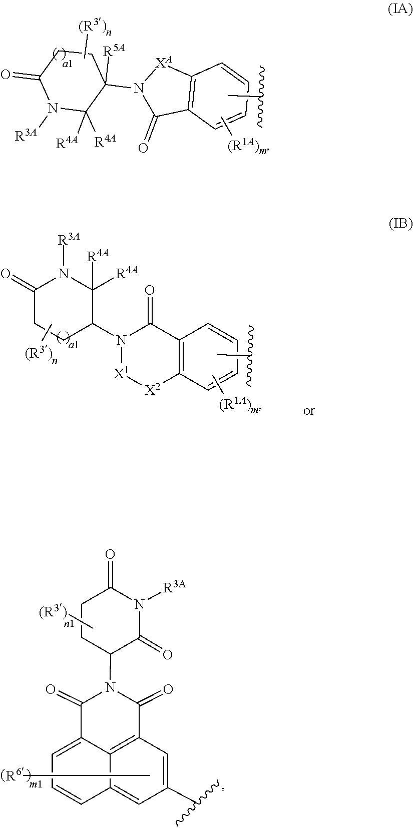

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

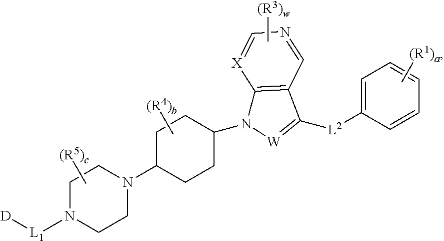

or a pharmaceutically acceptable salt, co-crystal, tautomer, stereoisomer, solvate, hydrate, polymorph, isotopically enriched derivative, or prodrug thereof; L1 is of the formula:

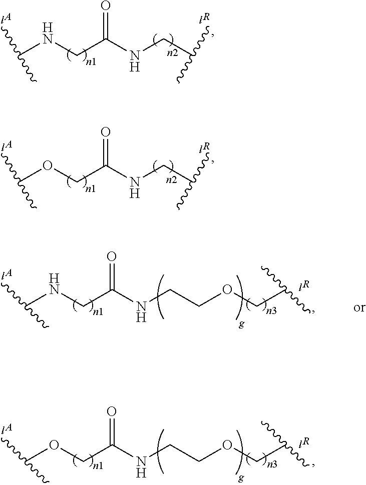

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

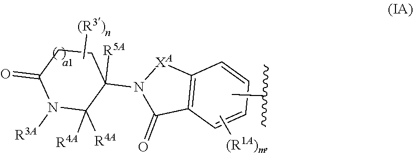

wherein X^{A}, R^{1A}, R^{3A}, R^{3′}, R^{4A}, R^{5A}, m, n, and a1 are as defined herein.

In certain embodiments, the compound of Formula (I) is of the formula:

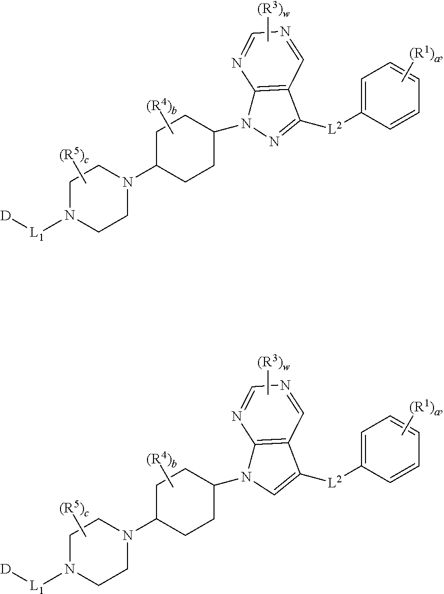

or a pharmaceutically acceptable salt, co-crystal, tautomer, stereoisomer, solvate, hydrate, polymorph, isotopically enriched derivative, or prodrug thereof; L1 is of the formula:

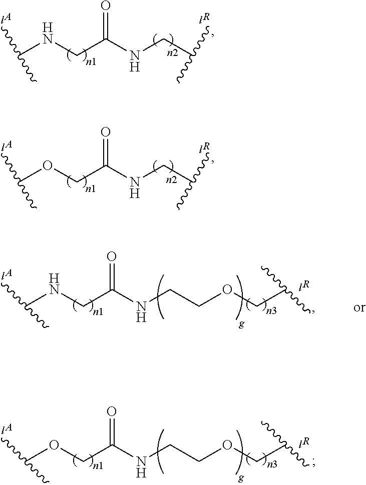

a is 1; b is 0; c is 0; and D is of the formula:

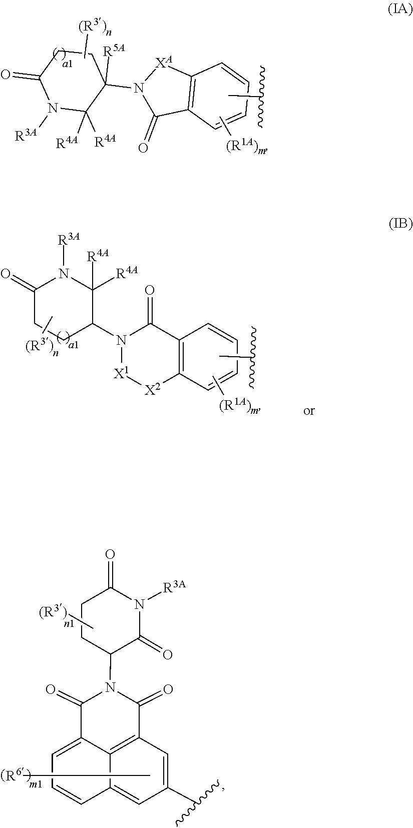

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

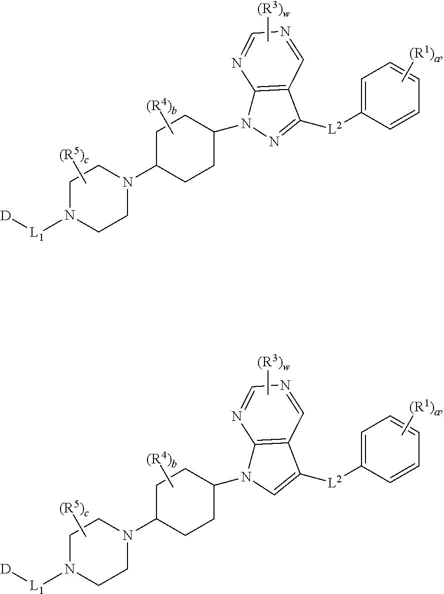

or a pharmaceutically acceptable salt, co-crystal, tautomer, stereoisomer, solvate, hydrate, polymorph, isotopically enriched derivative, or prodrug thereof; L1 is of the formula:

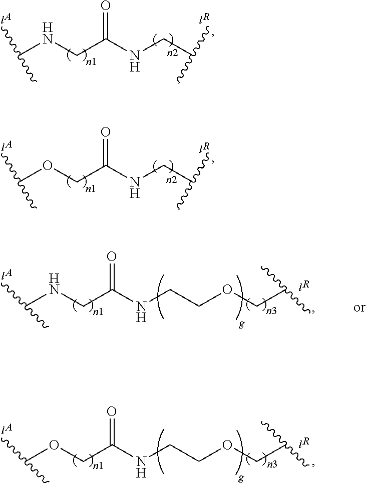

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

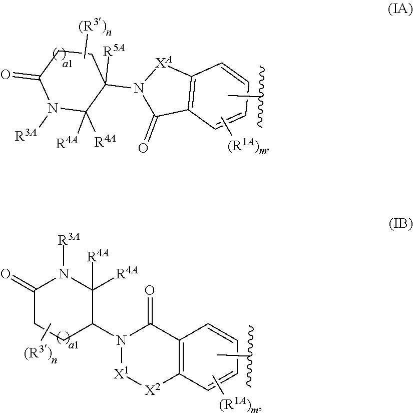

wherein —X¹—X²—, X^{A}, R¹, R^{3A}, R^{3′}, R^{4A}, R^{5A}, m, n, and a1 are as defined herein.

In certain embodiments, the compound of Formula (I) is of the formula:

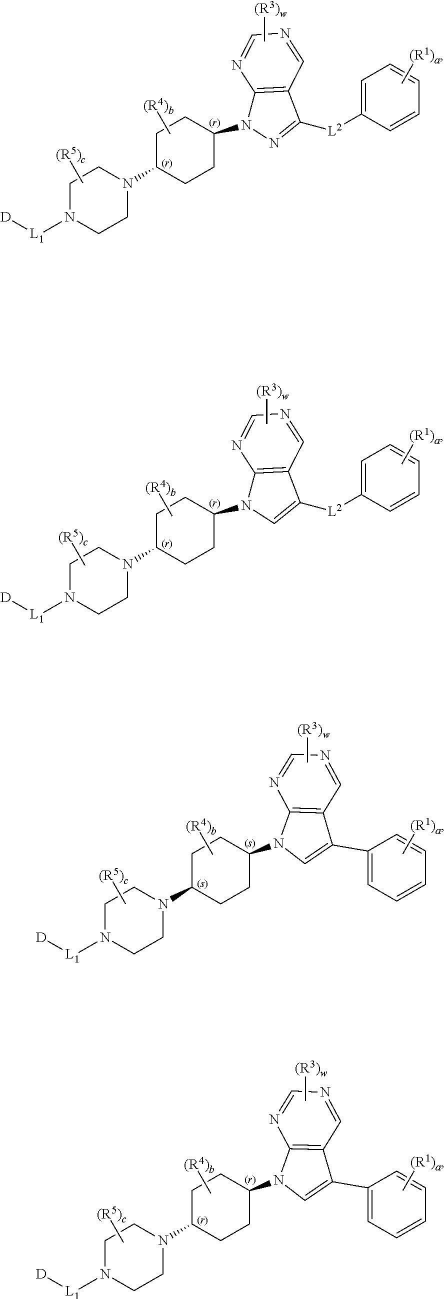

or a pharmaceutically acceptable salt, co-crystal, tautomer, stereoisomer, solvate, hydrate, polymorph, isotopically enriched derivative, or prodrug thereof; L1 is of the formula:

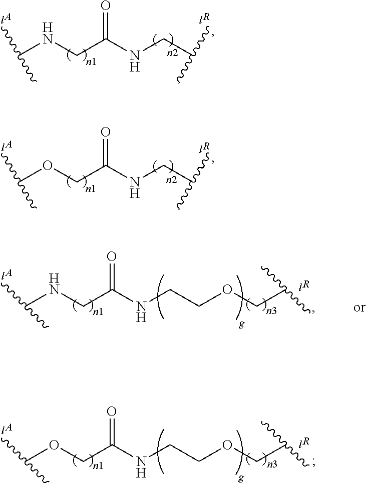

a is 1; b is 0; c is 0; and D is of the formula:

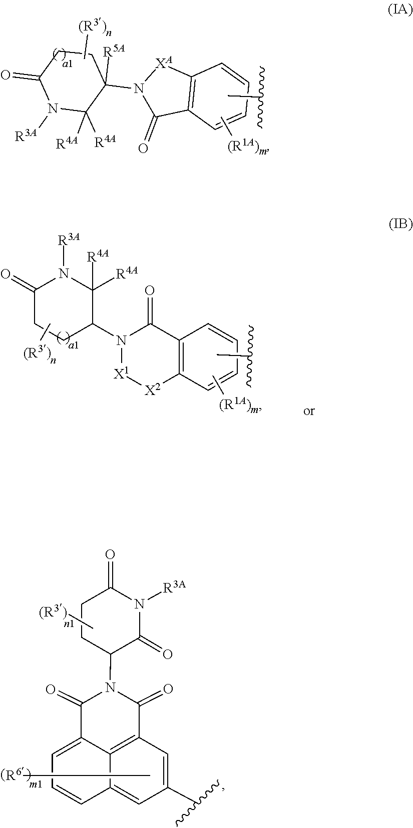

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

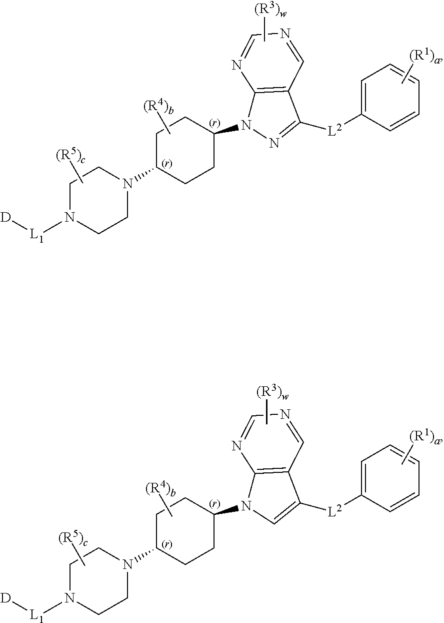

or a pharmaceutically acceptable salt, co-crystal, tautomer, stereoisomer, solvate, hydrate, polymorph, isotopically enriched derivative, or prodrug thereof; L1 is of the formula:

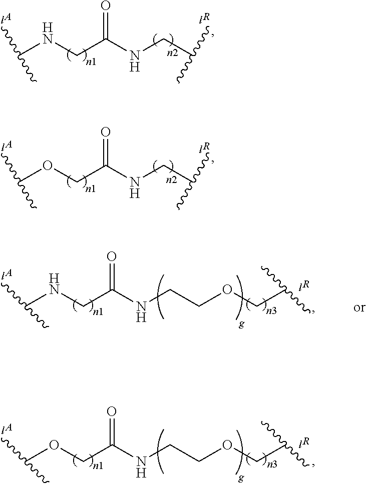

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

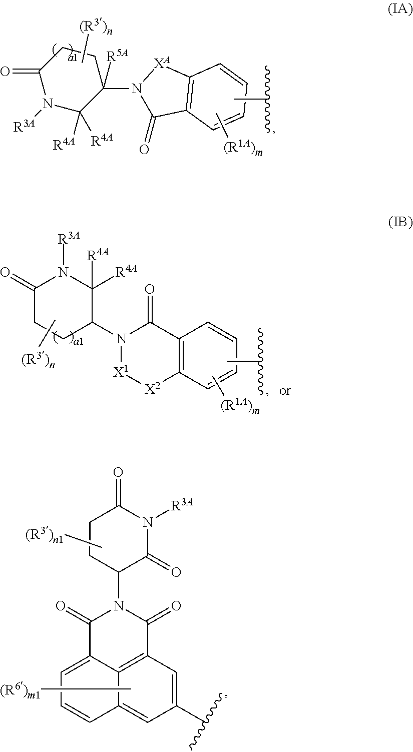

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

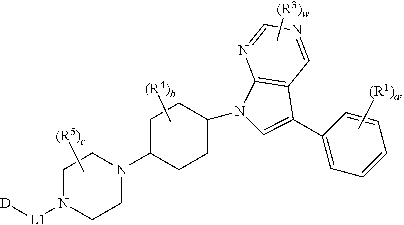

or a pharmaceutically acceptable salt, co-crystal, tautomer, stereoisomer, solvate, hydrate, polymorph, isotopically enriched derivative, or prodrug thereof; L1 is of the formula:

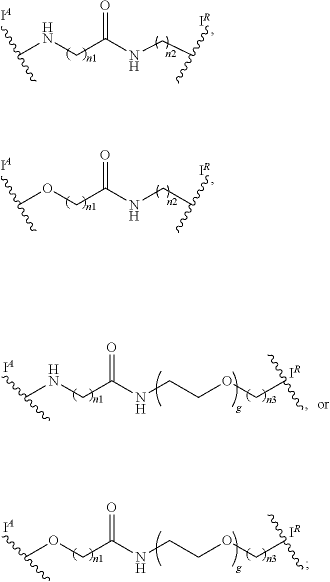

a is 1; b is 0; c is 0; and D is of the formula:

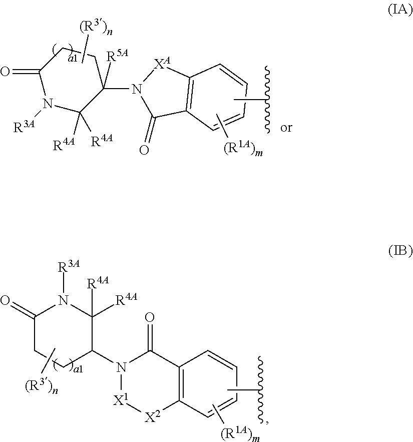

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

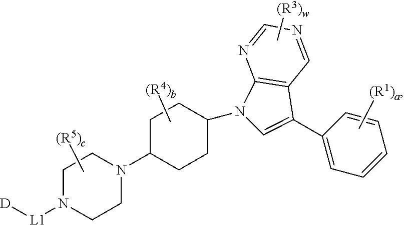

or a pharmaceutically acceptable salt, co-crystal, tautomer, stereoisomer, solvate, hydrate, polymorph, isotopically enriched derivative, or prodrug thereof; L1 is of the formula:

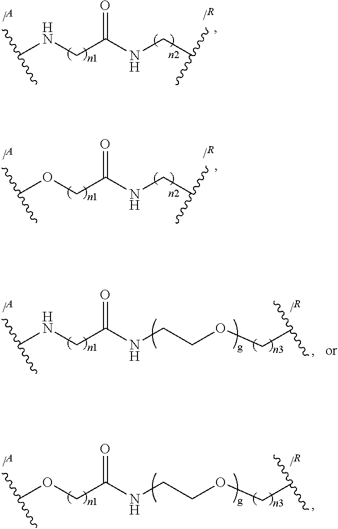

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

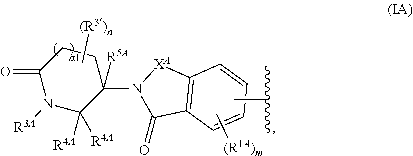

wherein X^{A}, R^{1A}, R^{3A}, R^{3′}, R^{4A}, R^{5A}, m, n, and a1 are as defined herein.

In certain embodiments, the compound of Formula (I) is of the formula:

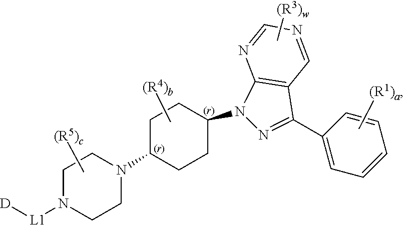

or a pharmaceutically acceptable salt, co-crystal, tautomer, stereoisomer, solvate, hydrate, polymorph, isotopically enriched derivative, or prodrug thereof; L1 is of the formula:

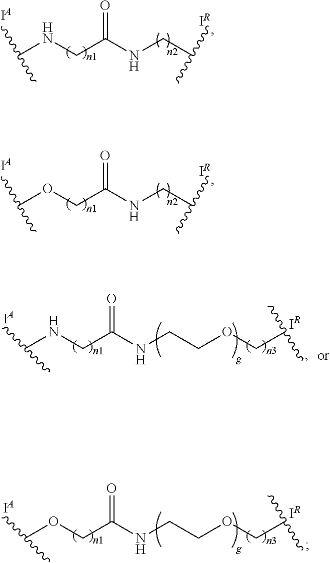

a is 1; b is 0; c is 0; and D is of the formula:

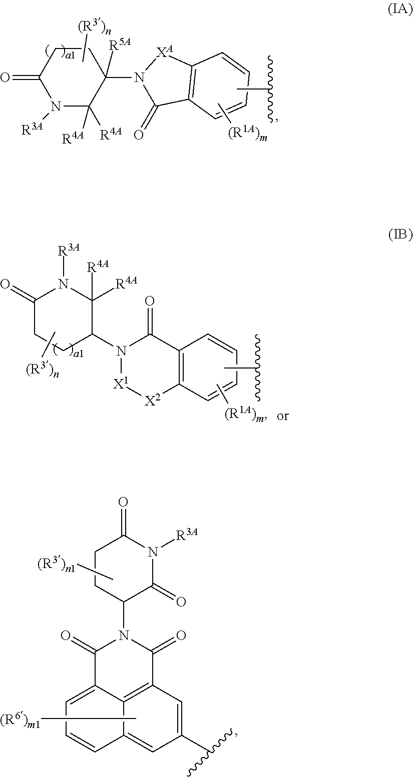

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

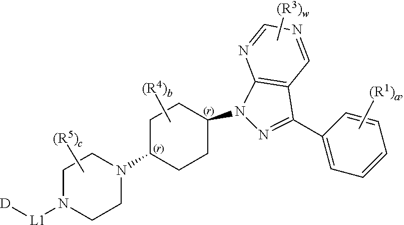

or a pharmaceutically acceptable salt, co-crystal, tautomer, stereoisomer, solvate, hydrate, polymorph, isotopically enriched derivative, or prodrug thereof; L1 is of the formula:

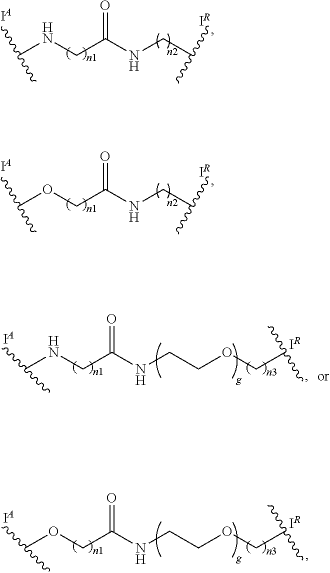

wherein: n1 is 1, 2, or 3; n2 is 4, 5, 6, 7, 8 or 9; n3 is 1, 2, 3, or 4; and g is 1, 2, 3, 4, or 5; a is 1; b is 0; c is 0; and D is of the formula:

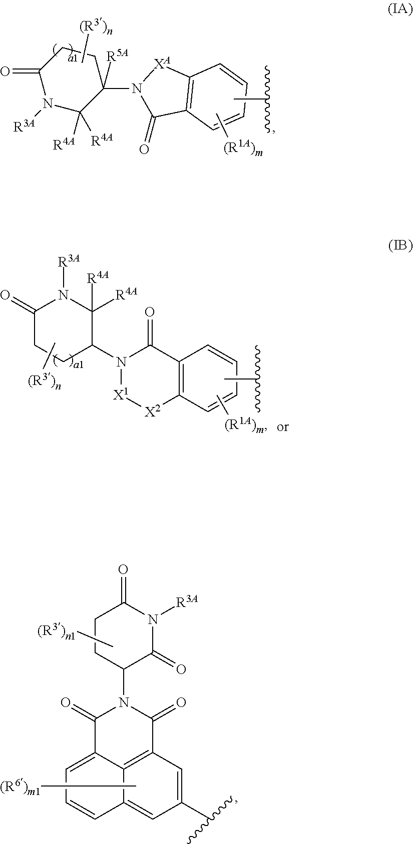

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

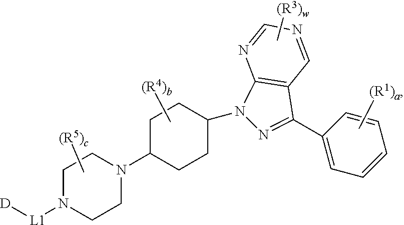

or a pharmaceutically acceptable salt, co-crystal, tautomer, stereoisomer, solvate, hydrate, polymorph, isotopically enriched derivative, or prodrug thereof; L1 is of the formula:

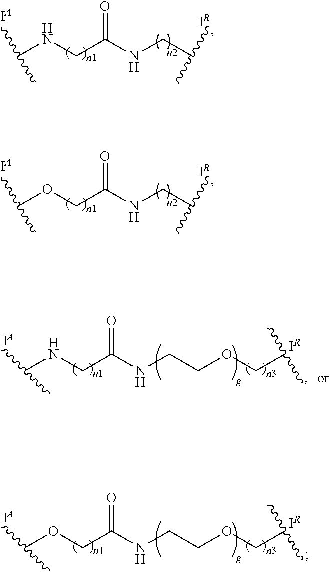

a is 1; b is 0; c is 0; and D is of the formula:

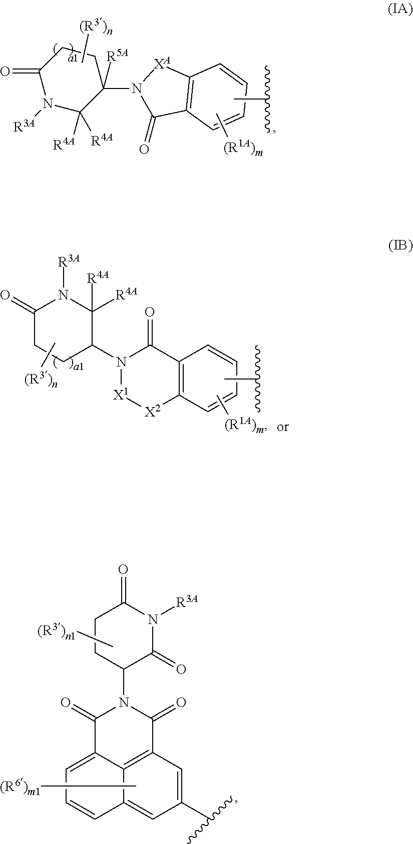

wherein —X¹—X²—, X^{A}, R^{1A}, R^{3A}, R^{3′}, R^{4A}, R^{5A}, m, n, n1, and a1. In certain embodiments, the compound of Formula (I) is of the formula:

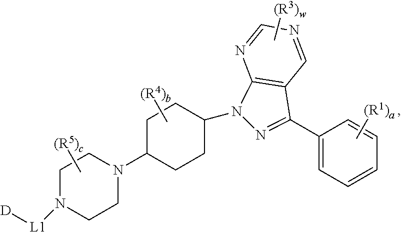

or a pharmaceutically acceptable salt, co-crystal, tautomer, stereoisomer, solvate, hydrate, polymorph, isotopically enriched derivative, or prodrug thereof; L1 is of the formula:

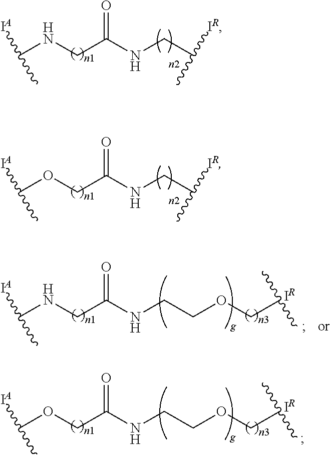

a is 1; b is 0; c is 0; and D is of the formula:

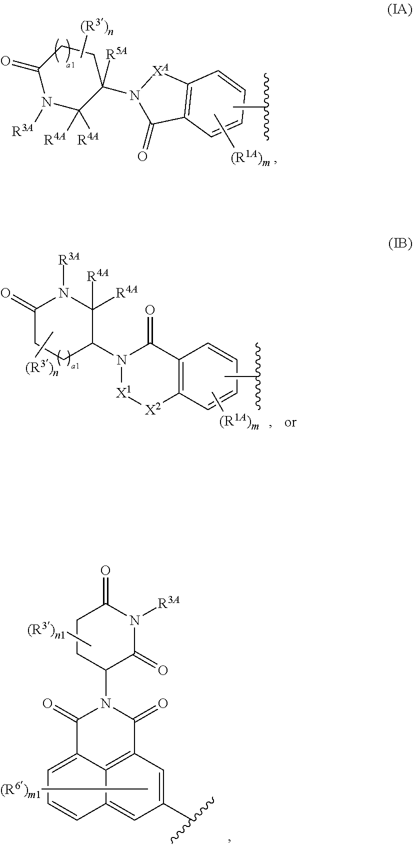

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

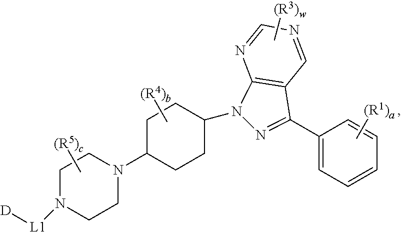

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

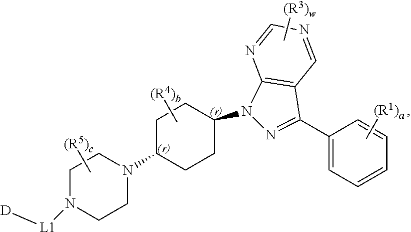

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

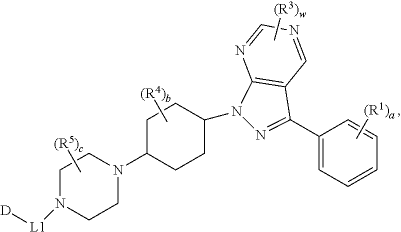

or a pharmaceutically acceptable salt, co-crystal, tautomer, stereoisomer, solvate, hydrate, polymorph, isotopically enriched derivative, or prodrug thereof; L1 is of the formula:

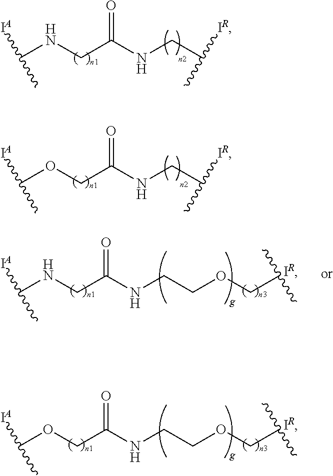

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

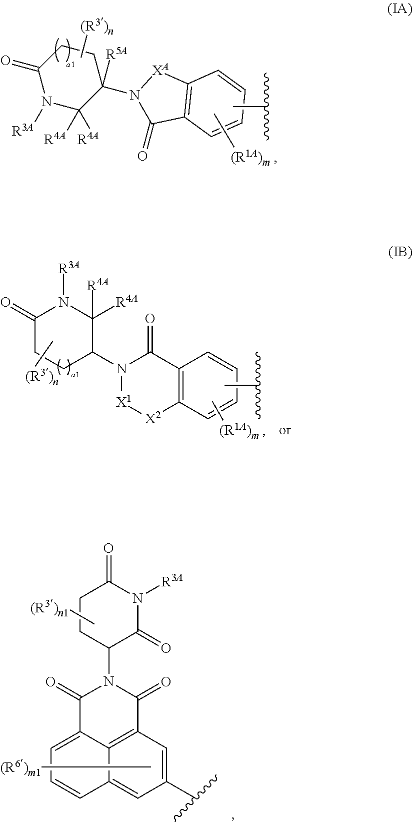

wherein —X¹—X²—, X^{A}, R^{1A}, R^{3A}, R^{3′}, R^{4A}, R^{5A}, m, n, n1, and a1 are as defined herein.

In certain embodiments, the compound of Formula (I) is of the formula:

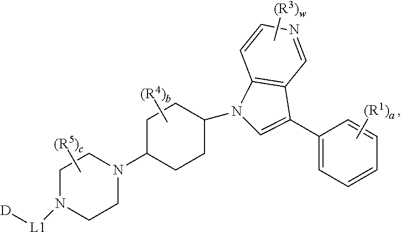

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

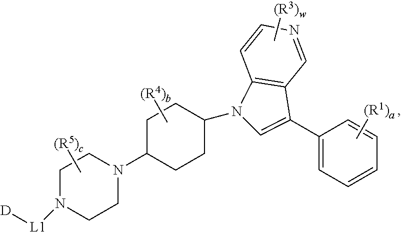

or a pharmaceutically acceptable salt, co-crystal, tautomer, stereoisomer, solvate, hydrate, polymorph, isotopically enriched derivative, or prodrug thereof; L1 is of the formula:

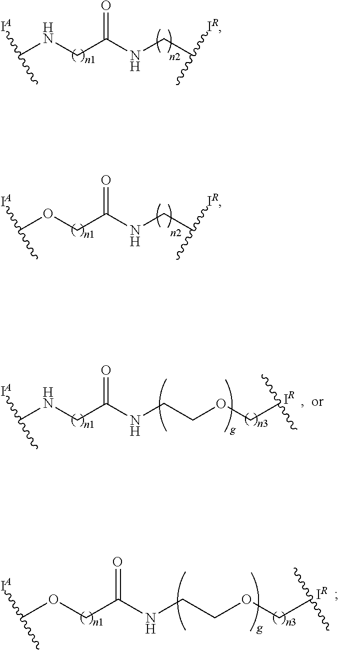

a is 1; b is 0; c is 0; and D is of the formula:

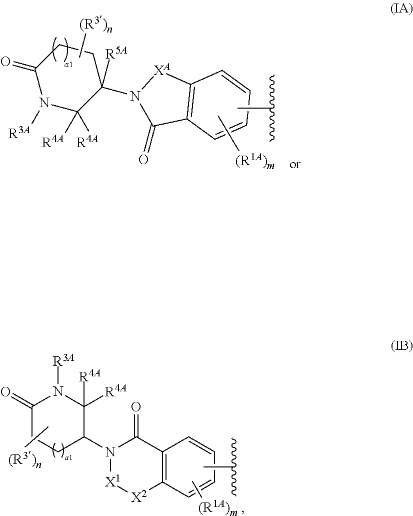

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

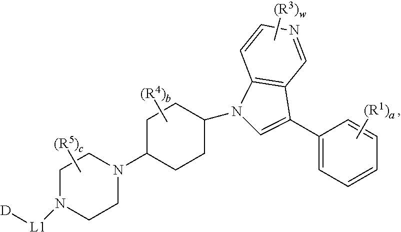

or a pharmaceutically acceptable salt, co-crystal, tautomer, stereoisomer, solvate, hydrate, polymorph, isotopically enriched derivative, or prodrug thereof; L1 is of the formula:

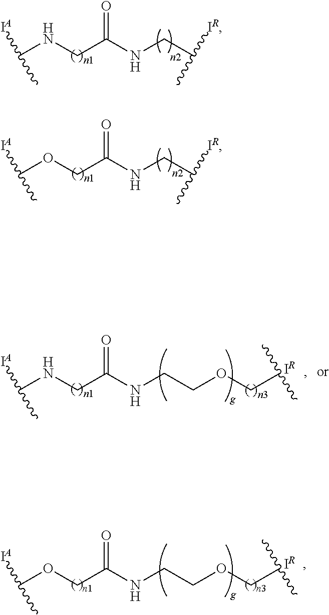

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

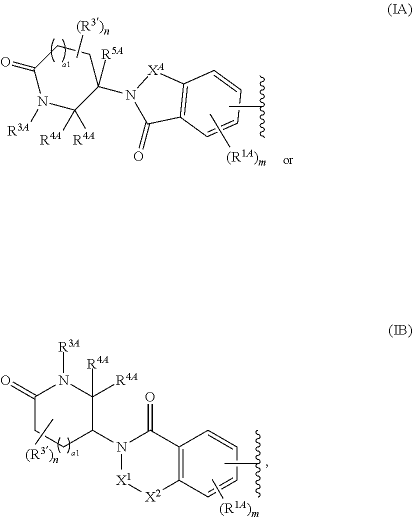

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

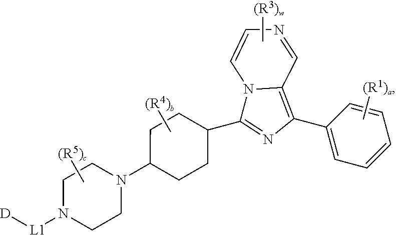

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

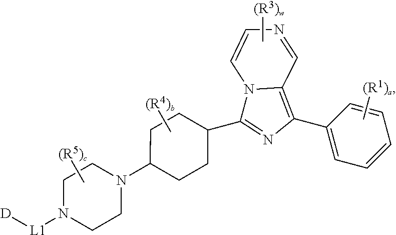

or a pharmaceutically acceptable salt, co-crystal, tautomer, stereoisomer, solvate, hydrate, polymorph, isotopically enriched derivative, or prodrug thereof; wherein a is 0, 1, or 2.

In certain embodiments, the compound of Formula (I) is of the formula:

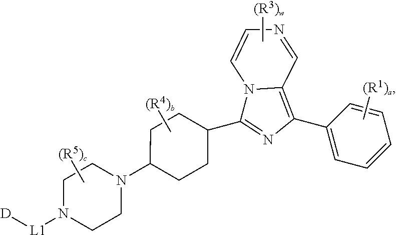

or a pharmaceutically acceptable salt, co-crystal, tautomer, stereoisomer, solvate, hydrate, polymorph, isotopically enriched derivative, or prodrug thereof; L1 is of the formula:

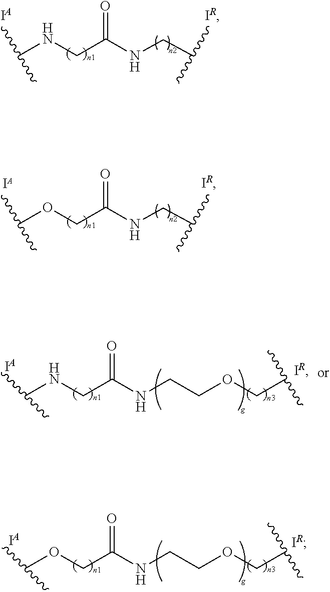

a is 1; b is 0; c is 0; and D is of the formula:

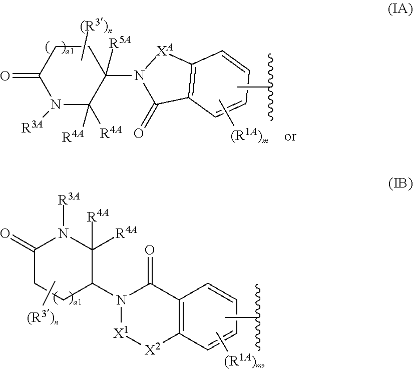

wherein —X¹—X²—, X^{A}, R^{1A}, R^{3A}, R³, R^{4A}, R^{5A}, m, n, and a1 are as defined herein.

In certain embodiments, the compound of Formula (I) is of the formula:

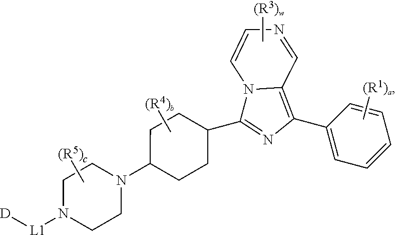

or a pharmaceutically acceptable salt, co-crystal, tautomer, stereoisomer, solvate, hydrate, polymorph, isotopically enriched derivative, or prodrug thereof; L1 is of the formula:

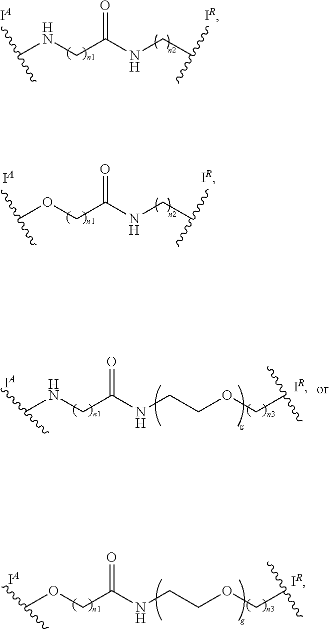

wherein: n is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

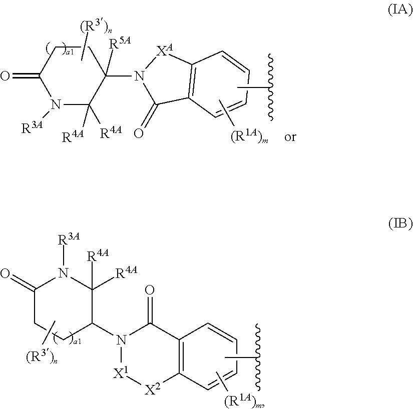

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

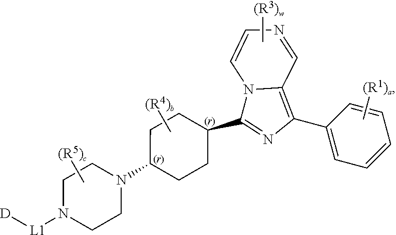

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

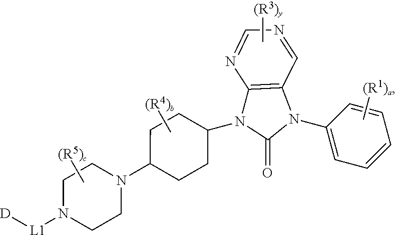

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

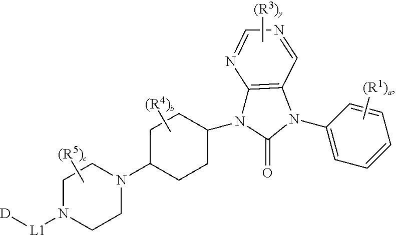

or a pharmaceutically acceptable salt, co-crystal, tautomer, stereoisomer, solvate, hydrate, polymorph, isotopically enriched derivative, or prodrug thereof; wherein a is 0, 1, or 2.

In certain embodiments, the compound of Formula (I) is of the formula:

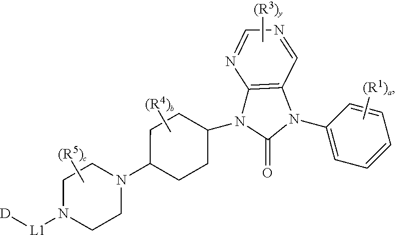

or a pharmaceutically acceptable salt, co-crystal, tautomer, stereoisomer, solvate, hydrate, polymorph, isotopically enriched derivative, or prodrug thereof; L1 is of the formula:

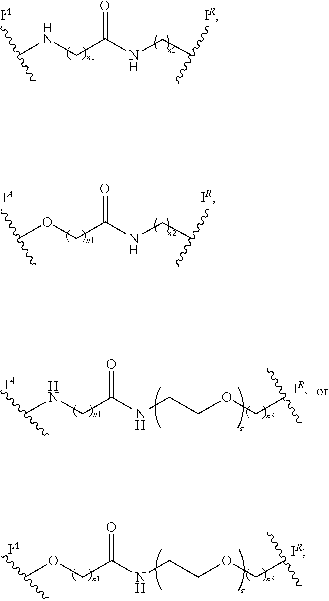

a is 1; b is 0; c is 0; and D is of the formula:

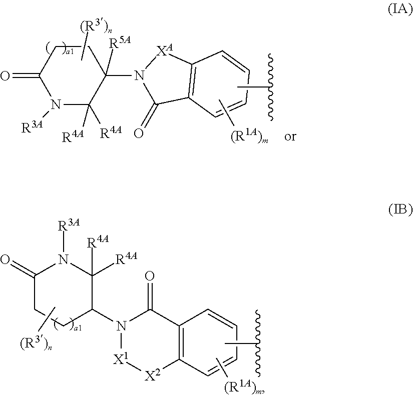

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

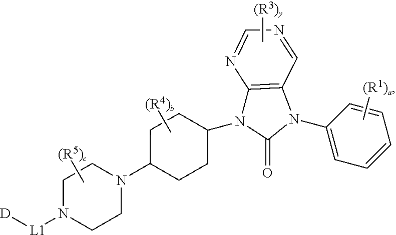

or a pharmaceutically acceptable salt, co-crystal, tautomer, stereoisomer, solvate, hydrate, polymorph, isotopically enriched derivative, or prodrug thereof; L1 is of the formula:

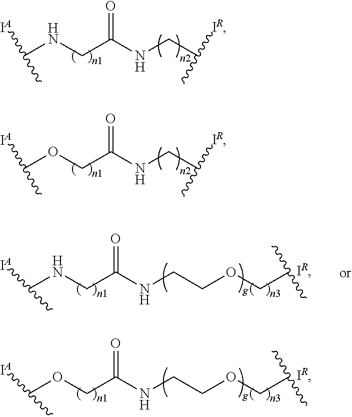

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

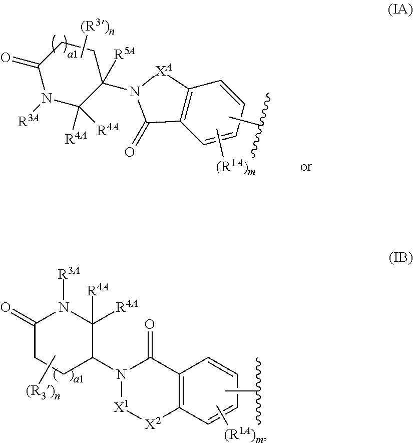

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

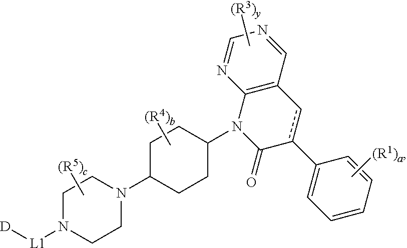

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

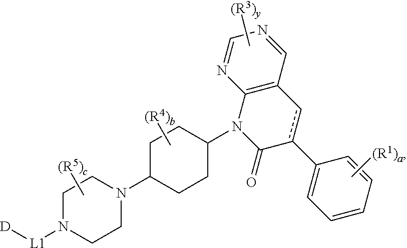

or a pharmaceutically acceptable salt, co-crystal, tautomer, stereoisomer, solvate, hydrate, polymorph, isotopically enriched derivative, or prodrug thereof, wherein a is 0, 1, or 2. In certain embodiments, the compound of Formula (I) is of the formula:

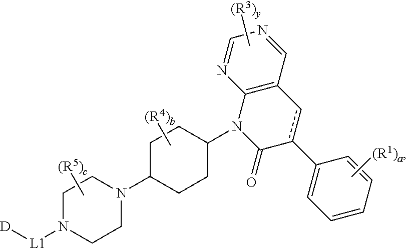

or a pharmaceutically acceptable salt, co-crystal, tautomer, stereoisomer, solvate, hydrate, polymorph, isotopically enriched derivative, or prodrug thereof; L1 is of the formula:

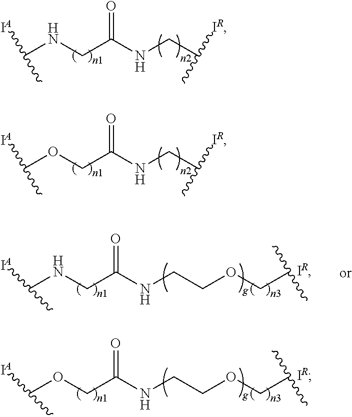

a is 1; b is 0; c is 0; and D is of the formula:

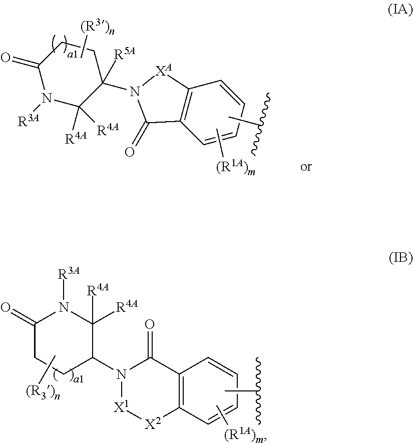

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

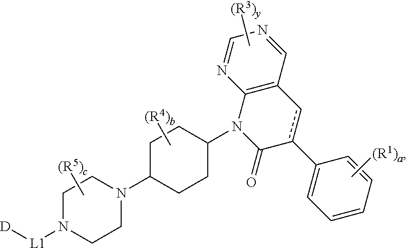

or a pharmaceutically acceptable salt, co-crystal, tautomer, stereoisomer, solvate, hydrate, polymorph, isotopically enriched derivative, or prodrug thereof; L1 is of the formula:

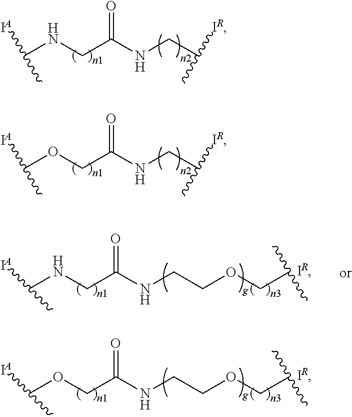

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

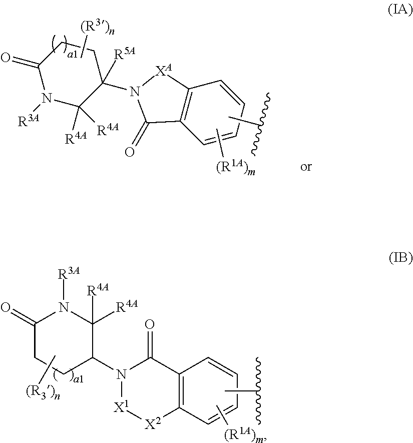

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

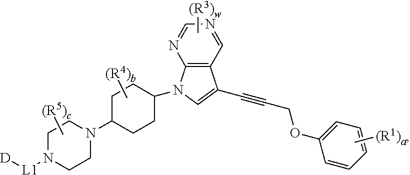

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

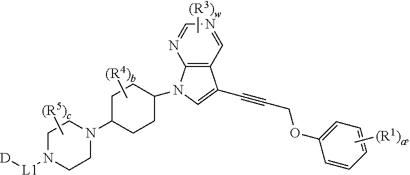

or a pharmaceutically acceptable salt, co-crystal, tautomer, stereoisomer, solvate, hydrate, polymorph, isotopically enriched derivative, or prodrug thereof; L1 is of the formula:

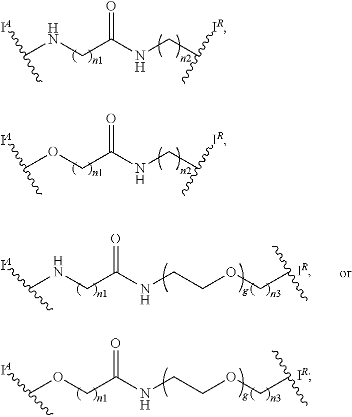

a is 1; b is 0; c is 0; and D is of the formula:

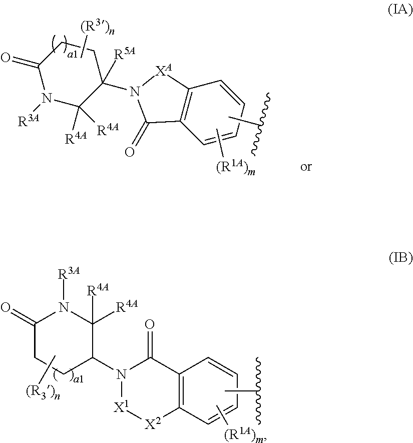

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

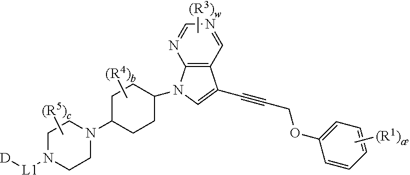

or a pharmaceutically acceptable salt, co-crystal, tautomer, stereoisomer, solvate, hydrate, polymorph, isotopically enriched derivative, or prodrug thereof; L1 is of the formula:

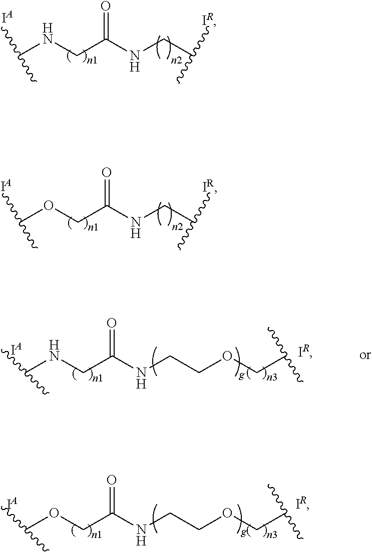

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

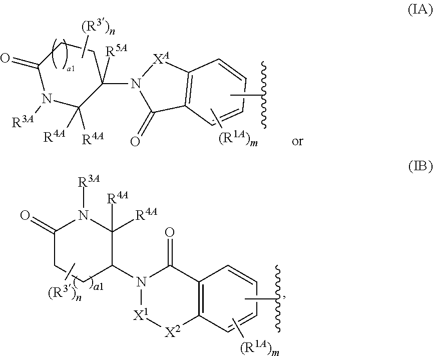

—X¹—X²—, X^{A}, R¹, R^{3A}, R³, R^{4A}, R^{5A}, m, n, and a1 are as defined herein.

In certain embodiments, the compound of Formula (I) is of the formula:

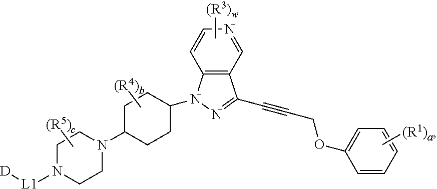

or a pharmaceutically acceptable salt, co-crystal, tautomer, stereoisomer, solvate, hydrate, polymorph, isotopically enriched derivative, or prodrug thereof. In certain embodiments, the compound of Formula (I) is of the formula:

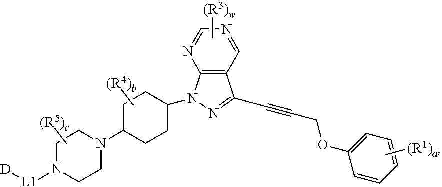

or a pharmaceutically acceptable salt, co-crystal, tautomer, stereoisomer, solvate, hydrate, polymorph, isotopically enriched derivative, or prodrug thereof; L1 is of the formula:

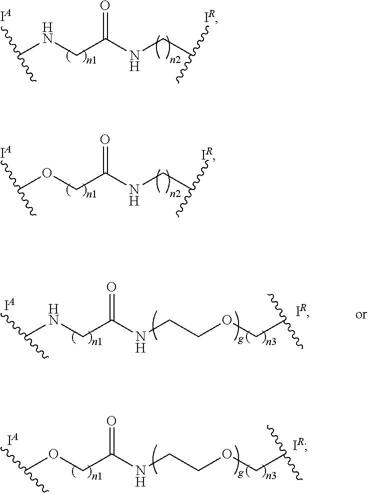

a is 1; b is 0; c is 0; and D is of the formula:

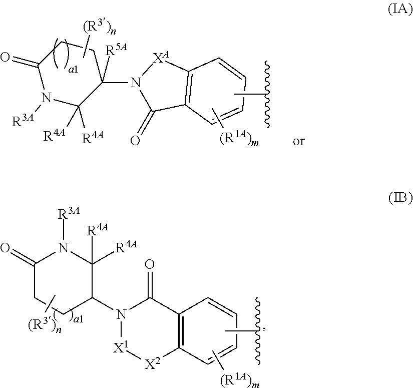

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

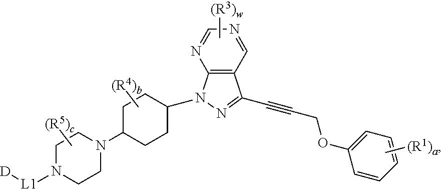

or a pharmaceutically acceptable salt, co-crystal, tautomer, stereoisomer, solvate, hydrate, polymorph, isotopically enriched derivative, or prodrug thereof; L1 is of the formula:

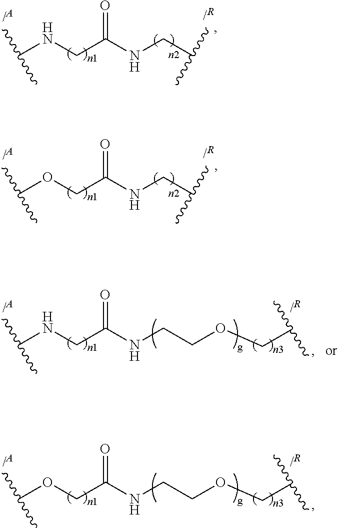

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

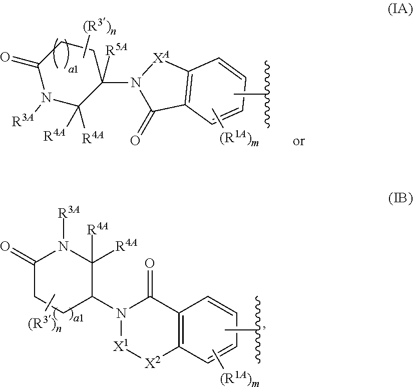

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

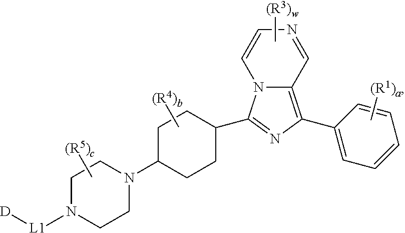

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

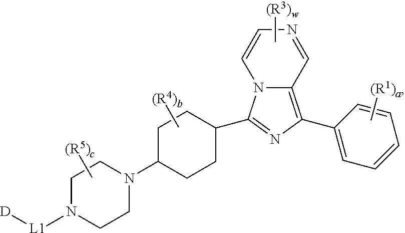

or a pharmaceutically acceptable salt, co-crystal, tautomer, stereoisomer, solvate, hydrate, polymorph, isotopically enriched derivative, or prodrug thereof; L1 is of the formula:

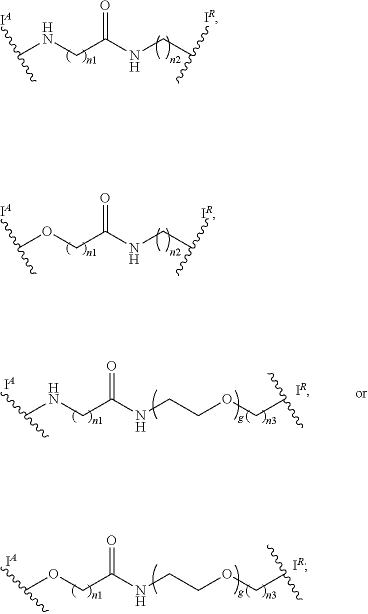

a is 1; b is 0; c is 0; and D is of the formula:

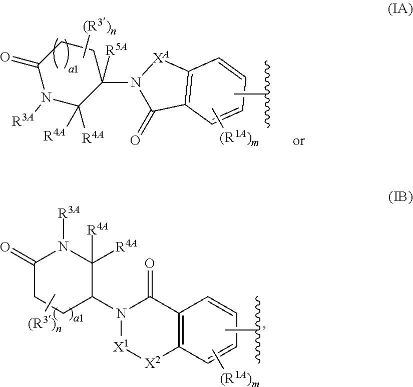

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

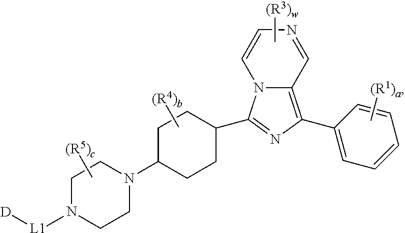

or a pharmaceutically acceptable salt, co-crystal, tautomer, stereoisomer, solvate, hydrate, polymorph, isotopically enriched derivative, or prodrug thereof; L1 is of the formula:

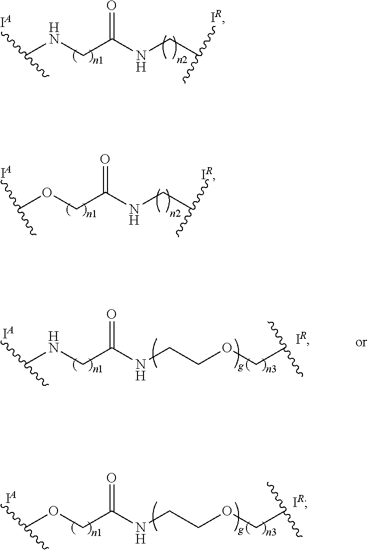

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

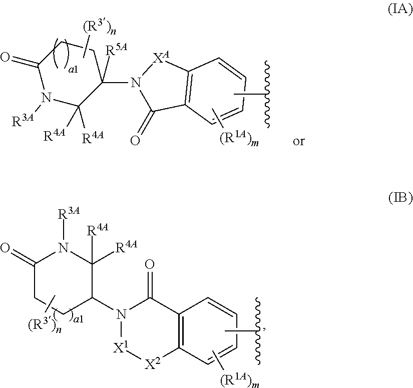

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

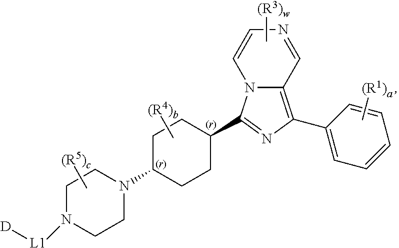

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

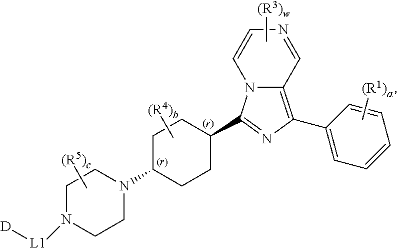

or a pharmaceutically acceptable salt, co-crystal, tautomer, stereoisomer, solvate, hydrate, polymorph, isotopically enriched derivative, or prodrug thereof; L1 is of the formula:

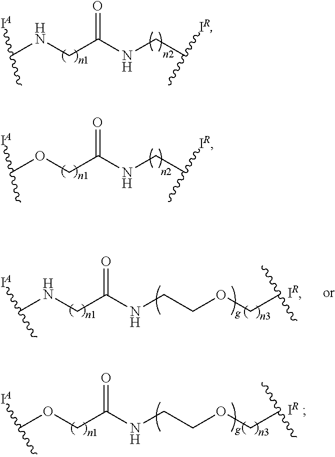

a is 1; b is 0; c is 0; and D is of the formula:

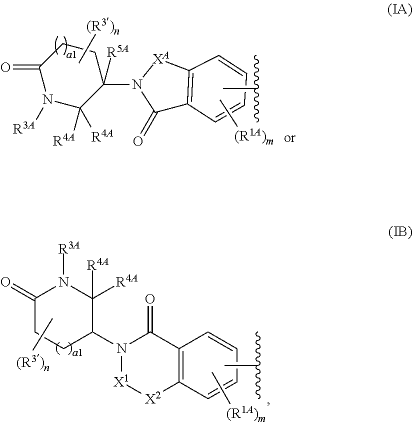

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

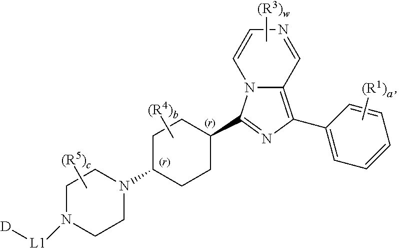

or a pharmaceutically acceptable salt, co-crystal, tautomer, stereoisomer, solvate, hydrate, polymorph, isotopically enriched derivative, or prodrug thereof; L1 is of the formula:

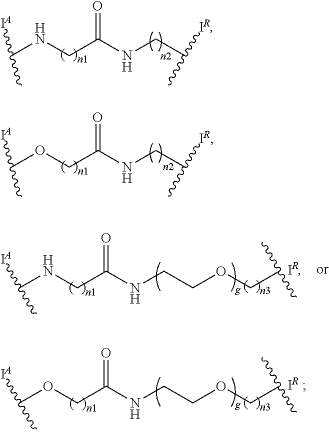

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

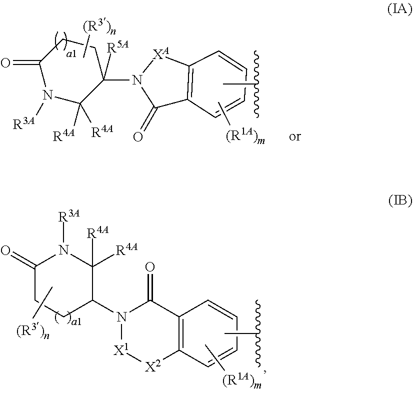

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

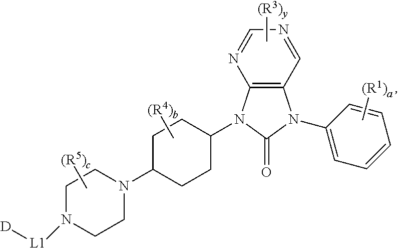

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

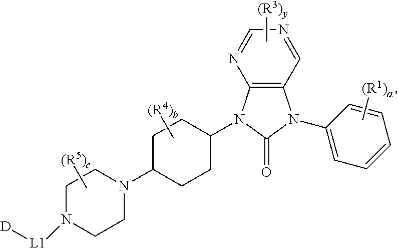

or a pharmaceutically acceptable salt, co-crystal, tautomer, stereoisomer, solvate, hydrate, polymorph, isotopically enriched derivative, or prodrug thereof; L1 is of the formula:

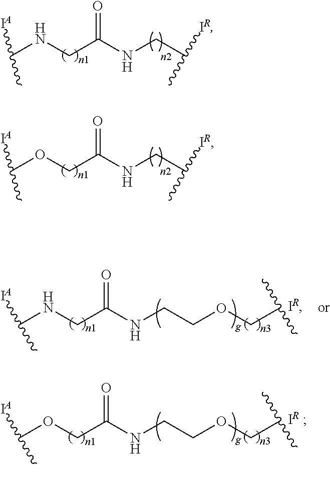

a is 1; b is 0; c is 0; and D is of the formula:

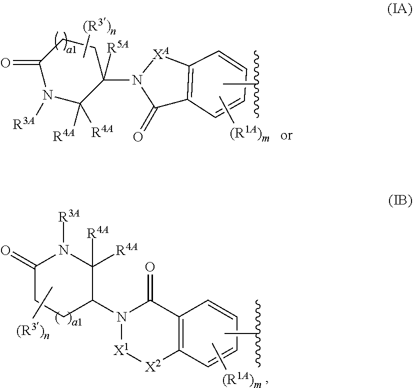

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

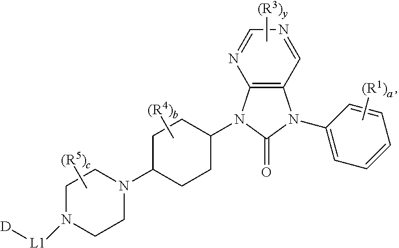

or a pharmaceutically acceptable salt, co-crystal, tautomer, stereoisomer, solvate, hydrate, polymorph, isotopically enriched derivative, or prodrug thereof; L1 is of the formula:

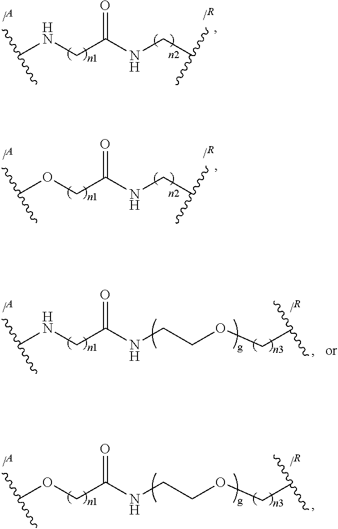

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

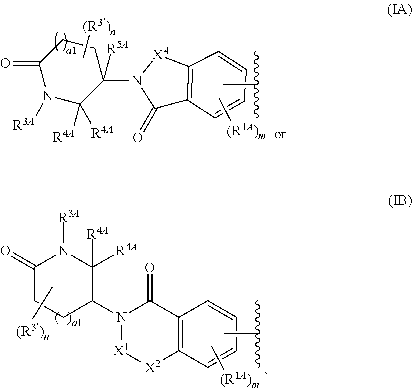

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

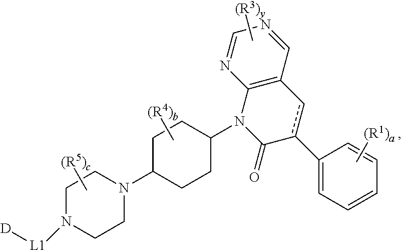

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

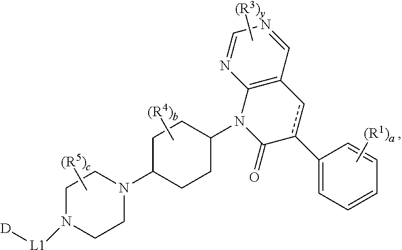

or a pharmaceutically acceptable salt, co-crystal, tautomer, stereoisomer, solvate, hydrate, polymorph, isotopically enriched derivative, or prodrug thereof; L1 is of the formula:

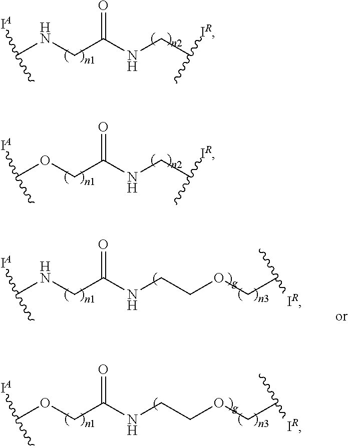

a is 1; b is 0; c is 0; and D is of the formula:

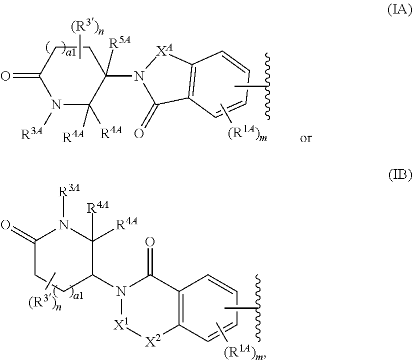

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

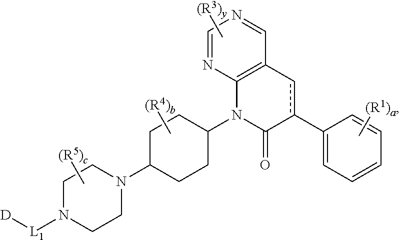

or a pharmaceutically acceptable salt, co-crystal, tautomer, stereoisomer, solvate, hydrate, polymorph, isotopically enriched derivative, or prodrug thereof; L1 is of the formula:

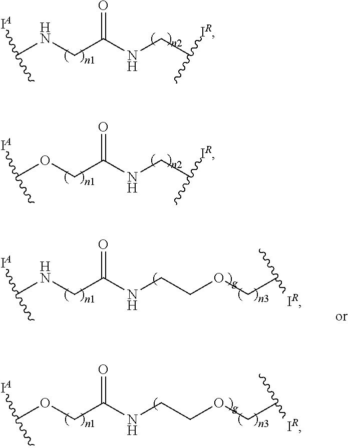

wherein: n1 is 1, 2, or 3; n2 is 4, 5, 6, 7, 8, or 9; n3 is 1, 2, 3, or 4; and g is 1, 2, 3, 4, or 5; a is 1; b is 0; c is 0; and D is of the formula:

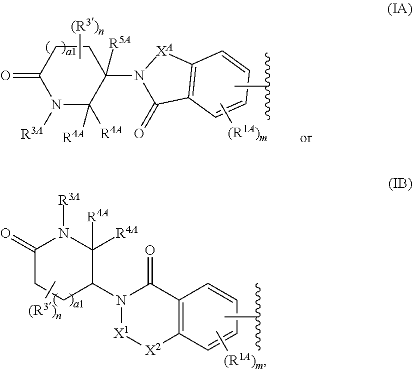

wherein —X¹—X²—, X^{A}, R^{1A}, R^{3A}, R^{3′}, R^{4A}, R^{5A}, m, n, and a1 are as defined herein.

In certain embodiments, the compound of Formula (I) is of the formula:

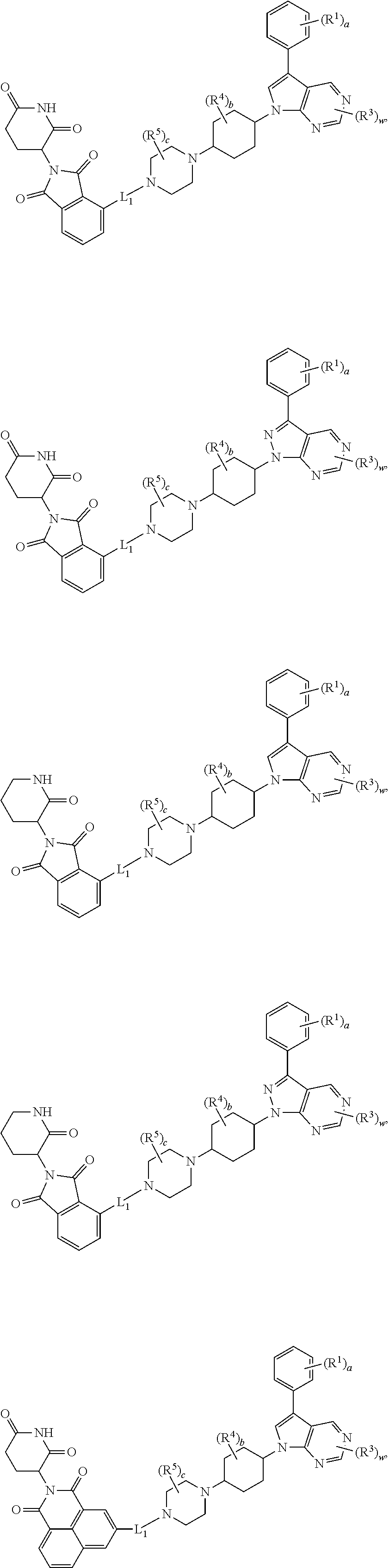

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

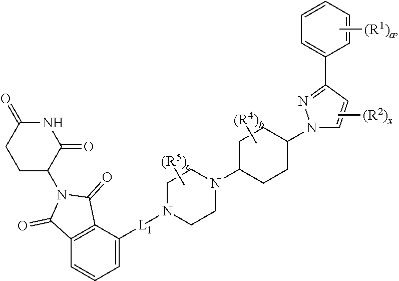

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

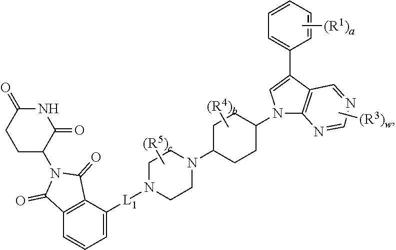

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

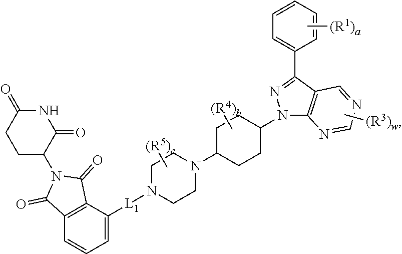

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

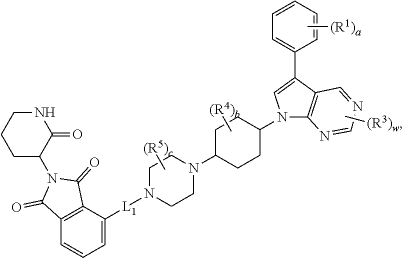

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

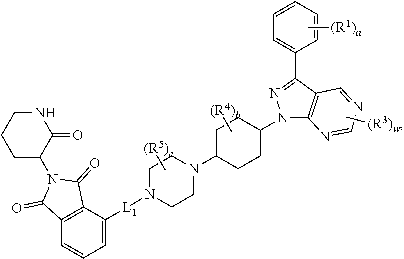

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

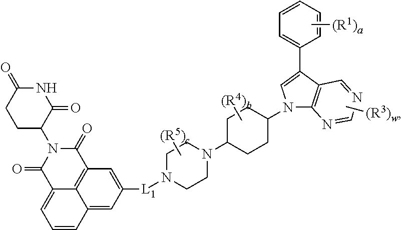

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

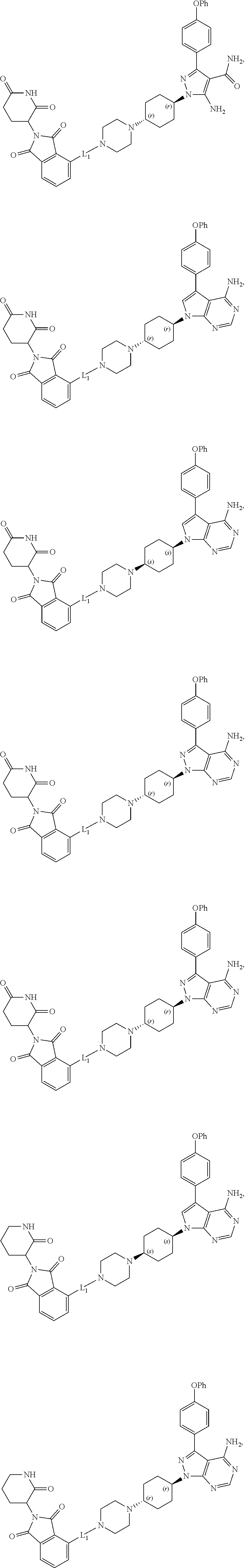

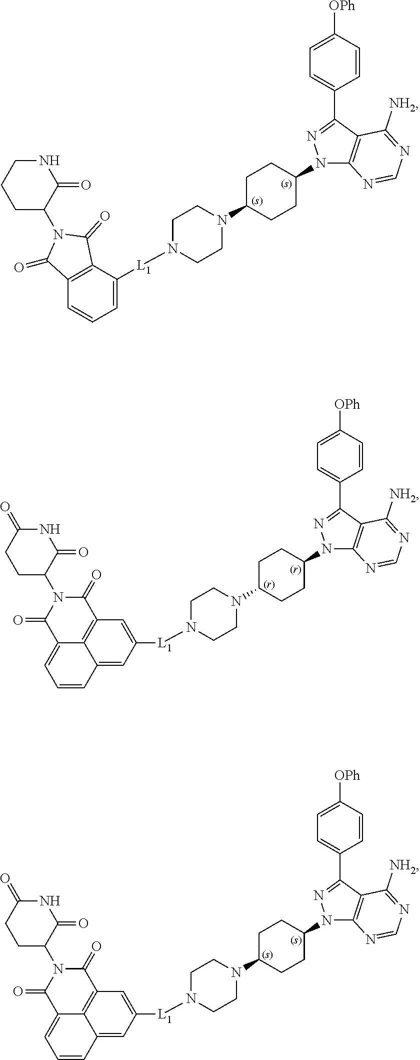

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

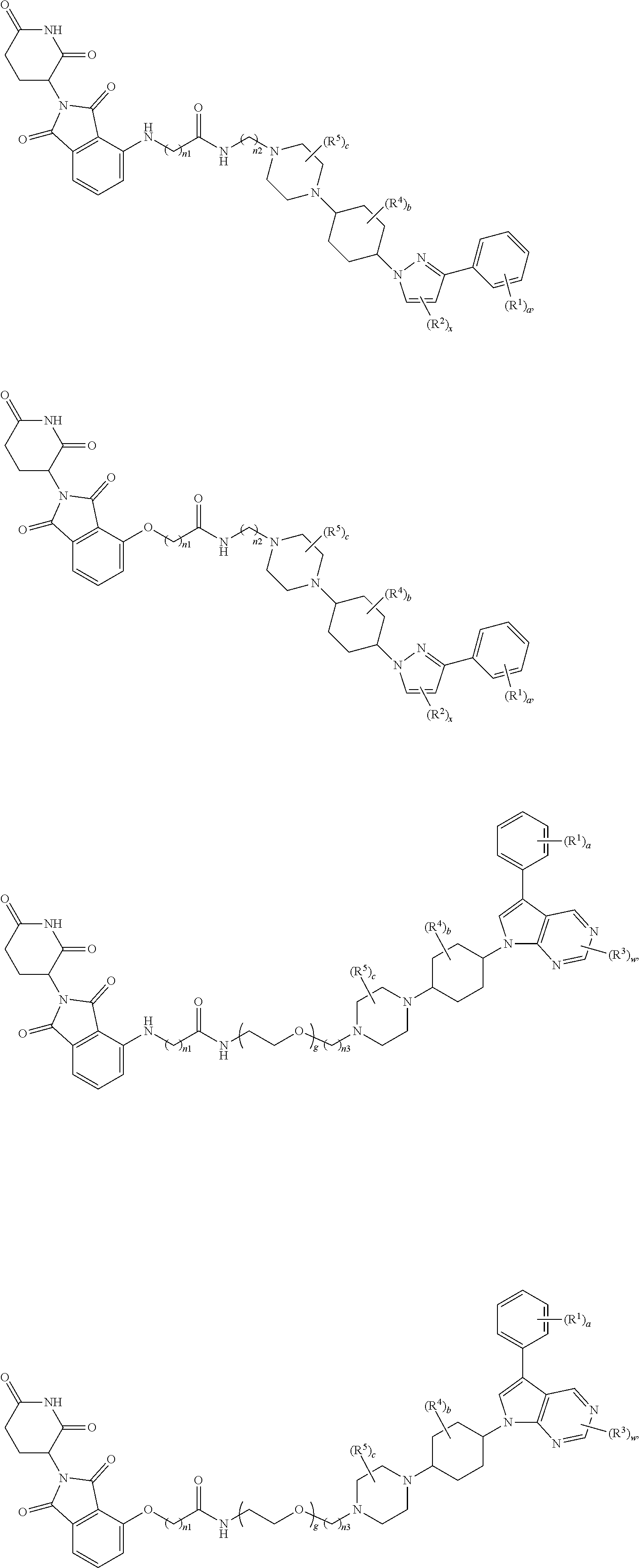

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

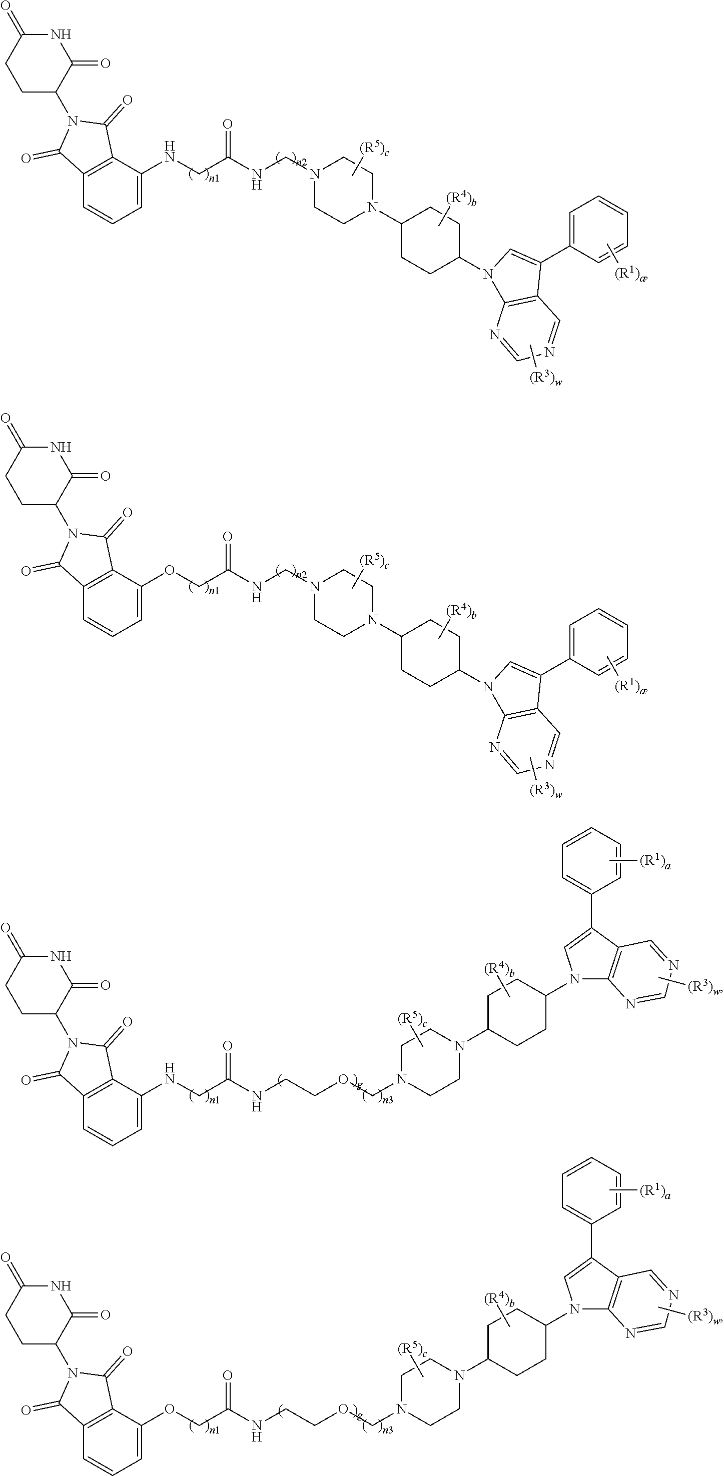

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

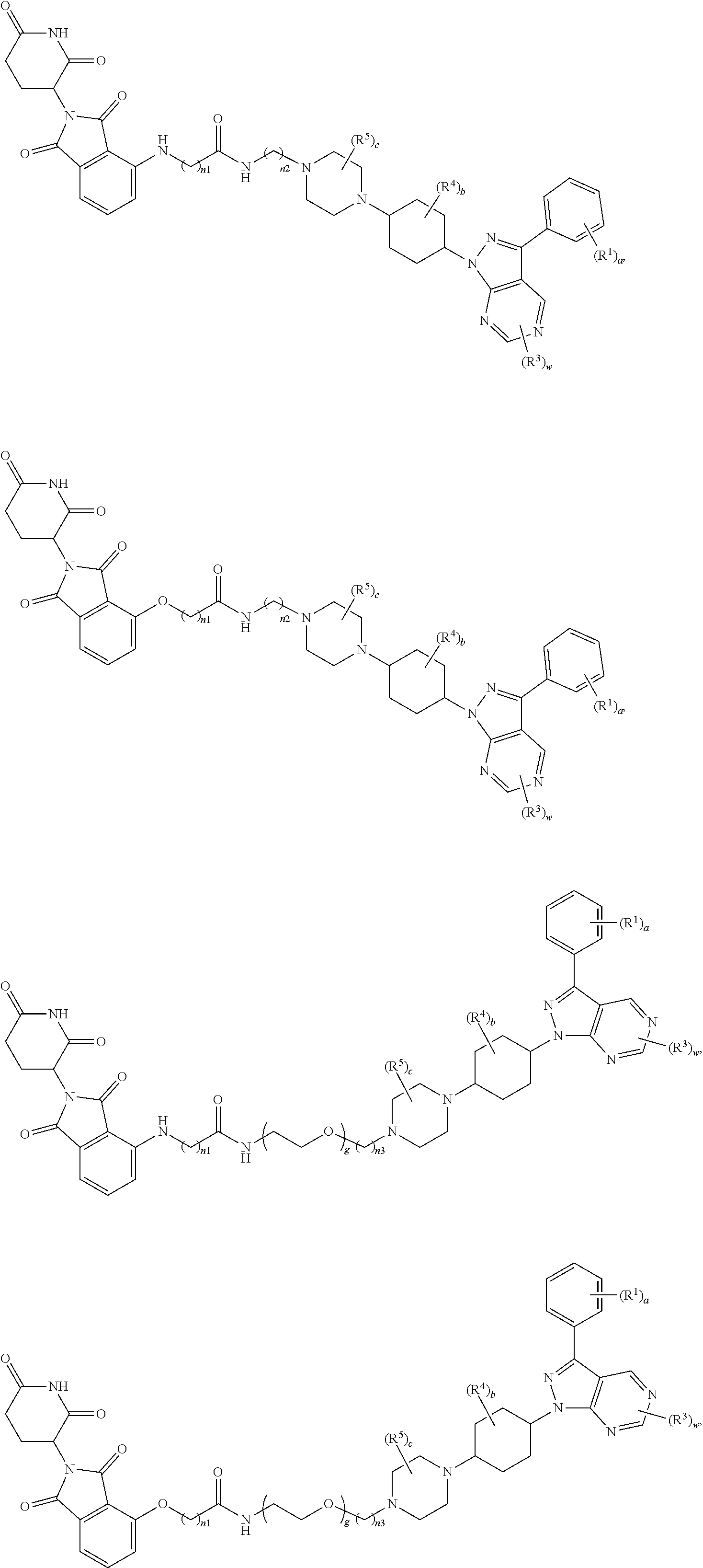

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

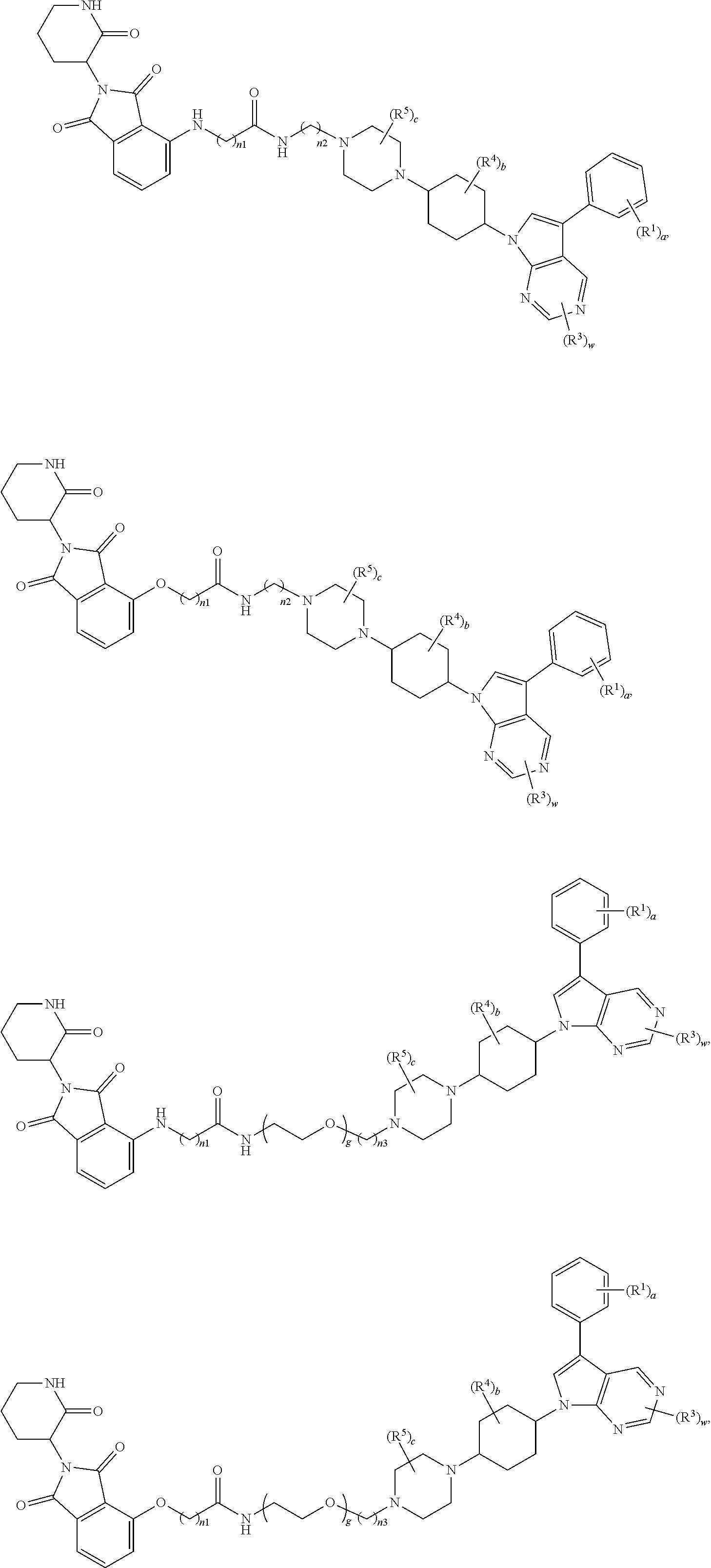

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

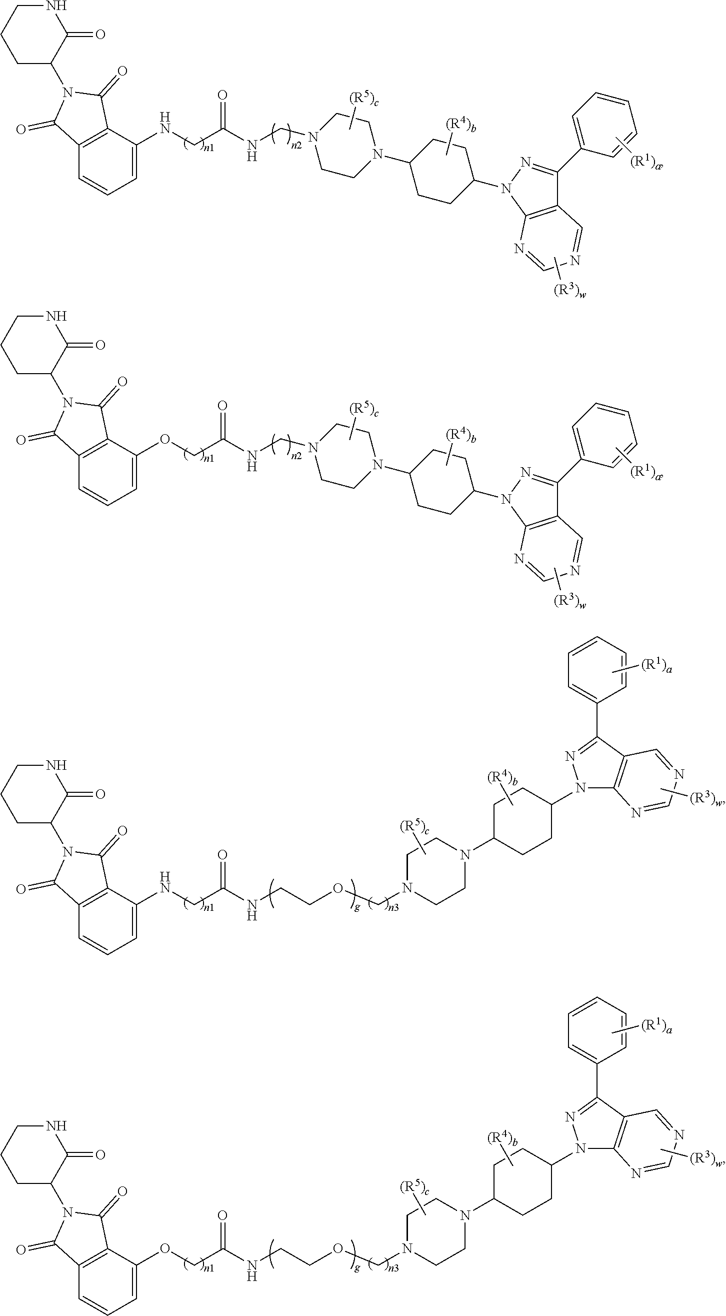

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

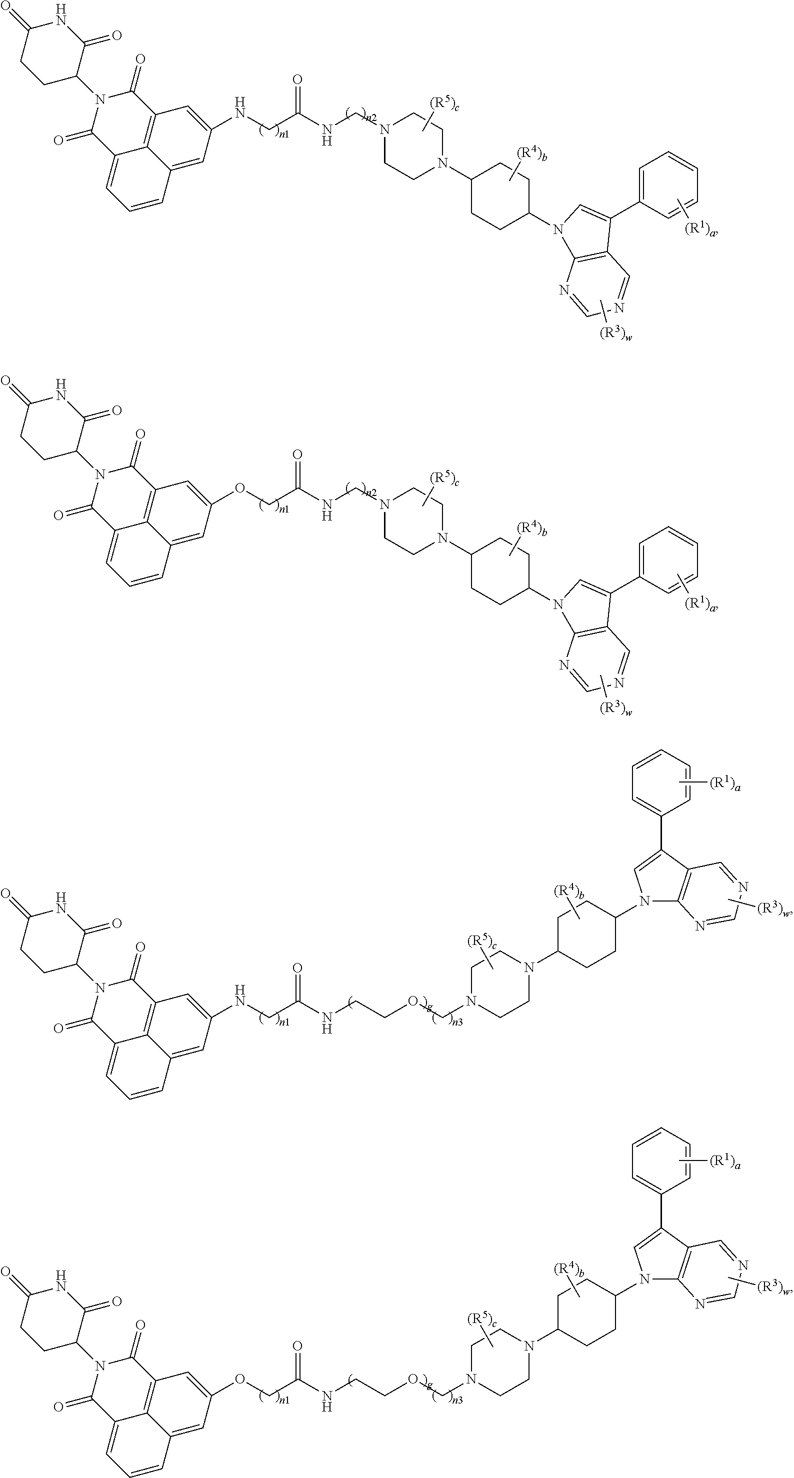

or a pharmaceutically acceptable salt, co-crystal, tautomer, stereoisomer, solvate, hydrate, polymorph, isotopically enriched derivative, or prodrug thereof.

In certain embodiments, the compound of Formula (I) is of the formula:

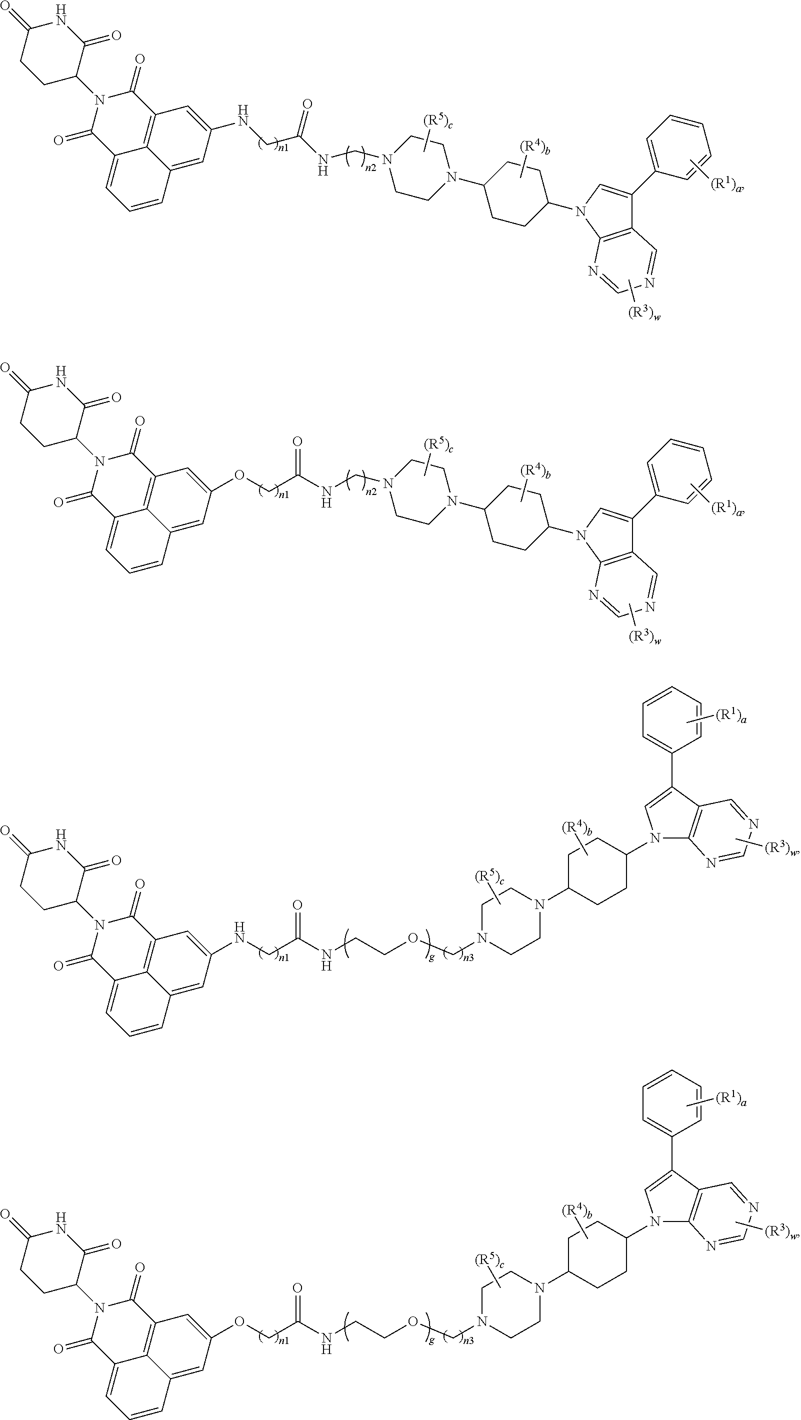

or a pharmaceutically acceptable salt, co-crystal, tautomer, stereoisomer, solvate, hydrate, polymorph, isotopically enriched derivative, or prodrug thereof; wherein n1 is 1, 2, 3, 4, 5, or 6; n2 is 1, 2, 3, 4, 5, 6, 7, 8, 9, or 10; n3 is 1, 2, 3, 4, 5, or 6; and g is 1, 2, 3, 4, 5, or 6.

In certain embodiments, the compound of Formula (I) is of the formula:

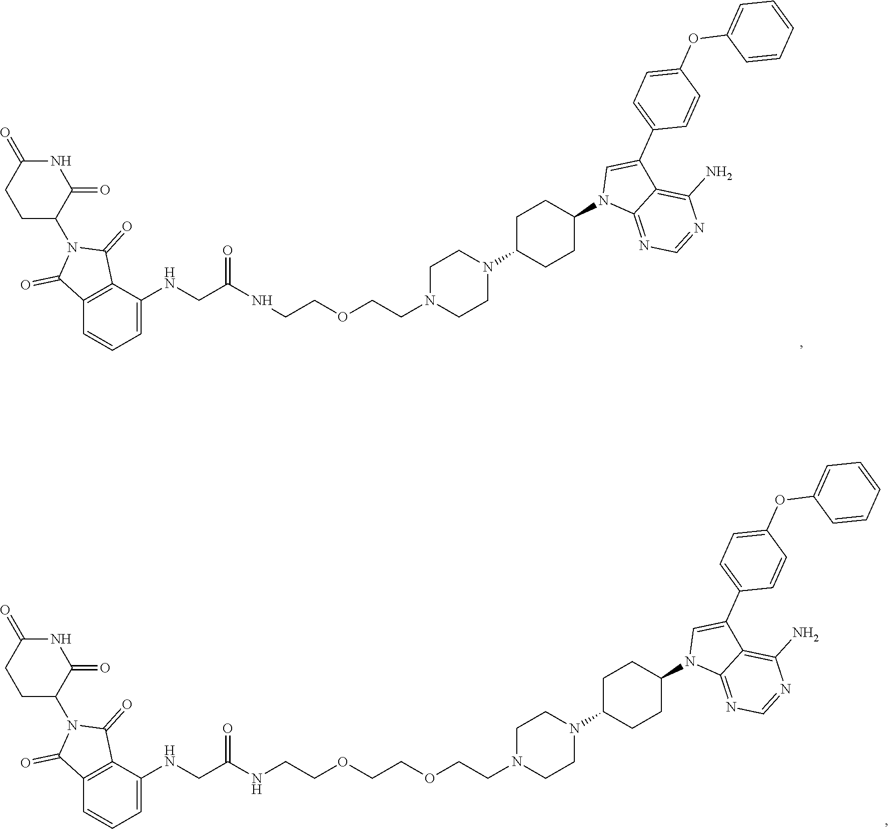 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCC(=O)NC3=O)C4=O)CC2)CC1]

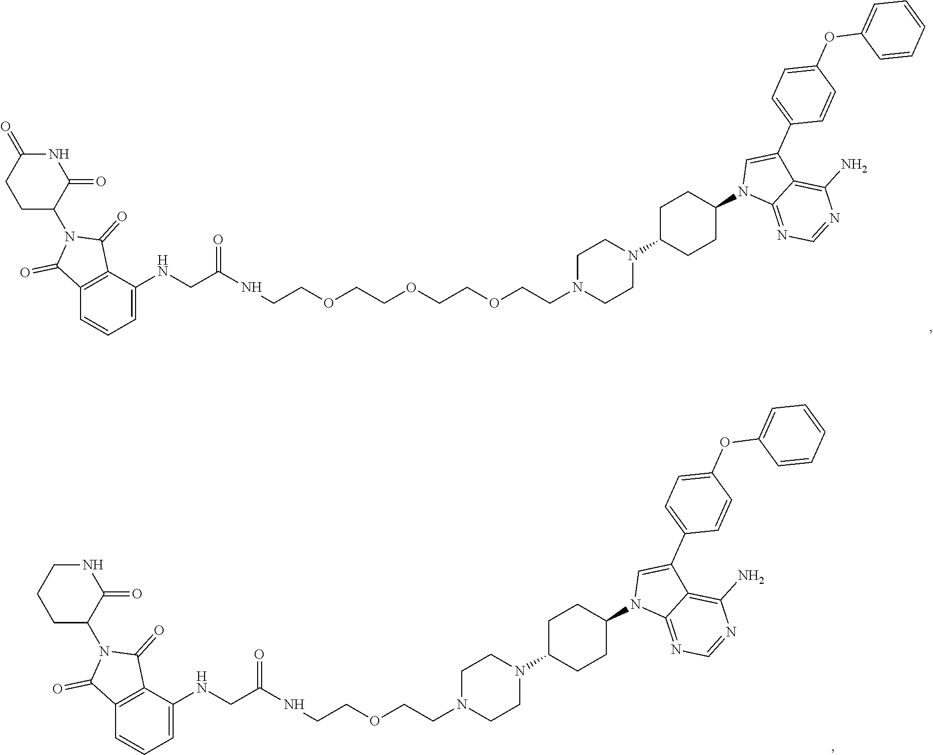 [SMILES: Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCNC(=O)CNc3cccc4c3C(=O)N(C3CCCNC3=O)C4=O)CC2)CC1]

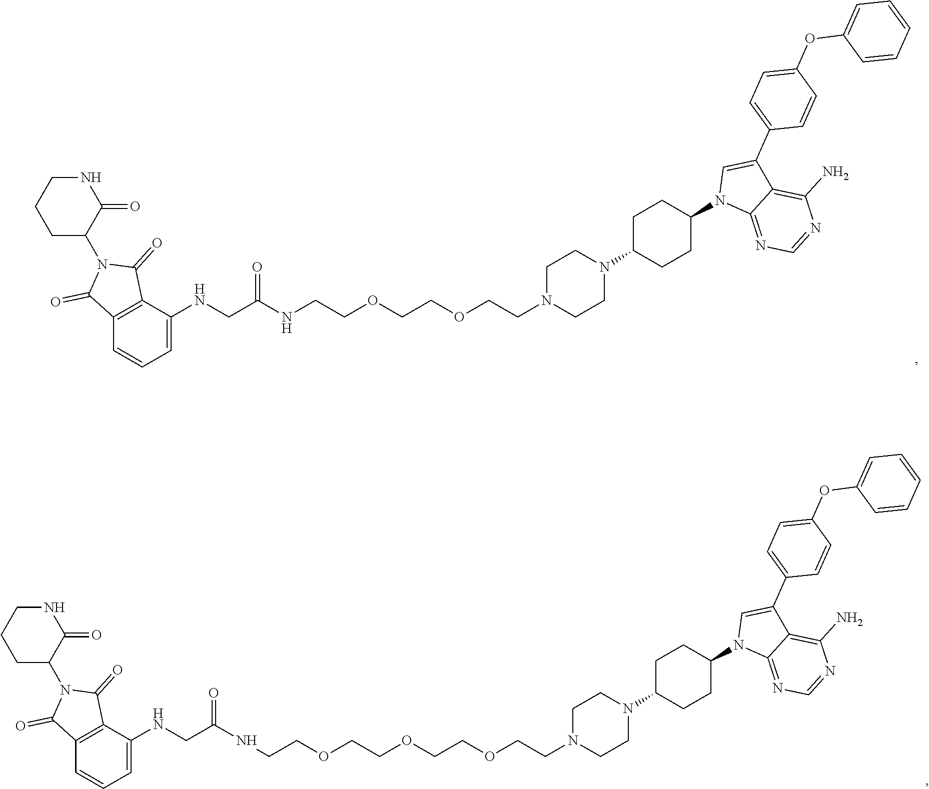 [SMILES: Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCCNC3=O)C4=O)CC2)CC1]

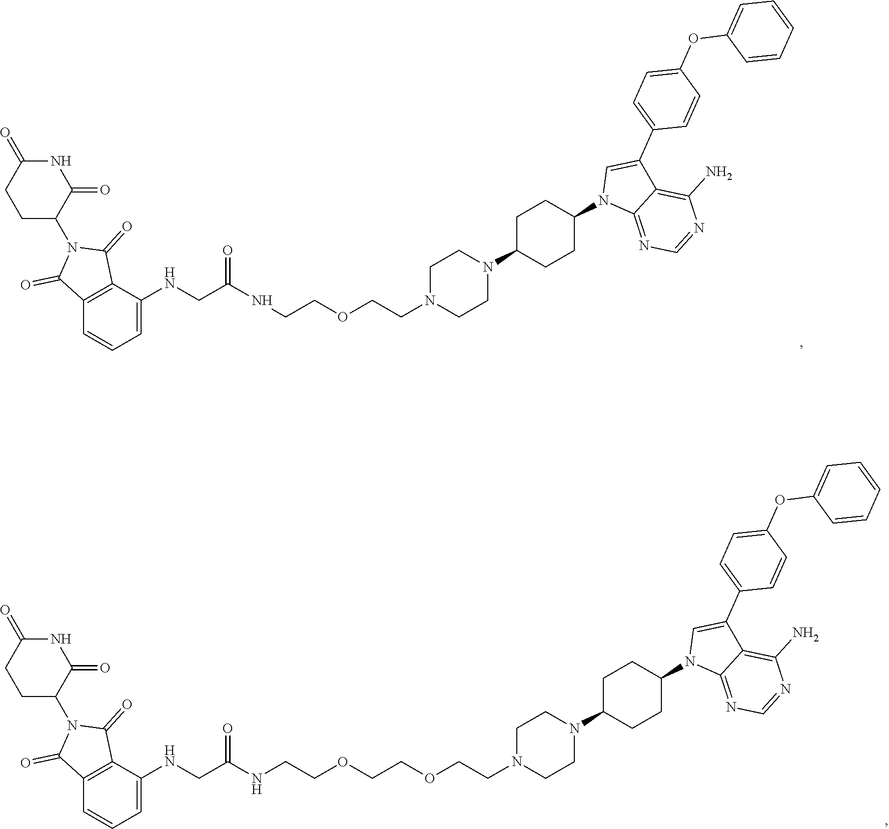 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCC(=O)NC3=O)C4=O)CC2)CC1]

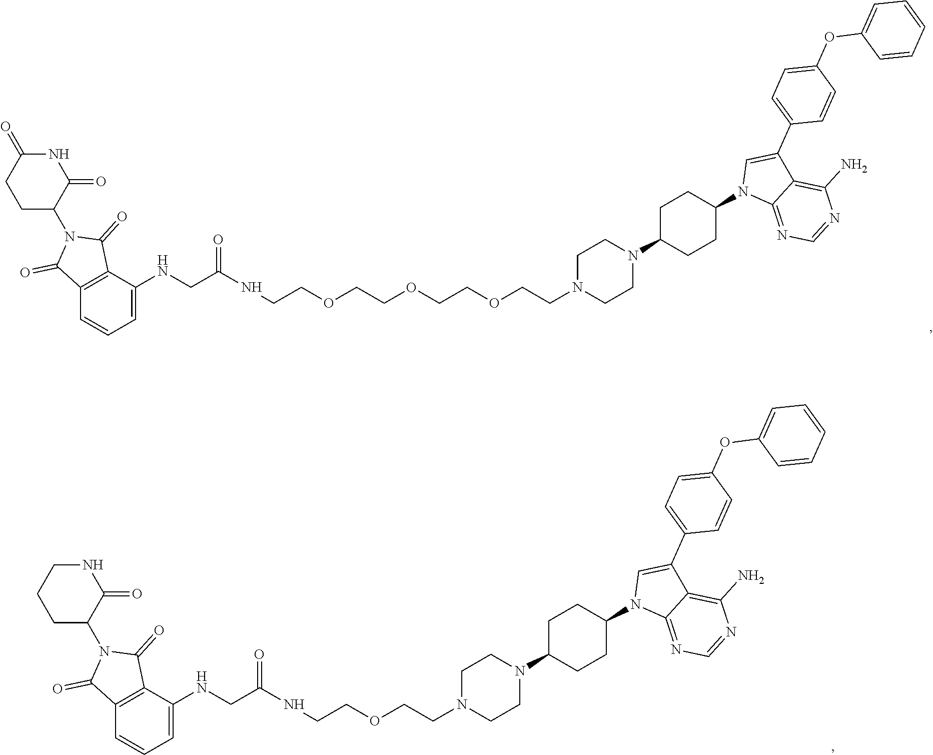 [SMILES: Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCNC(=O)CNc3cccc4c3C(=O)N(C3CCCNC3=O)C4=O)CC2)CC1]

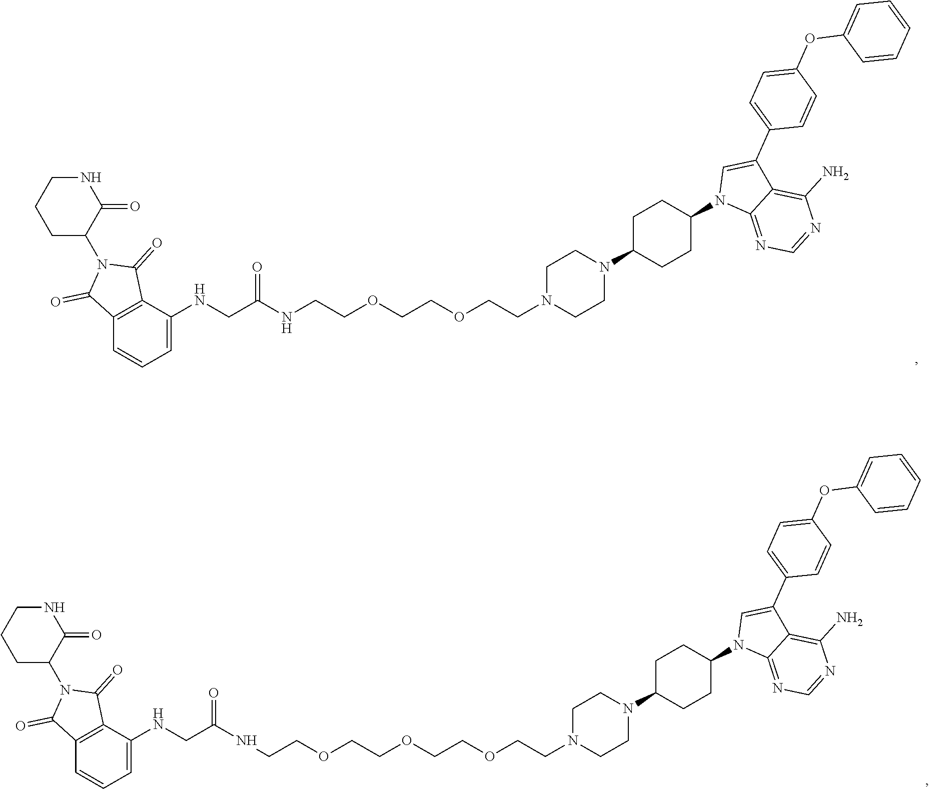 [SMILES: Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCCNC3=O)C4=O)CC2)CC1]

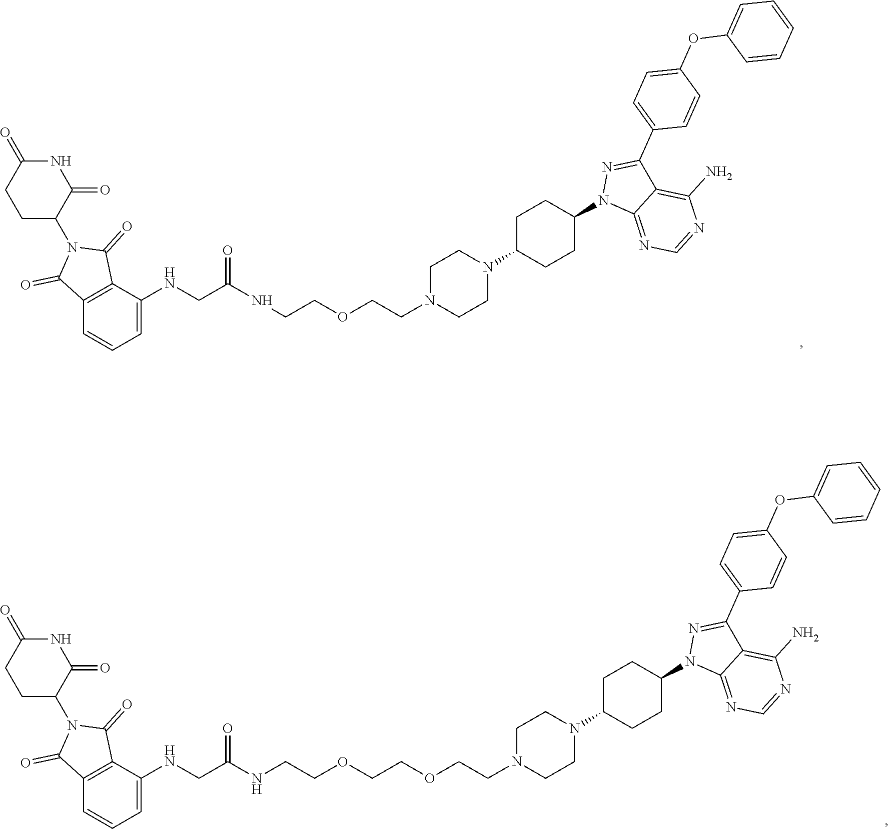 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCC(=O)NC3=O)C4=O)CC2)CC1]

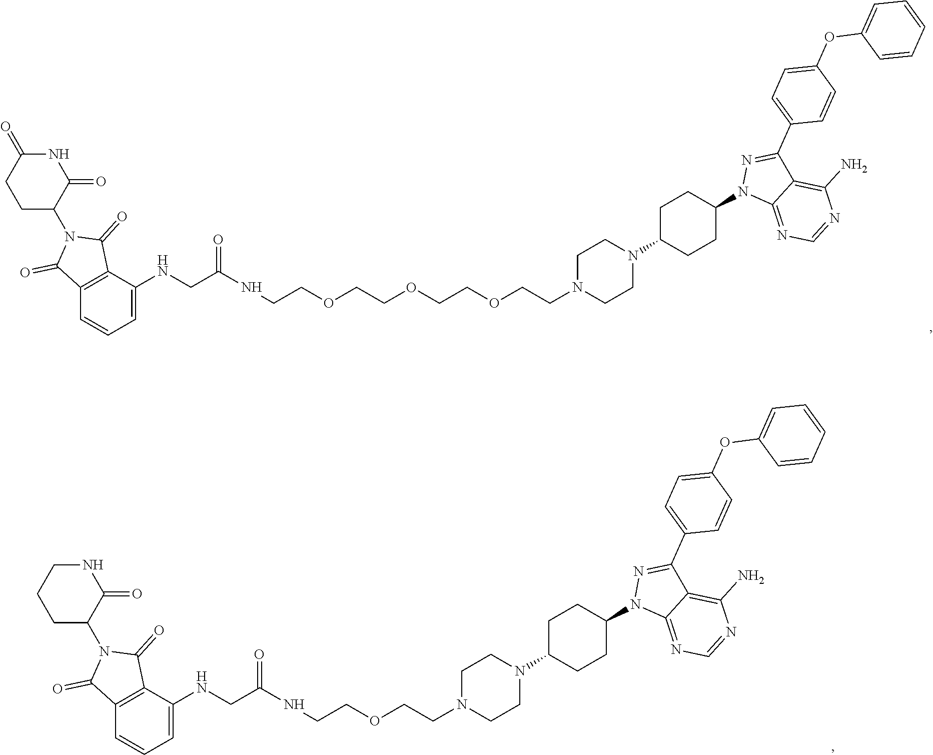 [SMILES: Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCNC(=O)CNc3cccc4c3C(=O)N(C3CCCNC3=O)C4=O)CC2)CC1]

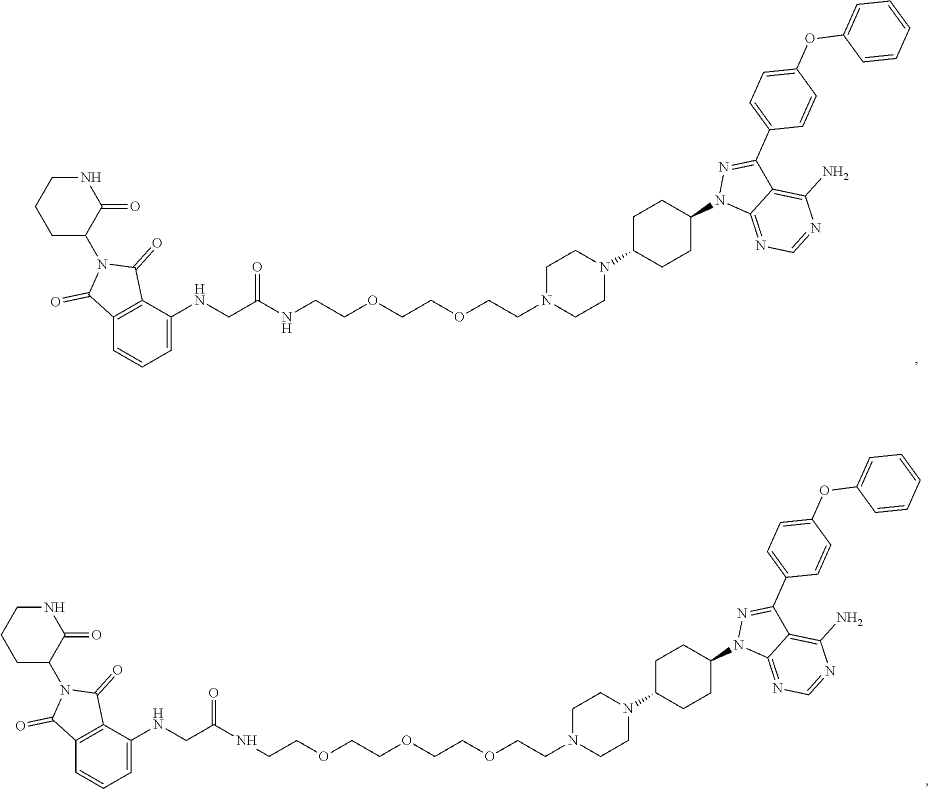 [SMILES: Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCCNC3=O)C4=O)CC2)CC1]

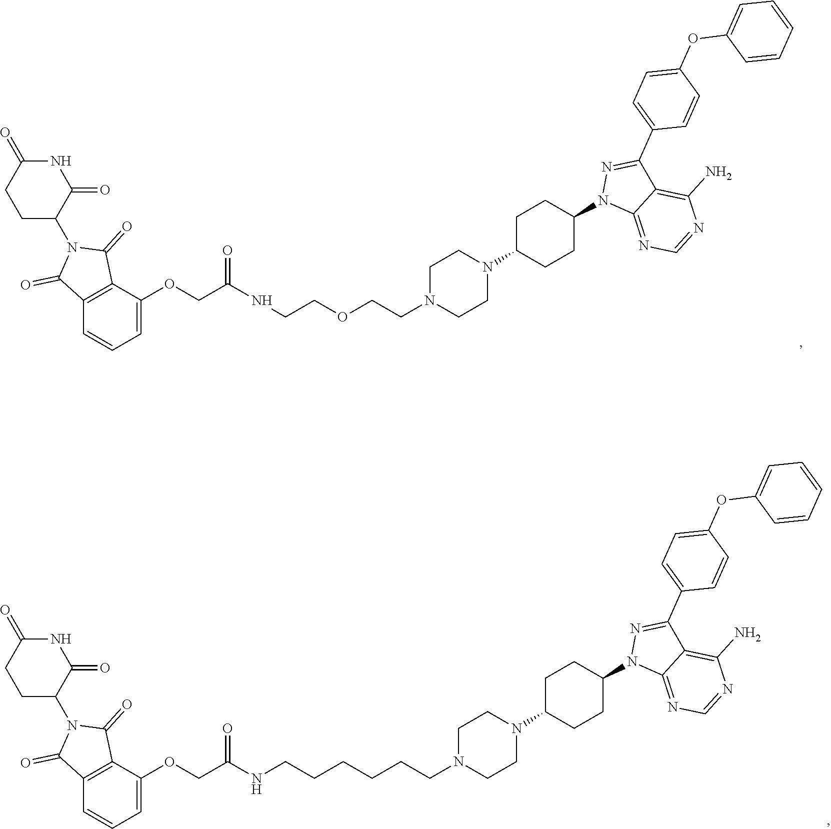 [SMILES: Nc1ncnc2c1c(-c1ccc(Oc3ccccc3)cc1)nn2[C@H]1CC[C@H](N2CCN(CCOCCNC(=O)COc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1]

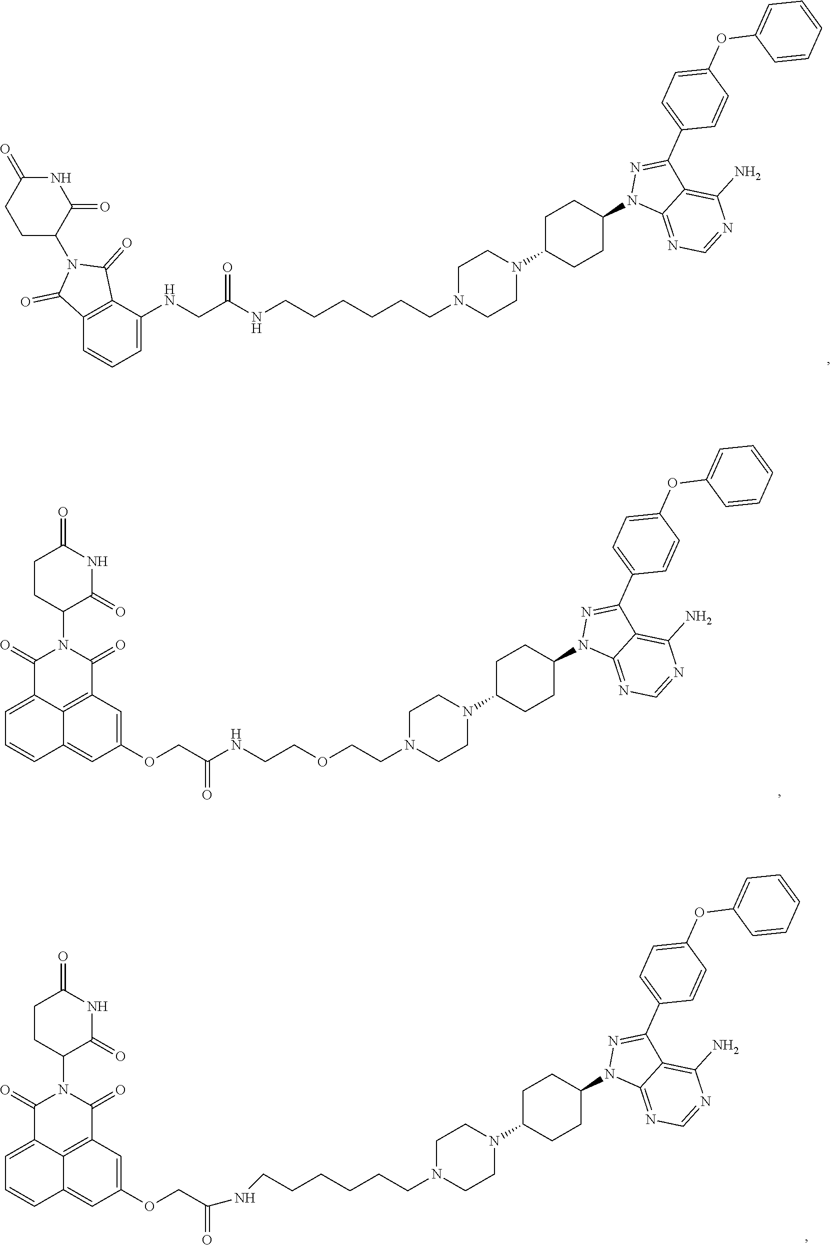 [SMILES: Nc1ncnc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCNC(=O)COc3cc4c5c(cccc5c3)C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)COc3cc4c5c(cccc5c3)C(=O)N(C3CCC(=O)NC3=O)C4=O)CC2)CC1]

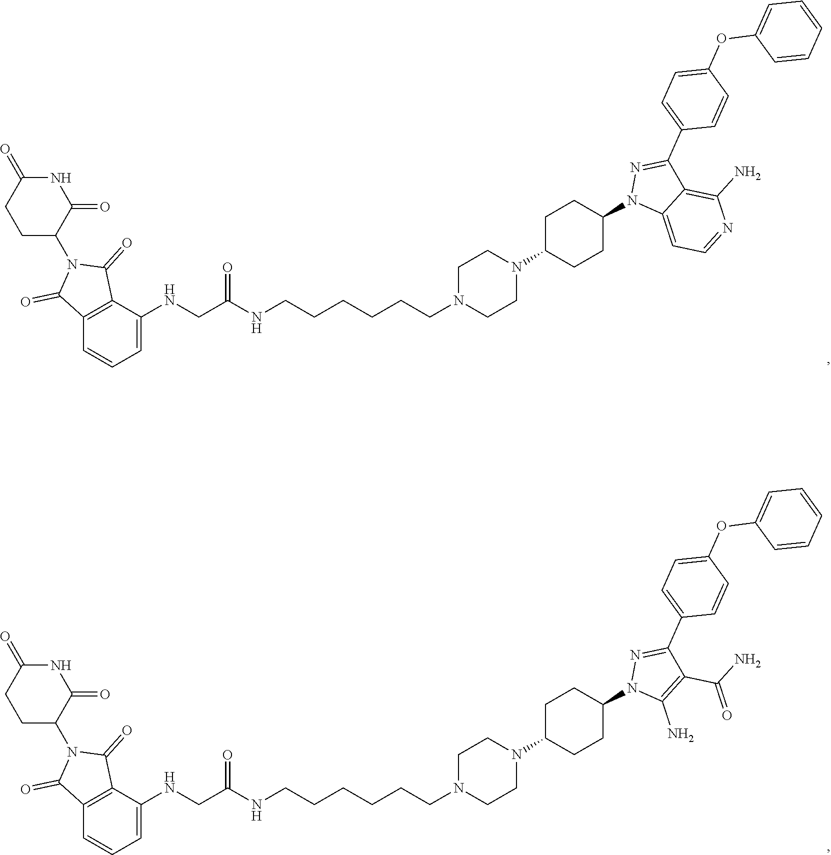 [SMILES: Nc1nccc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1 ; NC(=O)c1c(-c2ccc(Oc3ccccc3)cc2)nn([C@H]2CC[C@H](N3CCN(CCCCCCNC(=O)CNc4cccc5c4C(=O)N(C4CCC(=O)NC4=O)C5=O)CC3)CC2)c1N]

or a pharmaceutically acceptable salt, co-crystal, tautomer, stereoisomer, solvate, hydrate, polymorph, isotopically enriched derivative, or prodrug thereof.

In certain embodiments, the compound of Formula (I) is a compound provided in any one of the Examples below. In certain embodiments, the compound of Formula (I) is a compound provided in any one of the Examples below, or a pharmaceutically acceptable salt thereof. In certain embodiments, the compound of Formula (I) is a compound provided in Examples 1 or 2. In certain embodiments, the compound of Formula (I) is a compound provided in Table 1.

In certain embodiments, a compound described herein is a compound of Formula (I), or a pharmaceutically acceptable salt, solvate, hydrate, polymorph, co-crystal, tautomer, stereoisomer, isotopically labeled derivative, or prodrug thereof. In certain embodiments, a compound described herein is a compound of Formula (I), or a pharmaceutically acceptable salt thereof.

In some embodiments, the compound of Formula (I) selectively binds a kinase (e.g., HCK, BTK, FYN, SRC, BLK, LCK, CSK, ABL1, ABL2, LIMK1, LATS1) over other proteins in the proteome. In some embodiments, the compound of Formula (I) selectively binds HCK over another protein. In some embodiments, the compound of Formula (I) selectively binds a HCK over another kinase. In some embodiments, the compound of Formula (I) selectively binds a HCK over other cytoplasmic Src-family tyrosine kinases (SFK's). In some embodiments, the compound of Formula (I) selectively binds BTK over another protein. In some embodiments, the compound of Formula (I) selectively binds BTK over another kinase. In some embodiments, the compound of Formula (I) selectively binds BTK over other non-receptor tyrosine kinases. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In some embodiments, the compound of Formula (I) leads to the selective degradation of a kinase (e.g., HCK, BTK, FYN, SRC, BLK, LCK, CSK, ABL1, ABL2, LIMK1, LATS1) over other proteins in the proteome. In some embodiments, the compound of Formula (I) leads to the selective degradation of HCK over other proteins in the proteome. In some embodiments, the compound of Formula (I) leads to the selective degradation of a kinase (e.g., HCK, BTK) over other proteins in the proteome. In some embodiments, the compound of Formula (I) leads to the selective degradation of HCK over other kinases. In some embodiments, the compound of Formula (I) leads to the selective degradation of HCK over other SFK's. In some embodiments, the compound of Formula (I) leads to the selective degradation of a kinase (e.g., BTK) over other proteins in the proteome. In some embodiments, the compound of Formula (I) leads to the selective degradation of a kinase (e.g., BTK) over other kinases. In some embodiments, the compound of Formula (I) leads to the selective degradation of a kinase (e.g., BTK) over other non-receptor tyrosine kinases. In some embodiments, the compound of Formula (I) induces selective degradation of HCK over other kinases. In some embodiments, the compound of Formula (I) induces selective degradation of HCK over other SFK's. In some embodiments, the compound of Formula (I) induces selective degradation of BTK over other non-receptor tyrosine kinases. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In some embodiments, the compound of Formula (I) selectively binds E3 ligase over another protein. In certain embodiments, the selectivity is between about 2-fold and about 5-fold. In certain embodiments, the selectivity is between about 5-fold and about 10-fold. In certain embodiments, the selectivity is between about 10-fold and about 20-fold. In certain embodiments, the selectivity is between about 20-fold and about 50-fold. In certain embodiments, the selectivity is between about 50-fold and about 100-fold. In certain embodiments, the selectivity is between about 100-fold and about 200-fold. In certain embodiments, the selectivity is between about 200-fold and about 500-fold. In certain embodiments, the selectivity is between about 500-fold and about 1000-fold. In certain embodiments, the selectivity is at least about 1000-fold.

In certain embodiments, the compound of Formula (I) induces the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of the target kinase at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the compound of Formula (I) induces the degradation of up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% of HCK at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

In certain embodiments, the compound of Formula (I) increases the rate of degradation of the target kinase up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the compound of Formula (I) increases the rate of degradation of HCK up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less. In certain embodiments, the compound of Formula (I) increases the rate of degradation of BTK up to 10%, up to 15%, up to 20%, up to 25%, up to 30%, up to 35%, up to 40%, up to 45%, up to 50%, up to 55%, up to 60%, up to 65%, up to 70%, up to 75%, up to 80%, up to 85%, up to 90%, up to 95%, up to 99%, or up to 100% at a concentration of 100,000 nM or less, 50,000 nM or less, 20,000 nM or less, 10,000 nM or less, 5,000 nM or less, 3,500 nM or less, 2,500 nM or less, 1,000 nM or less, 900 nM or less, 800 nM or less, 700 nM or less, 600 nM or less, 500 nM or less, 400 nM or less, 300 nM or less, 200 nM or less, 100 nM or less, 90 nM or less, 80 nM or less, 70 nM or less, 60 nM or less, 50 nM or less, 40 nM or less, 30 nM or less, 20 nM or less, 10 nM or less, 5 nM or less, 4 nM or less, 3 nM or less, 2 nM or less, or 1 nM or less.

### Pharmaceutical Compositions, Kits, and Administration

The present disclosure provides pharmaceutical compositions comprising a compound of Formula (I), or a pharmaceutically acceptable salt, co-crystal, tautomer, stereoisomer, solvate, hydrate, polymorph, isotopically enriched derivative, or prodrug thereof, and optionally a pharmaceutically acceptable excipient. In certain embodiments, the pharmaceutical composition described herein comprises a compound of Formula (I), or a pharmaceutically acceptable salt thereof, and a pharmaceutically acceptable excipient.

In certain embodiments, the compound of Formula (I) is provided in an effective amount in the pharmaceutical composition. In certain embodiments, the effective amount is a therapeutically effective amount. In certain embodiments, the effective amount is a prophylactically effective amount. In certain embodiments, the effective amount is an amount effective for treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the effective amount is an amount effective for preventing a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the effective amount is an amount effective for treating cancer in a subject in need thereof. In certain embodiments, the effective amount is an amount effective for preventing cancer in a subject in need thereof. In certain embodiments, the effective amount is an amount effective for reducing the risk of developing a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof.

In certain embodiments, the subject is an animal. The animal may be of either sex and may be at any stage of development. In certain embodiments, the subject is a human. In certain embodiments, the subject is a non-human animal. In certain embodiments, the subject is a mammal. In certain embodiments, the subject is a non-human mammal. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal, such as a rodent (e.g., mouse, rat), dog, pig, or non-human primate. In certain embodiments, the animal is a genetically engineered animal. In certain embodiments, the animal is a transgenic animal (e.g., transgenic mice and transgenic pigs). In certain embodiments, the subject is a fish or reptile.

In certain embodiments, the effective amount is an amount effective for inducing the degradation of at least about 10%, at least about 15%, at least about 20%, at least about 25%, at least about 30%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% of the target kinase in a cell. In certain embodiments, the effective amount is an amount effective for inducing the degradation of at least about 10%, at least about 15%, at least about 20%, at least about 25%, at least about 30%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% of HCK in a cell. In certain embodiments, the effective amount is an amount effective for inducing the degradation of the target protein HCK in a cell by a range between a percentage described in this paragraph and another percentage described in this paragraph, inclusive. In certain embodiments, the effective amount is an amount effective for inducing the degradation of at least about 10%, at least about 15%, at least about 20%, at least about 25%, at least about 30%, at least about 35%, at least about 40%, at least about 42%, at least about 45%, at least about 50%, at least about 55%, at least about 60%, at least about 65%, at least about 70%, at least about 75%, at least about 80%, at least about 85%, at least about 90%, at least about 95%, at least about 98%, or at least about 99% of BTK in a cell. In certain embodiments, the effective amount is an amount effective for inducing the degradation of the target protein BTK in a cell by a range between a percentage described in this paragraph and another percentage described in this paragraph, inclusive.

The present disclosure provides pharmaceutical compositions comprising a compound that interacts with a E3 ubiquitin ligase (e.g., cereblon) and the target kinase (e.g., HCK, BTK) for use in treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. The present disclosure provides pharmaceutical compositions comprising a compound that interacts with a E3 ubiquitin ligase (e.g., cereblon) and HCK for use in treating a disease (e.g., a proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the composition is for use in treating a disease in a subject in need thereof. In certain embodiments, the composition is for use in treating a proliferative disease. In certain embodiments, the composition is for use in treating a disease is associated with a MYD88 mutation. In certain embodiments, the composition is for use in treating cancer. In certain embodiments, the composition is for use in treating lymphoma, leukemia, or cancer associated with hematopoietic cell kinase (HCK). In certain embodiments, the composition is for use in treating lymphoma, for example, non-Hodgkin's lymphoma (e.g., Waldenstrom macroglobulinemia, MYD88-mutated Waldenstrom macroglobulinemia) or Burkitt's lymphoma. In certain embodiments, the composition is for use in treating Waldenstrom macroglobulinemia. In certain embodiments, the composition is for use in treating MYD88-mutated Waldenstrom macroglobulinemia. In certain embodiments, the composition is for use in treating leukemia, for example, hairy cell leukemia or acute myeloid leukemia (AML)). In certain embodiments, the composition is for use in treating diffuse large B-cell lymphoma, for example, activated B-cell (ABC) diffuse large B-cell lymphoma or germinal center B-cell-like diffuse large B-cell lymphoma. In certain embodiments, the composition is for use in treating myelodysplastic syndrome (MDS), In certain embodiments, the composition is for use in treating multiple myeloma.

Pharmaceutical compositions can be prepared, packaged, and/or sold in bulk, as a single unit dose, and/or as a plurality of single unit doses. A “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage, such as one-half or one-third of such a dosage.

Relative amounts of the active ingredient, the pharmaceutically acceptable excipient, and/or any additional ingredients in a pharmaceutical composition described herein will vary, depending upon the identity, size, and/or condition of the subject treated and further depending upon the route by which the composition is to be administered. The composition may comprise between 0.1% and 100% (w/w) active ingredient.

Pharmaceutically acceptable excipients used in the manufacture of provided pharmaceutical compositions include inert diluents, dispersing and/or granulating agents, surface active agents and/or emulsifiers, disintegrating agents, binding agents, preservatives, buffering agents, lubricating agents, and/or oils. Excipients such as cocoa butter and suppository waxes, coloring agents, coating agents, sweetening, flavoring, and perfuming agents may also be present in the composition.

Exemplary diluents include calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, and mixtures thereof.

Exemplary granulating and/or dispersing agents include potato starch, corn starch, tapioca starch, sodium starch glycolate, clays, alginic acid, guar gum, citrus pulp, agar, bentonite, cellulose, and wood products, natural sponge, cation-exchange resins, calcium carbonate, silicates, sodium carbonate, cross-linked poly(vinyl-pyrrolidone) (crospovidone), sodium carboxymethyl starch (sodium starch glycolate), carboxymethyl cellulose, cross-linked sodium carboxymethyl cellulose (croscarmellose), methylcellulose, pregelatinized starch (starch 1500), microcrystalline starch, water insoluble starch, calcium carboxymethyl cellulose, magnesium aluminum silicate (Veegum), sodium lauryl sulfate, quaternary ammonium compounds, and mixtures thereof.

Exemplary surface active agents and/or emulsifiers include natural emulsifiers (e.g., acacia, agar, alginic acid, sodium alginate, tragacanth, chondrux, cholesterol, xanthan, pectin, gelatin, egg yolk, casein, wool fat, cholesterol, wax, and lecithin), colloidal clays (e.g., bentonite (aluminum silicate) and Veegum (magnesium aluminum silicate)), long chain amino acid derivatives, high molecular weight alcohols (e.g., stearyl alcohol, cetyl alcohol, oleyl alcohol, triacetin monostearate, ethylene glycol distearate, glyceryl monostearate, and propylene glycol monostearate, polyvinyl alcohol), carbomers (e.g., carboxy polymethylene, polyacrylic acid, acrylic acid polymer, and carboxyvinyl polymer), carrageenan, cellulosic derivatives (e.g., carboxymethylcellulose sodium, powdered cellulose, hydroxymethyl cellulose, hydroxypropyl cellulose, hydroxypropyl methylcellulose, methylcellulose), sorbitan fatty acid esters (e.g., polyoxyethylene sorbitan monolaurate (Tween® 20), polyoxyethylene sorbitan (Tween® 60), polyoxyethylene sorbitan monooleate (Tween® 80), sorbitan monopalmitate (Span® 40), sorbitan monostearate (Span® 60), sorbitan tristearate (Span® 65), glyceryl monooleate, sorbitan monooleate (Span® 80), polyoxyethylene esters (e.g., polyoxyethylene monostearate (Myrj® 45), polyoxyethylene hydrogenated castor oil, polyethoxylated castor oil, polyoxymethylene stearate, and Solutol®), sucrose fatty acid esters, polyethylene glycol fatty acid esters (e.g., Cremophor®), polyoxyethylene ethers, (e.g., polyoxyethylene lauryl ether (Brij® 30)), poly(vinyl-pyrrolidone), diethylene glycol monolaurate, triethanolamine oleate, sodium oleate, potassium oleate, ethyl oleate, oleic acid, ethyl laurate, sodium lauryl sulfate, Pluronic® F-68, poloxamer P-188, cetrimonium bromide, cetylpyridinium chloride, benzalkonium chloride, docusate sodium, and/or mixtures thereof.

Exemplary binding agents include starch (e.g., cornstarch and starch paste), gelatin, sugars (e.g., sucrose, glucose, dextrose, dextrin, molasses, lactose, lactitol, mannitol, etc.), natural and synthetic gums (e.g., acacia, sodium alginate, extract of Irish moss, panwar gum, ghatti gum, mucilage of isapol husks, carboxymethylcellulose, methylcellulose, ethylcellulose, hydroxyethylcellulose, hydroxypropyl cellulose, hydroxypropyl methylcellulose, microcrystalline cellulose, cellulose acetate, poly(vinyl-pyrrolidone), magnesium aluminum silicate (Veegum®), and larch arabogalactan), alginates, polyethylene oxide, polyethylene glycol, inorganic calcium salts, silicic acid, polymethacrylates, waxes, water, alcohol, and/or mixtures thereof.

Exemplary preservatives include antioxidants, chelating agents, antimicrobial preservatives, antifungal preservatives, antiprotozoan preservatives, alcohol preservatives, acidic preservatives, and other preservatives. In certain embodiments, the preservative is an antioxidant. In other embodiments, the preservative is a chelating agent.

Exemplary antioxidants include alpha tocopherol, ascorbic acid, acorbyl palmitate, butylated hydroxyanisole, butylated hydroxytoluene, monothioglycerol, potassium metabisulfite, propionic acid, propyl gallate, sodium ascorbate, sodium bisulfite, sodium metabisulfite, and sodium sulfite.

Exemplary chelating agents include ethylenediaminetetraacetic acid (EDTA) and salts and hydrates thereof (e.g., sodium edetate, disodium edetate, trisodium edetate, calcium disodium edetate, dipotassium edetate, and the like), citric acid and salts and hydrates thereof (e.g., citric acid monohydrate), fumaric acid and salts and hydrates thereof, malic acid and salts and hydrates thereof, phosphoric acid and salts and hydrates thereof, and tartaric acid and salts and hydrates thereof. Exemplary antimicrobial preservatives include benzalkonium chloride, benzethonium chloride, benzyl alcohol, bronopol, cetrimide, cetylpyridinium chloride, chlorhexidine, chlorobutanol, chlorocresol, chloroxylenol, cresol, ethyl alcohol, glycerin, hexetidine, imidurea, phenol, phenoxyethanol, phenylethyl alcohol, phenylmercuric nitrate, propylene glycol, and thimerosal.

Exemplary antifungal preservatives include butyl paraben, methyl paraben, ethyl paraben, propyl paraben, benzoic acid, hydroxybenzoic acid, potassium benzoate, potassium sorbate, sodium benzoate, sodium propionate, and sorbic acid.

Exemplary alcohol preservatives include ethanol, polyethylene glycol, phenol, phenolic compounds, bisphenol, chlorobutanol, hydroxybenzoate, and phenylethyl alcohol.

Exemplary acidic preservatives include vitamin A, vitamin C, vitamin E, beta-carotene, citric acid, acetic acid, dehydroacetic acid, ascorbic acid, sorbic acid, and phytic acid.

Other preservatives include tocopherol, tocopherol acetate, deteroxime mesylate, cetrimide, butylated hydroxyanisol (BHA), butylated hydroxytoluened (BHT), ethylenediamine, sodium lauryl sulfate (SLS), sodium lauryl ether sulfate (SLES), sodium bisulfite, sodium metabisulfite, potassium sulfite, potassium metabisulfite, Glydant® Plus, Phenonip©, methylparaben, Germall® 115, Germaben® II, Neolone®, Kathon®, and Euxyl®.

Exemplary buffering agents include citrate buffer solutions, acetate buffer solutions, phosphate buffer solutions, ammonium chloride, calcium carbonate, calcium chloride, calcium citrate, calcium glubionate, calcium gluceptate, calcium gluconate, D-gluconic acid, calcium glycerophosphate, calcium lactate, propanoic acid, calcium levulinate, pentanoic acid, dibasic calcium phosphate, phosphoric acid, tribasic calcium phosphate, calcium hydroxide phosphate, potassium acetate, potassium chloride, potassium gluconate, potassium mixtures, dibasic potassium phosphate, monobasic potassium phosphate, potassium phosphate mixtures, sodium acetate, sodium bicarbonate, sodium chloride, sodium citrate, sodium lactate, dibasic sodium phosphate, monobasic sodium phosphate, sodium phosphate mixtures, tromethamine, magnesium hydroxide, aluminum hydroxide, alginic acid, pyrogen-free water, isotonic saline, Ringer's solution, ethyl alcohol, and mixtures thereof.

Exemplary lubricating agents include magnesium stearate, calcium stearate, stearic acid, silica, talc, malt, glyceryl behanate, hydrogenated vegetable oils, polyethylene glycol, sodium benzoate, sodium acetate, sodium chloride, leucine, magnesium lauryl sulfate, sodium lauryl sulfate, and mixtures thereof.

Exemplary natural oils include almond, apricot kernel, avocado, babassu, bergamot, black current seed, borage, cade, camomile, canola, caraway, carnauba, castor, cinnamon, cocoa butter, coconut, cod liver, coffee, corn, cotton seed, emu, eucalyptus, evening primrose, fish, flaxseed, geraniol, gourd, grape seed, hazel nut, hyssop, isopropyl myristate, jojoba, kukui nut, lavandin, lavender, lemon, Litsea cubeba, macademia nut, mallow, mango seed, meadowfoam seed, mink, nutmeg, olive, orange, orange roughy, palm, palm kernel, peach kernel, peanut, poppy seed, pumpkin seed, rapeseed, rice bran, rosemary, safflower, sandalwood, sasquana, savoury, sea buckthorn, sesame, shea butter, silicone, soybean, sunflower, tea tree, thistle, tsubaki, vetiver, walnut, and wheat germ oils. Exemplary synthetic oils include, but are not limited to, butyl stearate, caprylic triglyceride, capric triglyceride, cyclomethicone, diethyl sebacate, dimethicone 360, isopropyl myristate, mineral oil, octyldodecanol, oleyl alcohol, silicone oil, and mixtures thereof.

Liquid dosage forms for oral and parenteral administration include pharmaceutically acceptable emulsions, microemulsions, solutions, suspensions, syrups and elixirs. In addition to the active ingredients, the liquid dosage forms may comprise inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (e.g., cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can include adjuvants such as wetting agents, emulsifying and suspending agents, sweetening, flavoring, and perfuming agents. In certain embodiments for parenteral administration, the conjugates described herein are mixed with solubilizing agents such as Cremophor®, alcohols, oils, modified oils, glycols, polysorbates, cyclodextrins, polymers, and mixtures thereof.

Injectable preparations, for example, sterile injectable aqueous or oleaginous suspensions can be formulated according to the known art using suitable dispersing or wetting agents and suspending agents. The sterile injectable preparation can be a sterile injectable solution, suspension, or emulsion in a nontoxic parenterally acceptable diluent or solvent, for example, as a solution in 1,3-butanediol. Among the acceptable vehicles and solvents that can be employed are water, Ringer's solution, U.S.P., and isotonic sodium chloride solution. In addition, sterile, fixed oils are conventionally employed as a solvent or suspending medium. For this purpose any bland fixed oil can be employed including synthetic mono- or di-glycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution, which, in turn, may depend upon crystal size and crystalline form. Alternatively, delayed absorption of a parenterally administered drug form may be accomplished by dissolving or suspending the drug in an oil vehicle.

Compositions for rectal or vaginal administration are typically suppositories which can be prepared by mixing the conjugates described herein with suitable non-irritating excipients or carriers such as cocoa butter, polyethylene glycol, or a suppository wax which are solid at ambient temperature but liquid at body temperature and therefore melt in the rectum or vaginal cavity and release the active ingredient.

Solid dosage forms for oral administration include capsules, tablets, pills, powders, and granules. In such solid dosage forms, the active ingredient is mixed with at least one inert, pharmaceutically acceptable excipient or carrier such as sodium citrate or dicalcium phosphate and/or (a) fillers or extenders such as starches, lactose, sucrose, glucose, mannitol, and silicic acid, (b) binders such as, for example, carboxymethylcellulose, alginates, gelatin, polyvinylpyrrolidinone, sucrose, and acacia, (c) humectants such as glycerol, (d) disintegrating agents such as agar, calcium carbonate, potato or tapioca starch, alginic acid, certain silicates, and sodium carbonate, (e) solution retarding agents such as paraffin, (f) absorption accelerators such as quaternary ammonium compounds, (g) wetting agents such as, for example, cetyl alcohol and glycerol monostearate, (h) absorbents such as kaolin and bentonite clay, and (i) lubricants such as talc, calcium stearate, magnesium stearate, solid polyethylene glycols, sodium lauryl sulfate, and mixtures thereof. In the case of capsules, tablets, and pills, the dosage form may include a buffering agent.

Solid compositions of a similar type can be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the art of pharmacology. They may optionally comprise opacifying agents and can be of a composition that they release the active ingredient(s) only, or preferentially, in a certain part of the intestinal tract, optionally, in a delayed manner. Examples of encapsulating compositions which can be used include polymeric substances and waxes. Solid compositions of a similar type can be employed as fillers in soft and hard-filled gelatin capsules using such excipients as lactose or milk sugar as well as high molecular weight polethylene glycols and the like.

The active ingredient can be in a micro-encapsulated form with one or more excipients as noted above. The solid dosage forms of tablets, dragees, capsules, pills, and granules can be prepared with coatings and shells such as enteric coatings, release controlling coatings, and other coatings well known in the pharmaceutical formulating art. In such solid dosage forms the active ingredient can be admixed with at least one inert diluent such as sucrose, lactose, or starch. Such dosage forms may comprise, as is normal practice, additional substances other than inert diluents, e.g., tableting lubricants and other tableting aids such a magnesium stearate and microcrystalline cellulose. In the case of capsules, tablets and pills, the dosage forms may comprise buffering agents. They may optionally comprise opacifying agents and can be of a composition that they release the active ingredient(s) only, or preferentially, in a certain part of the intestinal tract, optionally, in a delayed manner. Examples of encapsulating agents which can be used include polymeric substances and waxes.

Dosage forms for topical and/or transdermal administration of a compound described herein may include ointments, pastes, creams, lotions, gels, powders, solutions, sprays, inhalants, and/or patches. Generally, the active ingredient is admixed under sterile conditions with a pharmaceutically acceptable carrier or excipient and/or any needed preservatives and/or buffers as can be required. Additionally, the present disclosure contemplates the use of transdermal patches, which often have the added advantage of providing controlled delivery of an active ingredient to the body. Such dosage forms can be prepared, for example, by dissolving and/or dispensing the active ingredient in the proper medium. Alternatively or additionally, the rate can be controlled by either providing a rate controlling membrane and/or by dispersing the active ingredient in a polymer matrix and/or gel.

Suitable devices for use in delivering intradermal pharmaceutical compositions described herein include short needle devices. Intradermal compositions can be administered by devices which limit the effective penetration length of a needle into the skin. Alternatively or additionally, conventional syringes can be used in the classical mantoux method of intradermal administration. Jet injection devices which deliver liquid formulations to the dermis via a liquid jet injector and/or via a needle which pierces the stratum corneum and produces a jet which reaches the dermis are suitable. Ballistic powder/particle delivery devices which use compressed gas to accelerate the compound in powder form through the outer layers of the skin to the dermis are suitable.

Formulations suitable for topical administration include, but are not limited to, liquid and/or semi-liquid preparations such as liniments, lotions, oil-in-water and/or water-in-oil emulsions such as creams, ointments, and/or pastes, and/or solutions and/or suspensions. Topically administrable formulations may, for example, comprise from about 1% to about 10% (w/w) active ingredient, although the concentration of the active ingredient can be as high as the solubility limit of the active ingredient in the solvent. Formulations for topical administration may further comprise one or more of the additional ingredients described herein.

A pharmaceutical composition described herein can be prepared, packaged, and/or sold in a formulation suitable for pulmonary administration via the buccal cavity. Such a formulation may comprise dry particles which comprise the active ingredient and which have a diameter in the range from about 0.5 to about 7 nanometers, or from about 1 to about 6 nanometers. Such compositions are conveniently in the form of dry powders for administration using a device comprising a dry powder reservoir to which a stream of propellant can be directed to disperse the powder and/or using a self-propelling solvent/powder dispensing container such as a device comprising the active ingredient dissolved and/or suspended in a low-boiling propellant in a sealed container. Such powders comprise particles wherein at least 98% of the particles by weight have a diameter greater than 0.5 nanometers and at least 95% of the particles by number have a diameter less than 7 nanometers. Alternatively, at least 95% of the particles by weight have a diameter greater than 1 nanometer and at least 90% of the particles by number have a diameter less than 6 nanometers. Dry powder compositions may include a solid fine powder diluent such as sugar and are conveniently provided in a unit dose form.

Low boiling propellants generally include liquid propellants having a boiling point of below 65° F. at atmospheric pressure. Generally the propellant may constitute 50 to 99.9% (w/w) of the composition, and the active ingredient may constitute 0.1 to 20% (w/w) of the composition. The propellant may further comprise additional ingredients such as a liquid non-ionic and/or solid anionic surfactant and/or a solid diluent (which may have a particle size of the same order as particles comprising the active ingredient).

Pharmaceutical compositions described herein formulated for pulmonary delivery may provide the active ingredient in the form of droplets of a solution and/or suspension. Such formulations can be prepared, packaged, and/or sold as aqueous and/or dilute alcoholic solutions and/or suspensions, optionally sterile, comprising the active ingredient, and may conveniently be administered using any nebulization and/or atomization device. Such formulations may further comprise one or more additional ingredients including, but not limited to, a flavoring agent such as saccharin sodium, a volatile oil, a buffering agent, a surface active agent, and/or a preservative such as methylhydroxybenzoate. The droplets provided by this route of administration may have an average diameter in the range from about 0.1 to about 200 nanometers.

Formulations described herein as being useful for pulmonary delivery are useful for intranasal delivery of a pharmaceutical composition described herein. Another formulation suitable for intranasal administration is a coarse powder comprising the active ingredient and having an average particle from about 0.2 to 500 micrometers. Such a formulation is administered by rapid inhalation through the nasal passage from a container of the powder held close to the nares.

Formulations for nasal administration may, for example, comprise from about as little as 0.1% (w/w) to as much as 100% (w/w) of the active ingredient, and may comprise one or more of the additional ingredients described herein. A pharmaceutical composition described herein can be prepared, packaged, and/or sold in a formulation for buccal administration. Such formulations may, for example, be in the form of tablets and/or lozenges made using conventional methods, and may contain, for example, 0.1 to 20% (w/w) active ingredient, the balance comprising an orally dissolvable and/or degradable composition and, optionally, one or more of the additional ingredients described herein. Alternately, formulations for buccal administration may comprise a powder and/or an aerosolized and/or atomized solution and/or suspension comprising the active ingredient. Such powdered, aerosolized, and/or aerosolized formulations, when dispersed, may have an average particle and/or droplet size in the range from about 0.1 to about 200 nanometers, and may further comprise one or more of the additional ingredients described herein.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form for ease of administration and uniformity of dosage. It will be understood, however, that the total daily usage of the compositions described herein will be decided by a physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex, and diet of the subject; the time of administration, route of administration, and rate of excretion of the specific active ingredient employed; the duration of the treatment; drugs used in combination or coincidental with the specific active ingredient employed; and like factors well known in the medical arts.

The compounds and compositions provided herein can be administered by any route, including enteral (e.g., oral), parenteral, intravenous, intramuscular, intra-arterial, intramedullary, intrathecal, subcutaneous, intraventricular, transdermal, interdermal, rectal, intravaginal, intraperitoneal, topical (as by powders, ointments, creams, and/or drops), mucosal, nasal, bucal, sublingual; by intratracheal instillation, bronchial instillation, and/or inhalation; and/or as an oral spray, nasal spray, and/or aerosol. Specifically contemplated routes are oral administration, intravenous administration (e.g., systemic intravenous injection), regional administration via blood and/or lymph supply, and/or direct administration to an affected site. In general, the most appropriate route of administration will depend upon a variety of factors including the nature of the agent (e.g., its stability in the environment of the gastrointestinal tract), and/or the condition of the subject (e.g., whether the subject is able to tolerate oral administration). In certain embodiments, the compound or pharmaceutical composition described herein is suitable for topical administration to the eye of a subject.

The exact amount of a compound required to achieve an effective amount will vary from subject to subject, depending, for example, on species, age, and general condition of a subject, severity of the side effects or disorder, identity of the particular compound, mode of administration, and the like. An effective amount may be included in a single dose (e.g., single oral dose) or multiple doses (e.g., multiple oral doses). In certain embodiments, when multiple doses are administered to a subject or applied to a biological sample, tissue, or cell, any two doses of the multiple doses include different or substantially the same amounts of a compound described herein. In certain embodiments, when multiple doses are administered to a subject or applied to a biological sample, tissue, or cell, the frequency of administering the multiple doses to the subject or applying the multiple doses to the biological sample, tissue, or cell is three doses a day, two doses a day, one dose a day, one dose every other day, one dose every third day, one dose every week, one dose every two weeks, one dose every three weeks, or one dose every four weeks. In certain embodiments, the frequency of administering the multiple doses to the subject or applying the multiple doses to the biological sample, tissue, or cell is one dose per day. In certain embodiments, the frequency of administering the multiple doses to the subject or applying the multiple doses to the biological sample, tissue, or cell is two doses per day. In certain embodiments, the frequency of administering the multiple doses to the subject or applying the multiple doses to the biological sample, tissue, or cell is three doses per day. In certain embodiments, when multiple doses are administered to a subject or applied to a biological sample, tissue, or cell, the duration between the first dose and last dose of the multiple doses is one day, two days, four days, one week, two weeks, three weeks, one month, two months, three months, four months, six months, nine months, one year, two years, three years, four years, five years, seven years, ten years, fifteen years, twenty years, or the lifetime of the subject, biological sample, tissue, or cell. In certain embodiments, the duration between the first dose and last dose of the multiple doses is three months, six months, or one year. In certain embodiments, the duration between the first dose and last dose of the multiple doses is the lifetime of the subject, biological sample, tissue, or cell. In certain embodiments, a dose (e.g., a single dose, or any dose of multiple doses) described herein includes independently between 0.1 μg and 1 μg, between 0.001 mg and 0.01 mg, between 0.01 mg and 0.1 mg, between 0.1 mg and 1 mg, between 1 mg and 3 mg, between 3 mg and 10 mg, between 10 mg and 30 mg, between 30 mg and 100 mg, between 100 mg and 300 mg, between 300 mg and 1,000 mg, or between 1 g and 10 g, inclusive, of a compound described herein. In certain embodiments, a dose described herein includes independently between 1 mg and 3 mg, inclusive, of a compound described herein. In certain embodiments, a dose described herein includes independently between 3 mg and 10 mg, inclusive, of a compound described herein. In certain embodiments, a dose described herein includes independently between 10 mg and 30 mg, inclusive, of a compound described herein. In certain embodiments, a dose described herein includes independently between 30 mg and 100 mg, inclusive, of a compound described herein.

Dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

A compound or composition, as described herein, can be administered in combination with one or more additional pharmaceutical agents (e.g., therapeutically and/or prophylactically active agents). The compounds or compositions can be administered in combination with additional pharmaceutical agents that improve their activity (e.g., activity (e.g., potency and/or efficacy) in treating a disease in a subject in need thereof, in preventing a disease in a subject in need thereof, in inducing the degradation of a target kinase (e.g., HCK, BTK, FYN, SRC, BLK, LCK, CSK, ABL1, ABL2, LIMK1, LATS1), and/or in reducing the risk to develop a disease in a subject in need thereof), improve bioavailability, improve their ability to cross the blood-brain barrier, improve safety, reduce drug resistance, reduce and/or modify metabolism, inhibit excretion, and/or modify distribution in a subject, biological sample, tissue, or cell. It will also be appreciated that the therapy employed may achieve a desired effect for the same disorder, and/or it may achieve different effects. In certain embodiments, a pharmaceutical composition described herein including a compound described herein and an additional pharmaceutical agent exhibit a synergistic effect that is absent in a pharmaceutical composition including one of the compound and the additional pharmaceutical agent, but not both.

The compound or composition can be administered concurrently with, prior to, or subsequent to one or more additional pharmaceutical agents, which may be useful as, e.g., combination therapies. Pharmaceutical agents include therapeutically active agents. Pharmaceutical agents also include prophylactically active agents. Pharmaceutical agents include small organic molecules such as drug compounds (e.g., compounds approved for human or veterinary use by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells. In certain embodiments, the additional pharmaceutical agent is a pharmaceutical agent useful for treating and/or preventing a disease (e.g., proliferative disease (e.g., ovarian cancer, breast cancer, or prostate cancer)). Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the compound described herein with the additional pharmaceutical agent(s) and/or the desired therapeutic and/or prophylactic effect to be achieved. In general, it is expected that the additional pharmaceutical agent(s) in combination be utilized at levels that do not exceed the levels at which they are utilized individually. In some embodiments, the levels utilized in combination will be lower than those utilized individually.

The additional pharmaceutical agents include, but are not limited to, cytotoxic chemotherapeutic agents, epigenetic modifiers, glucocorticoids, immunotherapeutic agents, anti-proliferative agents, anti-cancer agents, cytotoxic agents, anti-angiogenesis agents, anti-inflammatory agents, immunosuppressants, anti-bacterial agents, anti-viral agents, cardiovascular agents, cholesterol-lowering agents, anti-diabetic agents, anti-allergic agents, contraceptive agents, pain-relieving agents, and a combination thereof. In certain embodiments, the additional pharmaceutical agent is an anti-proliferative agent (e.g., anti-cancer agent). In certain embodiments, the additional pharmaceutical agent is abiraterone acetate (e.g., ZYTIGA), ABVD, ABVE, ABVE-PC, AC, AC-T, ADE, ado-trastuzumab emtansine (e.g., KADCYLA), afatinib dimaleate (e.g., GILOTRIF), aldesleukin (e.g., PROLEUKIN), alemtuzumab (e.g., CAMPATH), anastrozole (e.g., ARIMIDEX), arsenic trioxide (e.g., TRISENOX), asparaginase Erwinia chrysanthemi (e.g., ERWINAZE), axitinib (e.g., INLYTA), azacitidine (e.g., MYLOSAR, VIDAZA), BEACOPP, belinostat (e.g., BELEODAQ), bendamustine hydrochloride (e.g., TREANDA), BEP, bevacizumab (e.g., AVASTIN), bicalutamide (e.g., CASODEX), bleomycin (e.g., BLENOXANE), blinatumomab (e.g., BLINCYTO), bortezomib (e.g., VELCADE), bosutinib (e.g., BOSULIF), brentuximab vedotin (e.g., ADCETRIS), busulfan (e.g., BUSULFEX, MYLERAN), cabazitaxel (e.g., JEVTANA), cabozantinib-s-malate (e.g., COMETRIQ), CAF, capecitabine (e.g., XELODA), CAPOX, carboplatin (e.g., PARAPLAT, PARAPLATIN), carboplatin-taxol, carfilzomib (e.g., KYPROLIS), carmustine (e.g., BECENUM, BICNU, CARMUBRIS), carmustine implant (e.g., GLIADEL WAFER, GLIADEL), ceritinib (e.g., ZYKADIA), cetuximab (e.g., ERBITUX), chlorambucil (e.g., AMBOCHLORIN, AMBOCLORIN, LEUKERAN, LINFOLIZIN), chlorambucil-prednisone, CHOP, cisplatin (e.g., PLATINOL, PLATINOL-AQ), clofarabine (e.g., CLOFAREX, CLOLAR), CMF, COPP, COPP-ABV, crizotinib (e.g., XALKORI), CVP, cyclophosphamide (e.g., CLAFEN, CYTOXAN, NEOSAR), cytarabine (e.g., CYTOSAR-U, TARABINE PFS), dabrafenib (e.g., TAFINLAR), dacarbazine (e.g., DTIC-DOME), dactinomycin (e.g., COSMEGEN), dasatinib (e.g., SPRYCEL), daunorubicin hydrochloride (e.g., CERUBIDINE), decitabine (e.g., DACOGEN), degarelix, denileukin diftitox (e.g., ONTAK), denosumab (e.g., PROLIA, XGEVA), Dinutuximab (e.g., UNITUXIN), docetaxel (e.g., TAXOTERE), doxorubicin hydrochloride (e.g., ADRIAMYCIN PFS, ADRIAMYCIN RDF), doxorubicin hydrochloride liposome (e.g., DOXIL, DOX-SL, EVACET, LIPODOX), enzalutamide (e.g., XTANDI), epirubicin hydrochloride (e.g., ELLENCE), EPOCH, erlotinib hydrochloride (e.g., TARCEVA), etoposide (e.g., TOPOSAR, VEPESID), etoposide phosphate (e.g., ETOPOPHOS), everolimus (e.g., AFINITOR DISPERZ, AFINITOR), exemestane (e.g., AROMASIN), FEC, fludarabine phosphate (e.g., FLUDARA), fluorouracil (e.g., ADRUCIL, EFUDEX, FLUOROPLEX), FOLFIRI, FOLFIRI-BEVACIZUMAB, FOLFIRI-CETUXIMAB, FOLFIRINOX, FOLFOX, FU-LV, fulvestrant (e.g., FASLODEX), gefitinib (e.g., IRESSA), gemcitabine hydrochloride (e.g., GEMZAR), gemcitabine-cisplatin, gemcitabine-oxaliplatin, goserelin acetate (e.g., ZOLADEX), Hyper-CVAD, ibritumomab tiuxetan (e.g., ZEVALIN), ibrutinib (e.g., IMBRUVICA), ICE, idelalisib (e.g., ZYDELIG), ifosfamide (e.g., CYFOS, IFEX, IFOSFAMIDUM), imatinib mesylate (e.g., GLEEVEC), imiquimod (e.g., ALDARA), ipilimumab (e.g., YERVOY), irinotecan hydrochloride (e.g., CAMPTOSAR), ixabepilone (e.g., IXEMPRA), lanreotide acetate (e.g., SOMATULINE DEPOT), lapatinib ditosylate (e.g., TYKERB), lenalidomide (e.g., REVLIMID), lenvatinib (e.g., LENVIMA), letrozole (e.g., FEMARA), leucovorin calcium (e.g., WELLCOVORIN), leuprolide acetate (e.g., LUPRON DEPOT, LUPRON DEPOT-3 MONTH, LUPRON DEPOT-4 MONTH, LUPRON DEPOT-PED, LUPRON, VIADUR), liposomal cytarabine (e.g., DEPOCYT), lomustine (e.g., CEENU), mechlorethamine hydrochloride (e.g., MUSTARGEN), megestrol acetate (e.g., MEGACE), mercaptopurine (e.g., PURINETHOL, PURIXAN), methotrexate (e.g., ABITREXATE, FOLEX PFS, FOLEX, METHOTREXATE LPF, MEXATE, MEXATE-AQ), mitomycin c (e.g., MITOZYTREX, MUTAMYCIN), mitoxantrone hydrochloride, MOPP, nelarabine (e.g., ARRANON), nilotinib (e.g., TASIGNA), nivolumab (e.g., OPDIVO), obinutuzumab (e.g., GAZYVA), OEPA, ofatumumab (e.g., ARZERRA), OFF, olaparib (e.g., LYNPARZA), omacetaxine mepesuccinate (e.g., SYNRIBO), OPPA, oxaliplatin (e.g., ELOXATIN), paclitaxel (e.g., TAXOL), paclitaxel albumin-stabilized nanoparticle formulation (e.g., ABRAXANE), PAD, palbociclib (e.g., IBRANCE), pamidronate disodium (e.g., AREDIA), panitumumab (e.g., VECTIBIX), panobinostat (e.g., FARYDAK), pazopanib hydrochloride (e.g., VOTRIENT), pegaspargase (e.g., ONCASPAR), peginterferon alfa-2b (e.g., PEG-INTRON), peginterferon alfa-2b (e.g., SYLATRON), pembrolizumab (e.g., KEYTRUDA), pemetrexed disodium (e.g., ALIMTA), pertuzumab (e.g., PERJETA), plerixafor (e.g., MOZOBIL), pomalidomide (e.g., POMALYST), ponatinib hydrochloride (e.g., ICLUSIG), pralatrexate (e.g., FOLOTYN), prednisone, procarbazine hydrochloride (e.g., MATULANE), radium 223 dichloride (e.g., XOFIGO), raloxifene hydrochloride (e.g., EVISTA, KEOXIFENE), ramucirumab (e.g., CYRAMZA), R-CHOP, recombinant HPV bivalent vaccine (e.g., CERVARIX), recombinant human papillomavirus (e.g., HPV) nonavalent vaccine (e.g., GARDASIL 9), recombinant human papillomavirus (e.g., HPV) quadrivalent vaccine (e.g., GARDASIL), recombinant interferon alfa-2b (e.g., INTRON A), regorafenib (e.g., STIVARGA), rituximab (e.g., RITUXAN), romidepsin (e.g., ISTODAX), ruxolitinib phosphate (e.g., JAKAFI), siltuximab (e.g., SYLVANT), sipuleucel-t (e.g., PROVENGE), sorafenib tosylate (e.g., NEXAVAR), STANFORD V, sunitinib malate (e.g., SUTENT), TAC, tamoxifen citrate (e.g., NOLVADEX, NOVALDEX), temozolomide (e.g., METHAZOLASTONE, TEMODAR), temsirolimus (e.g., TORISEL), thalidomide (e.g., SYNOVIR, THALOMID), thiotepa, topotecan hydrochloride (e.g., HYCAMTIN), toremifene (e.g., FARESTON), tositumomab and iodine I 131 tositumomab (e.g., BEXXAR), TPF, trametinib (e.g., MEKINIST), trastuzumab (e.g., HERCEPTIN), VAMP, vandetanib (e.g., CAPRELSA), VEIP, vemurafenib (e.g., ZELBORAF), vinblastine sulfate (e.g., VELBAN, VELSAR), vincristine sulfate (e.g., VINCASAR PFS), vincristine sulfate liposome (e.g., MARQIBO), vinorelbine tartrate (e.g., NAVELBINE), vismodegib (e.g., ERIVEDGE), vorinostat (e.g., ZOLINZA), XELIRI, XELOX, ziv-aflibercept (e.g., ZALTRAP), or zoledronic acid (e.g., ZOMETA). In certain embodiments, the additional pharmaceutical agent is ENMD-2076, PCI-32765, AC220, dovitinib lactate (e.g., TKI258, CHIR-258), BIBW 2992 (e.g., TOVOK™), SGX523, PF-04217903, PF-02341066, PF-299804, BMS-777607, ABT-869, MP470, BIBF 1120 (e.g., VARGATEF®), AP24534, JNJ-26483327, MGCD265, DCC-2036, BMS-690154, CEP-11981, tivozanib (e.g., AV-951), OSI-930, MM-121, XL-184, XL-647, and/or XL228), proteasome inhibitors (e.g., bortezomib (e.g., Velcade)), mTOR inhibitors (e.g., rapamycin, temsirolimus (e.g., CCI-779), everolimus (e.g., RAD-001), ridaforolimus, AP23573 (e.g., Ariad), AZD8055, BEZ235, BGT226, XL765, PF-4691502, GDC0980, SF1126, and OSI-027), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, or a combination thereof. In certain embodiments, the additional pharmaceutical agent is a cytotoxic chemotherapy (e.g., cytotoxic chemotherapeutic agent (e.g., gemcitabine, cytarabine, daunorubicin, doxorubicin, vincristine, 1-asparaginase, cyclophosphamide, or etoposide)). In certain embodiments, the additional pharmaceutical agent is an epigenetic modifier, such as azacitidine or romidepsin. In certain embodiments, the additional pharmaceutical agent is ruxolitinib, BBT594, CHZ868, CYT387, or BMS911543. In certain embodiments, the additional pharmaceutical agent is an inhibitor of a tyrosine kinase. In some embodiments, the additional pharmaceutical agent is a topoisomerase inhibitor, a MCL1 inhibitor, a BCL-2 inhibitor, a BCL-xL inhibitor, a BRD4 inhibitor, a BRCA1 inhibitor, BRCA2 inhibitor, HER1 inhibitor, HER2 inhibitor, a CDK9 inhibitor, a Jumonji histone demethylase inhibitor, or a DNA damage inducer. In some embodiments, the additional pharmaceutical agent is etoposide, obatoclax, navitoclax, JQ1, 4-(((5′-chloro-2′-(((1R,4R)-4-(((R)-1-methoxypropan-2-yl)amino)cyclohexyl)amino)-[2,4′-bipyridin]-6-yl)amino)methyl)tetrahydro-2H-pyran-4-carbonitrile, JIB04, or cisplatin. In certain embodiments, the additional pharmaceutical agent is a binder or inhibitor of a kinase (e.g., SFK (e.g., HCK)). In certain embodiments, the additional pharmaceutical agent is an antibody or a fragment thereof (e.g., monoclonal antibody). In certain embodiments, the additional pharmaceutical agent is a tyrosine kinase inhibitor. In certain embodiments, the additional pharmaceutical agent is selected from the group consisting of epigenetic or transcriptional modulators (e.g., DNA methyltransferase inhibitors, histone deacetylase inhibitors (HDAC inhibitors), lysine methyltransferase inhibitors), antimitotic drugs (e.g., taxanes and vinca alkaloids), hormone receptor modulators (e.g., estrogen receptor modulators and androgen receptor modulators), cell signaling pathway inhibitors (e.g., tyrosine protein kinase inhibitors), modulators of protein stability (e.g., proteasome inhibitors), Hsp90 inhibitors, glucocorticoids, all-trans retinoic acids, and other agents that promote differentiation. In certain embodiments, the additional pharmaceutical agent is a glucocorticoid (e.g., cortisol, cortisone, prednisone, methylprednisolone, dexamethasone, betamethasone, triamcinolone, fludrocortisone acetate, or deoxycorticosterone acetate). In certain embodiments, the additional therapy is an immunotherapy (e.g., an immunotherapeutic monoclonal antibody). In certain embodiments, the additional pharmaceutical agent is an immunomodulator. In certain embodiments, the additional pharmaceutical agent is an immune checkpoint inhibitor. In certain embodiments, the additional pharmaceutical agent is a programmed cell death 1 protein (PD-1) inhibitor. In certain embodiments, the additional pharmaceutical agent is a programmed cell death 1 protein ligand 1 (PD-L1) inhibitor. In certain embodiments, the additional pharmaceutical agent is a cytotoxic T-lymphocyte-associated protein 4 (CTLA-4) inhibitor. In certain embodiments, the additional pharmaceutical agent is a T-cell immunoglobulin domain and mucin domain 3 (TIM3) inhibitor, lymphocyte activation gene-3 (LAG3) inhibitor, V-set domain-containing T-cell activation inhibitor 1 (VTCN1 or B7-H4) inhibitor, cluster of differentiation 276 (CD276 or B7-H3) inhibitor, B and T lymphocyte attenuator (BTLA) inhibitor, galectin-9 (GAL9) inhibitor, checkpoint kinase 1 (Chk1) inhibitor, adenosine A2A receptor (A2AR) inhibitor, indoleamine 2,3-dioxygenase (IDO) inhibitor, killer-cell immunoglobulin-like receptor (KIR) inhibitor, or V-domain Ig suppressor of T cell activation (VISTA) inhibitor. In certain embodiments, the PD-1 inhibitor is nivolumab, pidilizumab, pembrolizumab, MEDI-0680, REGN2810, or AMP-224. In certain embodiments, the PD-L1 inhibitor is atezolizumab, durvalumab, BMS-936559, avelumab, or CA-170. In certain embodiments, the CTLA-4 inhibitor is ipilimumab or tremelimumab. In certain embodiments, the additional pharmaceutical agent is an aromatase inhibitor. In certain embodiments, the additional pharmaceutical agent is an PI3K inhibitor. In certain embodiments, the additional pharmaceutical agent is an mTOR inhibitor. In certain embodiments, the additional pharmaceutical agent is an endocrine therapy. In certain embodiments, the additional pharmaceutical agent is an anti-proliferative agent, for example, a proteasome inhibitor (e.g., bortezomib, carfilzomib, ixazomib) and/or a BCL-2 inhibitor (e.g., venetoclax). In certain embodiments, the additional pharmaceutical agent is a proteasome inhibitor (e.g., bortezomib, carfilzomib, ixazomib). In certain embodiments, the additional pharmaceutical agent is bortezomib. In certain embodiments, the additional pharmaceutical agent is a BCL-2 inhibitor (e.g., venetoclax). In certain embodiments, the additional pharmaceutical agent is venetoclax. In certain embodiments, the compounds described herein or pharmaceutical compositions thereof can be administered in combination with an anti-cancer therapy, including, but not limited to, surgery, radiation therapy, transplantation (e.g., stem cell transplantation, bone marrow transplantation), immunotherapy, and chemotherapy. In certain embodiments, the compounds described herein or pharmaceutical compositions thereof can be administered in combination with chemotherapy. In certain embodiments, the compounds described herein or pharmaceutical compositions thereof can be administered in combination with immunotherapy. In certain embodiments, the compounds described herein or pharmaceutical compositions thereof can be administered in combination with chemotherapy or immunotherapy. In certain embodiments, the compounds described herein or pharmaceutical compositions thereof can be administered in combination with a proteasome inhibitor (e.g., bortezomib, carfilzomib, ixazomib) and/or a BCL-2 inhibitor (e.g., venetoclax). In certain embodiments, the compounds described herein or pharmaceutical compositions thereof can be administered in combination with a proteasome inhibitor (e.g., bortezomib, carfilzomib, ixazomib). In certain embodiments, the compounds described herein or pharmaceutical compositions thereof can be administered in combination with the proteasome inhibitor bortezomib. In certain embodiments, the compounds described herein or pharmaceutical compositions thereof can be administered in combination with a BCL-2 inhibitor (e.g., venetoclax). In certain embodiments, the compounds described herein or pharmaceutical compositions thereof can be administered in combination with the BCL-2 inhibitor venetoclax.

Also encompassed by the disclosure are kits (e.g., pharmaceutical packs). The kits provided may comprise a pharmaceutical composition or compound described herein and a container (e.g., a vial, ampule, bottle, syringe, and/or dispenser package, or other suitable container). In some embodiments, provided kits may optionally further include a second container comprising a pharmaceutical excipient for dilution or suspension of a pharmaceutical composition or compound described herein. In some embodiments, the pharmaceutical composition or compound described herein provided in the first container and the second container are combined to form one unit dosage form.

Thus, in one aspect, provided are kits including a first container comprising a compound or pharmaceutical composition described herein. In certain embodiments, the kits are useful for treating a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the kits are useful for preventing a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof.

In certain embodiments, a kit described herein further includes instructions for using the compound or pharmaceutical composition included in the kit. A kit described herein may also include information as required by a regulatory agency such as the U.S. Food and Drug Administration (FDA). In certain embodiments, the information included in the kits is prescribing information. In certain embodiments, the kits and instructions provide for treating a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the kits and instructions provide for preventing a disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) in a subject in need thereof. In certain embodiments, the kits and instructions provide for inducing the degradation of a kinase (e.g., HCK, BTK) in a subject, biological sample, tissue, or cell. A kit described herein may include one or more additional pharmaceutical agents described herein as a separate composition. In certain embodiments, within the kit, the compounds described herein or pharmaceutical compositions thereof can be administered in combination with one or more additional pharmaceutical agents, for example, an anti-cancer therapy, including, but not limited to, surgery, radiation therapy, transplantation (e.g., stem cell transplantation, bone marrow transplantation), immunotherapy, and chemotherapy. In certain embodiments, the additional pharmaceutical agent is a proteasome inhibitor (e.g., bortezomib, carfilzomib, ixazomib). In certain embodiments, the additional pharmaceutical agent is a BCL-2 inhibitor (e.g., venetoclax).

### Methods of Treatment and Uses

The compounds described herein are capable of binding (e.g., reversibly binding or irreversibly binding) an E3 ubiquitin ligase (e.g., Cereblon) and the kinase (e.g., HCK) and inducing the degradation of the kinase (e.g., HCK, BTK, FYN, SRC, BLK, LCK, CSK, ABL1, ABL2, LIMK1, LATS1). The compounds described herein are capable of binding (e.g., reversibly binding or irreversibly binding) an E3 ubiquitin ligase (e.g., Cereblon) and the kinase (e.g., HCK) and inducing the degradation of the kinase (e.g., HCK, BTK). The present disclosure thus also provides methods of inducing the degradation of the tkinase (e.g., HCK) in a subject, biological sample, tissue, or cell. The compounds described herein are capable of binding (e.g., reversibly binding or irreversibly binding) an E3 ubiquitin ligase (e.g., Cereblon) and the kinase (e.g., HCK) and inducing the degradation of HCK and/or BTK. The present disclosure thus also provides methods of inducing the degradation of the kinase (e.g., HCK) in a subject, biological sample, tissue, or cell. The present disclosure thus also provides methods of inducing the degradation of HCK in a subject, biological sample, tissue, or cell. The present disclosure thus also provides methods of inducing the degradation of BTK in a subject, biological sample, tissue, or cell. The present disclosure further provides methods for the treatment of diseases, such as proliferative diseases in a subject in need thereof.

In certain embodiments, the application provides a method of binding an ubiquitin receptor E3 ubiquitin ligase (e.g., Cereblon) and promoting the degradation of the kinase (e.g., HCK, BTK). In certain embodiments, the application provides a method of binding an ubiquitin receptor E3 ubiquitin ligase (e.g., Cereblon) and promoting the degradation of HCK. In certain embodiments, the application provides a method of binding an ubiquitin receptor E3 ubiquitin ligase (e.g., Cereblon) and promoting the degradation of BTK. In another aspect, the present disclosure provides methods of inducing the degradation of the kinase (e.g., HCK, BTK) in a subject in need thereof, the methods comprise administering to the subject an effective amount of a compound or pharmaceutical composition described herein. In another aspect, the present disclosure provides methods of inducing the degradation of HCK in a subject in need thereof, the methods comprise administering to the subject an effective amount of a compound or pharmaceutical composition described herein. In another aspect, the present disclosure provides methods of inducing the degradation of the kinase (e.g., HCK) in a biological sample, tissue, or cell, the methods comprise contacting the biological sample, tissue, or cell with an effective amount of a compound or pharmaceutical composition described herein. In another aspect, the present disclosure provides methods of inducing the degradation of HCK in a biological sample, tissue, or cell, the methods comprise contacting the biological sample, tissue, or cell with an effective amount of a compound or pharmaceutical composition described herein. In another aspect, the present disclosure provides methods of inducing the degradation of BTK in a subject in need thereof, the methods comprise administering to the subject an effective amount of a compound or pharmaceutical composition described herein. In another aspect, the present disclosure provides methods of inducing the degradation of the kinase (e.g., BTK) in a biological sample, tissue, or cell, the methods comprise contacting the biological sample, tissue, or cell with an effective amount of a compound or pharmaceutical composition described herein. In another aspect, the present disclosure provides methods of inducing the degradation of BTK in a biological sample, tissue, or cell, the methods comprise contacting the biological sample, tissue, or cell with an effective amount of a compound or pharmaceutical composition described herein.

In certain embodiments, provided is a method of binding an E3 ubiquitin ligase (e.g., Cereblon) and the kinase (e.g., HCK) and selectively inducing the degradation of the kinase (e.g., HCK, BTK, FYN, SRC, BLK, LCK, CSK, ABL1, ABL2, LIMK1, LATS1). In certain embodiments, the application provides a method of binding an E3 ubiquitin ligase (e.g., Cereblon) and the kinase (e.g., HCK) and selectively inducing the degradation of the kinase (e.g., HCK, BTK).

In certain embodiments, provided is use of a bifunctional compound that binds an E3 ubiquitin ligase (e.g., Cereblon) and the kinase (e.g., HCK, BTK, FYN, SRC, BLK, LCK, CSK, ABL1, ABL2, LIMK1, LATS1) provides a strategy for treating diseases associated with a kinase (e.g., HCK) (e.g. proliferative diseases), as research tools for studying the role of HCK in the cell, or as research tools for studying diseases associated with a kinase (e.g., HCK) (e.g. proliferative diseases). In certain embodiments, provided is use of a bifunctional compound that binds an E3 ubiquitin ligase (e.g., Cereblon) and the kinase (e.g., HCK) provides a strategy for treating diseases associated with a kinase (e.g., HCK) (e.g. proliferative diseases), as research tools for studying the role of HCK in the cell, or as research tools for studying diseases associated with a kinase (e.g., HCK) (e.g. proliferative diseases).

The present disclosure also provides a compound of Formula (I), or a pharmaceutically acceptable salt, co-crystal, tautomer, stereoisomer, solvate, hydrate, polymorph, isotopically enriched derivative, or prodrug, or composition thereof, for use in the treatment of diseases, such as proliferative diseases, in a subject in need thereof.

The present disclosure also provides uses of a compound of Formula (I), or a pharmaceutically acceptable salt, co-crystal, tautomer, stereoisomer, solvate, hydrate, polymorph, isotopically enriched derivative, or prodrug, or composition thereof, in the manufacture of a medicament for the treatment of diseases, such as proliferative diseases, in a subject in need thereof. The present disclosure also provides uses of a compound of Formula (I), or a pharmaceutically acceptable salt, co-crystal, tautomer, stereoisomer, solvate, hydrate, polymorph, isotopically enriched derivative, or prodrug, or composition thereof, for the treatment of diseases, such as proliferative diseases, in a subject in need thereof.

In certain embodiments, the methods of the disclosure comprise administering to the subject an effective amount of a compound of Formula (I), or a pharmaceutically acceptable salt, co-crystal, tautomer, stereoisomer, solvate, hydrate, polymorph, isotopically enriched derivative, or prodrug, or composition thereof. In some embodiments, the effective amount is a therapeutically effective amount. In some embodiments, the effective amount is a prophylactically effective amount.

In certain embodiments, the subject being treated is an animal. The animal may be of either sex and may be at any stage of development. In certain embodiments, the subject is a mammal. In certain embodiments, the subject being treated is a human. In certain embodiments, the subject is a domesticated animal, such as a dog, cat, cow, pig, horse, sheep, or goat. In certain embodiments, the subject is a companion animal, such as a dog or cat. In certain embodiments, the subject is a livestock animal, such as a cow, pig, horse, sheep, or goat. In certain embodiments, the subject is a zoo animal. In another embodiment, the subject is a research animal such as a rodent (e.g., mouse, rat), dog, pig, or non-human primate. In certain embodiments, the animal is a genetically engineered animal. In certain embodiments, the animal is a transgenic animal.

Certain methods described herein may comprise administering one or more additional pharmaceutical agent(s) in combination with the compounds described herein. The additional pharmaceutical agent(s) may be administered at the same time as the compound of Formula (I), or at different times than the compound of Formula (I). For example, the compound of Formula (I) and any additional pharmaceutical agent(s) may be on the same dosing schedule or different dosing schedules. All or some doses of the compound of Formula (I) may be administered before all or some doses of an additional pharmaceutical agent, after all or some does an additional pharmaceutical agent, within a dosing schedule of an additional pharmaceutical agent, or a combination thereof. The timing of administration of the compound of Formula (I) and additional pharmaceutical agents may be different for different additional pharmaceutical agents.

In certain embodiments, the additional pharmaceutical agent comprises an agent useful in the treatment of diseases, such as proliferative diseases, in a subject in need thereof. In certain embodiments, the additional pharmaceutical agent is useful in the treatment of a proliferative disease. In certain embodiments, the additional pharmaceutical agent is useful in the treatment of an inflammatory disease. In certain embodiments, the additional pharmaceutical agent is useful in the treatment of proliferative diseases. In certain embodiments, the additional pharmaceutical agent is an anti-proliferative agent. In certain embodiments, the additional pharmaceutical agent is a proteasome inhibitor (e.g., bortezomib, carfilzomib, ixazomib) and/or a BCL-2 inhibitor (e.g., venetoclax). In certain embodiments, the additional pharmaceutical agent is a proteasome inhibitor (e.g., bortezomib, carfilzomib, ixazomib). In certain embodiments, the additional pharmaceutical agent is a BCL-2 inhibitor (e.g., venetoclax). In certain embodiments, the compounds described herein or pharmaceutical compositions thereof can be administered in combination with a proteasome inhibitor (e.g., bortezomib, carfilzomib, ixazomib) and/or a BCL-2 inhibitor (e.g., venetoclax). In certain embodiments, the additional pharmaceutical agent is bortezomib. In certain embodiments, the additional pharmaceutical agent is a BCL-2 inhibitor (e.g., venetoclax). In certain embodiments, the additional pharmaceutical agent is venetoclax. In certain embodiments, the compounds described herein or pharmaceutical compositions thereof can be administered in combination with an anti-cancer therapy, including, but not limited to, surgery, radiation therapy, transplantation (e.g., stem cell transplantation, bone marrow transplantation), immunotherapy, and chemotherapy.

In another aspect, the present disclosure provides methods for inducing the degradation of a kinase (e.g., HCK, BTK, FYN, SRC, BLK, LCK, CSK, ABL1, ABL2, LIMK1, LATS1), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof. In another aspect, the present disclosure provides methods for inducing the degradation of a kinase (e.g., HCK, BTK), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof. In another aspect, the present disclosure provides methods for inducing the degradation of a kinase (e.g., HCK, BTK), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof. In another aspect, the present disclosure provides methods for inducing the degradation of a kinase (e.g., HCK), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof. In another aspect, the present disclosure provides methods for inducing the degradation of a kinase (e.g., BTK), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof.

In another aspect, the present disclosure provides methods for binding an E3 ubiquitin ligase and promoting the degradation and/or ubiquitination of a kinase (e.g., HCK, BTK), the method comprising administering to the subject a compound of Formula (I), or a pharmaceutically acceptable salt, co-crystal, tautomer, stereoisomer, solvate, hydrate, polymorph, isotopically enriched derivative, or prodrug, or composition thereof.

All types of biological samples described herein or known in the art are contemplated as being within the scope of the invention. In certain embodiments, the disease (e.g., proliferative disease (e.g., non-Hodgkin's lymphoma, Burkitt's lymphoma, Waldenstrom macroglobulinemia, MYD88-mutated Waldenstrom macroglobulinemia, diffuse large B-cell lymphoma (e.g., activated B-cell diffuse large B-cell lymphoma, germinal center B-cell-like diffuse large B-cell lymphoma), myelodysplastic syndrome (MDS), leukemia (e.g., acute myeloid leukemia (AML)), multiple myeloma), inflammatory disease, autoimmune disease, or other diseases associated with MYD88 mutations) to be treated or prevented using the compounds described herein is cancer. All types of cancers disclosed herein or known in the art are contemplated as being within the scope of the invention. In certain embodiments, the proliferative disease is cancer. In certain embodiments, the cancer is lymphoma. In certain embodiments, the lymphoma is non-Hodgkin's lymphoma. In certain embodiments, the non-Hodgkin's lymphoma is Waldenstrom macroglobulinemia. In certain embodiments, the non-Hodgkin's lymphoma is MYD88-mutated Waldenstrom macroglobulinemia. In certain embodiments, the lymphoma is diffuse large B-cell lymphoma. In certain embodiments, the lymphoma is diffuse large B-cell lymphoma, for example, activated B-cell (ABC) diffuse large B-cell lymphoma or germinal center B-cell-like diffuse large B-cell lymphoma. In certain embodiments, the cancer is leukemia (e.g., hairy cell leukemia or acute myeloid leukemia (AML)). In certain embodiments, the cancer is associated with hematopoietic cell kinase (HCK). In certain embodiments, the cancer is myelodysplastic syndrome (MDS), In certain embodiments, the cancer is multiple myeloma.

In still another aspect, the present disclosure provides the pharmaceutical compositions described herein for use in binding an E3 ubiquitin ligase and HCK, BTK, FYN, SRC, BLK, LCK, CSK, ABL1, ABL2, LIMK1, or LATS1, and promoting the degradation of a kinase (e.g., HCK, BTK, FYN, SRC, BLK, LCK, CSK, ABL1, ABL2, LIMK1, LATS1); and treating and/or preventing proliferative diseases. In still another aspect, the present disclosure provides the pharmaceutical compositions described herein for use in binding an E3 ubiquitin ligase and HCK and promoting the degradation of a kinase (e.g., HCK, BTK); and treating and/or preventing proliferative diseases.

### EXAMPLES

In order that the present disclosure may be more fully understood, the following examples are set forth. The synthetic and biological examples described in this application are offered to illustrate the compounds, pharmaceutical compositions, and methods provided herein and are not to be construed in any way as limiting their scope.

The compounds provided herein can be prepared from readily available starting materials using the following general methods and procedures or methods known in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Compounds of Formula (I) may be prepared using synthetic schemes and procedures recognized by one of ordinary skill in the art. Compounds of Formula (I) may be prepared using the synthetic schemes and procedures described in detail below.

### Example 1

### Synthesis of Exemplary HCK Degrader Compounds

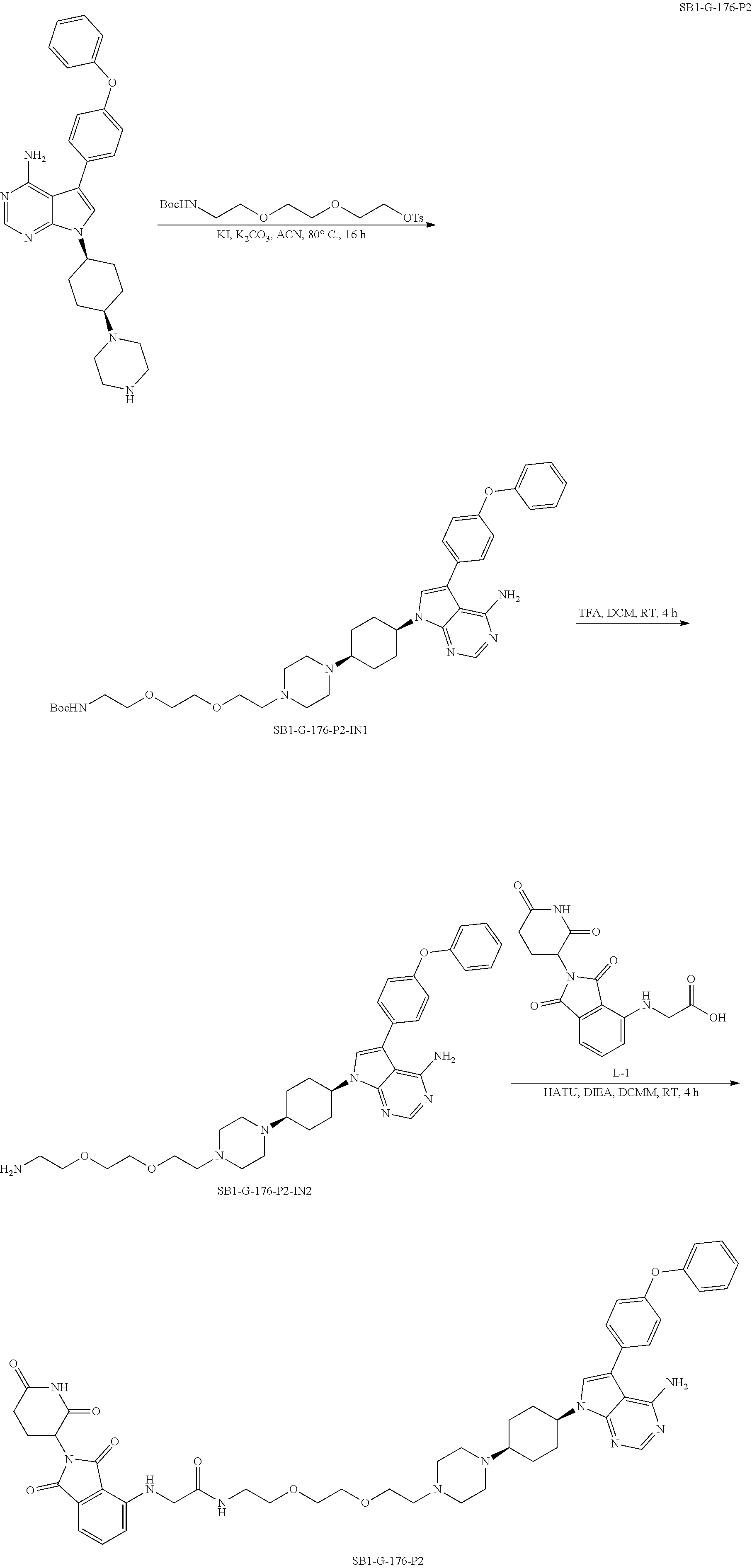

### tert-butyl 2-(2-(2-(4-((1s,4s)-4-(4-amino-5-(4-phenoxyphenyl)-7H-pyrrolo[2,3-d]pyrimidin-7-yl)cyclohexyl) piperazin-1-yl)ethoxy)ethoxy)ethylcarbamate (SB1-G-176-P2-IN1)

The mixture of 5-(4-phenoxyphenyl)-7-((1s,4s)-4-(piperazin-1-yl)cyclohexyl)-7H-pyrrolo[2,3-d]pyrimidin-4-amine (293 mg, 0.63 mmol), 2,2-dimethyl-4-oxo-3,8,11-trioxa-5-azatridecan-13-yl 4-methylbenzenesulfonate (320 mg, 0.79 mmol), K₂CO₃ (215 mg, 1.56 mmol), KI (11.6 mg, 0.07 mmol) and ACN (15 mL) was stirred and heated to 80° C. for 16 h, after completion, diluted with brine (300 mL), extracted with DCM (300 mL×3), the organic phase was washed with brine (200 mL), dried with Na₂SO₄, filtered, removed the solvent, the residue was purified by silica gel column chromatography (DCM:MeOH=10:1) to get SB1-G-176-IN1 (faint yellow solid, 402 mg, yield 84%). LCMS (m/z): 700 [M+H]+. 5-(4-phenoxyphenyl)-7-((1s,4s)-4-(piperazin-1-yl)cyclohexyl)-7H-pyrrolo[2,3-d]pyrimidin-4-amine was synthesized following similar procedure in Bioorganic and Medicinal Chemistry Letters, 2002, 12, p 1683-1686.

### 7-((1s,4s)-4-(4-(2-(2-(2-aminoethoxy)ethoxy)ethyl)piperazin-1-yl)cyclohexyl)-5-(4-phenoxyphenyl)-7H-pyrrolo[2,3-d]pyrimidin-4-amine (SB1-G-176-IN2)

The mixture of SB1-G-176-P2-IN1 (402 mg, 0.574 mmol), TFA (5 mL) and DCM (10 mL) was stirred at RT for 4 h, after completion, concentrated to remove the organic solvent, diluted with water (100 mL), washed with EA (100 mL×3), Na₂CO₃(s) was added into aqueous phase and stirred until PH=9-10, then the aqueous phase extracted with DCM (200 mL×3), the organic phase was washed with brine (150 mL), dried with Na₂SO₄, filtered, removed the solvent, the residue (SB1-G-176-P2-IN2, faint yellow solid, 272 mg, yield 78%) was put into next step without further purification. LCMS (m/z): 600 [M+H]+.

### N-(2-(2-(2-(4-((1s,4s)-4-(4-amino-5-(4-phenoxyphenyl)-7H-pyrrolo[2,3-d]pyrimidin-7-yl)cyclohexyl) piperazin-1-yl)ethoxy)ethoxy)ethyl)-2-(2-(2,6-dioxopiperidin-3-yl)-1,3-dioxoisoindolin-4-ylamino) acetamide (SB1-G-176-P2)

The mixture of SB1-G-176-P2-IN2 (83 mg, 0.138 mmol), L-1 (35 mg, 0.106 mmol), HATU (81 mg, 0.212 mmol), DIPEA (137 mg, 1.06 mmol), and DCM (5 mL) was stirred at RT for 4 h, after completion, diluted with water (50 mL), the aqueous phase was extracted with DCM (100 mL×2), dried with Na₂SO₄, filtered, concentrated to remove the organic solvent, the residue was purified with Prep-HPLC (C18 column, CH₃CN/H₂O, containing 0.05% NH₄HCO₃) to get SB1-G-176-P2 (yellow solid, 17.6 mg). LCMS (m/z): 913 [M+H]+; ¹H NMR (CDCl₃, 400 MHz): δ 8.33 (s, 1H), 7.51 (t, J=8.0 Hz, 1H), 7.45 (d, J=8.0 Hz, 2H), 7.38 (t, J=8 Hz, 2H), 7.20 (d, J=8.0 Hz, 2H), 7.14 (t, J=8.0 Hz, 1H), 7.08 (t, J=8.0 Hz, 5H), 6.83 (d, J=8.0 Hz, 1H), 6.73 (t, J=6.0 Hz, 1H), 5.19 (s, 2H), 4.92 (dd, J₁=4.0 Hz, J₂=12.0 Hz, 1H), 4.78 (m, 1H), 3.98 (d, J=8.0 Hz, 2H), 3.57 (m, 9H), 3.43 (m, 1H), 2.80 (m, 4H), 2.61 (m, 8H), 2.27 (s, 1H), 2.12 (m, 6H), 1.82 (m, 3H), 1.63 (t, J=12.0 Hz, 2H).

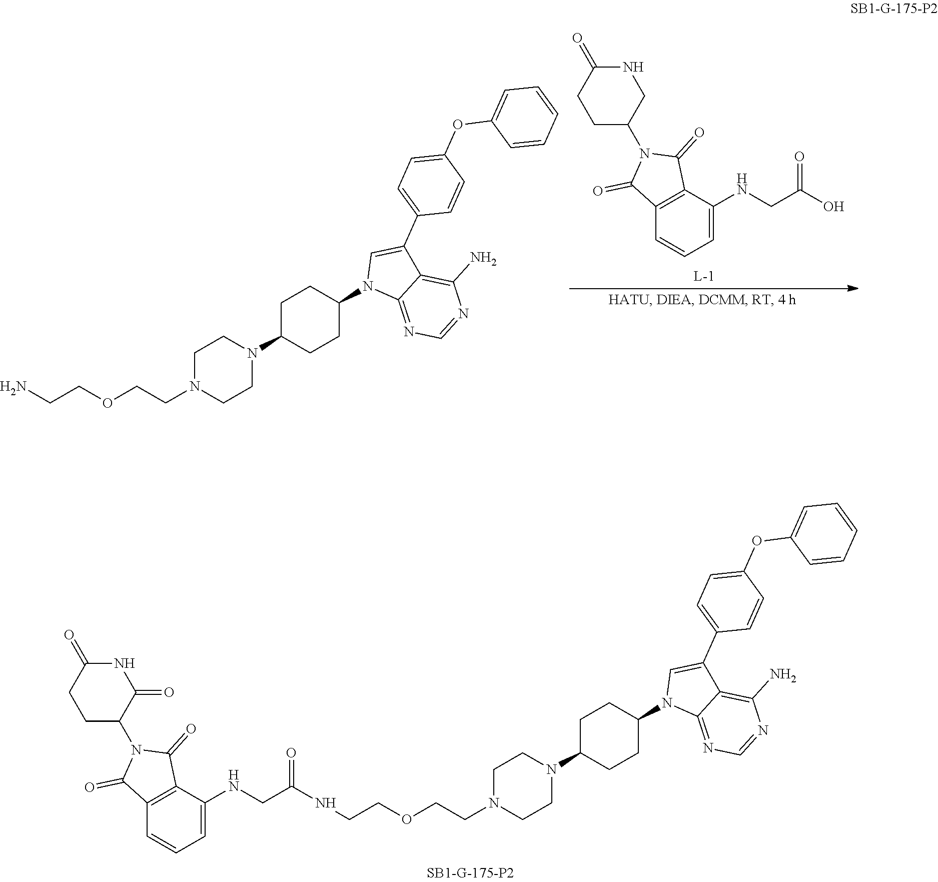

SB1-G-175-P2 was synthesized with similar procedures as SB1-G-176-P2.

LCMS (m/z): 869 [M+H]⁺; ¹H NMR (CDCl₃, 400 MHz): δ 9.59 (s, 1H), 8.34 (s, 1H), 7.51 (t, J=8.0 Hz, 1H), 7.45 (d, J=8.0 Hz, 2H), 7.38 (t, J=8 Hz, 2H), 7.19 (d, J=8.0 Hz, 1H), 7.14 (t, J=8.0 Hz, 2H), 7.08 (t, J=8.0 Hz, 5H), 6.80 (d, J=8.0 Hz, 1H), 6.71 (t, J=6.0 Hz, 1H), 5.24 (s, 2H), 4.91 (dd, J₁=4.0 Hz, J₂=12.0 Hz, 1H), 4.76 (m, 1H), 3.96 (d, J=8.0 Hz, 2H), 3.52 (m, 6H), 2.81 (m, 3H), 2.63 (m, 9H), 2.15 (m, 7H), 1.62 (d, J=15.0 Hz, 3H), 1.27 (s, 1H).

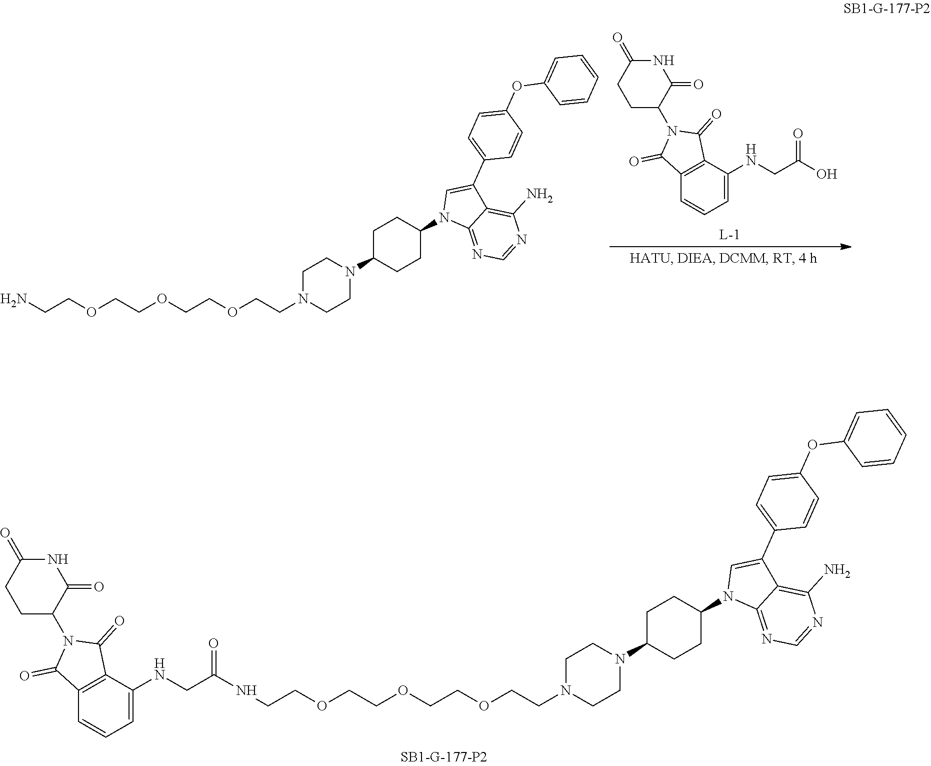

SB1-G-177-P2 was synthesized with similar procedures as SB1-G-176-P2.

SB1-G-177-P2 was synthesized with similar procedures as SB1-G-176-P2.C-MS (ESI) m/z: 957 [M+H]⁺; ¹H-NMR (CDCl₃, 400 MHz): δ (ppm) 8.34 (s, 1H), 7.66 (d, J=6.1 Hz, 1H), 7.50 (dd, J=8.5, 7.2 Hz, 1H), 7.48-7.41 (m, 2H), 7.41-7.32 (m, 2H), 7.21-7.15 (m, 2H), 7.12-7.02 (m, 5H), 6.84 (d, J=8.5 Hz, 1H), 6.75 (t, J=6.1 Hz, 1H),

5.22 (s, 2H), 4.92 (dd, J=12.1, 5.4 Hz, 1H), 4.78 (s, 1H), 4.00 (dd, J=6.0, 1.8 Hz, 2H), 3.64-3.50 (m, 8H), 2.85 (m, 4H), 2.79-2.67 (m, 3H), 2.63-2.54 (m, 4H), 2.20 (s, 2H), 2.14 (dd, J=7.4, 4.8 Hz, 4H), 2.10 (s, 3H), 2.06 (d, J=11.2 Hz, 6H), 1.79 (d, J=11.9 Hz, 2H), 1.61 (d, J=12.6 Hz, 2H).

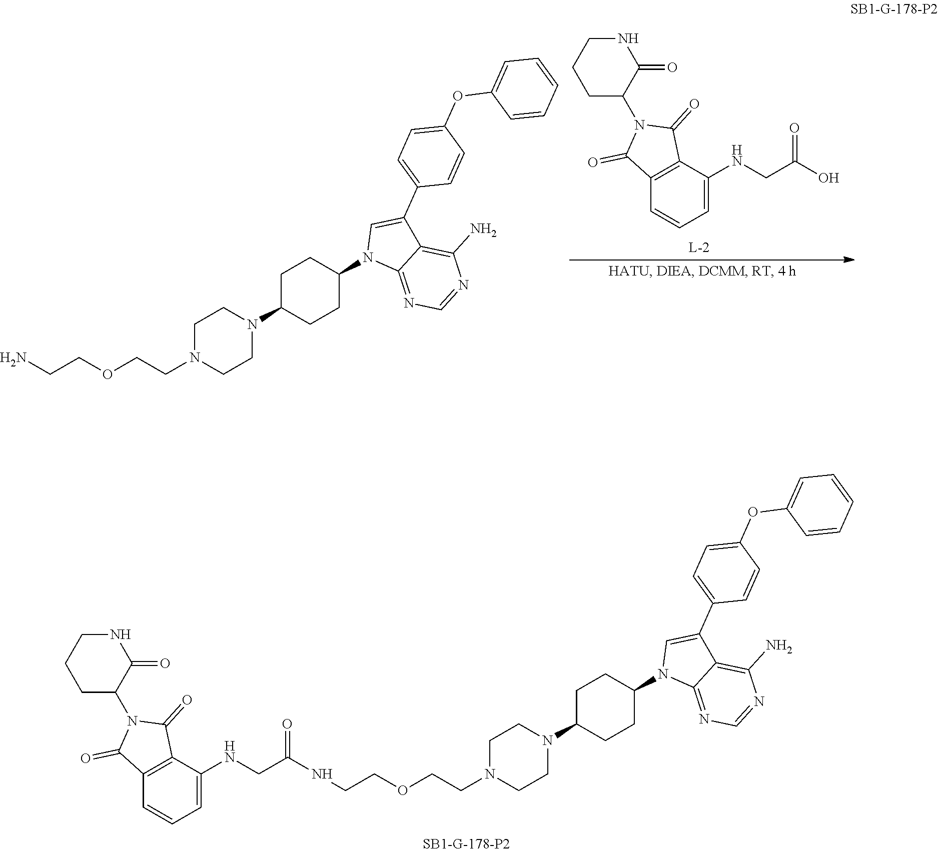

SB1-G-178-P2 was synthesized with similar procedures as SB1-G-176-P2. LCMS (m/z): 855 [M+H]⁺; ¹H NMR (CDCl₃, 400 MHz): δ 8.32 (s, 1H), 7.49 (m, 3H), 7.38 (t, J=8.0 Hz, 2H), 7.17 (t, J=8.0 Hz, 2H), 7.10 (m, 6H), 6.77 (d, J=8.0 Hz, 1H), 6.71 (t, J=6.0 Hz, 1H), 6.00 (s, 1H), 5.11 (s, 2H), 4.80 (m, 1H), 4.70 (dd, J₁=8.0 Hz, J₂=12.0 Hz, 1H), 3.94 (d, J=8.0 Hz, 2H), 3.50 (m, 7H), 3.38 (s, 1H), 2.42 (m, 10H), 2.25 (s, 1H), 2.10 (m, 6H), 1.93 (m, 1H), 1.83 (m, 3H), 1.63 (t, J=15.0 Hz, 2H).

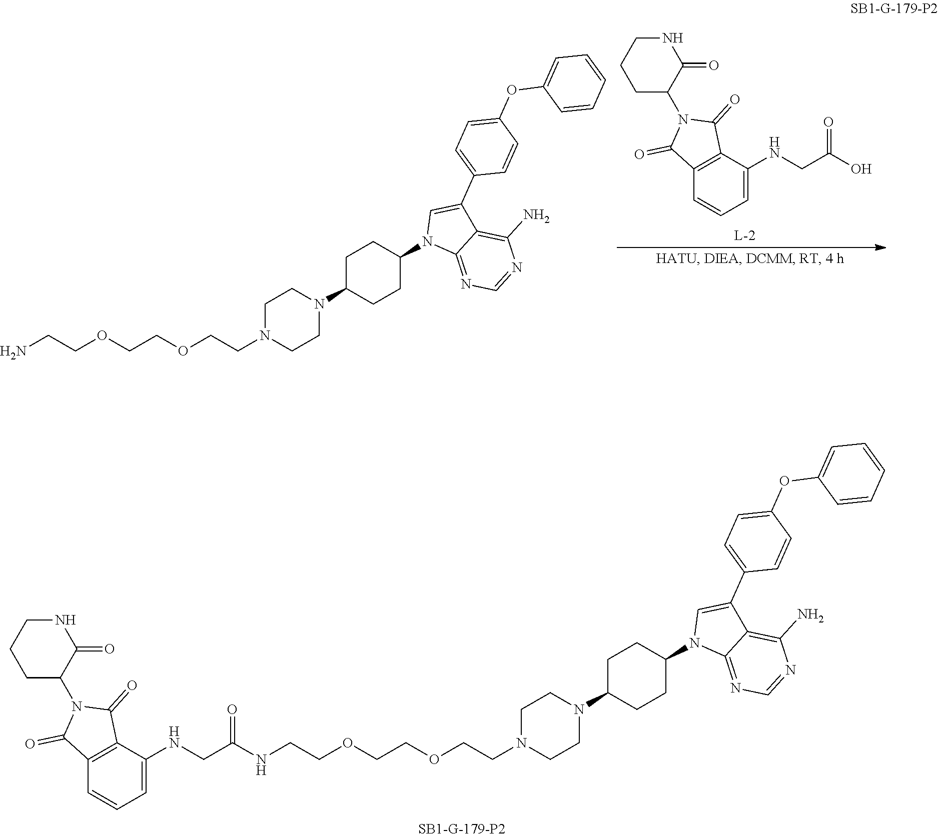

SB1-G-179-P2 was synthesized with similar procedures as SB1-G-176-P2.

LCMS (m/z): 899 [M+H]⁺; ¹H NMR (CDCl₃, 400 MHz): δ 8.32 (s, 1H), 7.48 (m, 3H), 7.38 (t, J=8.0 Hz, 2H), 7.16 (m, 3H), 7.08 (t, J=8.0 Hz, 5H), 6.75 (m, 2H), 6.21 (s, 1H), 5.13 (s, 2H), 4.80 (m, 1H), 4.70 (dd, J₁=8.0 Hz, J₂=12.0 Hz, 1H), 3.89 (d, J=8.0 Hz, 2H), 3.52 (m, 11H), 3.39 (s, 1H), 2.58 (m, 9H), 2.36 (m, 1H), 2.26 (s, 1H), 2.9 (m, 8H), 1.82 (d, J=8.0 Hz, 2H), 1.63 (t, J=15.0 Hz, 2H).

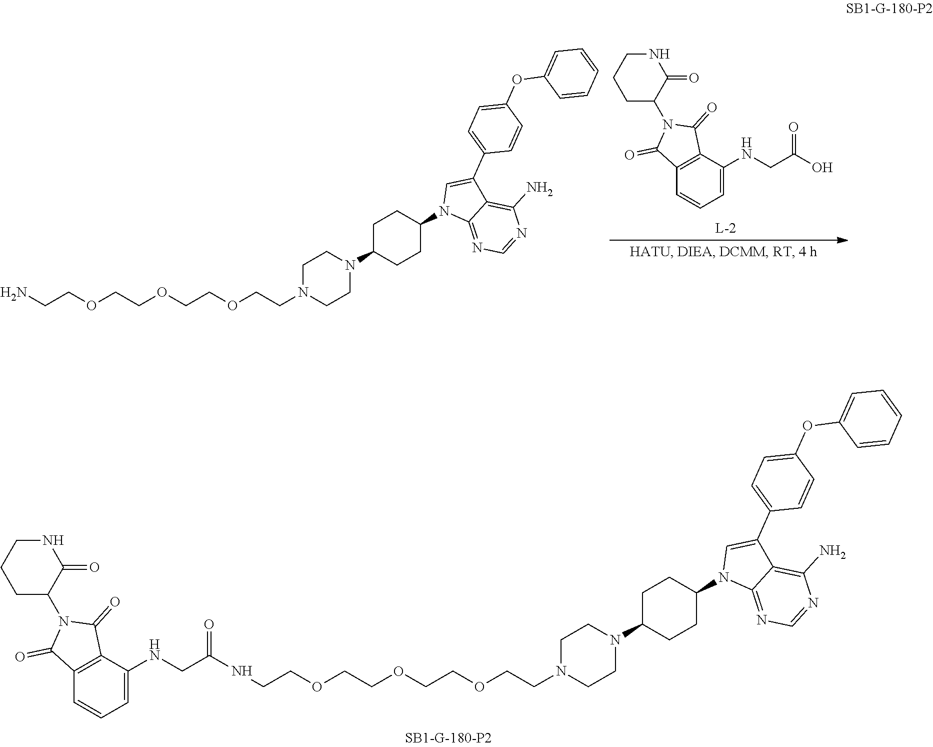

SB1-G-180-P2 was synthesized with similar procedures as SB1-G-176-P2.

LC-MS (ESI) m/z: 943.3 [M+H]⁺; ¹H-NMR (CDCl₃, 400 MHz): δ (ppm) 8.30 (s, 1H), 7.55 (t, J=5.2 Hz, 1H), 7.48-7.44 (m, 3H), 7.37 (d, J=8.0 Hz, 2H), 7.16-7.12 (m, 2H), 7.09-7.06 (m, 5H), 6.79-6.74 (m, 2H), 6.31 (m, 1H), 5.16 (s, 2H), 4.82-4.67 (m, 2H), 3.96 (d, J=6.0 Hz, 2H), 3.62-3.59 (m, 6H), 3.55-3.36 (m, 10H), 2.63-2.56 (m, 7H), 2.43-2.36 (m, 2H), 2.22-2.20 (m, 2H), 2.11-1.95 (m, 8H), 1.81-1.79 (m, 2H), 1.64-1.58 (m, 2H).

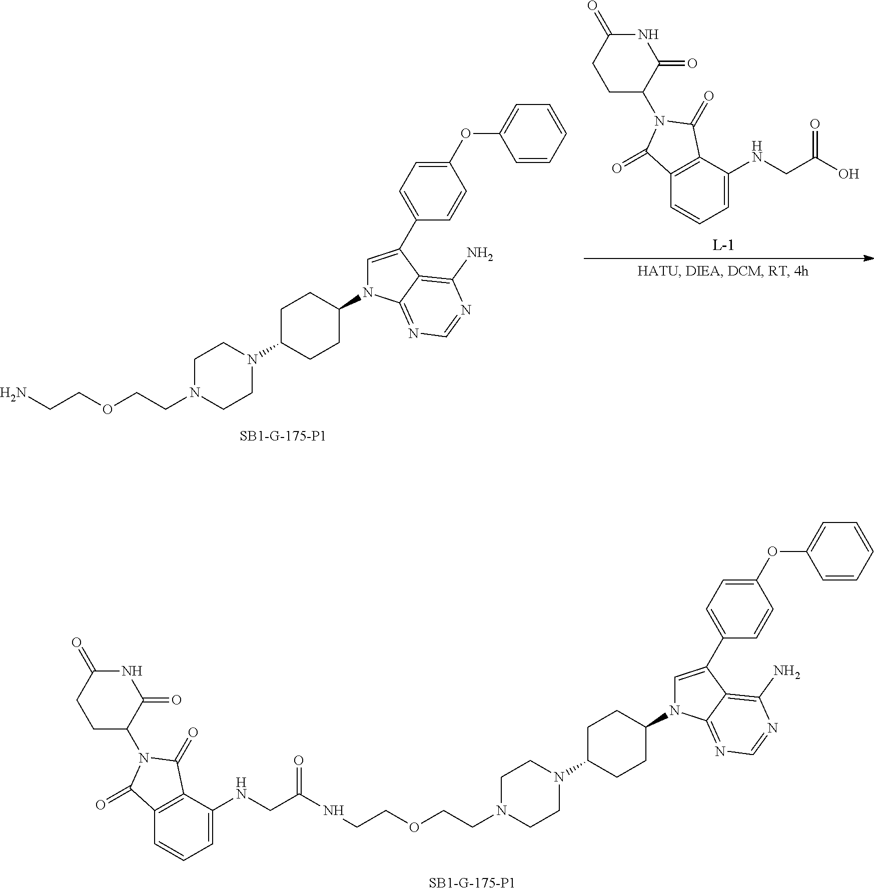 [SMILES: NCCOCCN1CCN([C@H]2CC[C@H](n3cc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)cn2[C@H]1CC[C@H](N2CCN(CCOCCNC(=O)CNc3cccc4c3C(=O)N(C3CCC(=O)NC3=O)C4=O)CC2)CC1]

SB1-G-175-P1 was synthesized with similar procedures as SB1-G-176-P2.

LC-MS (ESI) m/z: 869.5 [M+H]⁺; ¹H-NMR (CDCl₃, 400 MHz): δ (ppm) 8.34 (s, 1H), 7.54 (t, J=7.6 Hz, 1H), 7.42 (d, J=8.4 Hz, 2H), 7.37 (t, J=7.6 Hz, 2H), 7.21 (d, J=7.2 Hz, 1H), 7.14 (t, J=7.2 Hz, 1H), 7.08-7.06 (m, 5H), 6.99 (s, 1H), 6.84 (d, J=8.4 Hz, 1H), 6.68 (t, J=6.4 Hz, 1H), 5.18 (s, 2H), 4.96-4.92 (m, 1H), 4.69-4.67 (m, 1H), 3.99-3.96 (m, 2H), 3.55-3.45 (m, 6H), 2.91-2.74 (m, 4H), 2.51-2.42 (m, 6H), 2.21-2.14 (m, 3H), 2.10-1.98 (m, 3H), 1.83-1.75 (m, 4H), 1.63-1.54 (m, 3H).

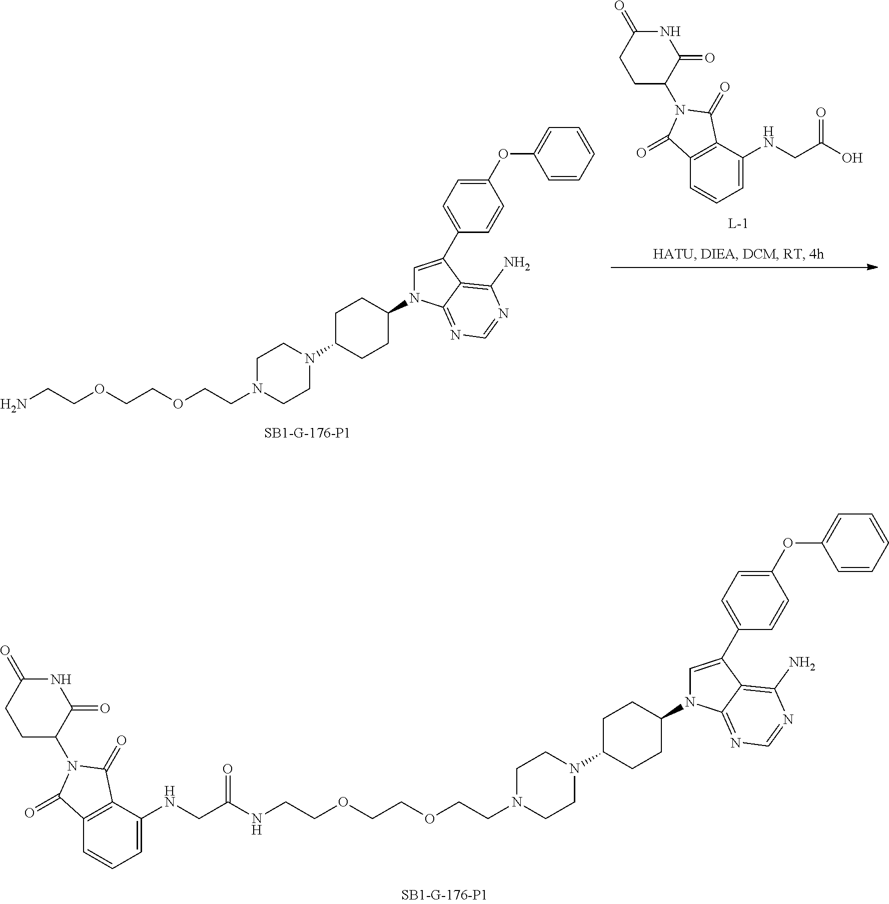 [SMILES: NCCOCCOCCN1CCN([C@H]2CC[C@H](n3cc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCC(=O)NC3=O)C4=O)CC2)CC1]

SB1-G-176-P1 was synthesized with similar procedures as SB1-G-176-P2.

LC-MS (ESI) m/z: 913.1 [M+H]⁺; ¹H-NMR (CDCl₃, 400 MHz): δ (ppm) 8.33 (s, 1H), 7.53 (t, J=7.6 Hz, 1H), 7.42 (d, J=8.4 Hz, 2H), 7.37 (t, J=7.6 Hz, 2H), 7.21 (d, J=7.2 Hz, 1H), 7.14 (t, J=7.2 Hz, 1H), 7.09-7.06 (m, 4H), 6.99 (s, 1H), 6.83 (d, J=8.4 Hz, 1H), 6.73 (t, J=5.6 Hz, 1H), 5.15 (s, 2H), 4.95-4.90 (m, 1H), 4.62-4.73 (m, 1H), 3.98-3.97 (m, 2H), 3.66-3.51 (m, 10H), 3.39-3.36 (m, 2H), 2.89-2.73 (m, 4H), 2.65-2.44 (m, 8H), 2.20-2.03 (m, 6H), 1.64-1.55 (m, 3H), 1.28-1.26 (m, 2H).

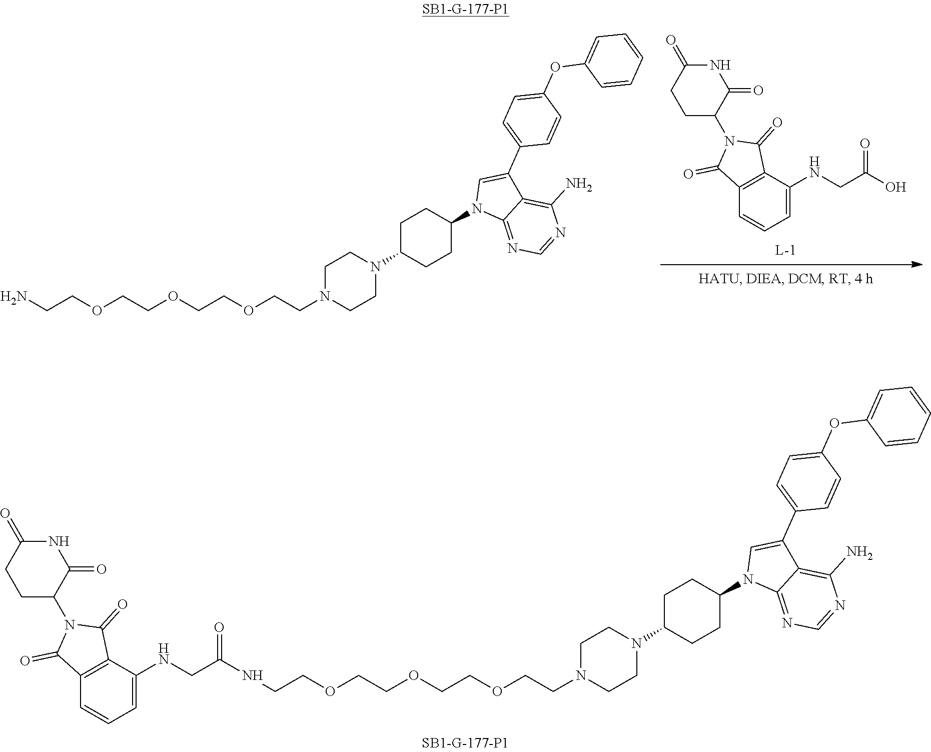

SB1-G-177-P1 was synthesized with similar procedures as SB1-G-176-P2.

LC-MS (ESI) m/z: 957.1 [M+H]⁺; ¹H-NMR (CDCl₃, 400 MHz): δ (ppm) 8.31 (s, 1H), 7.56-7.49 (m, 2H), 7.43 (d, J=11.2 Hz, 2H), 7.39-7.34 (m, 2H), 7.18-7.12 (m, 2H), 7.09-7.06 (m, 4H), 6.99 (s, 1H), 6.84 (d, J=8.4 Hz, 1H), 6.79 (t, J=6.0 Hz, 1H), 5.25 (s, 2H), 4.91-4.87 (m, 1H), 4.65 (m, 1H), 4.00 (d, J=6.0 Hz, 2H), 3.62-3.59 (m, 6H), 3.59-3.57 (m, 4H), 3.53-3.44 (m, 6H), 2.88-2.73 (m, 4H), 2.61-2.58 (m, 4H), 2.43-2.37 (m, 2H), 2.18-2.15 (m, 4H), 2.10-2.01 (m, 4H), 1.77-1.74 (m, 2H), 1.57-1.52 (m, 2H).

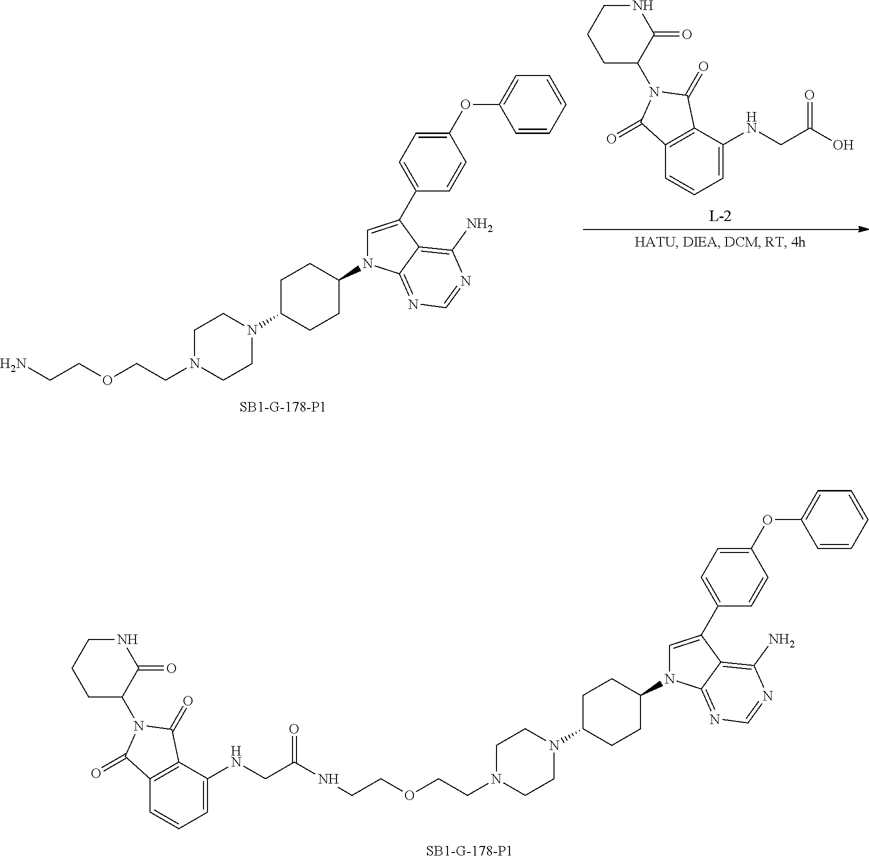 [SMILES: NCCOCCN1CCN([C@H]2CC[C@H](n3cc(-c4ccc(Oc5ccccc5)cc4)c4c(N)ncnc43)CC2)CC1 ; O=C(O)CNc1cccc2c1C(=O)N(C1CCCNC1=O)C2=O ; Nc1ncnc2c1c(-c1ccc(Oc3ccccc3)cc1)cn2[C@H]1CC[C@H](N2CCN(CCOCCNC(=O)CNc3cccc4c3C(=O)N(C3CCCNC3=O)C4=O)CC2)CC1]

SB1-G-178-P1 was synthesized with similar procedures as SB1-G-176-P2.

LC-MS (ESI) m/z: 855.1 [M+H]⁺; ¹H-NMR (CDCl₃, 400 MHz): δ (ppm) 8.31 (s, 1H), 7.49 (t, J=7.6 Hz, 1H), 7.43 (d, J=8.8 Hz, 2H), 7.38 (t, J=7.6 Hz, 2H), 7.18 (d, J=7.2 Hz, 1H), 7.14 (t, J=7.2 Hz, 1H), 7.09-7.06 (m, 5H), 7.01 (s, 1H), 6.78 (d, J=8.4 Hz, 1H), 6.70 (t, J=6.0 Hz, 1H), 6.34 (m, 1H), 5.15 (s, 2H), 4.73-4.64 (m, 2H), 3.94 (d, J=6.0 Hz, 2H), 3.54-3.38 (m, 7H), 2.49-2.47 (m, 4H), 2.40-2.34 (m, 2H), 2.21-2.18 (m, 2H), 2.10-1.99 (m, 4H), 1.98-1.88 (m, 7H), 1.84-1.75 (m, 3H), 1.62-1.56 (m, 2H).

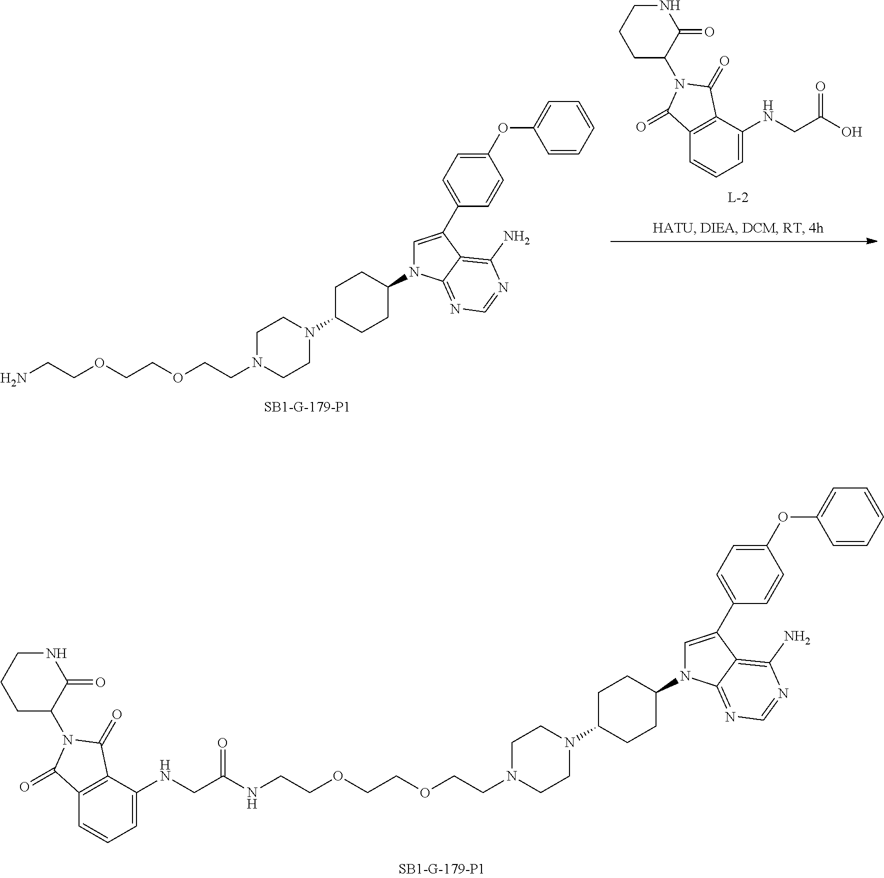 [SMILES: NCCOCCOCCN1CCN([C@H]2CC[C@H](n3cc(-c4ccc(Oc5ccccc5)cc4)c4c(N)ncnc43)CC2)CC1 ; O=C(O)CNc1cccc2c1C(=O)N(C1CCCNC1=O)C2=O ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCCNC3=O)C4=O)CC2)CC1]

SB1-G-179-P1 was synthesized with similar procedures as SB1-G-176-P2. LC-MS (ESI) m/z: 899.3 [M+H]⁺; ¹H-NMR (CDCl₃, 400 MHz): δ (ppm) 8.31 (s, 1H), 7.49 (t, J=8.0 Hz, 1H), 7.43 (d, J=8.4 Hz, 2H), 7.38 (t, J=7.6 Hz, 2H), 7.18 (d, J=7.2 Hz, 1H), 7.14 (t, J=7.2 Hz, 1H), 7.09-7.06 (m, 4H), 7.02-7.00 (m, 2H), 6.78 (d, J=8.8 Hz, 1H), 6.73 (t, J=6.0 Hz, 1H), 6.19 (s, 1H), 5.11 (s, 2H), 4.73-4.67 (m, 2H), 3.95 (d, J=6.0 Hz, 2H), 3.57 (t, J=5.6 Hz, 2H), 3.54-3.41 (m, 10H), 2.60-2.54 (m, 4H), 2.44-2.37 (m, 2H), 2.21-2.18 (m, 2H), 2.10-2.05 (m, 5H), 1.84-1.70 (m, 4H), 1.63-1.57 (m, 2H), 1.27-1.25 (m, 2H).

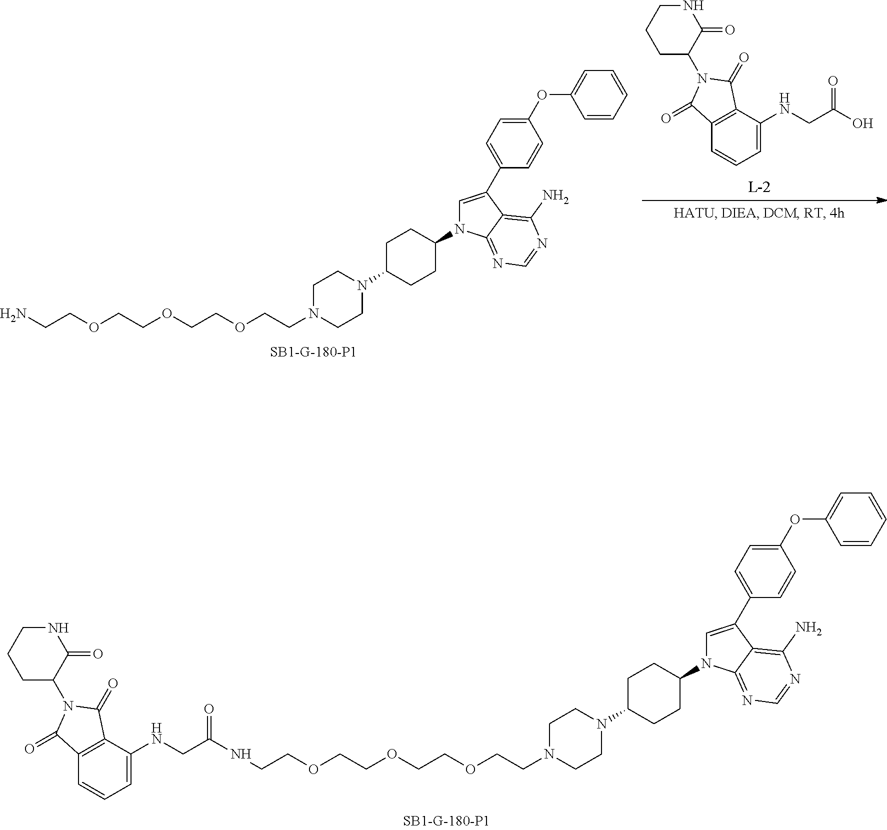 [SMILES: NCCOCCOCCOCCN1CCN([C@H]2CC[C@H](n3cc(-c4ccc(Oc5ccccc5)cc4)c4c(N)ncnc43)CC2)CC1 ; O=C(O)CNc1cccc2c1C(=O)N(C1CCCNC1=O)C2=O ; Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCCNC3=O)C4=O)CC2)CC1]

SB1-G-180-P1 was synthesized with similar procedures as SB1-G-176-P2.

LC-MS (ESI) m/z: 943.4 [M+H]⁺; ¹H-NMR (CDCl₃, 400 MHz): δ (ppm) 8.30 (s, 1H), 7.49-7.41 (m, 4H), 7.39-7.35 (m, 2H), 7.16-7.12 (m, 2H), 7.10-7.06 (m, 4H), 7.01 (s, 1H), 6.78 (d, J=8.4 Hz, 1H), 6.76 (d, J=6.0 Hz, 1H), 6.33 (m, 1H), 5.18 (s, 2H), 4.70-4.65 (m, 2H), 3.96 (d, J=5.6 Hz, 2H), 3.59-3.58 (m, 6H), 3.55-3.52 (m, 6H), 3.48-3.47 (m, 3H), 3.38-3.36 (m, 1H), 2.60-2.57 (m, 5H), 2.43-2.34 (m, 2H), 2.31-2.27 (m, 5H), 2.19-2.16 (m, 2H), 2.10-1.90 (m, 5H), 1.82-1.72 (m, 2H), 1.59-1.50 (m, 2H).

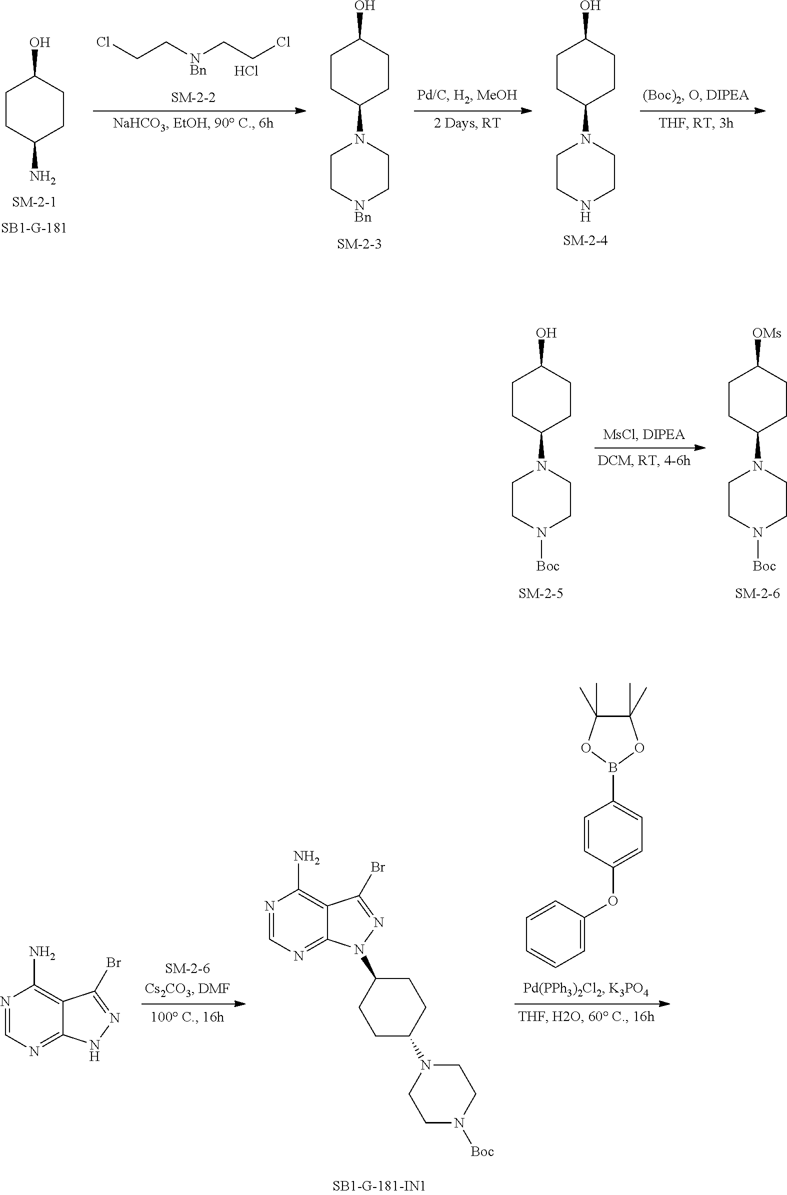

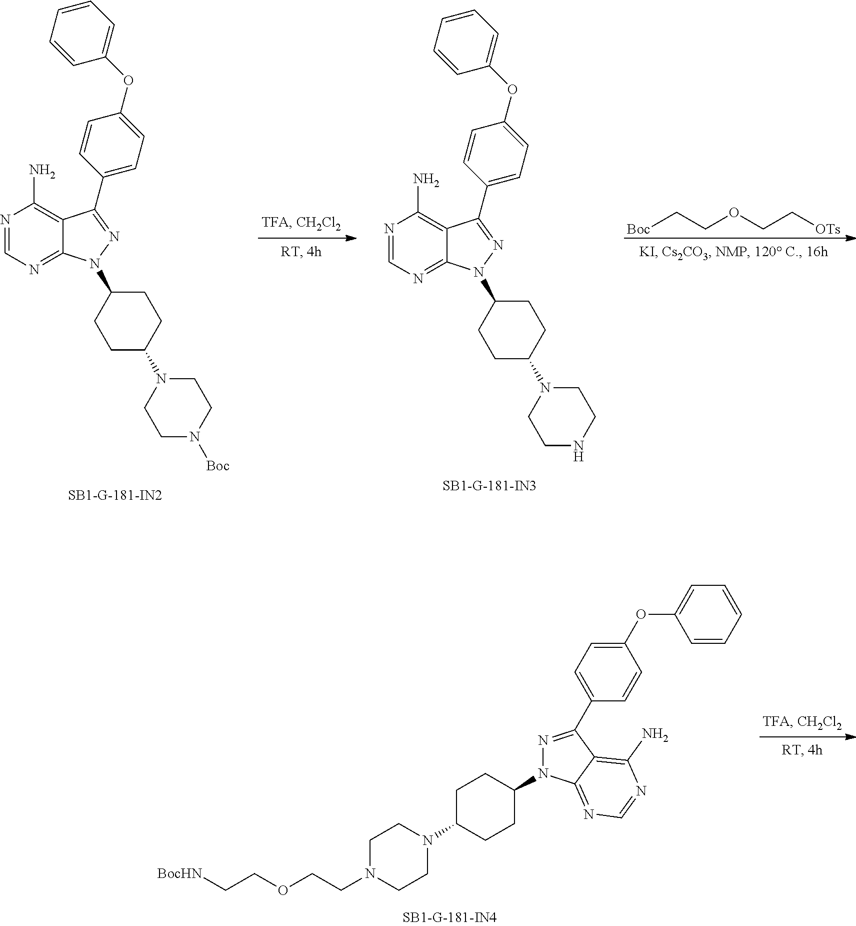 [SMILES: CC(C)(C)OC(=O)N1CCN([C@H]2CC[C@H](n3nc(-c4ccc(Oc5ccccc5)cc4)c4c(N)ncnc43)CC2)CC1 ; Nc1ncnc2c1c(-c1ccc(Oc3ccccc3)cc1)nn2[C@H]1CC[C@H](N2CCNCC2)CC1 ; Cc1ccc(S(=O)(=O)OCCOCCC(=O)OC(C)(C)C)cc1 ; CC(C)(C)OC(=O)NCCOCCN1CCN([C@H]2CC[C@H](n3nc(-c4ccc(Oc5ccccc5)cc4)c4c(N)ncnc43)CC2)CC1]

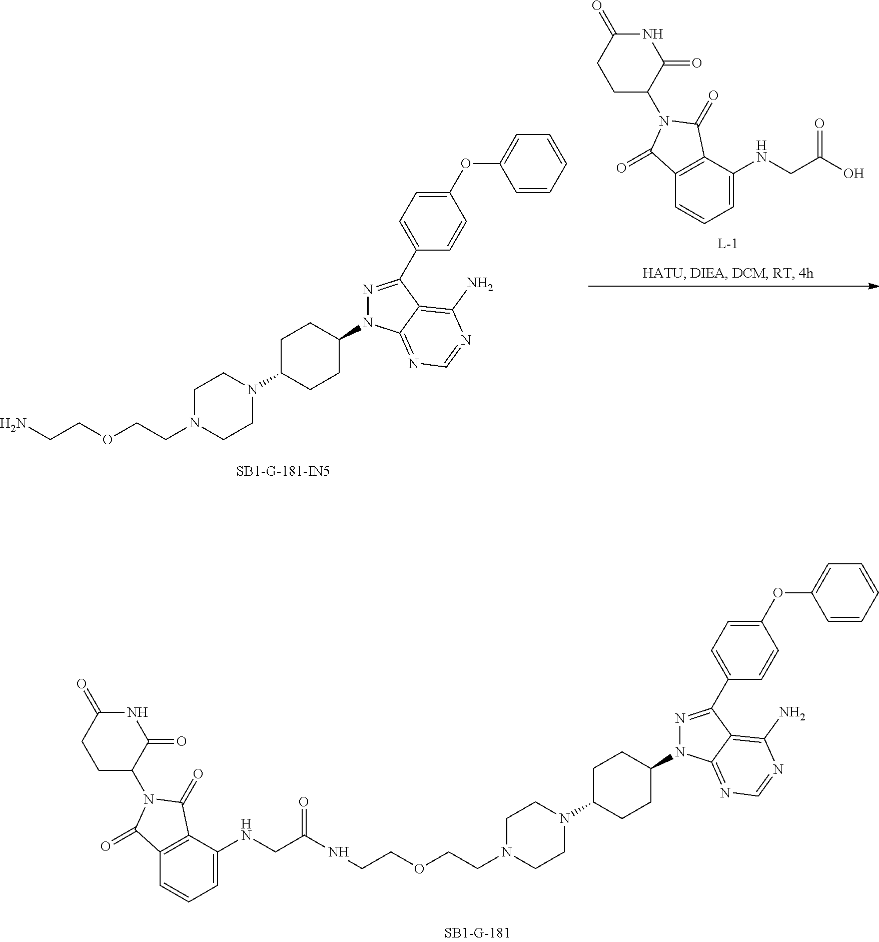 [SMILES: NCCOCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1]

### (1s,4s)-4-(4-benzylpiperazin-1-yl)cyclohexanol (SM-2-3)

The mixture of SM-2-1 (12.0 g, 79.14 mmol), SM-2-2 (21.26 g, 79.14 mmol), NaHCO₃ (13.30 mg, 158.28 mmol) and EtOH (300 mL) was stirred and heated to 90° C. for 6 h, after completion, filtered, washed with DCM (300 mL), concentrated to remove the solvent, the residue was purified by silica gel column chromatography (DCM:MeOH=30:1) to get SM-2-3 (off-white solid, 21 g yield 97%). LCMS (m/z): 275 [M+H]⁺.

### (1s,4s)-4-(piperazin-1-yl)cyclohexanol (SM-2-4)

The mixture of SM-2-3 (21 g, 76.53 mmol), Pd/C (2.1 g) and EtOH (250 mL) was stirred at RT for 16 h under H₂ atmosphere, after completion, filtered with celite, removed the solvent, the residue (SM-2-4, 15.0 g crude product) was put into next steps. LCMS (m/z): 185 [M+H]⁺.

### tert-butyl 4-((1s,4s)-4-hydroxycyclohexyl)piperazine-1-carboxylate (SM-2-5)

The mixture of SM-2-4 (15.0 g, 81.5 mmol), (Boc)₂₀ (26.69 g, 122.3 mmol), DIPEA (31.5 g, 244.2 mmol) and THF (300 mL) was stirred at RT for 3 h, after completion, concentrated to remove the organic solvent, diluted with brine (200 mL), extracted with DCM (500 mL, 300 mL×2), the organic phase was washed with brine (200 mL), dried with Na₂SO₄, filtered, removed the solvent, the residue was purified by silica gel column chromatography (DCM:MeOH=10:0-10:1) to get SM-2-5 (white solid, 10 g+5 g (crude), yield 65%). LCMS (m/z): 285 [M+H]⁺.

### tert-butyl 4-((1s,4s)-4-(methylsulfonyloxy)cyclohexyl)piperazine-1-carboxylate (SM-2-6)

The mixture of SM-2-5 (15.0 g, 52.82 mmol), MsCl (9.08 g, 79.23 mmol), DIPEA (20.44 g, 158.5 mmol) and DCM (500 mL) was then added, stirred at RT for 6 h, after completion, quenched and diluted with water (350 mL), the aqueous phase was extracted with DCM (300 mL×2), the organic phase was combined, dried with Na₂SO₄, filtered, concentrated to remove the organic solvent, the residue was purified by TLC (spreading agent: DCM:MeOH=9:1) to get SM-2-6 (off-white, 9.5 g+9 g (crude), yield 50%). LCMS (m/z): 363 [M+H]⁺

### tert-butyl4-((1r,4r)-4-(4-amino-3-iodo-1H-pyrazolo[3,4-d]pyrimidin-1-yl)cyclohexyl)piperazine-1-carboxylate (SB1-G-181-IN1)

The mixture of 3-bromo-1H-pyrazolo[3,4-d]pyrimidin-4-amine (8.05 g, 37.61 mmol), SM-2-6 (17.73 g, 48.90 mmol), Cs₂CO₃ (30.63 g, 94.03 mmol) and DMF (200 mL) was stirred and heated to 100° C. for 16 h, after completion, remove the organic solvent, the mixture was diluted with 500 mL DCM, and washed with brine (500 mL×2), then the organic phase was dried with Na₂SO₄, filtered, remove the solvent, the residue (SB1-G-181-IN1 (light yellow solid, 5.65 g, yield 29%)) was put into next step without further purification. LCMS (m/z): 528 [M+H]⁺.

### tert-butyl-4-((1r,4r)-4-(4-amino-3-(4-phenoxyphenyl)-1H-pyrazolo[3,4-d]pyrimidin-1-yl)cyclohexyl) piperazine-1-carboxylate (SB1-G-181-IN2)

The mixture of SB1-G-181-IN1 (5.65 g, 10.72 mmol), 4,4,5,5-tetramethyl-2-(4-phenoxyphenyl)-1,3,2-dioxaborolane (4.76 g, 16.07 mmol), Pd(PPh₃)₂Cl₂ (0.75 g, 1.07 mmol), K₃PO₄ (4.55 g, 21.44 mmol), THF (300 mL) and H₂O (20 mL) was stirred and heated to 60° C. overnight, after completion, concentrated to remove the solvent, extracted with DCM (500 mL×2), the organic phase was washed with brine (200 mL×2), dried with Na₂SO₄, filtered, removed the solvent, the residue was purified by silica gel column chromatography (DCM:MeOH=30:1-10:1) to get SB1-G-181-IN2 (off-white solid, 4.10 g, yield 67%). LCMS (m/z): 570 [M+H]⁺. ¹H NMR (DMSO-d₆, 400 MHz): δ 8.23 (s, 1H), 7.65 (d, J=8.0 Hz, 2H), 7.44 (t, J=8.0 Hz, 2H), 7.16 (m, 5H), 4.65 (m, 1H), 3.30 (s, 4H), 2.51 (m, 2H), 2.48 (m, 4H), 2.03 (m, 4H), 1.91 (d, J=12.0 Hz, 3H), 1.50 (m, 2H), 1.40 (s, 9H).

### 3-(4-phenoxyphenyl)-1-((1r,4r)-4-(piperazin-1-yl)cyclohexyl)-1H-pyrazolo[3,4-d]pyrimidin-4-amine (SB1-G-181-IN3)

The mixture of SB1-G-181-IN2 (2.9 g, 5.09 mmol), TFA (30 mL) and DCM (60 mL) was stirred at RT 4 h, after completion, concentrated to remove the organic solvent, diluted with water (500 mL), washed with EA (300 mL×3), Na₂CO₃(s) was added into aqueous phase and stirred until PH=9-10, then the aqueous phase extracted with DCM (500 mL×3), the organic phase was washed with brine (300 mL), dried with Na₂SO₄, filtered, removed the solvent, the residue (SB1-G-181-IN3, faint yellow solid, 2.15 g, yield 90%) was put into next step without further purification. LCMS (m/z): 470 [M+H]⁺.

### tert-butyl-2-(2-(4-((1r,4r)-4-(4-amino-3-(4-phenoxyphenyl)-1H-pyrazolo[3,4-d]pyrimidin-1-yl)cyclohexyl) piperazin-1-yl)ethoxy)ethylcarbamate (SB1-G-181-IN4)

The mixture of SB1-G-181-IN3 (700 mg, 1.49 mmol), 2-(2-((tert-butoxycarbonyl)amino)ethoxy)ethyl 4-methylbenzenesulfonate (805 mg, 2.24 mmol), Cs₂CO₃ (969 mg, 2.98 mmol), KI (24.9 mg, 0.15 mmol) and NMP (15 mL) was stirred and heated to 120° C. for 16 h, after completion, diluted with brine (500 mL), extracted with DCM (500 mL×3), the organic phase was washed with brine (200 mL), dried with Na₂SO₄, filtered, removed the solvent, the residue was purified by silica gel column chromatography (DCM:MeOH=10:1) to get SB1-G-181-IN4 (white solid, 660 mg, yield 67%). LCMS (m/z): 657 [M+H]⁺.

### 1-((1r,4r)-4-(4-(2-(2-aminoethoxy)ethyl)piperazin-1-yl)cyclohexyl)-3-(4-phenoxyphenyl)-1H-pyrazolo[3,4-d]pyrimidin-4-amine (SB1-G-181-IN5)

The mixture of SB1-G-181-IN4 (660 mg, 1.01 mmol), TFA (5 mL) and DCM (10 mL) was stirred at RT for 4 h, after completion, concentrated to remove the organic solvent, diluted with water (100 mL), washed with EA (100 mL×3), Na₂CO₃(s) was added into aqueous phase and stirred until PH=9-10, then the aqueous phase extracted with DCM (200 mL×3), the organic phase was washed with brine (150 mL), dried with Na₂SO₄, filtered, removed the solvent, the residue (SB1-G-181-IN5, faint yellow solid, 520 mg, yield 92%) was put into next step without further purification. LCMS (m/z): 557 [M+H]⁺.

### N-(2-(2-(4-((1r,4r)-4-(4-amino-3-(4-phenoxyphenyl)-1H-pyrazolo[3,4-d]pyrimidin-1-yl)cyclohexyl) piperazin-1-yl)ethoxy)ethyl)-2-(2-(2,6-dioxopiperidin-3-yl)-1,3-dioxoisoindolin-4-ylamino)acetamide (SB1-G-181)

The mixture of SB1-G-181-IN5 (99 mg, 0.177 mmol), L-1 (45 mg, 0.136 mmol), HATU (104 mg, 0.272 mmol), DIPEA (175 mg, 1.36 mmol), and DCM (5 mL) was stirred at RT for 4 h, after completion, diluted with water (50 mL), the aqueous phase was extracted with DCM (100 mL×2), dried with Na₂SO₄, filtered, concentrated to remove the organic solvent, the residue was purified with Prep-HPLC (C18 column, CH₃CN/H₂O, containing 0.05% NH₄HCO₃) to get SB1-G-181 (yellow solid, 22.0 mg). LCMS (m/z): 870 [M+H]⁺; ¹H NMR (DMSO-d₆, 400 MHz): δ 8.41 (s, 1H), 7.64 (d, J=8.0 Hz, 2H), 7.55 (t, J=8.0 Hz, 1H), 7.39 (t, J=8 Hz, 2H), 7.22 (d, J=8.0 Hz, 1H), 7.16 (dd, J₁=8.0 Hz, J₂=16.0 Hz, 4H), 7.08 (d, J=8.0 Hz, 2H), 6.86 (d, J=8.0 Hz, 1H), 6.71 (t, J=6.0 Hz, 1H), 5.54 (s, 2H), 4.94 (dd, J₁=8.0 Hz, J₂=12.0 Hz, 1H), 4.78 (m, 1H), 4.01 (m, 2H), 3.52 (m, 6H), 2.87 (m, 2H), 2.76 (m, 6H), 2.14 (m, 8H), 1.59 (m, 5H), 1.25 (s, 2H).

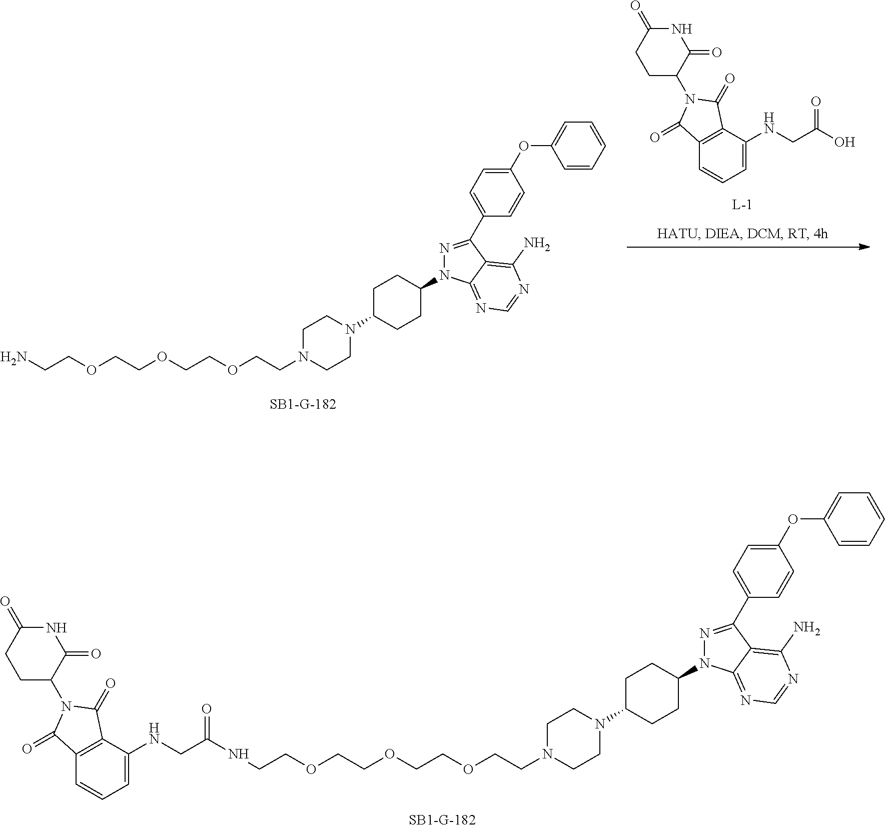 [SMILES: NCCOCCOCCOCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCC(=O)NC3=O)C4=O)CC2)CC1]

SB1-G-182 was synthesized with similar procedures as SB1-G-181.

LCMS (m/z): 958 [M+H]⁺; ¹H NMR (DMSO-d₆, 400 MHz): δ 8.38 (s, 1H), 7.64 (d, J=8.0 Hz, 2H), 7.53 (t, J=8.0 Hz, 1H), 7.39 (t, J=8 Hz, 3H), 7.20 (d, J=4.0 Hz, 1H), 7.15 (t, J=8.0 Hz, 3H), 7.08 (d, J=8.0 Hz, 2H), 6.85 (d, J=12.0 Hz, 1H), 6.77 (t, J=6.0 Hz, 1H), 5.54 (s, 2H), 4.92 (dd, J₁=4.0 Hz, J₂=12.0 Hz, 1H), 4.74 (m, 1H), 3.99 (d, J=4.0 Hz, 2H), 3.60 (m, 8H), 3.51 (m, 7H), 2.91 (d, J=12.0 Hz, 1H), 2.84 (m, 1H), 2.75 (m, 2H), 2.61 (m, 5H), 2.47 (s, 1H), 2.11 (m, 9H), 1.52 (d, J=8.0 Hz, 3H), 1.25 (s, 1H).

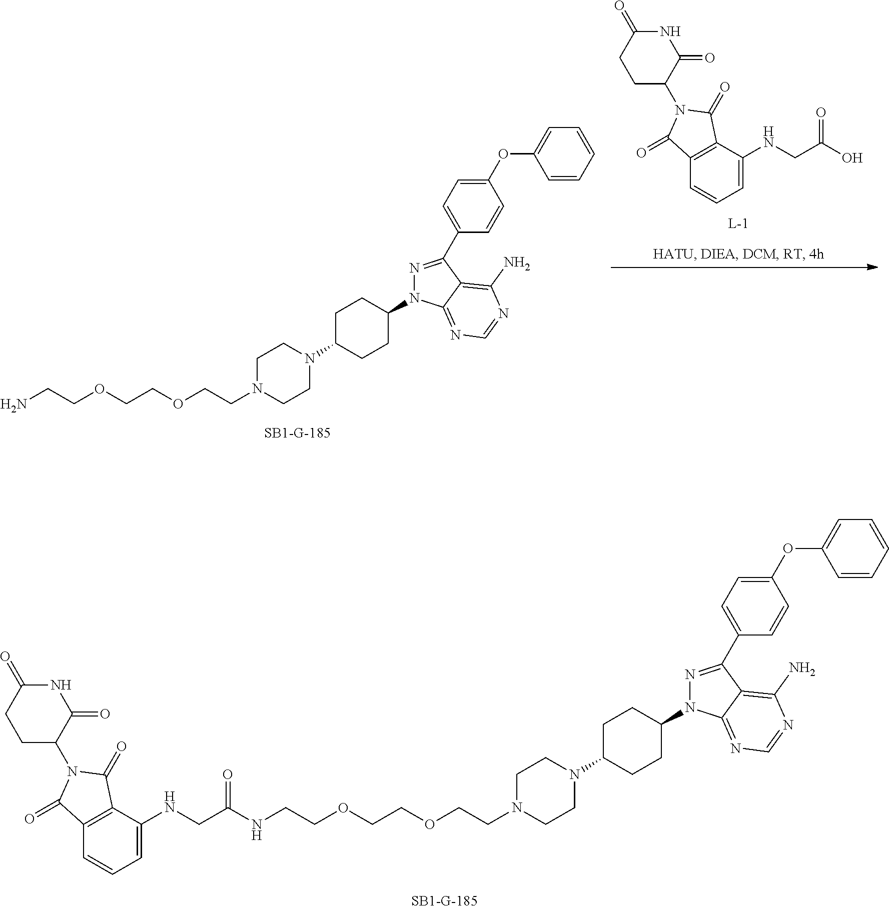 [SMILES: NCCOCCOCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCC(=O)NC3=O)C4=O)CC2)CC1]

SB1-G-185 was synthesized with similar procedures as SB1-G-181.

LCMS (m/z): 914 [M+H]⁺; ¹H NMR (DMSO-d₆, 400 MHz): δ 8.38 (s, 1H), 7.64 (d, J=8.0 Hz, 2H), 7.53 (t, J=8.0 Hz, 1H), 7.39 (t, J=8 Hz, 2H), 7.29 (d, J=8.0 Hz, 1H), 7.20 (m, 1H), 7.15 (t, J=8.0 Hz, 3H), 7.07 (d, J=8.0 Hz, 2H), 6.84 (d, J=8.0 Hz, 1H), 6.75 (t, J=6.0 Hz, 1H), 5.55 (s, 2H), 4.93 (dd, J₁=4.0 Hz, J₂=12.0 Hz, 1H), 4.74 (m, 1H), 4.00 (m, 2H), 3.56 (m, 10H), 3.40 (m, 1H), 2.81 (m, 4H), 2.57 (m, 7H), 2.15 (m, 8H), 1.53 (m, 2H), 1.29 (m, 1H).

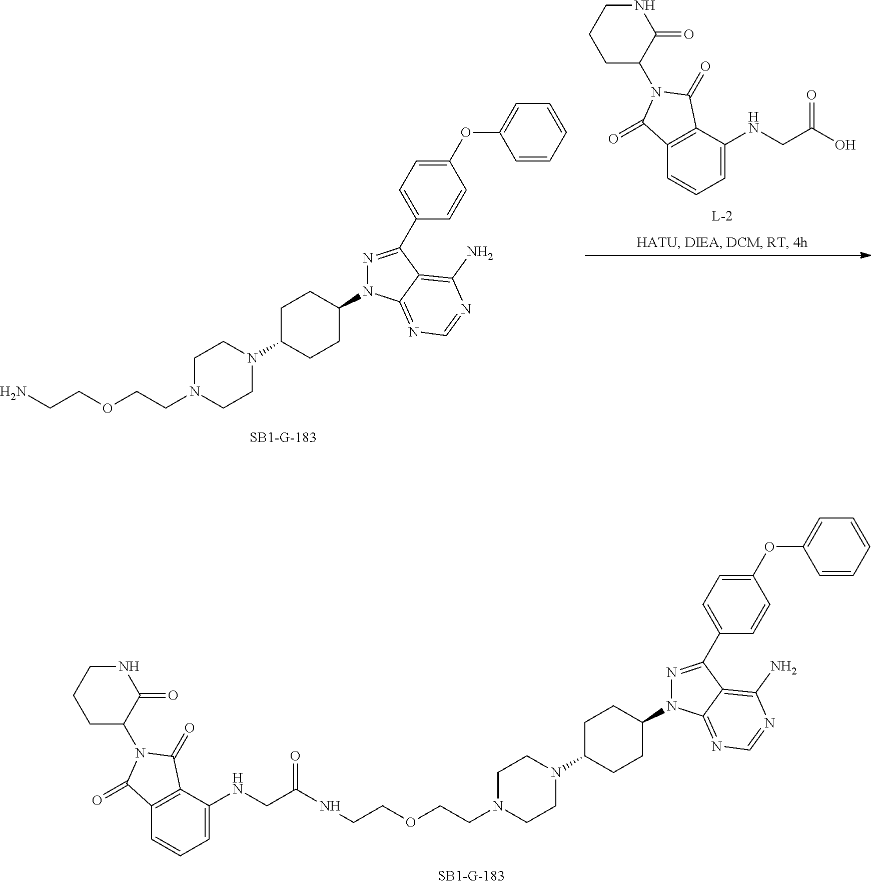 [SMILES: NCCOCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCCNC1=O)C2=O ; Nc1ncnc2c1c(-c1ccc(Oc3ccccc3)cc1)nn2[C@H]1CC[C@H](N2CCN(CCOCCNC(=O)CNc3cccc4c3C(=O)N(C3CCCNC3=O)C4=O)CC2)CC1]

SB1-G-183 was synthesized with similar procedures as SB1-G-181.

LCMS (m/z): 856 [M+H]⁺; ¹H NMR (DMSO-d₆, 400 MHz): δ 8.36 (s, 1H), 7.64 (d, J=8.0 Hz, 2H), 7.48 (t, J=8.0 Hz, 1H), 7.39 (t, J=8 Hz, 2H), 7.16 (m, 4H), 7.07 (d, J=8.0 Hz, 3H), 6.78 (d, J=8.0 Hz, 1H), 6.71 (t, J=6.0 Hz, 1H), 6.41 (s, 1H), 5.58 (s, 2H), 4.73 (m, 2H), 3.95 (d, J=4.0 Hz, 2H), 3.50 (m, 7H), 3.39 (s, 1H), 2.51 (m, 6H), 2.37 (m, 1H), 2.13 (m, 9H), 1.97 (s, 5H), 1.54 (d, J=12.0 Hz, 2H).

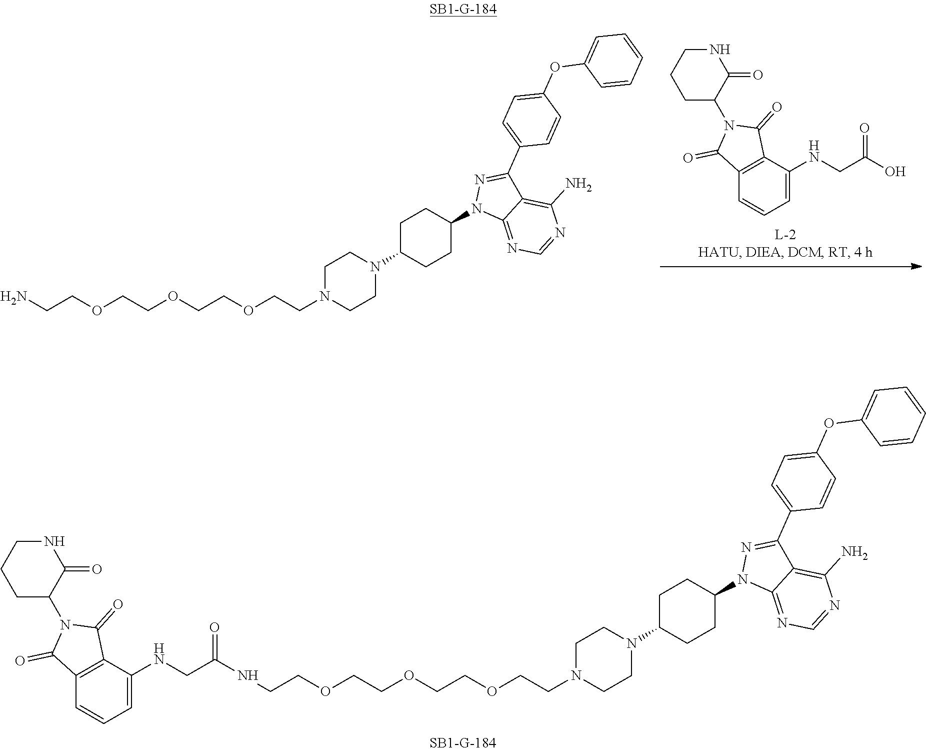

SB1-G-184 was synthesized with similar procedures as SB1-G-181.

LCMS (m/z): 944 [M+H]⁺; ¹H NMR (DMSO-d₆, 400 MHz): δ 8.36 (s, 1H), 7.64 (d, J=8.0 Hz, 2H), 7.53 (t, J=8.0 Hz, 1H), 7.39 (t, J=8 Hz, 2H), 7.27 (s, 1H), 7.16 (m, 4H), 7.07 (d, J=8.0 Hz, 2H), 6.77 (m, 2H), 6.21 (s, 1H), 5.53 (s, 2H), 4.72 (m, 2H), 3.96 (d, J=4.0 Hz, 2H), 3.60 (m, 7H), 3.51 (m, 9H), 3.40 (s, 1H), 2.47 (m, 9H), 2.12 (m, 9H), 1.52 (d, J=8.0 Hz, 2H), 1.28 (m, 2H).

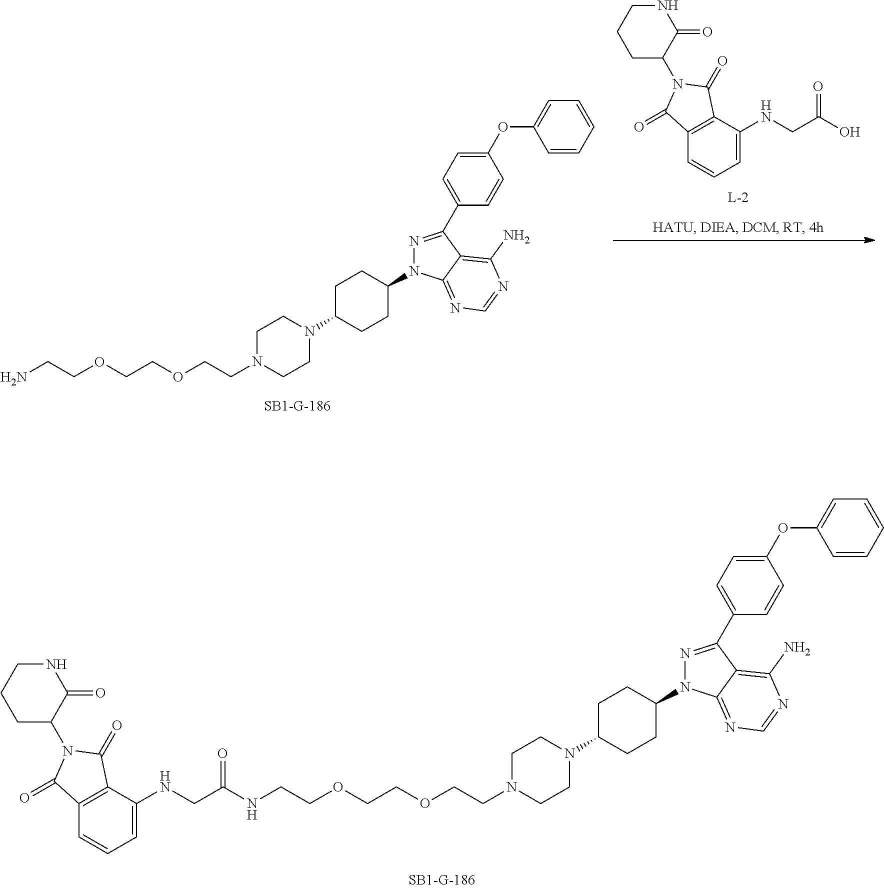 [SMILES: NCCOCCOCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCCNC1=O)C2=O ; Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCCNC3=O)C4=O)CC2)CC1]

SB1-G-186 was synthesized with similar procedures as SB1-G-181.

LCMS (m/z): 900 [M+H]⁺; ¹H NMR (DMSO-d₆, 400 MHz): δ 8.36 (s, 1H), 7.64 (d, J=8.0 Hz, 2H), 7.49 (t, J=8.0 Hz, 1H), 7.39 (t, J=8 Hz, 2H), 7.13 (m, 7H), 6.76 (m, 2H), 6.31 (s, 1H), 5.52 (s, 2H), 4.73 (m, 2H), 3.89 (d, J=4.0 Hz, 2H), 3.54 (m, 12H), 3.40 (s, 1H), 2.93 (m, 1H), 2.49 (m, 8H), 2.07 (m, 10H), 1.53 (dd, J₁=8.0 Hz, J₂=20.0 Hz, 2H), 1.29 (m, 2H).

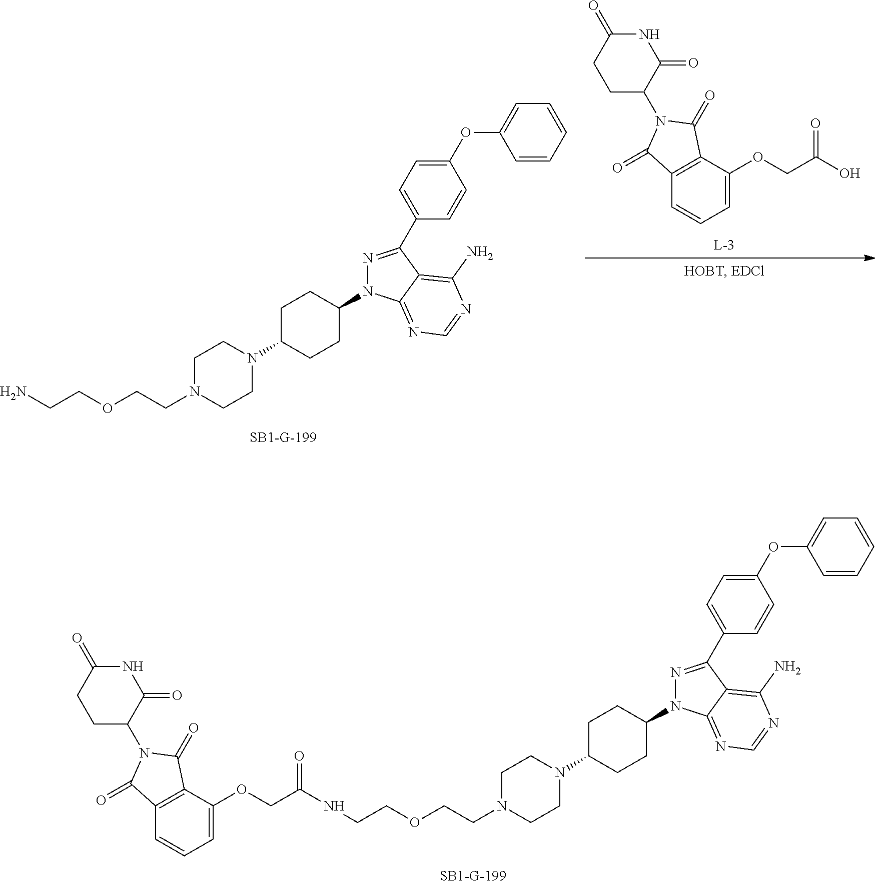 [SMILES: NCCOCCN1CCN([C@H]2CC[C@H](n3nc(-c4ccc(Oc5ccccc5)cc4)c4c(N)ncnc43)CC2)CC1 ; O=C(O)COc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCOCCNC(=O)COc3cccc4c3C(=O)N(C3CCC(=O)NC3=O)C4=O)CC2)CC1]

SB1-G-199 was synthesized with similar procedures as SB1-G-181.

LCMS (m/z): 871.0 [M+H]⁺; 1H NMR (DMSO, 400 MHz): δ 11.15 (s, 1H), 8.27-8.23 (m, 2H), 7.95-7.93 (m, 1H), 7.83 (t, J=8.0 Hz, 1H), 7.65 (d, J=8.8 Hz, 2H), 7.51 (d, J=7.2 Hz, 1H), 7.45-7.41 (m, 3H), 7.20-7.11 (m, 5H), 5.14-5.09 (m, 1H), 4.78 (s, 2H), 4.61 (s, 1H), 3.61-3.43 (m, 8H), 2.89-2.87 (m, 1H), 2.62-2.55 (m, 4H), 2.46-2.31 (m, 7H), 2.05-1.88 (m, 7H), 1.42-1.39 (m, 2H).

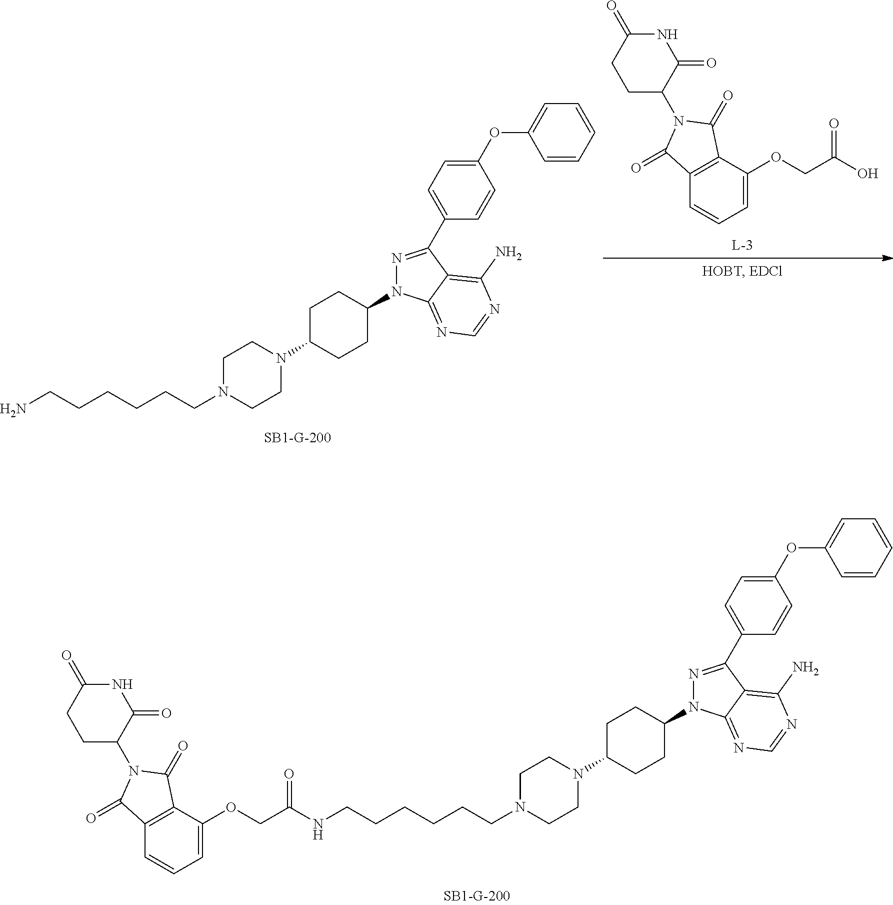 [SMILES: NCCCCCCN1CCN([C@H]2CC[C@H](n3nc(-c4ccc(Oc5ccccc5)cc4)c4c(N)ncnc43)CC2)CC1 ; O=C(O)COc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1]

SB1-G-200 was synthesized with similar procedures as SB1-G-181.

LCMS (m/z): 883.1 [M+H]⁺; 1H NMR (DMSO, 400 MHz): δ 11.18 (s, 1H), 8.30 (s, 1H), 8.23 (s, 1H), 7.92 (t, J=5.6 Hz, 1H), 7.82 (t, J=7.6 Hz, 1H), 7.65 (d, J=8.8 Hz, 2H), 7.50 (d, J=7.2 Hz, 1H), 7.45-7.39 (m, 3H), 7.20-7.11 (m, 5H), 5.14-5.10 (m, 1H), 4.76 (s, 2H), 4.66-4.61 (m, 1H), 3.16-3.12 (m, 2H), 2.90-2.87 (m, 1H), 2.62-2.50 (m, 6H), 2.37-2.21 (m, 7H), 2.06-1.92 (m, 7H), 1.49-1.39 (m, 6H), 1.31-1.26 (m, 4H).

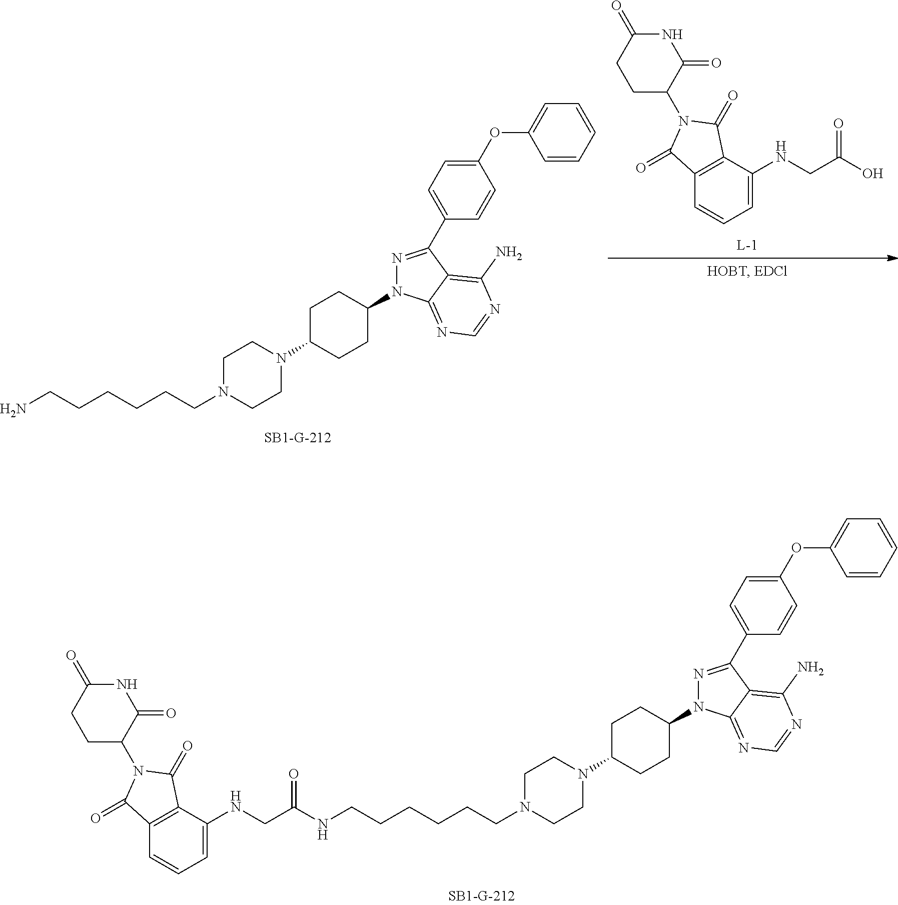 [SMILES: NCCCCCCN1CCN([C@H]2CC[C@H](n3nc(-c4ccc(Oc5ccccc5)cc4)c4c(N)ncnc43)CC2)CC1 ; O=C(O)CNc1cccc2c1C(=O)N(C1CCC(=O)NC1=O)C2=O ; Nc1ncnc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1]

SB1-G-212 was synthesized with similar procedures as SB1-G-181.

LCMS (m/z): 882.5 [M+H]⁺; 1H NMR (DMSO, 400 MHz): δ 11.11 (s, 1H), 8.30 (s, 1H), 8.23 (s, 1H), 8.08 (m, 1H), 7.65 (d, J=8.4 Hz, 2H), 7.59 (t, J=8.0 Hz, 1H), 7.43 (t, J=8.0 Hz, 2H), 7.20-7.06 (m, 6H), 6.95 (t, J=5.6 Hz, 1H), 6.86 (d, J=8.4 Hz, 1H), 5.07 (dd, J₁=12.8 Hz, J₂=5.2 Hz, 1H), 4.64-4.62 (m, 1H), 3.92 (d, J=5.2 Hz, 2H), 3.43 (m, 4H), 3.11-3.06 (m, 2H), 2.94-2.84 (m, 1H), 2.62-2.53 (m, 4H), 2.38-2.23 (m, 7H), 2.03-1.92 (m, 7H), 1.46-1.38 (m, 6H), 1.24 (m, 4H).

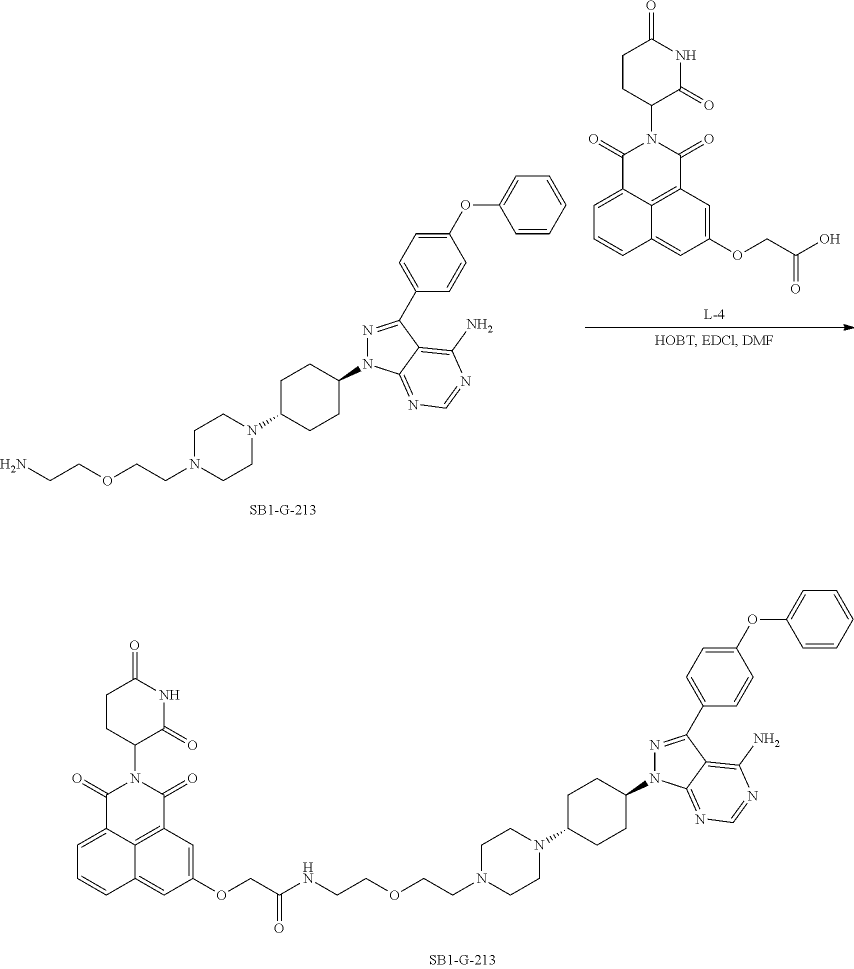

SB1-G-213 was synthesized with similar procedures as SB1-G-181.

LCMS (m/z): 921.3 [M+H]⁺; 1H NMR (DMSO, 400 MHz): δ 11.03 (s, 1H), 8.42-8.28 (m, 4H), 8.22 (s, 1H), 8.17 (d, J=2.4 Hz, 1H), 7.98 (dd, J₁=7.2 Hz, J=2.4 Hz, 1H), 7.85 (q, J=7.6 Hz, 1H), 7.65 (d, J=7.6 Hz, 2H), 7.46-7.41 (m, 2H), 7.20-7.11 (m, 5H), 5.85-5.81 (m, 1H), 4.75 (s, 2H), 4.57-4.54 (m, 1H), 3.48-3.43 (m, 4H), 3.37-3.35 (m, 2H), 2.97-2.87 (m, 1H), 2.67-2.54 (m, 2H), 2.43-2.25 (m, 11H), 2.22-1.82 (m, 8H), 1.40-1.29 (m, 2H).

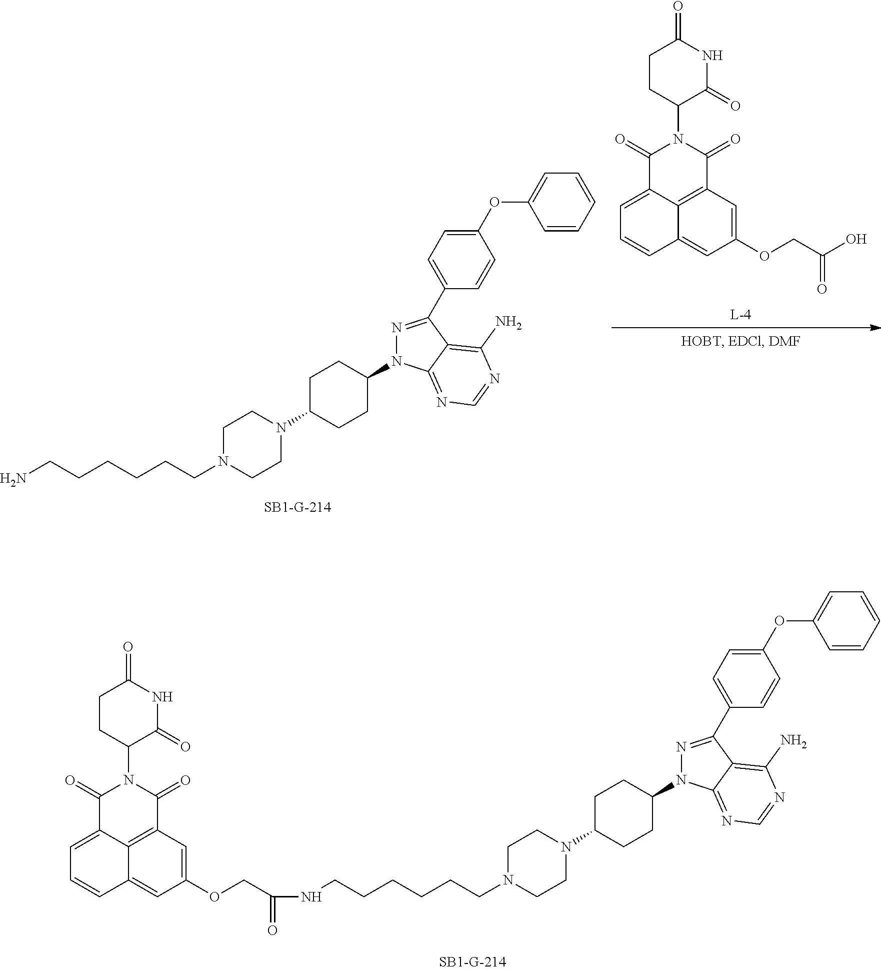

SB1-G-214 was synthesized with similar procedures as SB1-G-181.

LCMS (m/z): 882.5 [M+H]⁺; 1H NMR (DMSO, 400 MHz): δ 10.92 (m, 1H), 8.42 (d, J=7.2 Hz, 1H), 8.36 (d, J=8.4 Hz, 1H), 8.31-8.25 (m, 2H), 8.23 (s, 1H), 8.16 (d, J=2.4 Hz, 1H), 7.94 (dd, J₁=8.0 Hz, J₂=2.4 Hz, 1H), 7.87-7.84 (m, 1H), 7.65 (d, J=8.8 Hz, 2H), 7.45-7.41 (m, 2H), 7.20-7.11 (m, 5H), 5.86-5.83 (m, 1H), 4.74 (s, 2H), 4.64-4.61 (m, 1H), 3.14 (t, J=6.4 Hz, 2H), 2.95 (m, 1H), 2.61-2.54 (m, 2H), 2.37-2.28 (m, 5H), 2.17-2.12 (m, 2H), 2.07-1.90 (m, 8H), 1.44-1.40 (m, 4H), 1.40-1.18 (m, 7H).

### Example 2

### Comparison of Exemplary HCK Degrader Compounds

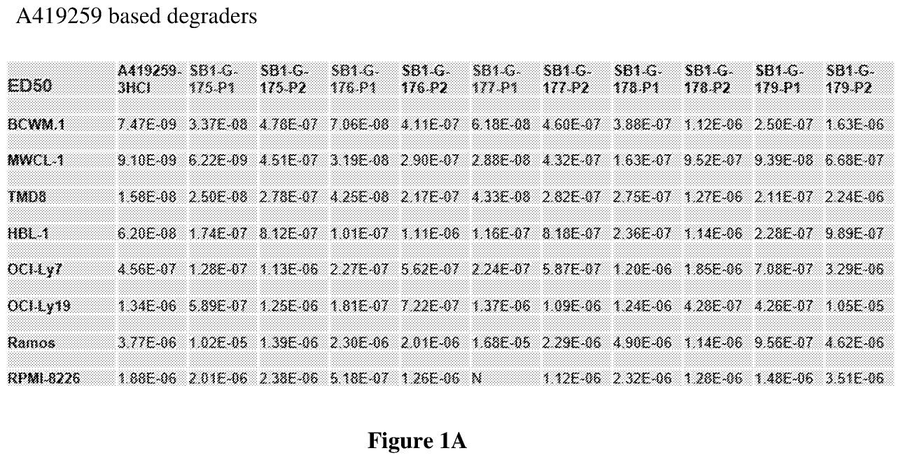

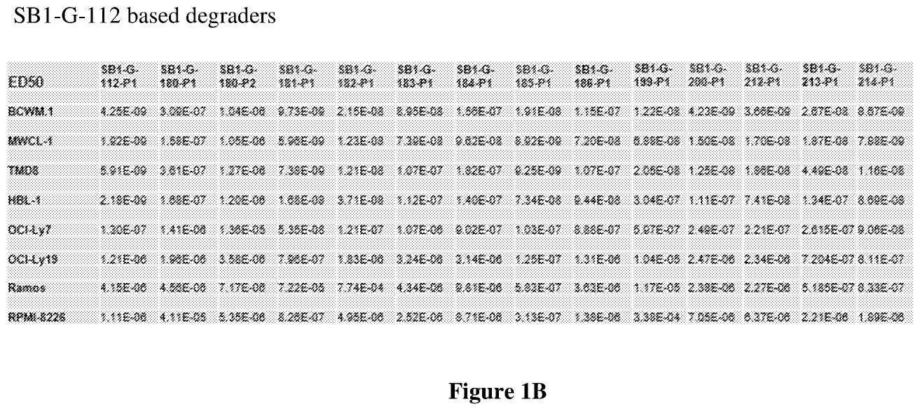

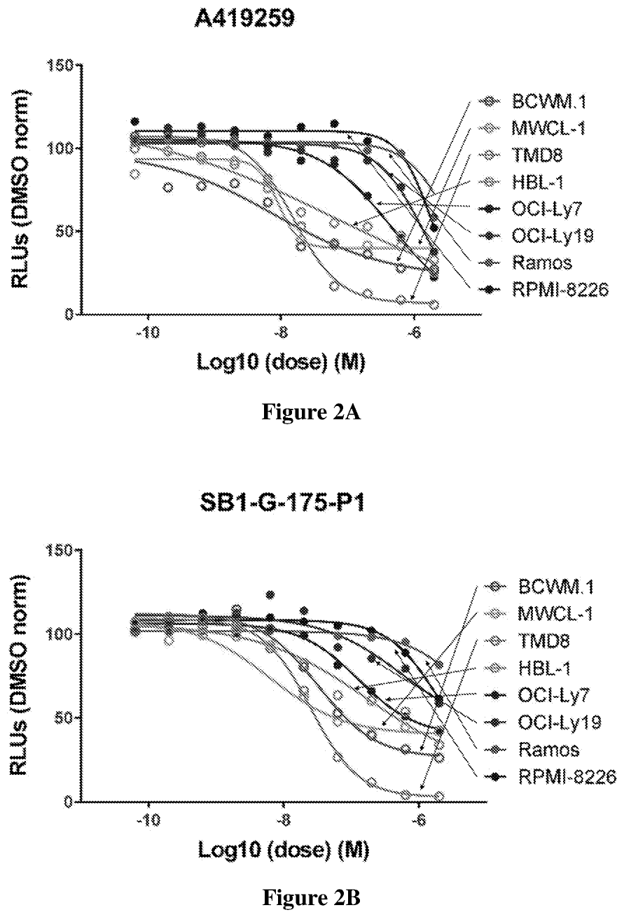

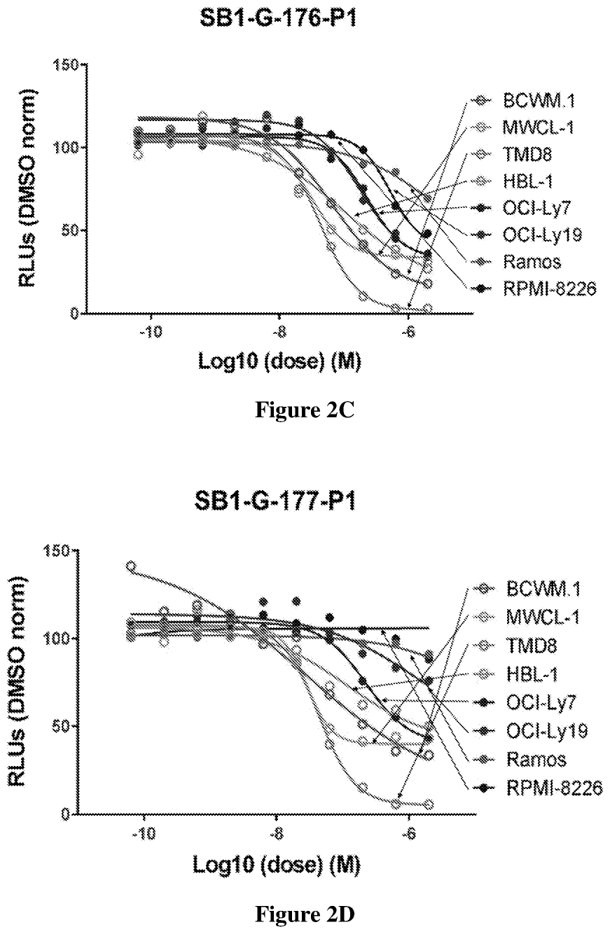

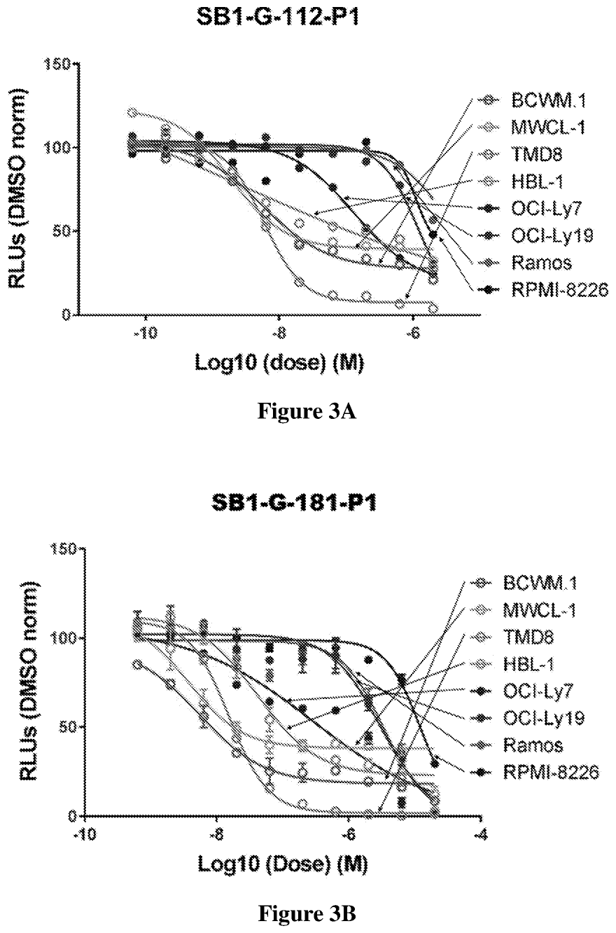

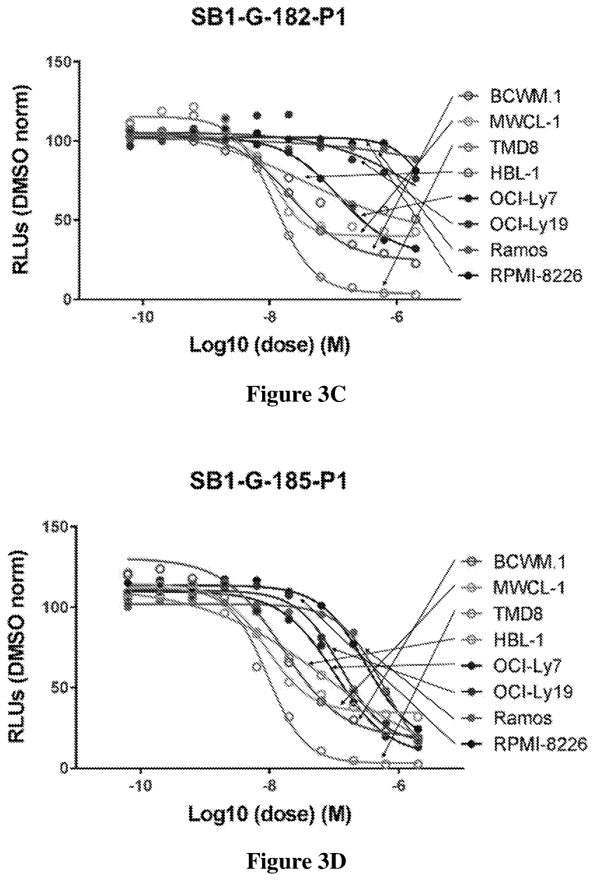

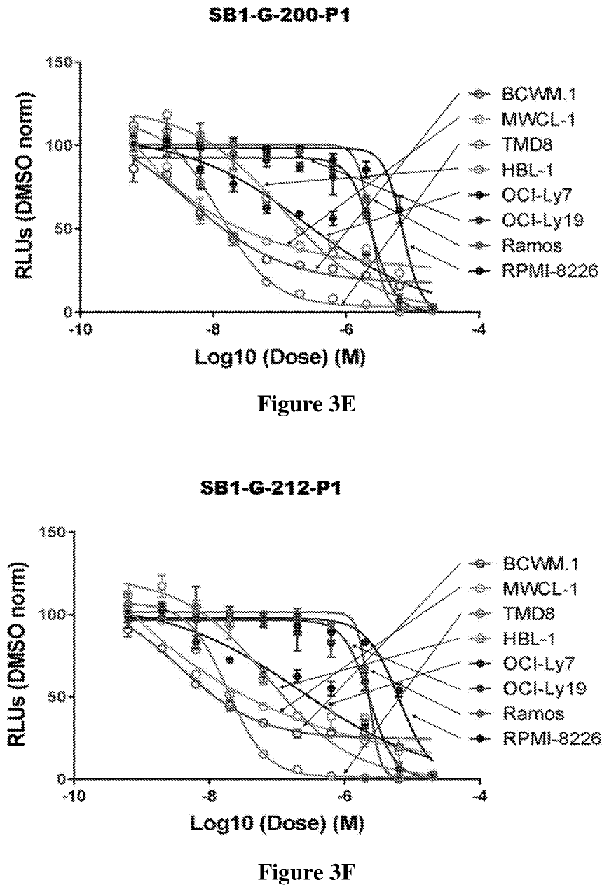

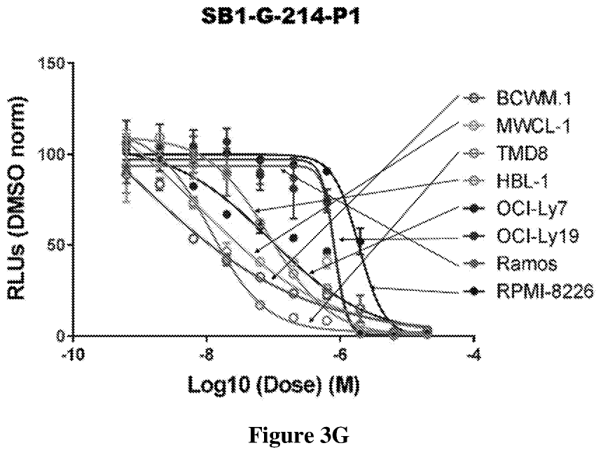

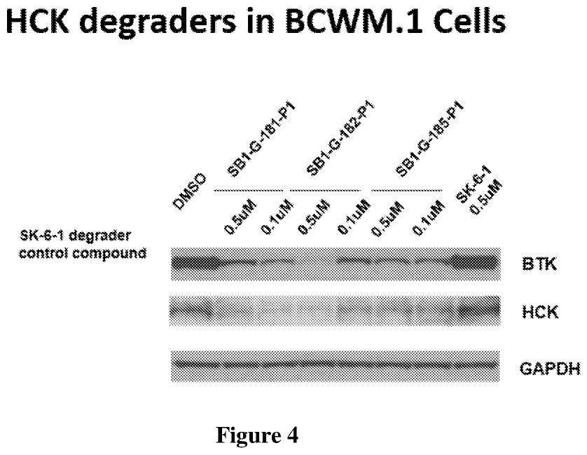

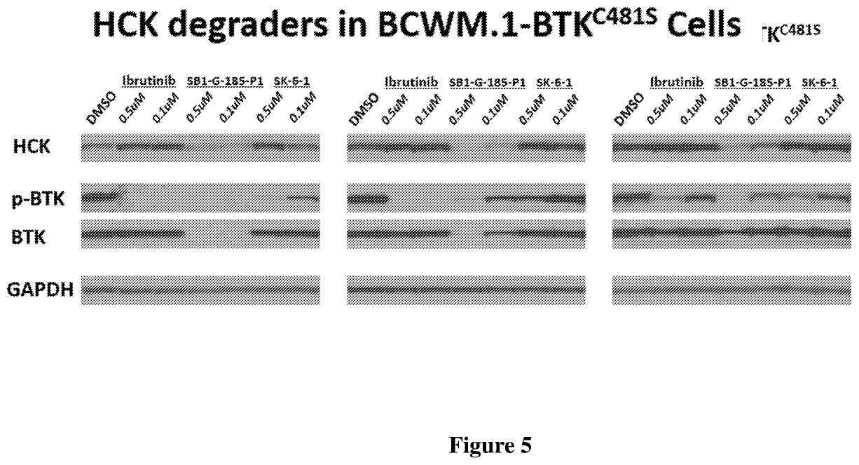

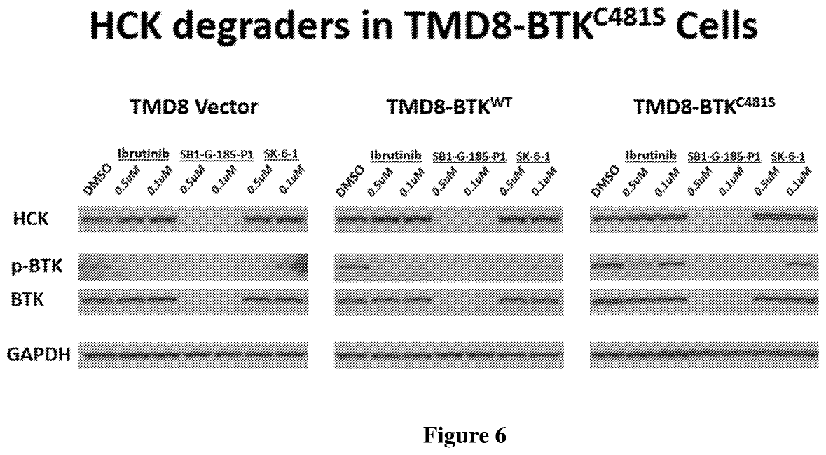

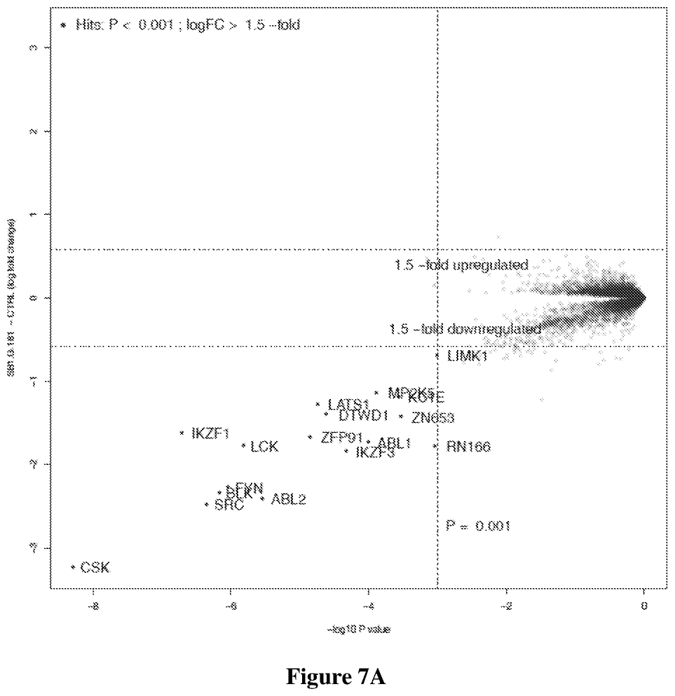

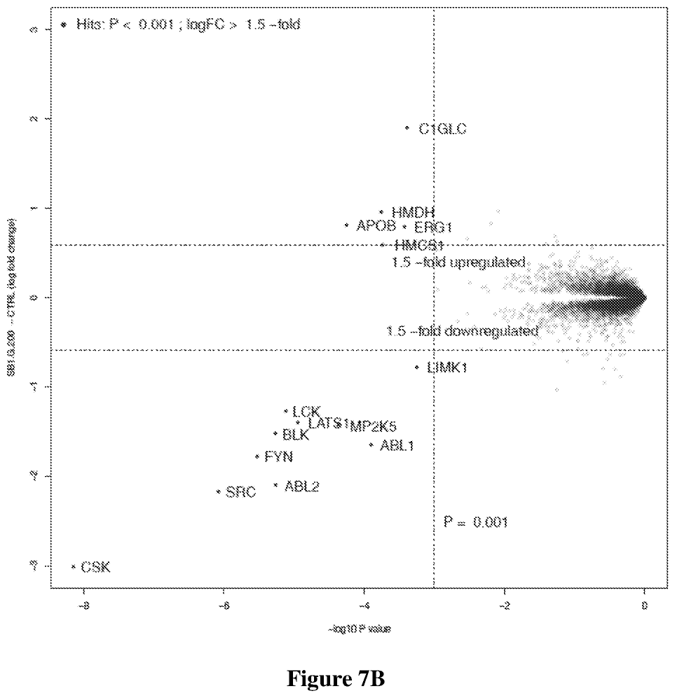

The cellular efficacy of exemplary compounds was tested by assaying A419259 and SB1-G-112 based degrader compounds in MYD88 mutated WM cell lines (BCWM.1, MWCL-1), ABC DLBCL cell lines (TMD-8, HBL-1), and MYD88 wild-type GCB DLBCL cell lines (OCI-Ly7, OCI-Ly19), Burkitt's lymphoma cell line (Ramos), as well as multiple myeloma cell line (RPMI-8226). FIG. 1. The CellTiter-Glo© Luminescent cell viability assay (Promega, Madison Wis.) was used to assess the dose-response of exemplary inhibitors or degraders. Cells were seeded into 384 well plates with the EL406 Combination Washer Dispenser (BioTek Instruments, Inc.) and the exemplary inhibitors were injected into culture media with the JANUS Automated Workstation (PerkinElmer Inc., Waltham Mass.). Cells were incubated with exemplary inhibitors or degraders for 72 hours at 37° C. Luminescent measurements to assess cell viability were performed using the 2104 Envision® Multilabel Reader (PerkinElmer Inc.). See FIGS. 1-3.

Western blots were performed for the detection of protein degradation following the cell treatments with HCK degraders using antibodies for HCK (Cell Signaling), BTK (Cell Signaling) or Phospho-BTK (Tyr223) (Abcam). GAPDH was used for determination of protein loading. See FIGS. 4-6.

TABLE 1
Exemplary HCK Degrader Compounds

| Compound | |
|---|---|
| Name | Structure |
| SB1-G-175-P1 | 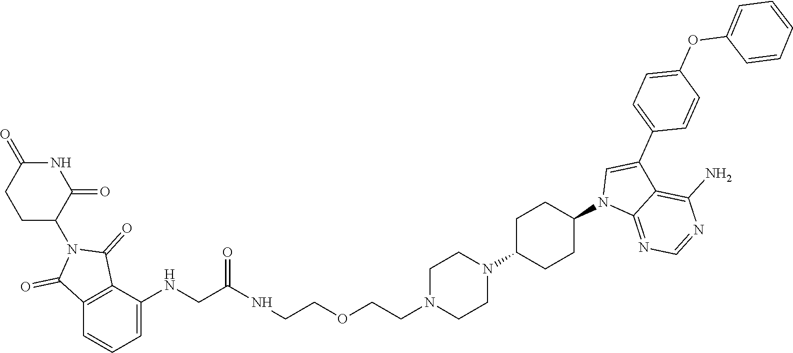 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCC(=O)NC3=O)C4=O)CC2)CC1] |
| SB1-G-176-P1 | 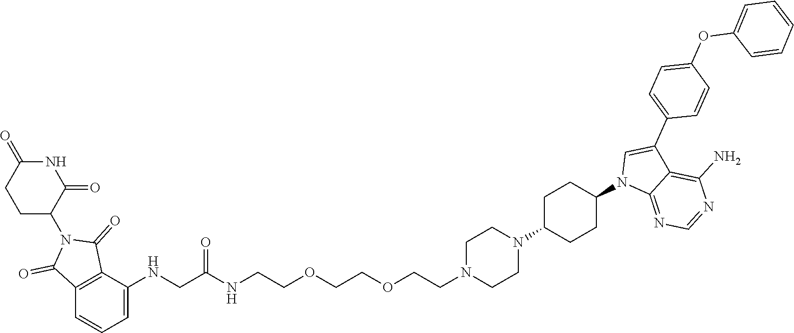 [SMILES: Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCC(=O)NC3=O)C4=O)CC2)CC1] |
| SB1-G-177-P1 | 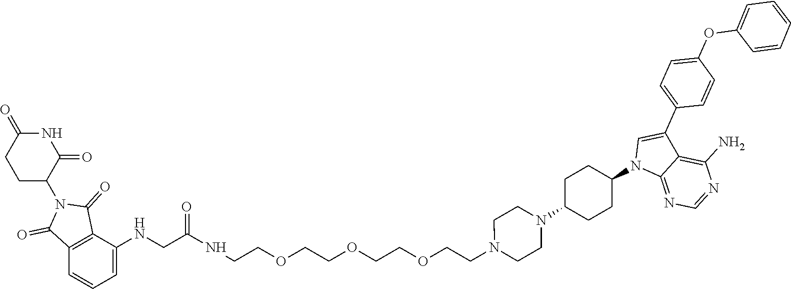 [SMILES: Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCC(=O)NC3=O)C4=O)CC2)CC1] |
| SB1-G-178-P1 | 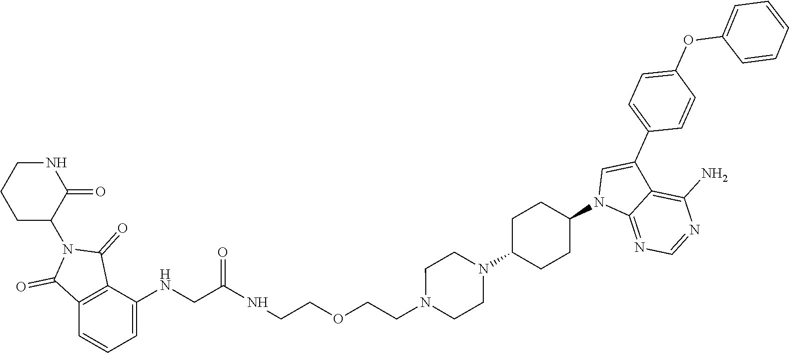 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCCNC3=O)C4=O)CC2)CC1] |
| SB1-G-179-P1 | 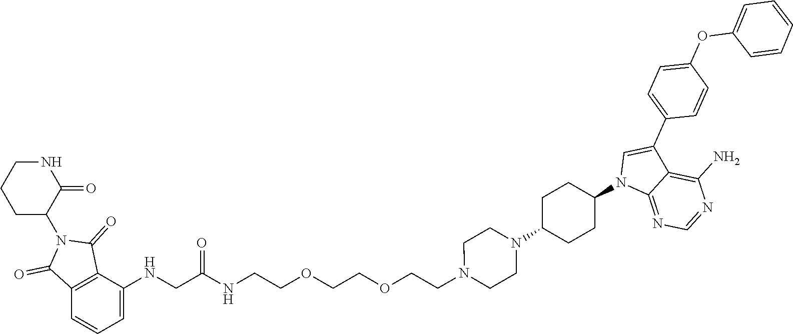 [SMILES: Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCCNC3=O)C4=O)CC2)CC1] |
| SB1-G-180-P1 | 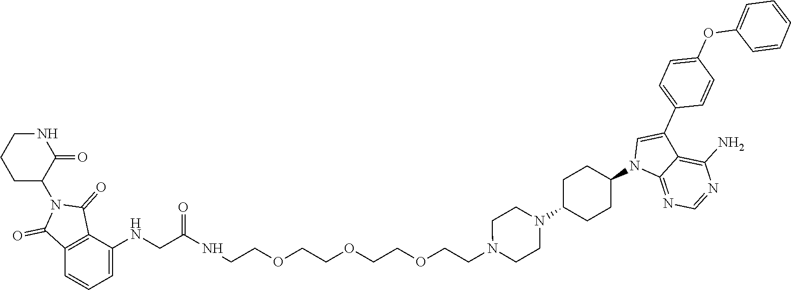 [SMILES: Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCCNC3=O)C4=O)CC2)CC1] |
| SB1-G-175-P2 | 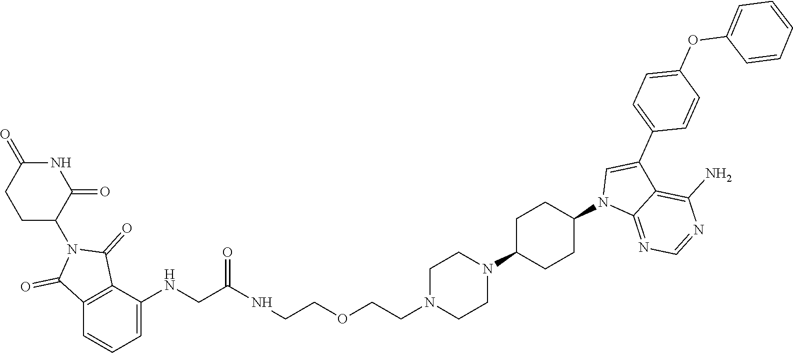 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCC(=O)NC3=O)C4=O)CC2)CC1] |
| SB1-G-176-P2 | 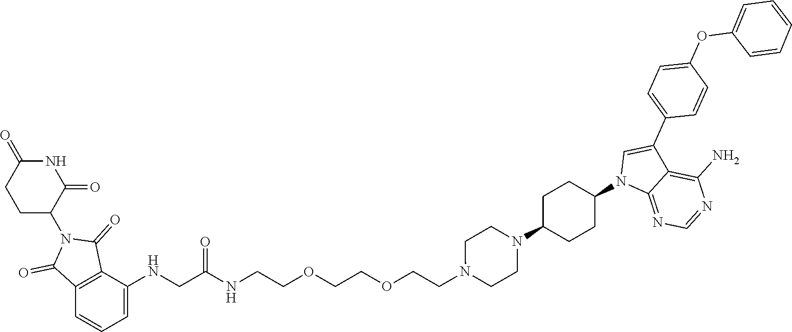 [SMILES: Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCC(=O)NC3=O)C4=O)CC2)CC1] |
| SB1-G-177-P2 | 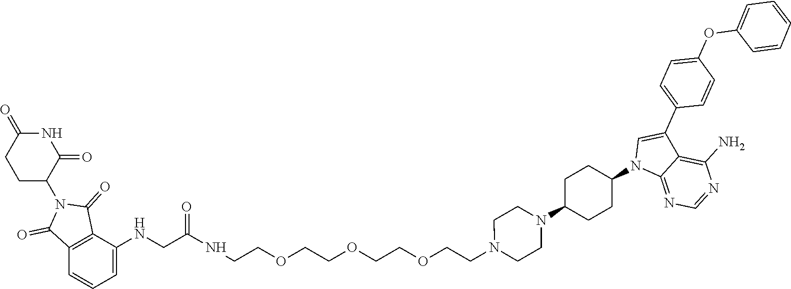 [SMILES: Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCC(=O)NC3=O)C4=O)CC2)CC1] |
| SB1-G-178-P2 | 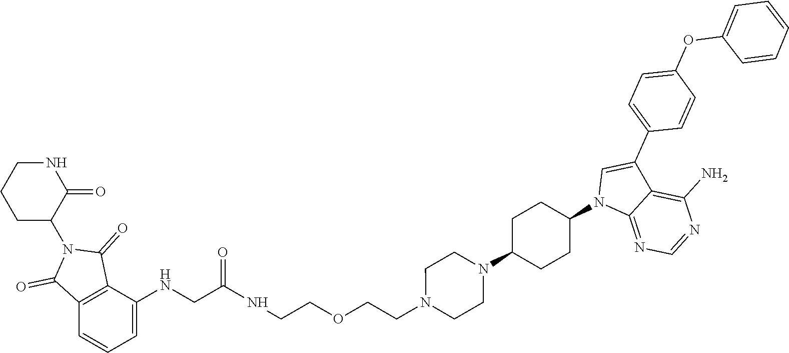 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCCNC3=O)C4=O)CC2)CC1] |
| SB1-G-179-P2 | 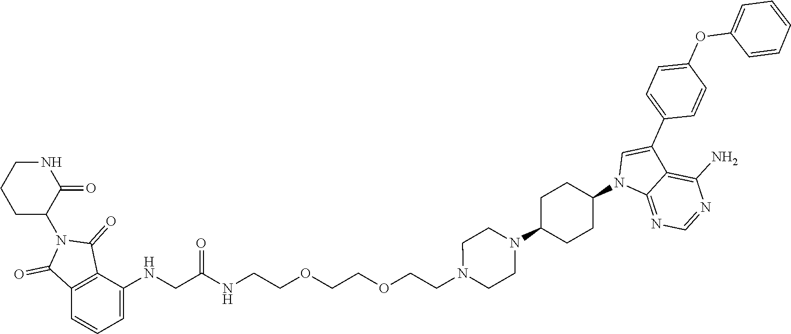 [SMILES: Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCCNC3=O)C4=O)CC2)CC1] |
| SB1-G-180-P2 | 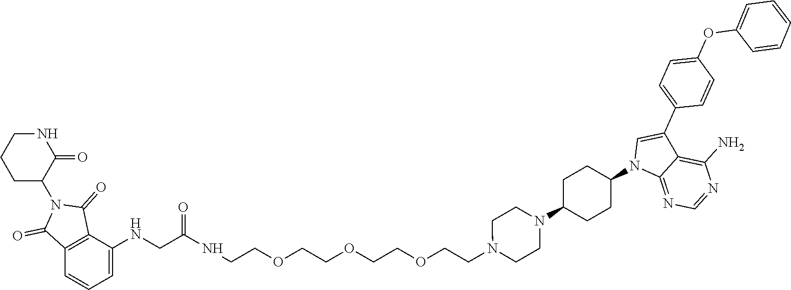 [SMILES: Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCCNC3=O)C4=O)CC2)CC1] |
| SB1-G-181 | 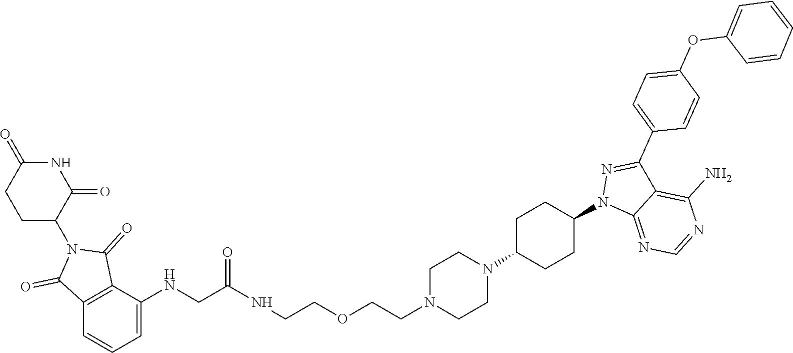 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1] |
| SB1-G-182 | 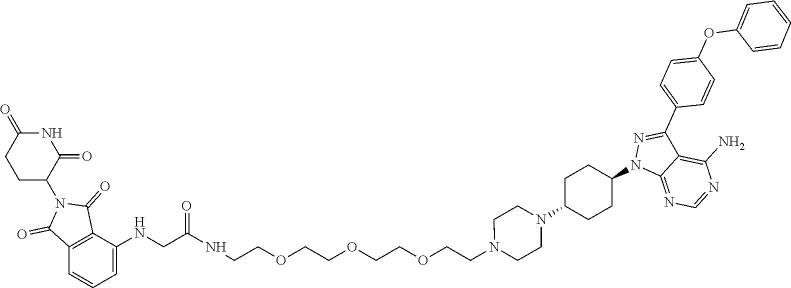 [SMILES: Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCC(=O)NC3=O)C4=O)CC2)CC1] |
| SB1-G-183 | 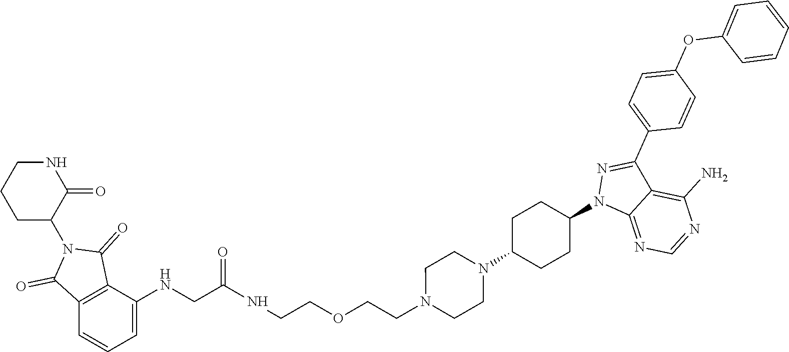 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCCNC3=O)C4=O)CC2)CC1] |
| SB1-G-184 | 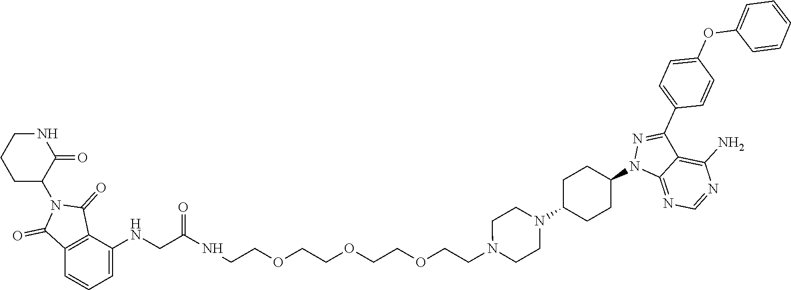 [SMILES: Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCCNC3=O)C4=O)CC2)CC1] |
| SB1-G-185 | 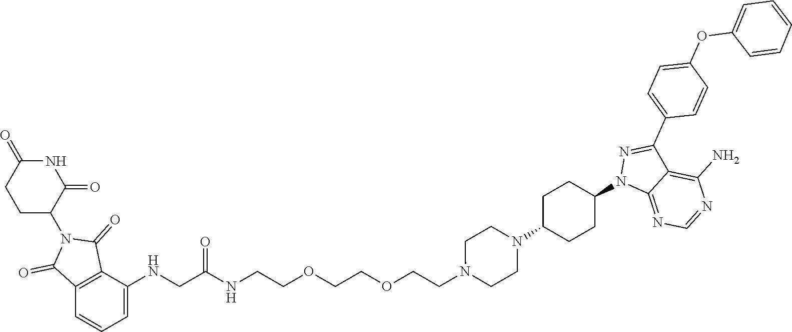 [SMILES: Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCC(=O)NC3=O)C4=O)CC2)CC1] |
| SB1-G-186 | 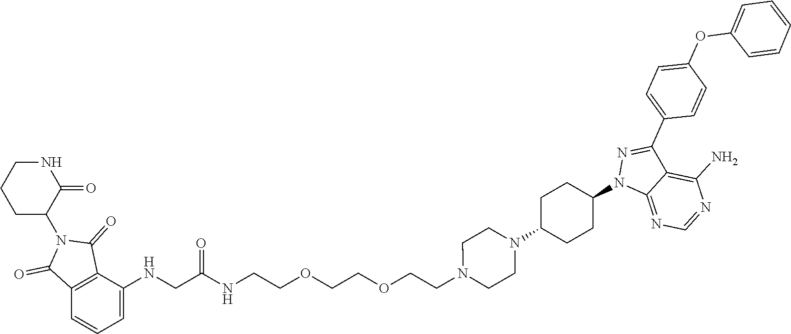 [SMILES: Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCCNC3=O)C4=O)CC2)CC1] |
| SB1-G-199 | 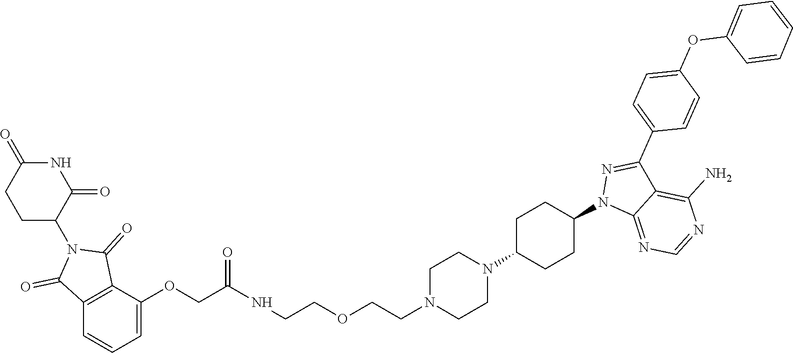 [SMILES: Nc1ncnc2c1c(-c1ccc(Oc3ccccc3)cc1)nn2[C@H]1CC[C@H](N2CCN(CCOCCNC(=O)COc3cccc4c3C(=O)N(C3CCC(=O)NC3=O)C4=O)CC2)CC1] |
| SB1-G-200 | 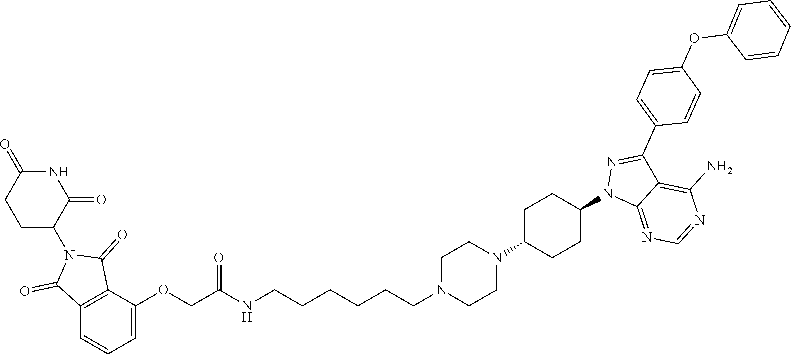 [SMILES: Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1] |
| SB1-G-212 | 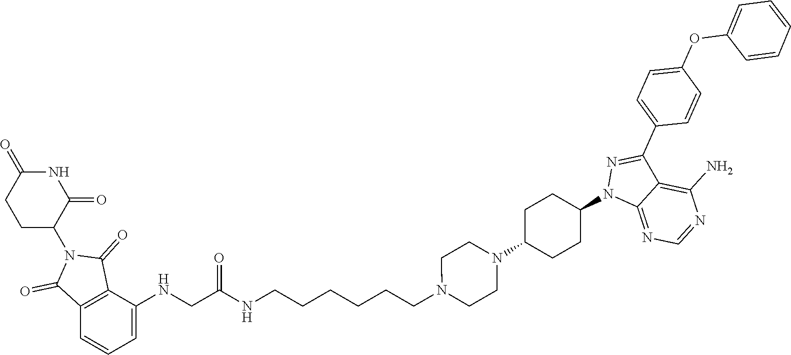 [SMILES: Nc1ncnc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1] |
| SB1-G-213 | 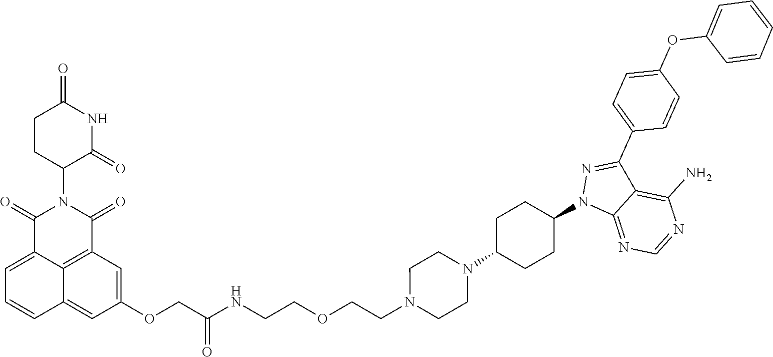 [SMILES: Nc1ncnc2c1c(-c1ccc(Oc3ccccc3)cc1)nn2[C@H]1CC[C@H](N2CCN(CCOCCNC(=O)COc3cc4c5c(cccc5c3)C(=O)N(C3CCC(=O)NC3=O)C4=O)CC2)CC1] |
| SB1-G-214 | 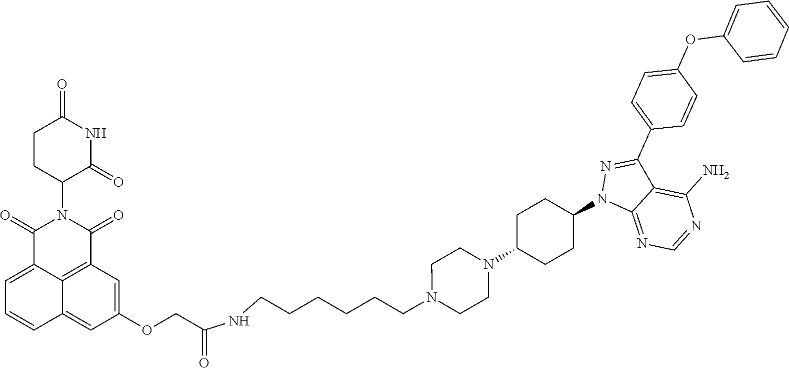 [SMILES: Nc1ncnc2c1c(-c1ccc(Oc3ccccc3)cc1)nn2[C@H]1CC[C@H](N2CCN(CCCCCCNC(=O)COc3cc4c5c(cccc5c3)C(=O)N(C3CCC(=O)NC3=O)C4=O)CC2)CC1] |
| SB1-G-215 | 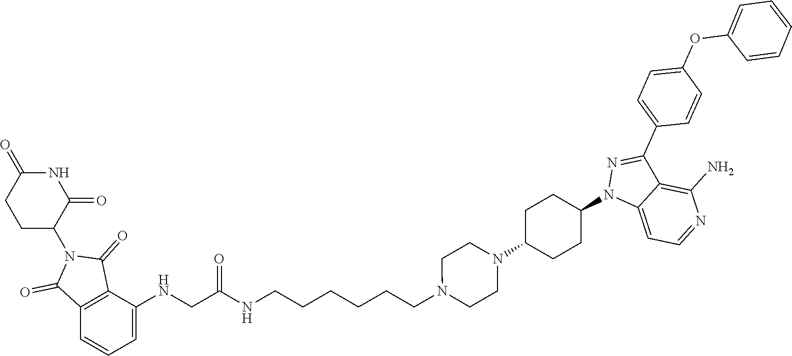 [SMILES: Nc1nccc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1] |
| SB1-G-216 | 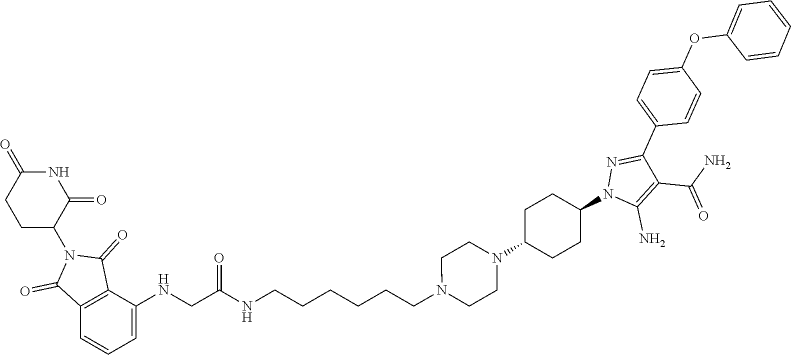 [SMILES: NC(=O)c1c(-c2ccc(Oc3ccccc3)cc2)nn([C@H]2CC[C@H](N3CCN(CCCCCCNC(=O)CNc4cccc5c4C(=O)N(C4CCC(=O)NC4=O)C5=O)CC3)CC2)c1N] |

### EQUIVALENTS AND SCOPE

In the claims articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the disclosure, or aspects described herein, is/are referred to as comprising particular elements and/or features, certain embodiments described herein or aspects described herein consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different embodiments described herein, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular embodiment of the present disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment described herein can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the present disclosure, as defined in the following claims.

1. A compound of Formula (I): 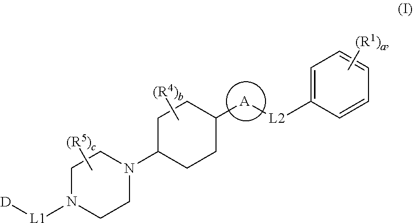
or a pharmaceutically acceptable salt, co-crystal, tautomer, stereoisomer, solvate, hydrate, polymorph, isotopically enriched derivative, or prodrug thereof, wherein:
each instance of R¹ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;
R^{D1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{D1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{D1a} are taken together with their intervening atoms to form a substituted or unsubstituted heterocyclic or substituted or unsubstituted heteroaryl ring;
each instance of R² is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;
each instance of R³ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;
each instance of R⁴ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;
each instance of R⁵ is independently halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{D1}, —N(R^{D1a})₂, —SR^{D1}, —NO₂, or —SCN;
L1 is a linker;
L2 is a bond or 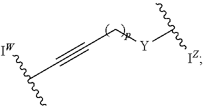
Ring A is of formula: 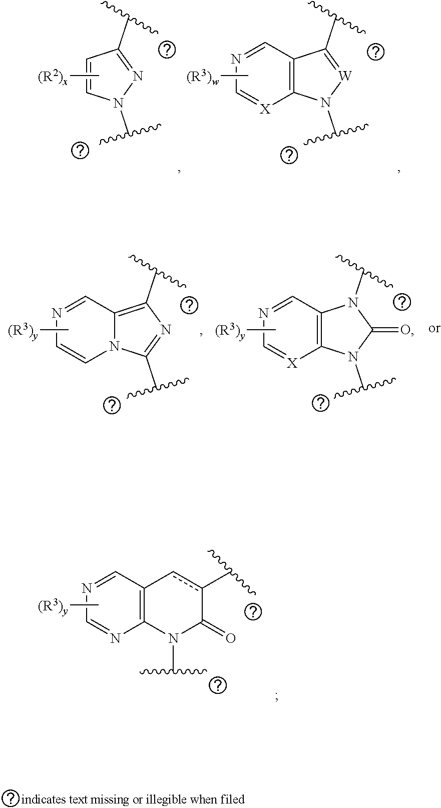 [symbol]
is a single bond or a double bond, as valency permits;
W is ═C(R^{A})— or ═N—;
X is ═C(R^{A})— or ═N—;
Y is O, —N(R^{Y})—, or S;
each instance of R^{A} is independently hydrogen, halogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, —CN, —OR^{A1}, —N(R^{A1a})₂ or —SR^{A1};
R^{A1} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, an oxygen protecting group when attached to an oxygen atom, or a sulfur protecting group when attached to a sulfur atom;
each occurrence of R^{A1a} is hydrogen, optionally substituted acyl, optionally substituted alkyl, optionally substituted alkenyl, optionally substituted alkynyl, optionally substituted carbocyclyl, optionally substituted heterocyclyl, optionally substituted aryl, optionally substituted heteroaryl, or a nitrogen protecting group; or optionally two instances of R^{A1a} are taken together with their intervening atoms to form a substituted or unsubstituted heterocyclic or substituted or unsubstituted heteroaryl ring;
R^{T} is hydrogen, optionally substituted acyl, optionally substituted alkyl, optionally substituted alkenyl, optionally substituted alkynyl, or a nitrogen protecting group;
a is 0, 1, 2, 3, 4, or 5;
b is 0, 1, 2, 3, 4, 5, 6, 7, 8, 9, or 10;
c is 0, 1, 2, 3, 4, 5, 6, 7, or 8;
p is 0, 1, 2, or 3;
w is 0, 1, 2, 3, or 4;
x is 0, 1, or 2;
y is 0, 1, 2, or 3;
D is an E3 ubiquitin ligase binding moiety;
l^{X} indicates the point of attachment to the moiety of formula 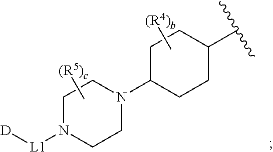
l^{Y} indicates the point of attachment to L2
l^{W} indicates the point of attachment to Ring A; and
l^{Z} indicates the point of attachment to the moiety of formula

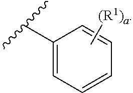

2. The compound of claim 1, wherein Ring A is of formula:

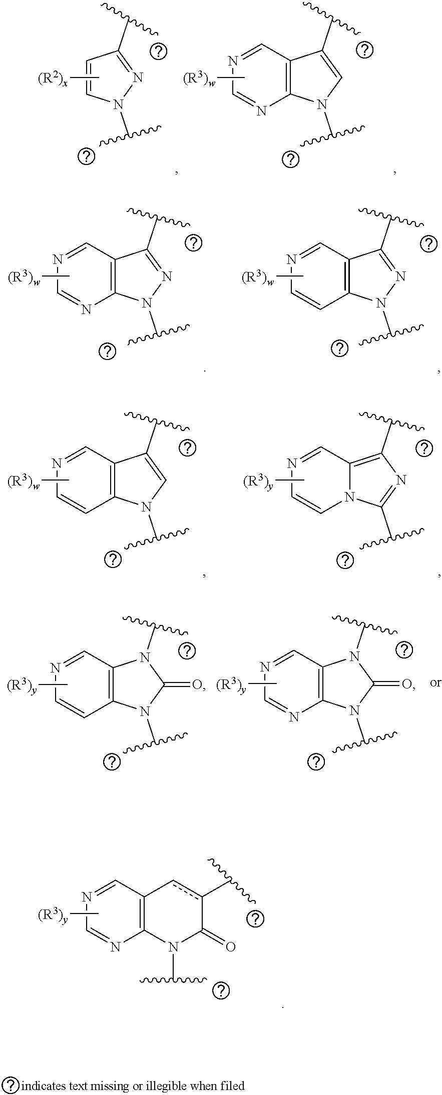

3-13. (canceled)

14. The compound of claim 1, wherein the compound is of formula: 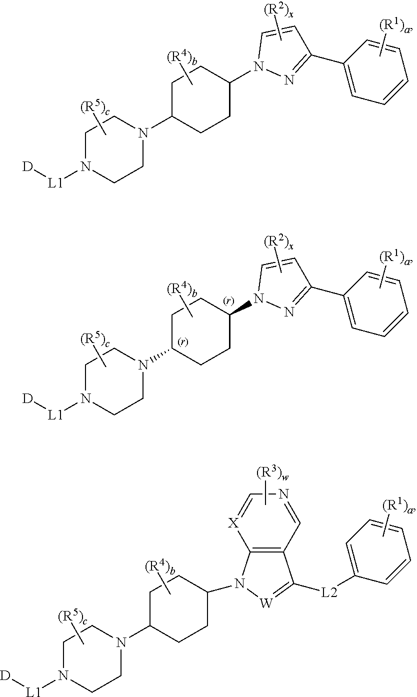 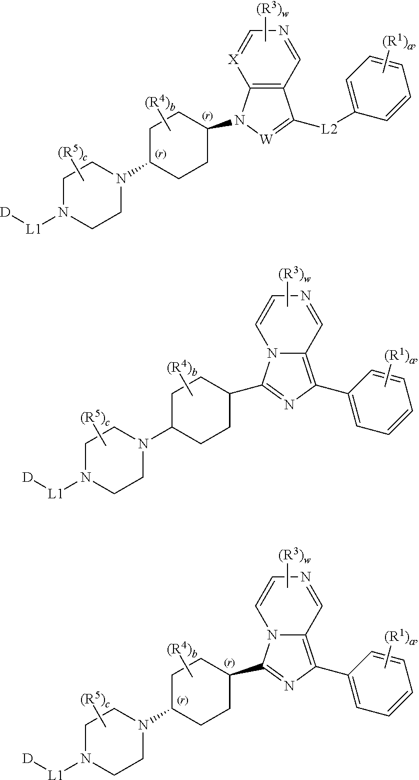 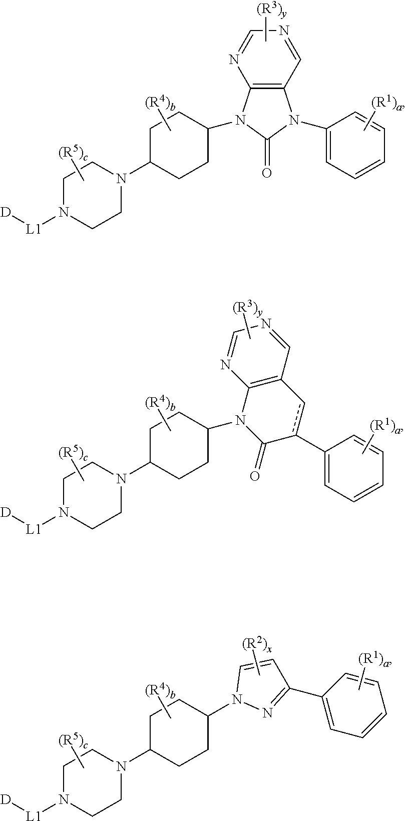 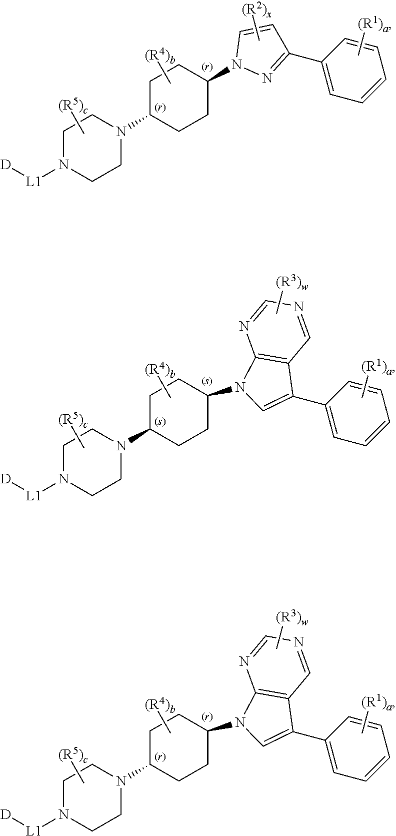 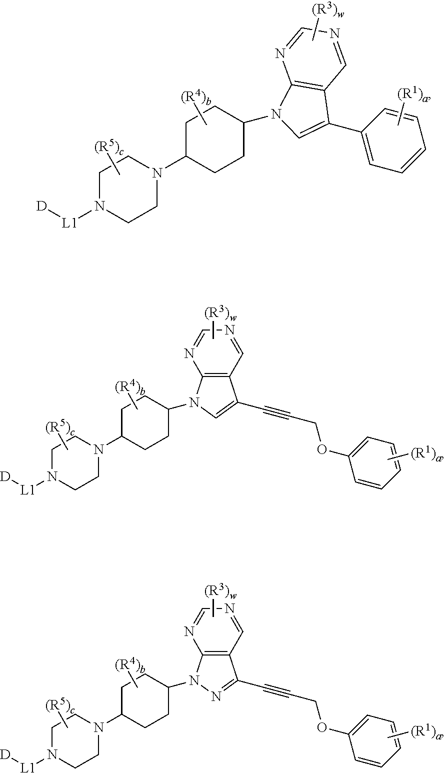 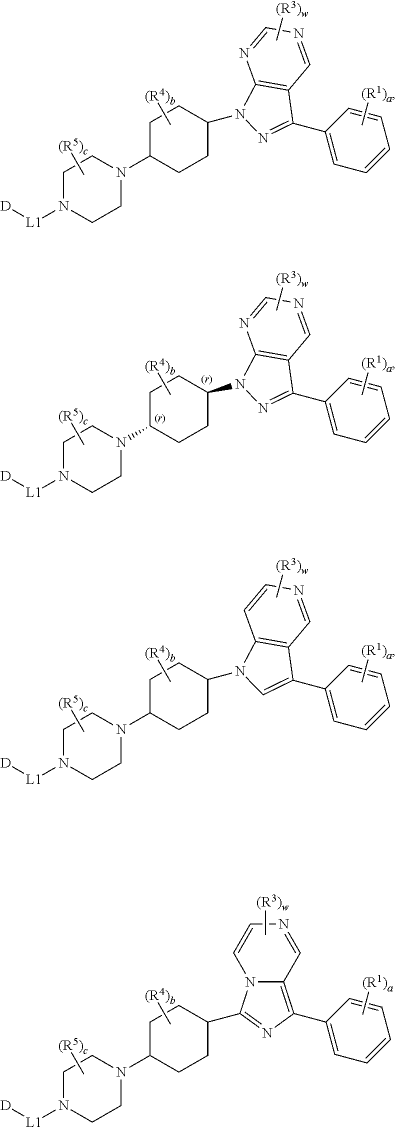
wherein a is 0, 1, or 2, 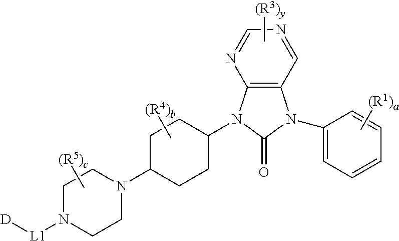
wherein a is 0, 1, or 2,
or 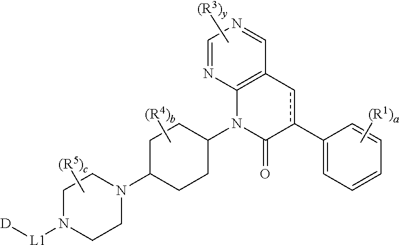
wherein a is 0, 1, or 2,
or a pharmaceutically acceptable salt, co-crystal, tautomer, stereoisomer, solvate, hydrate, polymorph, isotopically enriched derivative, or prodrug thereof.

15.-34. (canceled)

35. The compound of claim 1, or a pharmaceutically acceptable salt, co-crystal, tautomer, stereoisomer, solvate, hydrate, polymorph, isotopically enriched derivative, or prodrug thereof, wherein D is of the formula: 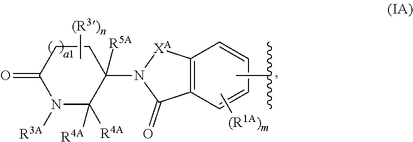
wherein:
X^{A} is C(O) or C(R^{3A})₂;
each R^{1A} is independently halogen, —OH, C₁-C₆ alkyl, or C₁-C₆ alkoxy;
each R^{3A} is independently hydrogen or C₁-C₃ alkyl;
each R^{3′} is independently C₁-C₃ alkyl;
each R^{4A} is independently hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and 0;
R^{5A} is H, C₁-C₃ alkyl, or halogen;
m is 0, 1, 2 or 3;
n is 0, 1, or 2; and
a1 is 0 or 1;
or wherein D is of the formula: 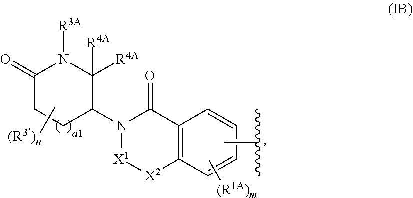
wherein:
—X¹—X²— is —C(R^{3A})═N— or —C(R^{3A})₂—C(R^{3A})₂—;
each R^{1A} is independently halogen, —OH, C₁-C₆ alkyl, or C₁-C₆ alkoxy;
R^{3A} is hydrogen or C₁-C₃ alkyl;
each R^{3′} is independently C₁-C₃ alkyl;
each R^{4A} is independently hydrogen or C₁-C₃ alkyl; or two R^{4A}, together with the carbon atom to which they are attached, form a C(O), C₃-C₆ carbocycle, or a 4-, 5-, or 6-membered heterocycle comprising 1 or 2 heteroatoms selected from N and O;
R^{5A} is H, C₁-C₃ alkyl, or halogen;
m is 0, 1, 2, or 3;
n is 0, 1, or 2; and
a1 is 0 or 1:
or wherein D is of the formula: 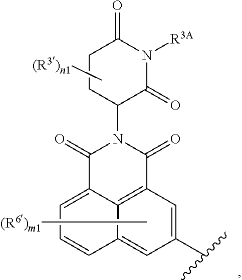
wherein:
R^{3A} is hydrogen or C₁-C₃ alkyl;
each R^{3′} is independently C₁-C₃ alkyl;
each R^{6′} is independently halogen, —OH, C₁-C₆ alkyl, or C₁-C₆ alkoxy;
n1 is 0, 1, 2, 3, 4, or 5; and
m1 is 0, 1, 2, 3, 4, or 5.

36. (canceled)

37. The compound of claim 35, or a pharmaceutically acceptable salt, co-crystal, tautomer, stereoisomer, solvate, hydrate, polymorph, isotopically enriched derivative, or prodrug thereof, wherein R^{3A} is hydrogen.

38.-44. (canceled)

45. The compound of claim 1, or a pharmaceutically acceptable salt, co-crystal, tautomer, stereoisomer, solvate, hydrate, polymorph, isotopically enriched derivative, or prodrug thereof, wherein D is of the formula:

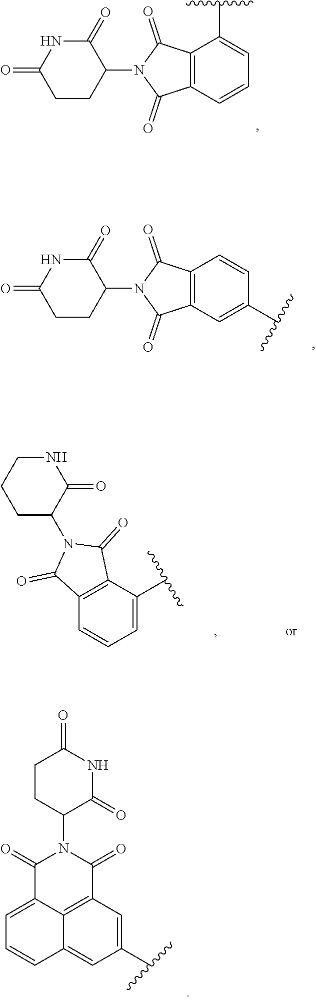 [SMILES: *c1cccc2c1C(=O)N(C1CCC(=O)NC1=O)C2=O ; *c1ccc2c(c1)C(=O)N(C1CCC(=O)NC1=O)C2=O ; *c1cccc2c1C(=O)N(C1CCCNC1=O)C2=O ; *c1cc2c3c(cccc3c1)C(=O)N(C1CCC(=O)NC1=O)C2=O]

46. The compound of claim 1, or a pharmaceutically acceptable salt, co-crystal, tautomer, stereoisomer, solvate, hydrate, polymorph, isotopically enriched derivative, or prodrug thereof, wherein D is of the formula: 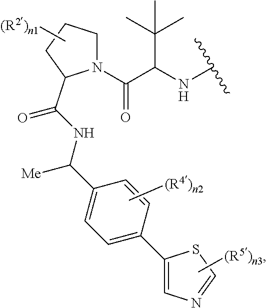
wherein:
each R^{2′} is independently halogen, —OH, C₁-C₆ alkyl, or C₁-C₆ alkoxy;
each R^{4′} is independently halogen, —OH, C₁-C₆ alkyl, or C₁-C₆ alkoxy;
each R^{5′} is independently halogen, —OH, C₁-C₆ alkyl, or C₁-C₆ alkoxy;
n1′ is 0, 1, 2, 3, 4, 5, or 6;
n2′ is 0, 1, 2, 3, or 4; and
n3′ is 0, 1, or 2; or wherein D is of the formula:

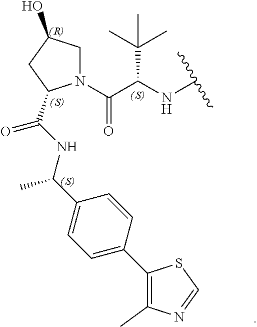

47.-52. (canceled)

53. The compound of claim 1, wherein at least one instance of R¹ is —O(optionally substituted phenyl) or —O(unsubstituted phenyl).

54. (canceled)

55. The compound of claim 1, wherein at least one instance of R³ is —NH₂.

56.-58. (canceled)

59. The compound of claim 1, wherein the compound is of formula: 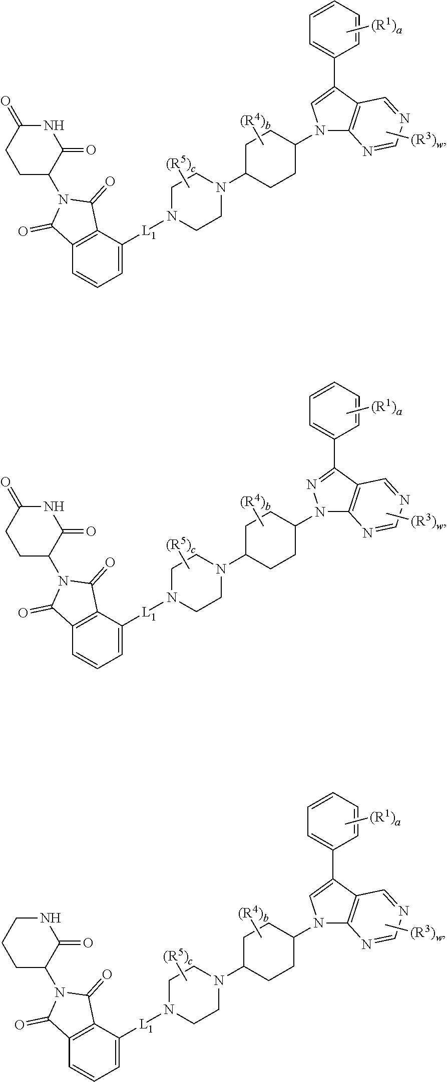 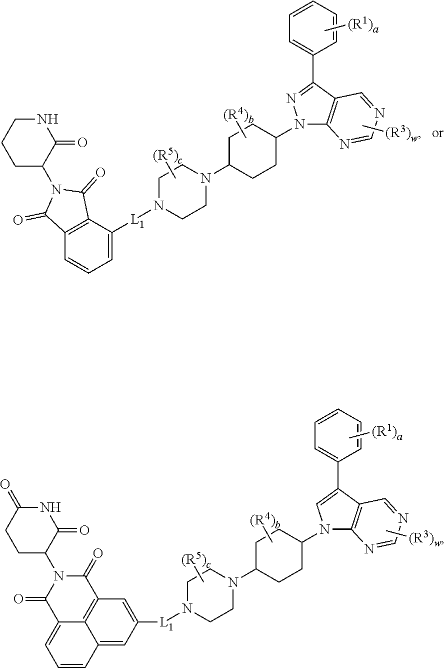
or a pharmaceutically acceptable salt, co-crystal, tautomer, stereoisomer, solvate, hydrate, polymorph, isotopically enriched derivative, or prodrug thereof.

60.-63. (canceled)

64. The compound of claim 1, wherein the compound is of formula: 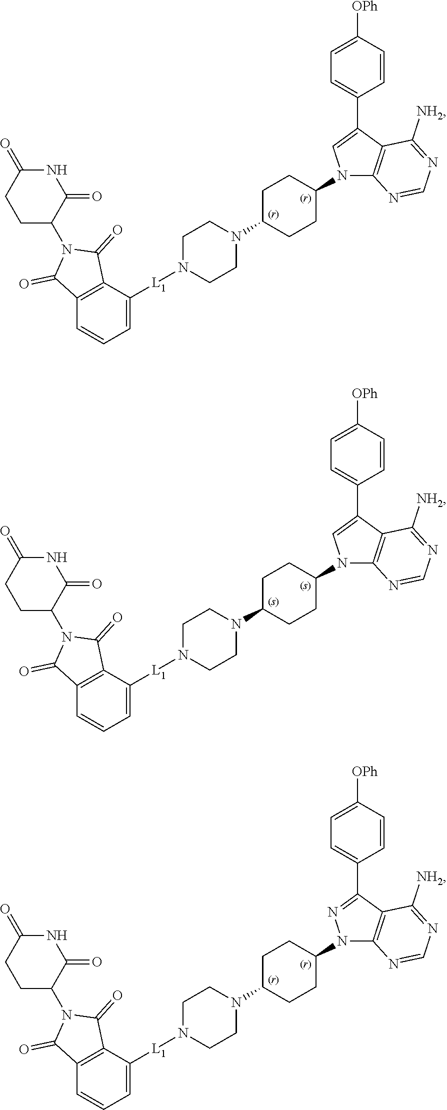 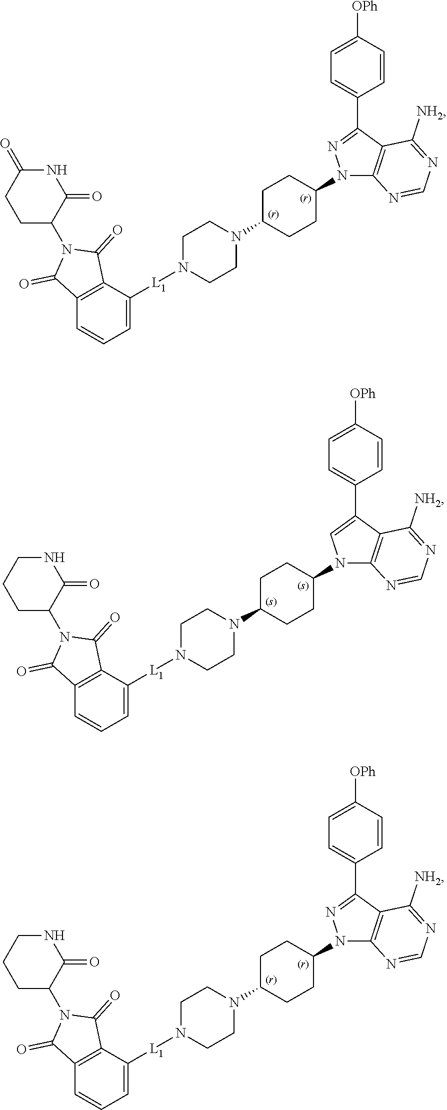 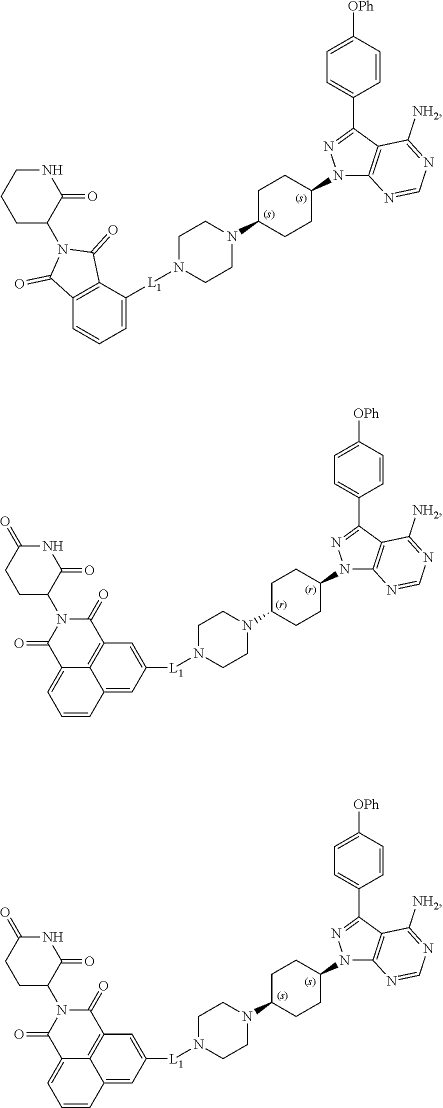
or a pharmaceutically acceptable salt, co-crystal, tautomer, stereoisomer, solvate, hydrate, polymorph, isotopically enriched derivative, or prodrug thereof.

65.-66. (canceled)

67. The compound of claim 1, or a pharmaceutically acceptable salt, co-crystal, tautomer, stereoisomer, solvate, hydrate, polymorph, isotopically enriched derivative, or prodrug thereof, wherein L1 is an unsubstituted C₁₋₂₄ hydrocarbon chain, optionally wherein one or more chain atoms of the hydrocarbon chain are independently replaced with —C(═O)—, —O—, —NR^{b}—, or a cyclic moiety, wherein R^{b} is independently hydrogen, substituted or unsubstituted C₁₋₆ alkyl, or a nitrogen protecting group.

68.-73. (canceled)

74. The compound of claim 1, or a pharmaceutically acceptable salt, co-crystal, tautomer, stereoisomer, solvate, hydrate, polymorph, isotopically enriched derivative, or prodrug thereof, wherein L1 is of the formula: 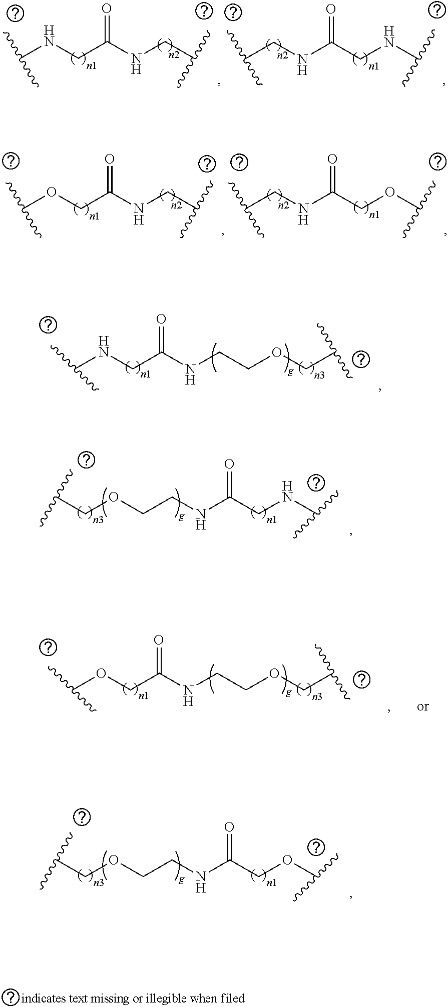
l^{R} indicates the point of attachment to the moiety of formula: 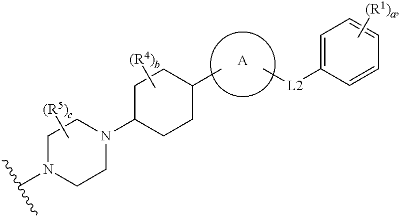
and l^{A} indicates the point of attachment to D;
n1 is 1, 2, 3, 4, 5, or 6;
n2 is 1, 2, 3, 4, 5, 6, 7, 8, 9, or 10;
n3 is 1, 2, 3, 4, 5, or 6; and
g is 1, 2, 3, 4, 5, or 6.

75.-81. (canceled)

82. The compound of claim 1, or a pharmaceutically acceptable salt, co-crystal, tautomer, stereoisomer, solvate, hydrate, polymorph, isotopically enriched derivative, or prodrug thereof, wherein L1 is of the formula:

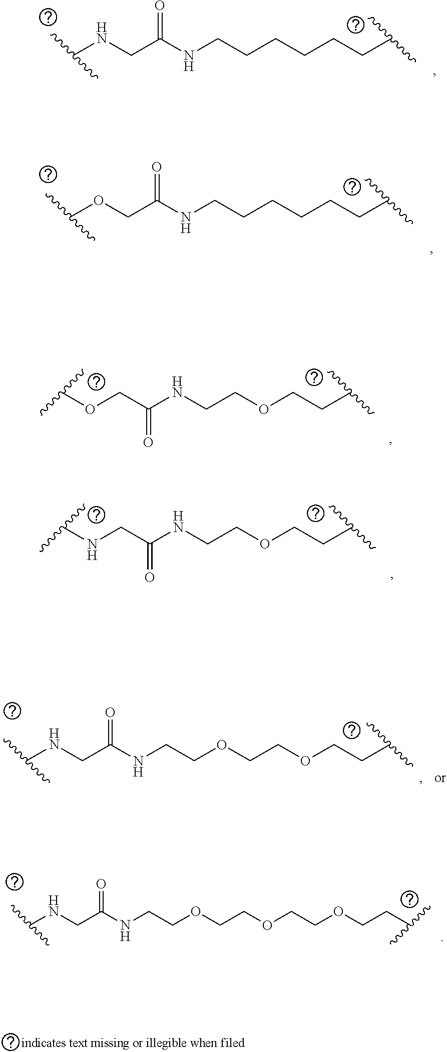 [SMILES: *CCCCCCNC(=O)CN* ; *CCCCCCNC(=O)CO* ; *CCOCCNC(=O)CO* ; *CCOCCNC(=O)CN* ; *CCOCCOCCNC(=O)CN* ; *CCOCCOCCOCCNC(=O)CN*]

83. The compound of claim 1, wherein the compound is of the formula: 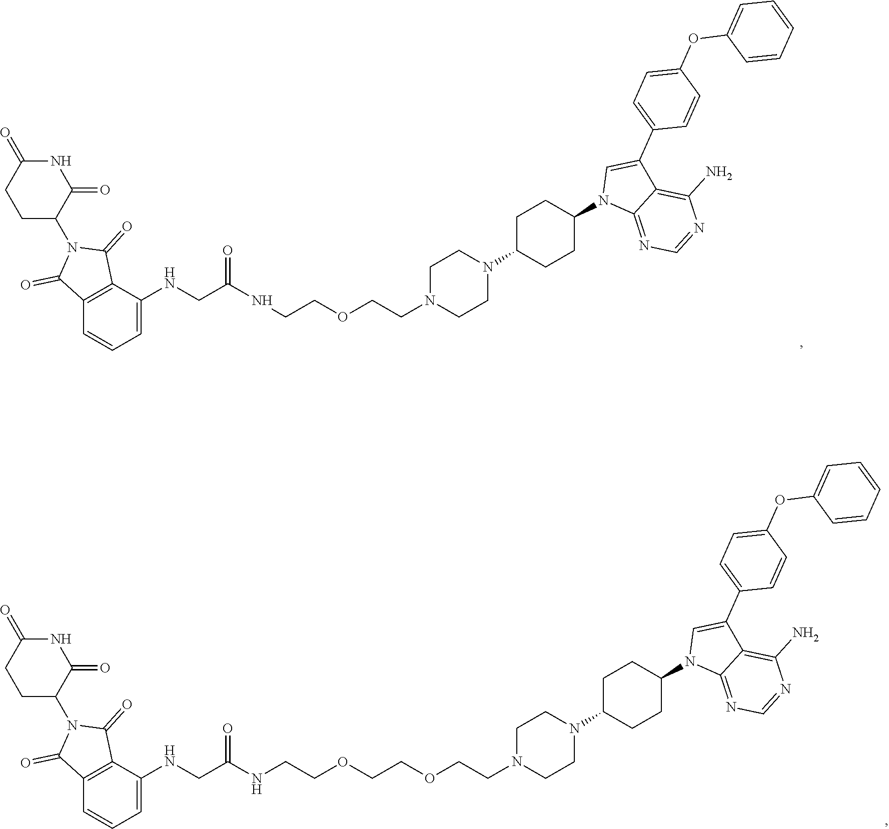 [SMILES: Nc1ncnc2c1c(-c1ccc(Oc3ccccc3)cc1)cn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCC(=O)NC3=O)C4=O)CC2)CC1] 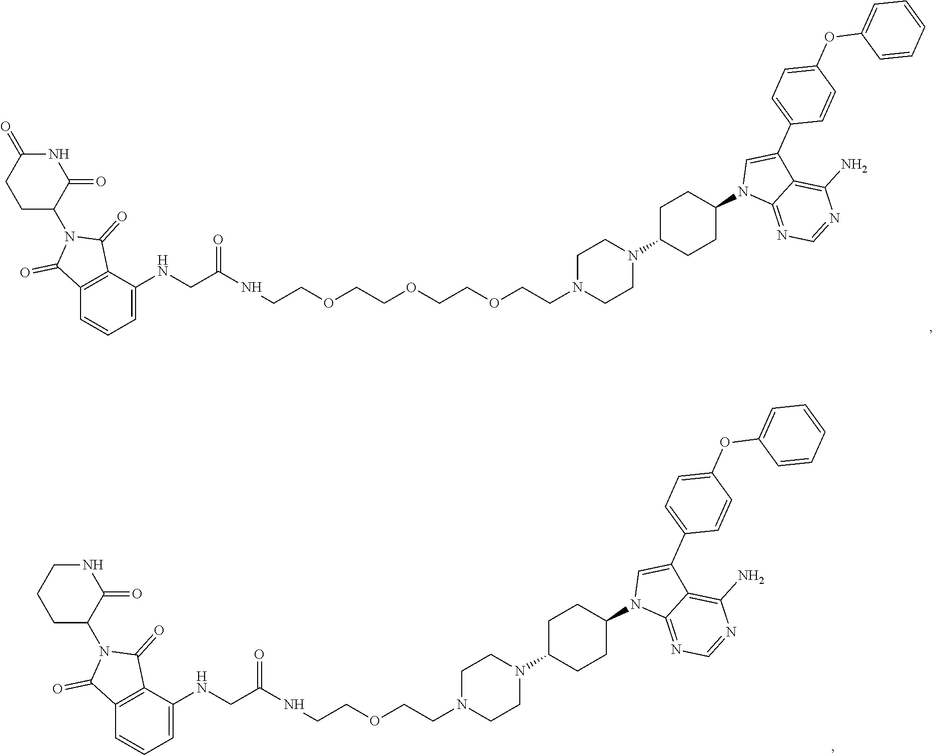 [SMILES: Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@H](N2CCN(CCOCCNC(=O)CNc3cccc4c3C(=O)N(C3CCCNC3=O)C4=O)CC2)CC1] 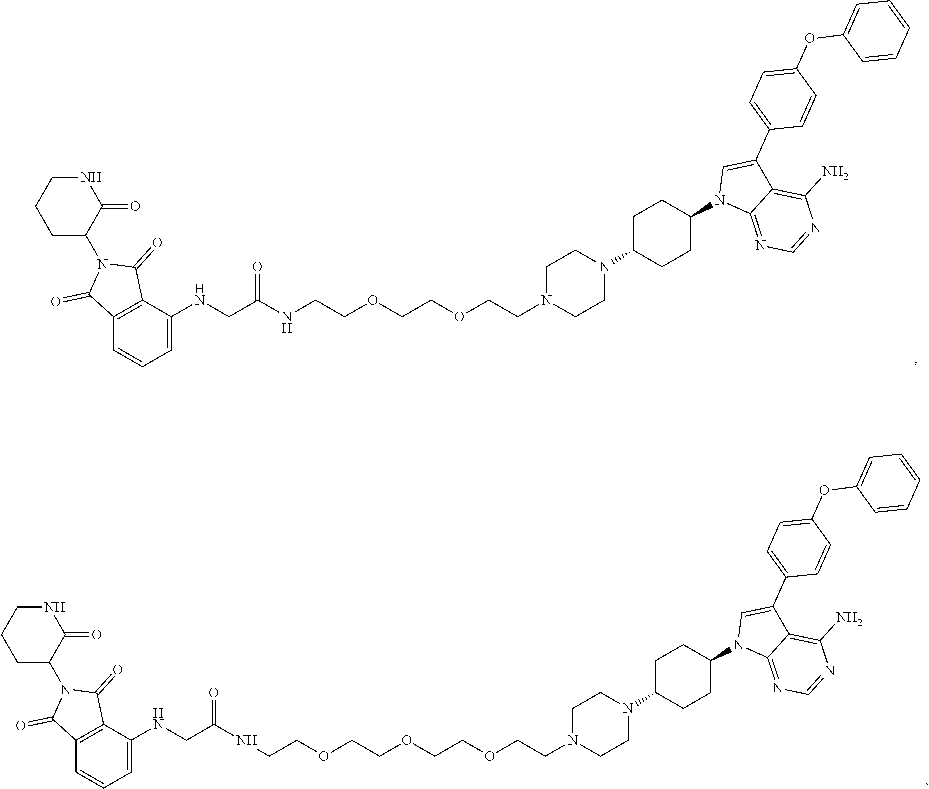 [SMILES: Nc1ncnc2c1c(-c1ccc(Oc3ccccc3)cc1)cn2[C@H]1CC[C@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@H](N2CCN(CCOCCOCCOCCNC(=O)CNc3cccc4c3C(=O)N(C3CCCNC3=O)C4=O)CC2)CC1] 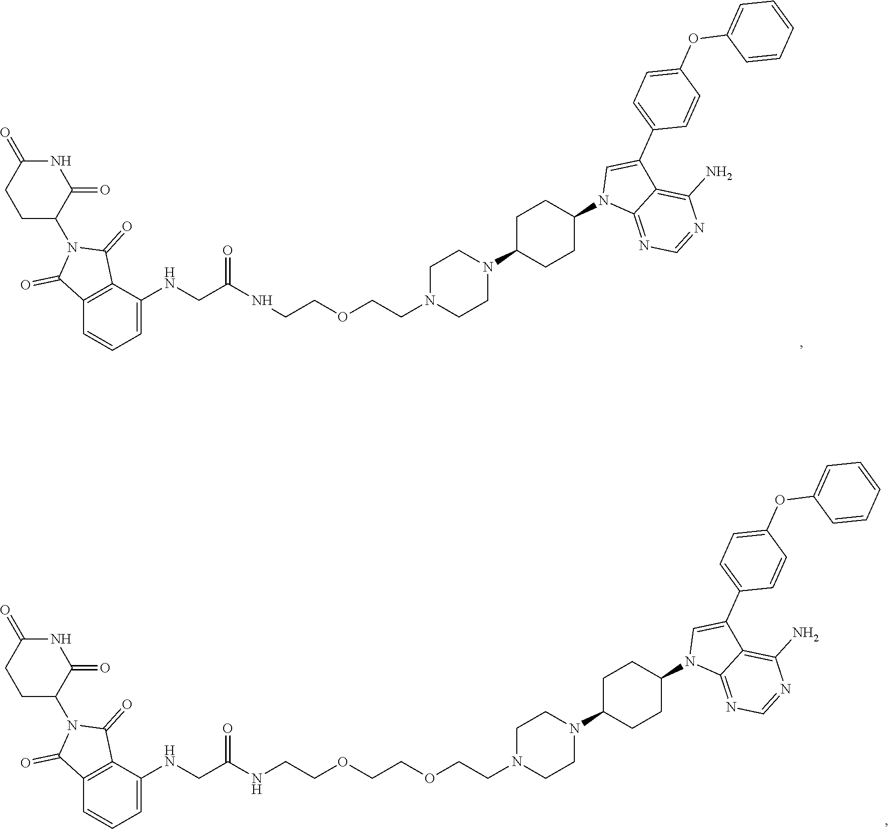 [SMILES: Nc1ncnc2c1c(-c1ccc(Oc3ccccc3)cc1)cn2[C@H]1CC[C@@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCC(=O)NC3=O)C4=O)CC2)CC1] 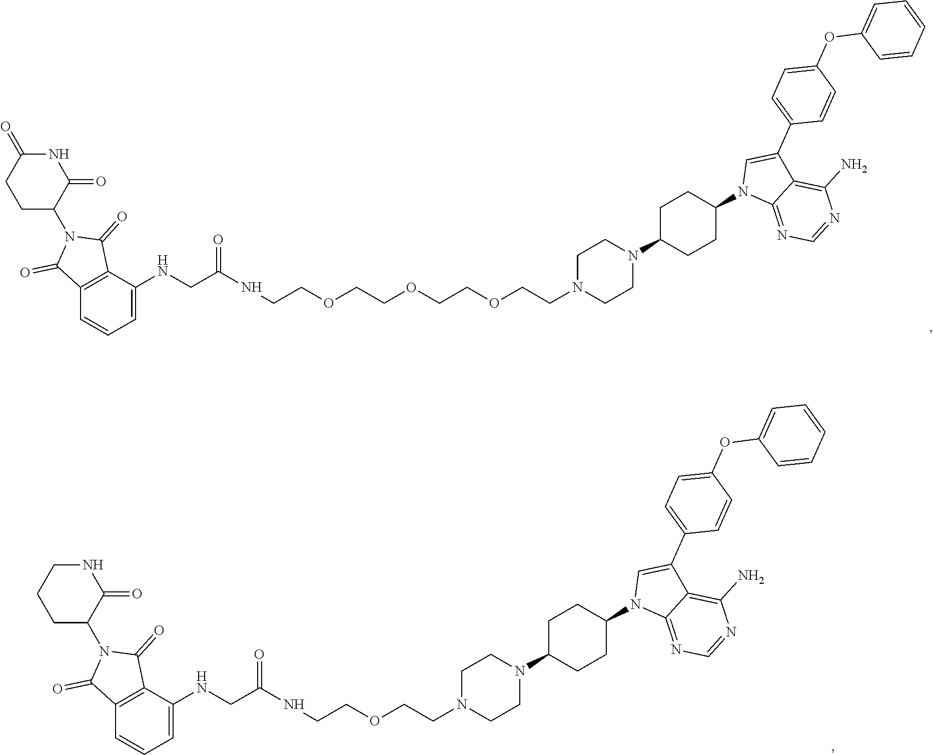 [SMILES: Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)cn2[C@H]1CC[C@@H](N2CCN(CCOCCNC(=O)CNc3cccc4c3C(=O)N(C3CCCNC3=O)C4=O)CC2)CC1] 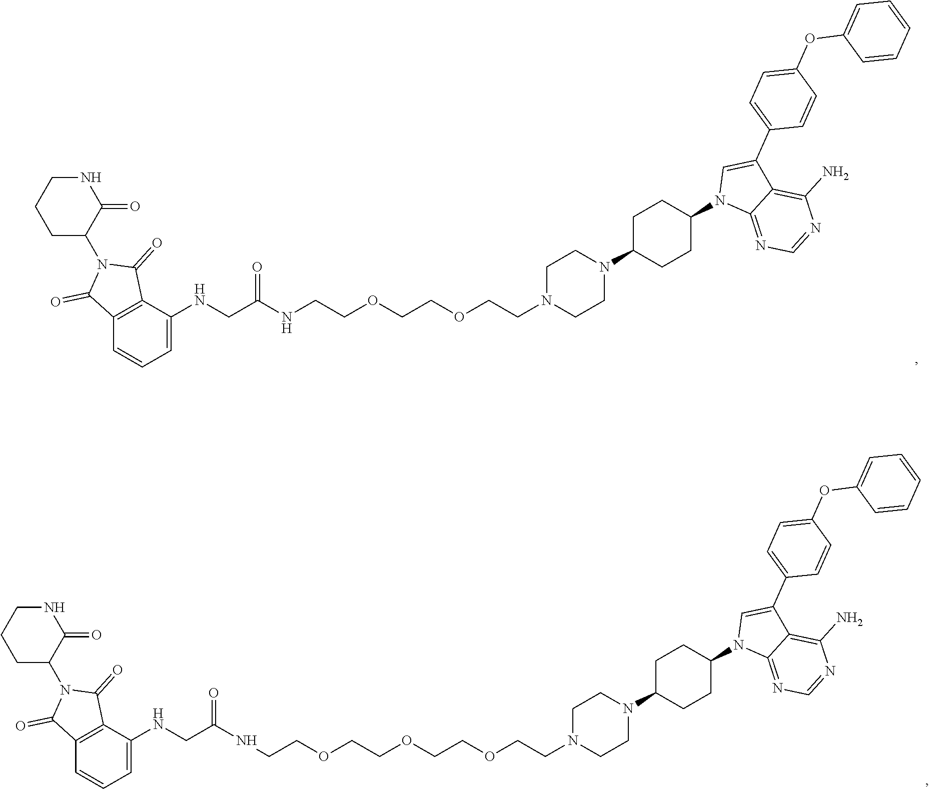 [SMILES: Nc1ncnc2c1c(-c1ccc(Oc3ccccc3)cc1)cn2[C@H]1CC[C@@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)cn2[C@H]1CC[C@@H](N2CCN(CCOCCOCCOCCNC(=O)CNc3cccc4c3C(=O)N(C3CCCNC3=O)C4=O)CC2)CC1] 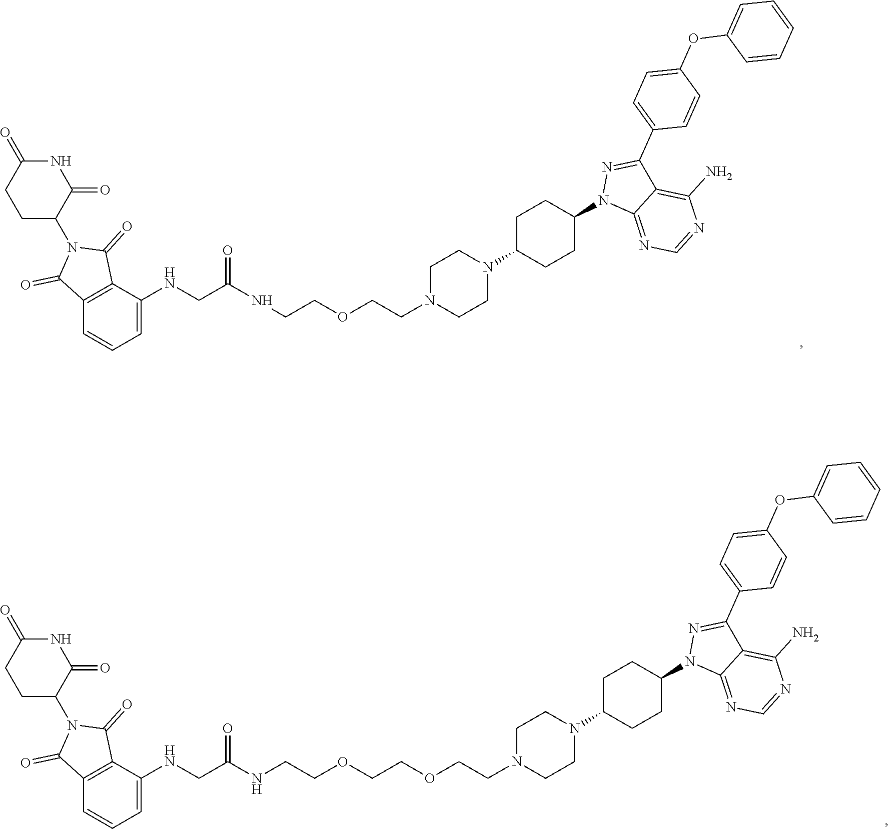 [SMILES: Nc1ncnc2c1c(-c1ccc(Oc3ccccc3)cc1)nn2[C@H]1CC[C@H](N2CCN(CCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCC(=O)NC3=O)C4=O)CC2)CC1] 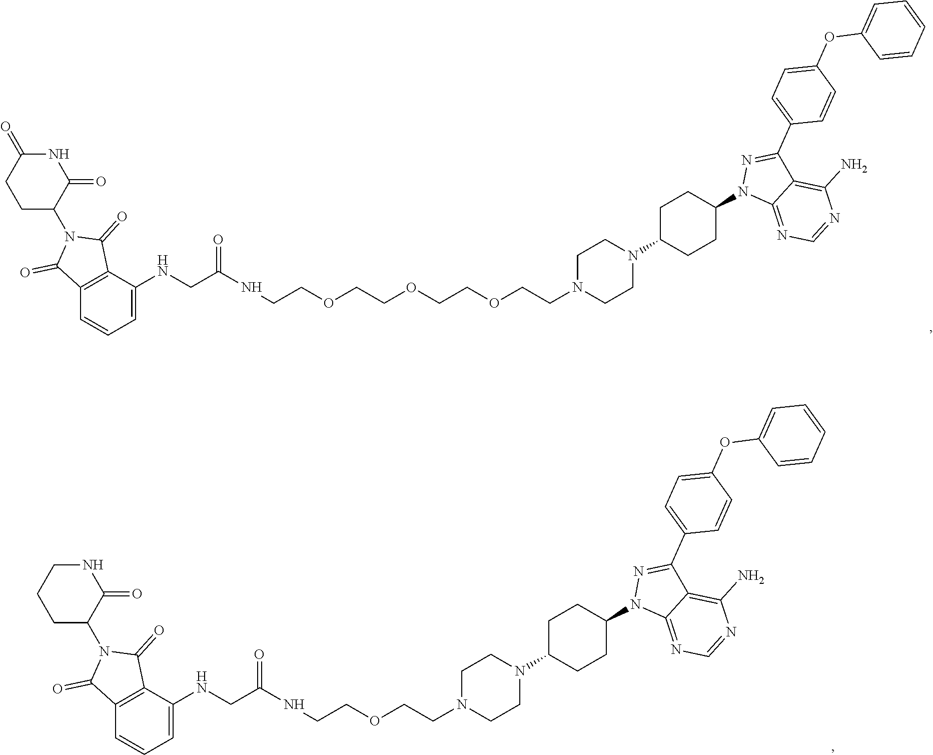 [SMILES: Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCNC(=O)CNc3cccc4c3C(=O)N(C3CCCNC3=O)C4=O)CC2)CC1] 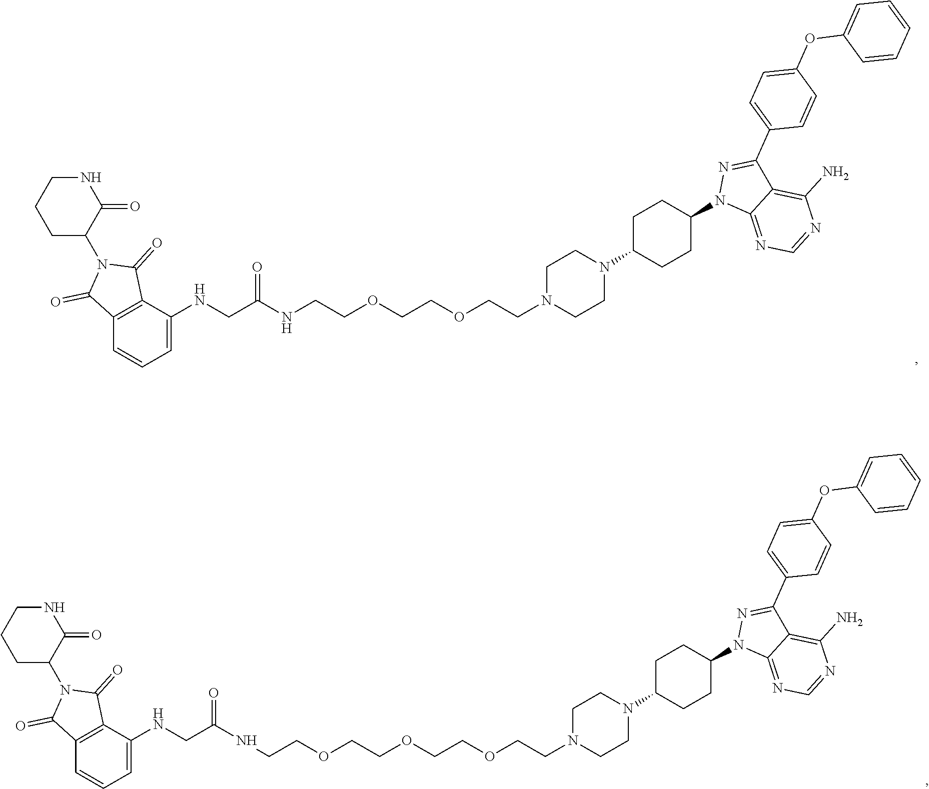 [SMILES: Nc1ncnc2c1c(-c1ccc(Oc3ccccc3)cc1)nn2[C@H]1CC[C@H](N2CCN(CCOCCOCCNC(=O)CNc3cccc4c3C(=O)N(C3CCCNC3=O)C4=O)CC2)CC1 ; Nc1ncnc2c1c(-c1ccc(Oc3ccccc3)cc1)nn2[C@H]1CC[C@H](N2CCN(CCOCCOCCOCCNC(=O)CNc3cccc4c3C(=O)N(C3CCCNC3=O)C4=O)CC2)CC1] 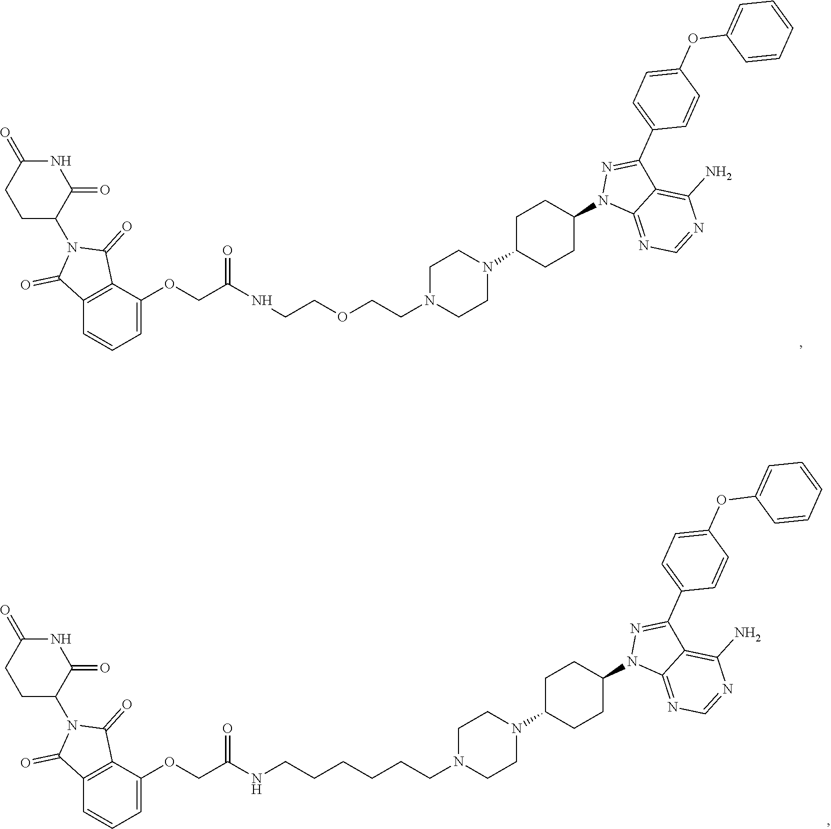 [SMILES: Nc1ncnc2c1c(-c1ccc(Oc3ccccc3)cc1)nn2[C@H]1CC[C@H](N2CCN(CCOCCNC(=O)COc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)COc3cccc4c3C(=O)N(C3CCC(=O)NC3=O)C4=O)CC2)CC1] 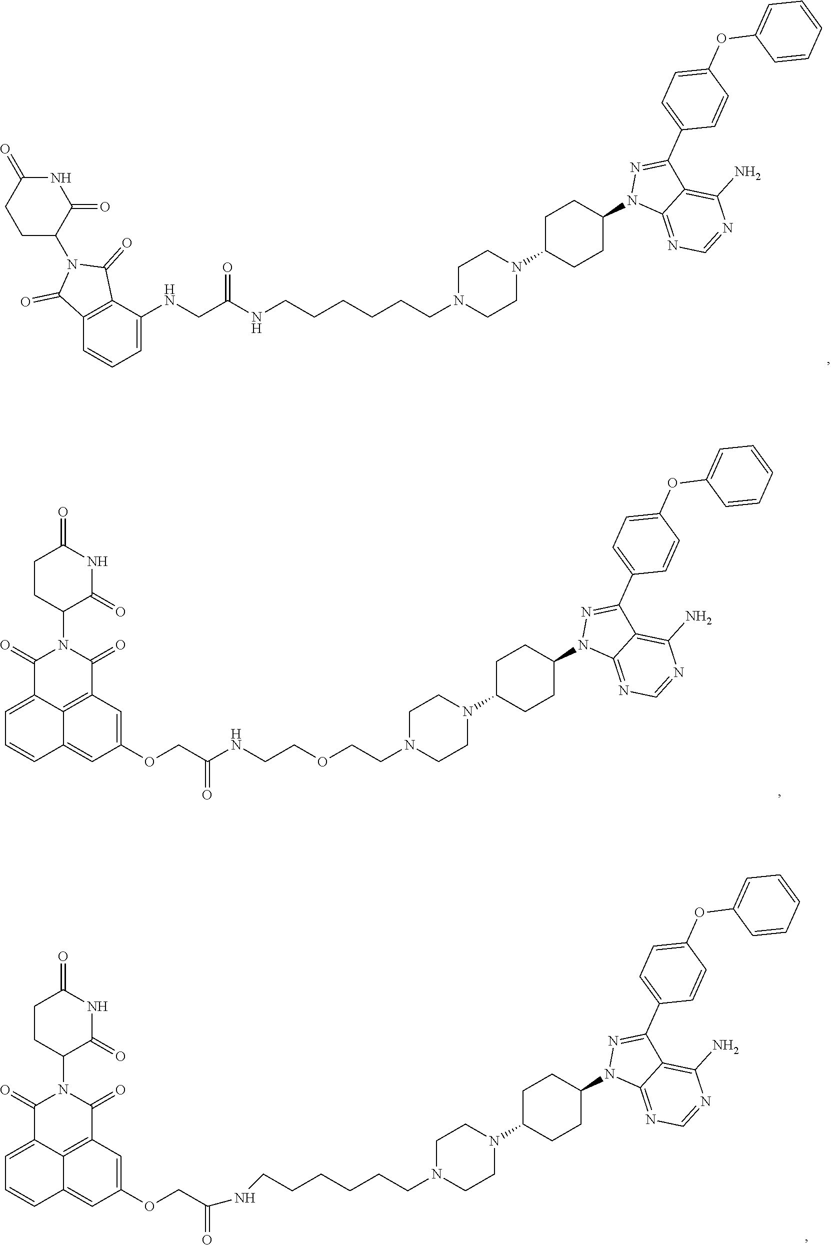 [SMILES: Nc1ncnc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCOCCNC(=O)COc3cc4c5c(cccc5c3)C(=O)N(C3CCC(=O)NC3=O)C4=O)CC2)CC1 ; Nc1ncnc2c1c(-c1ccc(Oc3ccccc3)cc1)nn2[C@H]1CC[C@H](N2CCN(CCCCCCNC(=O)COc3cc4c5c(cccc5c3)C(=O)N(C3CCC(=O)NC3=O)C4=O)CC2)CC1] 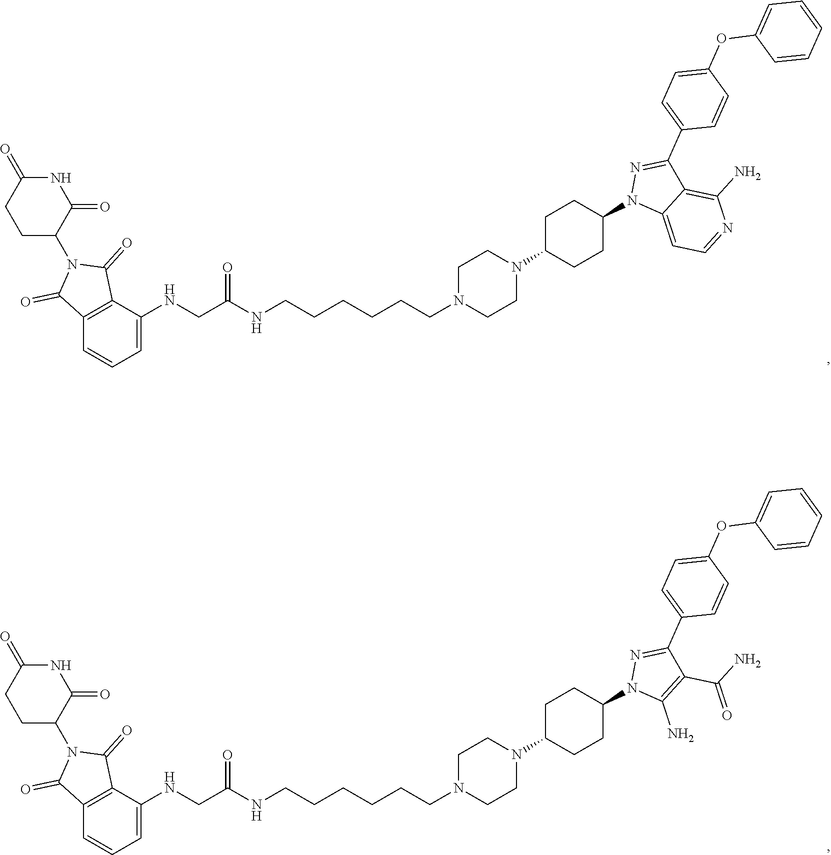 [SMILES: Nc1nccc2c1c(-c1ccc(Oc3ccccc3)cc1)nn2[C@H]1CC[C@H](N2CCN(CCCCCCNC(=O)CNc3cccc4c3C(=O)N(C3CCC(=O)NC3=O)C4=O)CC2)CC1 ; NC(=O)c1c(-c2ccc(Oc3ccccc3)cc2)nn([C@H]2CC[C@H](N3CCN(CCCCCCNC(=O)CNc4cccc5c4C(=O)N(C4CCC(=O)NC4=O)C5=O)CC3)CC2)c1N]
or a pharmaceutically acceptable salt, co-crystal, tautomer, stereoisomer, solvate, hydrate, polymorph, isotopically enriched derivative, or prodrug thereof.

84. A pharmaceutical composition comprising a therapeutically effective amount of the compound of claim 37, or a pharmaceutically acceptable salt, co-crystal, tautomer, stereoisomer, solvate, hydrate, polymorph, isotopically enriched derivative, or prodrug thereof, and optionally a pharmaceutically acceptable excipient.

85.-86. (canceled)

87. A method of treating a disease in a subject in need thereof, wherein the disease is cancer, the method comprising administering to the subject a therapeutically effective amount of the compound of claim 37, or a pharmaceutically acceptable salt, co-crystal, tautomer, stereoisomer, solvate, hydrate, polymorph, isotopically enriched derivative, or prodrug thereof.

88.-89. (canceled)

90. The method of claim 87, wherein the cancer is lymphoma, leukemia, or multiple myeloma.

91. The method of claim 90, wherein the lymphoma, leukemia, or multiple myeloma is non-Hodgkin's lymphoma, Waldenstrom macroglobulinemia, MYD88-mutated Waldenstrom macroglobulinemia, diffuse large B-cell lymphoma, activated B-cell (ABC) diffuse large B-cell lymphoma, germinal center B-cell-like diffuse large B-cell lymphoma, or Burkitt's lymphoma.

92.-101. (canceled)

102. A method of inducing the degradation of hematopoietic cell kinase (HCK) and/or Bruton's tyrosine kinase (BTK) in a subject, the method comprising:
administering to the subject a therapeutically effective amount of the compound of claim 37, or a pharmaceutically acceptable salt, co-crystal, tautomer, stereoisomer, solvate, hydrate, polymorph, isotopically enriched derivative, or prodrug thereof.

103-105. (canceled)

106. The method of claim 87 further comprising administering to the subject a therapeutically effective amount of an additional pharmaceutical agent in combination with the compound, or a pharmaceutically acceptable salt, co-crystal, tautomer, stereoisomer, solvate, hydrate, polymorph, isotopically enriched derivative, or prodrug thereof.

107.-115. (canceled)


SEQUENCE LISTING (3 sequences)

SEQ ID NO: 1 - protein, 352 aa, Homo sapiens [sequence omitted]

SEQ ID NO: 2 - protein, 949 aa, Homo sapiens
MEEGNNNEEVIHLNNFHCHRGQEWINLRDGPITISDSSDEERIPMLVTPAPQQHEEEDLDDDVILTETNKPQRSRPNLIKPAAQWQDLKRLGEERPKKSRAAFESDKSSYFSVCNNPLFDSGAQDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRFYDFGPIHMPLEHNLPMHFGPQPRHRF

SEQ ID NO: 3 - protein, 866 aa, Homo sapiens
MEEGNNNEEVIHLNNFHCHRGQEWINLRDGPITISDSSDEERIPMLVTPAPQQHEEEDLDDDVILTEDDSEDDYGEFLDLGPPGISEFTKPSGQTEREPKPGPSHNQAANDIVNPRSEQKVIILEEGSLLYTESDPLETQNQSSEDSETELLSNLGESAALADDQAIEEDCWLDHPYFQSLNQQPREITNQVVPQERQPEAELGRLLFQHEFPGPAFPRPEPQQGGISGPSSPQPAHPLGEFEDQQLASDDEEPGPAFPMQESQEPNLENIWGQEAAEVDQELVELLVKETEARFPDVANGFIEEIIHFKNYYDLNVLCNFLLENPDYPKREDRIIINPSSSLLASQDETKLPKIDFFDYSKLTPLDQRCFIQAADLLMADFKVLSSQDIKWALHELKGHYAITRKALSDAIKKWQELSPETSGKRKKRKQMNQYSYIDFKFEQGDIKIEKRMFFLENKRRHCRSYDRRALLPAVQQEQEFYEQKIKEMAEHEDFLLALQMNEEQYQKDGQLIECRCCYGEFPFEELTQCADAHLFCKECLIRYAQEAVFGSGKLELSCMEGSCTCSFPTSELEKVLPQTILYKYYERKAEEEVAAAYADELVRCPSCSFPALLDSDVKRFSCPNPHCRKETCRKCQGLWKEHNGLTCEELAEKDDIKYRTSIEEKMTAARIRKCHKCGTGLIKSEGCNRMSCRCGAQMCYLCRVSINGYDHFCQHPRSPGAPCQECSRCSLWTDPTEDDEKLIEEIQKEAEEEQKRKNGENTFKRIGPPLEKPVEKVQRVEALPRPVPQNLPQPQMPPYAFAHPPFPLPPVRPVFNNFPLNMGPIPAPYVPPLPNVRVNYDFGPIHMPLEHNLPMHFGPQPRHRF